(19) United States
(12) Patent Application Publication
(10) Pub. No.: US 2022/0389009 A1
(43) Pub. Date: Dec. 8, 2022

(54) SUBSTITUTED 1,6-NAPHTHYRIDINE INHIBITORS OF CDK5

(71) Applicant: Goldfinch Bio, Inc., Cambridge, MA (US)

(72) Inventors: Goran Malojcic, Boston, MA (US); Matthew H. Daniels, Somerville, MA (US); Brett D. Williams, Boston, MA (US); Maolin Yu, West Roxbury, MA (US); Mark W. Ledeboer, Acton, MA (US); Jean-Christophe P. Harmange, Andover, MA (US); Jenna L. Wang, Shelton, CT (US)

(21) Appl. No.: 17/765,904

(22) PCT Filed: Oct. 1, 2020
(86) PCT No.: PCT/US20/53756

§ 371 Date: Apr. 1, 2022

Related U.S. Application Data
(60) Provisional application No. 63/050,384, filed on Jul. 10, 2020.
(60) Provisional application No. 62/908,952, filed on Oct. 1, 2019.

(51) Int. Cl. C07D 471/04

(52) U.S. Cl. CPC C07D 471/04

(57) ABSTRACT

Disclosed are compounds having structural formula I, and related salts and pharmaceutical compositions. Also disclosed are therapeutic methods, e.g., of treating diseases and conditions such as kidney disease, kidney failure, kidney stones, or polycystic kidney disease, using the compounds of formula (I), and related salts and pharmaceutical compositions.

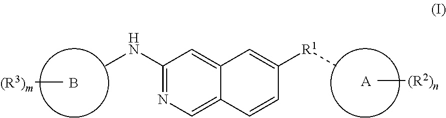

### RELATED APPLICATIONS

Tins application claims the benefit of priority to U.S. Provisional Patent Application. No. 62/908,952, filed Oct. 1, 2019; and U.S. Provisional Patent Application No. 63/050,384, filed Jul. 10, 2020.

### BACKGROUND

Cyclin-dependent kinases (CDKs) belong to a family of proline-directed serine/threonine kinases that play important roles in controlling cell cycle progression and transcriptional control. Cyclin-dependent kinase 5 (CDK5), a proline-directed serine/threonine kinase, is unique due to its indispensable role in neuronal development and function. CDK5 is unusual because it is not typically activated upon binding with a cyclin and does not require T-loop phosphorylation for activation, even though it has high amino acid sequence homology with other CDKs. While it was previously thought that CDK5 only interacted with p35 or p39 and their cleaved counterparts. Recent evidence suggests that CDK5 can interact with certain cyclins, amongst oilier proteins, which modulate CDK5 activity levels. Recent findings report molecular interactions that regulate CDK5 activity and CDK5 associated pathways implicated in various diseases. Also covered herein is the growing body of evidence for CDK5 in contributing to the onset and progression of tumorigenesis.

CDK5 plays a diverse physiological role in neural cells, including neuronal migration (Xie et al., 2003) and axon guidance (Connell-Crowley et al., 2000) during early neural development as well as synapse formation and synaptic plasticity (Cheung et al., 2006; Lai and Ip, 2009). However, more recently, CDK5 has also been found to play important roles outside the central nervous system such as pain signaling that involves the sensory pathways (Pareek et al., 2006), and in modulating glucose-stimulated insulin levels in pancreatic beta cells, (Wei et al., 2005). Due to its key physiological roles, uncontrolled activity of CDK5 has been linked to various diseases/disorders such that CDK5 has emerged as a potential molecular target for therapeutic drugs. In neurons, CDK5 deregulation triggers neuronal apoptosis (Cheung and Ip, 2004), suggesting, that aberrant regulation of CDK5 activity is responsible for the progression of neurodegenerative diseases such as Alzheimer's disease (AD) and Parkinson's disease (PD). Aberrant CDK5 activity, for example, is also linked to cancer development, progression and metastasis such as prostate and thyroid carcinoma (Strock et al., 2006 Lin et al., 2007).

The two major pathological hallmarks of AD are the accumulation of senile plaques and neurofibrillary tangles in the diseased brain. The deregulation of CDK5 is caused by the presence of p25, a cleavage product of p35 generated under pathological conditions (Patrick et al., 1999). Accumulation of p25 protein is found in the brains of AD patients (Patrick et al., 1999). Recent findings also indicate that CDK5 is one of the key kinases that regulate the formation of senile plaques (Monaco, 2004) and neurofibrillary tangles (Cruz et al., 2003).

Another major neurodegenerative disease that links to CDK5 is Parkinson's disease (PD). Pathologically, PD is characterized by motor impairment due to the progressive death of selected populations of neurons, especially the dopaminergic neurons in the substantia nigra pars compacta (Muntane et al., 2008). In a PD mouse model induced by 1-methyl-4-phenyl-1,2,3,6-tetrahydropyridine (MPTP), elevated expression and activity of CDK5 have been reported to be correlated with dopaminergic neurons cell death (Smith et al., 2003; Qu et al., 2007). Moreover, it is of interest to note that inhibition of CDK5 results in an increase in dopamine release, which may help ameliorate PD progression (Chergui et al., 2004). CDK5 has also been implicated in a plethora of other neurodegenerative diseases and neurological disorders such as Huntington's disease (Anne et al, 2007), Amyotrophic Lateral Sclerosis (ALS; Bajaj et al., 1998) and ischemic injury (Wang et al., 2003).

Aberrant CDK5 activity has also been linked to the pathogenesis of diabetes mellitus (type-2 diabetes). p35, the activator of CDK5, is present in pancreatic beta cells and its activity negatively modulates insulin release in response to glucose (Wei and Tomizawa, 2007). A sustained increase in p35 protein and CDK5 activity is reported in murine pancreatic beta cells upon high glucose exposure (Ubeda et al., 2006). Moreover, inhibition of CDK5 activity by chemical inhibitors increases insulin secretion in cultured beta cells and in a mouse model of diabetes in a glucose-dependent manner (Ubeda et al., 2006). These findings are consistent with the observation that p35^{-/-} mice exhibit enhanced insulin secretion upon glucose challenge (Wei et al., 2005). CDK5 is thought to act through the regulation of the Ca²⁺ channel activity or regulation of insulin gene expression during glucotoxicity (Wei et al., 2005, Ubeda et al., 2006). Thus, CDK5 inhibitors could be potential therapeutic agents for the treatment of type-2 diabetes (Kitani et al., 2007).

CDK5 has also been emerging as a major potential target for analgesic drugs. CDK5/p35 has been indirectly linked to nociceptive pathways. For example, CDK5 regulates the activation of mitogen activated protein kinase (MAPK) in nociceptive neurons potentially modifying the hyperalgesia that results in increased MAPK activity. CDK5 has also been implicated in other pain pathways such as calcium calmodulin kinase II, delta FosB, the NMDA receptor and the P/Q type voltage-dependent calcium channel. Furthermore, studies suggest that CDK5 inhibitors may be of benefit in the management of acute pain. CDK5/p35 is shown to be involved in the processing of pain while its inhibition reduces the responsiveness of normal pain pathways (Pareek et al., 2006; Pareek and Kulkarni, 2006). CDK5 also regulates mitogen-activated protein kinase1/2 (MEK1/2)/1M activity through a negative feedback loop during a peripheral inflammatory response (Pareek. and Kulkarni, 2006), in addition, transient receptor potential vanilloid 1 (TRPV1), a ligand-gated cation channel that is activated by heat, protons and capsaicin, was recently identified as a substrate of CDK5 (Pareek et al, 2007). Since phosphorylation of TRPV1 by CDK5 regulates the functions of TRPV1 during pain signaling, it is believed that CDK5 could serve as a new molecular target for developing analgesic drugs.

More recently, CDK5 was identified as playing a critical role in controlling ciliary length and tubular epithelial differentiation. Pharmacological or genetic reduction of CDK5 lead to effective and sustained arrest of PKD. CDK5 might act on primary cilia, at least in part, by modulating microtubule dynamics. It was suggested that new therapeutic approaches aimed at restoration of cellular differentiation are likely to yield effective treatments for cystic kidney diseases (Husson et al 2016) Further, CDK5 was shown to be detrimental and promotes tubulointerstitial fibrosis (TIF) via the extracellular signal-regulated kinase 1/2 (ERK1/2)/peroxisome proliferator-activated receptor gamma (PPRAy) pathway in DN. These findings demonstrate a novel mechanism that CDK5 increases tubulointerstitial fibrosis by activating the ERK1/2/PPARy pathway and EMT in DN. Hence, CDK5 might have therapeutic potential in diabetic nephropathy. (Bai et al. 2016).

Hence, there is a need for additional inhibitors of CDK5.

### SUMMARY

In one aspect, the invention features compounds that are inhibitors of CDK5. In some embodimens, the compound of the invention is a compound having structural formula (I):

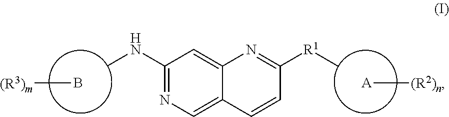

or a pharmaceutically acceptable salt thereof, wherein:

ring A is a monocyclic or bicyclic cycloalkyl or a monocyclic or bicyclic saturated heterocyclyl;

ring B is monocyclic or bicyclic aryl, a monocyclic or bicyclic heteroaryl, or a monocyclic or bicyclic heterocyclyl;

R¹ is —N(R⁵)—, —C(O)—, —S—, —S(O)—, —S(O)₂—, —[C(R⁴)₂]₁₋₂—, —[C(R⁴)₂]₀₋₁—CH═, —N(R⁵)—S(O)₂—, —S(O)₂—N(R⁵), —C(R⁴)₂—N(R⁵)—, —N(R⁵)—C(R⁴)₂—, —C(R⁴)₂—S(O)₂—, —C(═N—OH)—, —C(═N—O—C₁-C₄ alkyl)-, or —S(O)₂—C(R⁴)₂—;

each R² is independently halo, —OH, —C₁-C₆ alkyl, —C₁-C₆ haloalkyl, —C₁-C₆ hydroxyalkyl, —(C₀-C₄ alkylene) C(O)—OH, —(C₀-C₄ alkylene) C(O)—O—C₁-C₄ alkyl, —(C₀-C₄ alkylene)-O—C₁-C₄ alkyl, —(C₀-C₄ alkylene)-O—C₁-C₄ hydroxyalkyl, —(C₀-C₄ alkylene) C(O)—N(R⁶)₂, —(C₀-C₄ alkylene) N(R⁶)₂, or —(C₀-C₄ alkylene) saturated heterocyclyl, wherein the saturated heterocyclyl is optionally substituted with halo, —OH, or —CH₃;

each R³ is independently halo; —CN; —OH; —N(R⁶)₂; —C₁-C₄ alkyl; —O—C₁-C₄ alkyl; —O—C₁-C₄ alkylene-C(O)—N(R⁶)₂; —C(O)—O—C₁-C₄ alkyl; —C(O)—N(R⁶)₂; —S(O)₂—N(R⁶)₂; —S(O)₂—C₁-C₄ alkyl; C₂-C₄ alkynyl optionally substituted with one or more —OH; 1,2,4-triazol-1-ylmethyl; morpholinylmethyl; cyclopropyl; ═O; —CH₂CH₂—C(O)—O—CH₃; —N(R⁶)—S(O)₂—CH₃; an optionally substituted aryl; an optionally substituted heteroaryl; or an optionally substituted heterocyclyl, wherein any alkyl portion of R³ is optionally substituted with one or more of halo, —CN, or —N(R⁶)₂, or —OH;
each R⁴ is independently hydrogen, halo, —OH, —CN, —N(R⁶)₂, —C₁-C₄ alkyl optionally substituted with one or more of —OH, halo, —CN, or —N(R⁶)₂; or O—C₁-C₄ alkyl optionally substituted with one or more of —OH, halo, —CN, or —N(R⁶)₂;

or one R⁴ is taken together with a ring carbon atom in ring A to form a cycloalkyl or heterocyclyl ring that is spirofused, fused or bridged to ring A;

or two R⁴ bound to the same carbon atom are taken together to form ═CH₂—(C₀-C₃ alkyl), a C₃-C₆ cycloalkyl, or a C₄-C₇ heterocyclyl;

R⁵ is hydrogen; C₁-C₄ alkyl optionally substituted with one or more of —CN, —OH, —COOH, C(O)—O—C₁-C₄ alkyl, or pyrazolyl; —S(O)₂—C₁-C₄ alkyl; —C(O)C(O)OH; —COOH; or —C(O)—O—C₁-C₄ alkyl;

or R⁵ is taken together with a ring carbon atom in ring A to form a heterocyclyl ring that is spirofused, fused or bridged to ring A;

each R⁶ is independently hydrogen or —C₁-C₄ alkyl;

m is 0, 1, 2, 3, 4, 5, or 6;

n is 0, 1, 2, 3, 4, 5, or 6; and

“ [symbol] ” represents a single bond or a double bond.

In one aspect, the invention relates to pharmaceutical compositions comprising a compound disclosed herein and a pharmaceutically acceptable carrier.

In one aspect, the invention relates to methods of treating a disease or condition characterized by aberrant CDK5 overactivity, comprising the step of administering to a subject in need thereof a therapeutically effective amount of a compound or composition disclosed herein. In some embodiments, the disease or condition is a disease or condition of the kidney. In some embodiments, the disease is polycystic kidney disease. In some embodiments, the disease or condition is a ciliopathy. The methods are effective for a variety of subjects including mammals, e.g., humans and other animals, such as laboratory animals, e.g., mice, rats, rabbits, or monkeys, or domesticated and farm animals, e.g., cats, dogs, goats, sheep, pigs, cows, or horses. In some embodiments, the subject is a human.

The invention provides several advantages. The prophylactic and therapeutic methods described herein are effective in treating kidney disease and ciliopathies, and have minimal, if any, side effects. Further, methods described herein are effective to identify compounds that treat or reduce risk of developing a kidney disease, such as polycystic kidney disease, or a ciliopathy.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the detailed description, and from the claims.

### BRIEF DESCRIPTION OF THE DRAWINGS

FIG. 1 shows NMR and MS data for exemplary compounds of the invention.

### DETAILED DESCRIPTION

### Definitions

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, trifluoromethoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen (e.g., fluoro), a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

Unless otherwise specified, “alkylene” by itself or as part of another substituent refers to a saturated straight-chain or branched divalent group having the stated number of carbon atoms and derived from the removal of two hydrogen atoms from the corresponding alkane. Examples of straight chained and branched alkylene groups include —CH₂-(methylene), —CH₂—CH₂-(ethylene), —CH₂—CH₂—CH₂-(propylene), —C(CH₃)₂—, —CH₂—CH(CH₃)—, —CH₂—CH₂—CH₂—CH₂—, —CH₂—CH₂—CH₂—CH₂—CH₂-(pentylene), —CH₂—CH(CH₃)—CH₂—, and —CH₂—C(CH₃)₂—CH₂—.

The term “C_{X-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y} alkyl” refers to substituted or unsubstituted saturated hydrocarbon groups, including straight-chain alkyl and branched-chain alkyl groups that contain from x to y carbons in the chain, including haloalkyl groups. Preferred haloalkyl groups include trifluoromethyl, difluoromethyl, 2,2,2-trifluoroethyl, and pentafluoroethyl. Co alkyl indicates a hydrogen where the group is in a terminal position, a bond if internal. The terms “C_{2-y} alkenyl” and “C_{2-y} alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

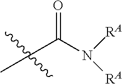

wherein each R^{A} independently represent a hydrogen or hydrocarbyl group, or two R^{A} are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

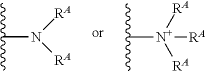

wherein each R^{A} independently represents a hydrogen or a hydrocarbyl group, or two R^{A} are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably, the ring is a 6- or 10-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, aryls, heteroaryls, and/or heterocyclyls. Aryl groups include benzene, naphthalene, phenanthrene, phenol, aniline, and the like.

The term “carbamate” is art-recognized and refers to a group

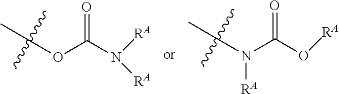

wherein each R^{A} independently represent hydrogen or a hydrocarbyl group, such as an alkyl group, or both R^{A} taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R^{A}, wherein R^{A} represents a hydrocarbyl group.

The term “carboxy”, as used herein, refers to a group represented by the formula —CO₂H.

The term “ester”, as used herein, refers to a group —C(O)OR^{A} wherein R^{A} represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, aryls, heteroaryls, and/or heterocyclyls. Heterocyclyl groups include, for example, piperidine, piperazine, pyrrolidine, tetrahydropyran, tetrahydrofuran, morpholine, lactones, lactams, and the like.

The term “heterocyclylalkyl” or “heterocycloalkyl”, as used herein, refers to an alkyl group substituted with a heterocycle group.

The term “hydrocarbyl”, as used herein, refers to a group that is bonded through a carbon atom that does not have a ═0 or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

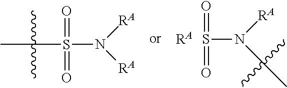

wherein each R^{A} independently represents hydrogen or hydrocarbyl, such as alkyl, or both R^{A} taken together with the intervening atom(s) complete a heterocycle having from 4 to 8 atoms in the ring structure.

The term “sulfoxide” is art-recognized and refers to the group —S(O)—R^{A}, wherein R^{A} represents a hydrocarbyl.

The term “sulfonate” is art-recognized and refers to the group SO₃H, or a pharmaceutically acceptable salt thereof.

The term “sulfone” is art-recognized and refers to the group —S(O)₂—R^{A}, wherein R^{A} represents a hydrocarbyl.

The term “thioalkyl”, as used herein, refers to an alkyl group substituted with a thiol group.

The term “thioester”, as used herein, refers to a group —C(O)SR^{A} or —SC(O)R^{A} wherein R^{A} represents a hydrocarbyl.

The term “thioether”, as used herein, is equivalent to an ether, wherein the oxygen is replaced with a sulfur.

The term “urea” is art-recognized and may be represented by the general formula

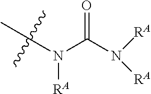

wherein each R^{A} independently represents hydrogen or a hydrocarbyl, such as alkyl, or any occurrence of R^{A} taken together with another and the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3rd Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

As used herein, a therapeutic that “prevents” or “reduces the risk of developing” a disease, disorder, or condition refers to a compound that, in a statistical sample, reduces the occurrence of the disease, disorder, or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The phrases “conjoint administration” and “administered conjointly” refer to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

The term “prodrug” is intended to encompass compounds which, under physiologic conditions, are converted into the therapeutically active agents of the present invention. A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds of the invention in a formulation represented above can be replaced with the corresponding suitable prodrug, e.g., wherein a hydroxyl in the parent compound is presented as an ester or a carbonate or carboxylic acid present in the parent compound is presented as an ester.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, a “small molecule” refers to an organic, inorganic, or organometallic compound typically having a molecular weight of less than about 1000. In some embodiments, a small molecule is an organic compound, with a size on the order of 1 nm. In some embodiments, small molecule drugs of the invention encompass oligopeptides and other biomolecules having a molecular weight of less than about 1000.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

Compounds of the Invention

One aspect of the invention provides methods of treating a disease or a condition characterized by aberrant CDK5 overactivity, comprising the step of administering to a subject in need thereof a therapeutically effective amount of a compound disclosed herein. In some embodiments, the compound is a small molecule inhibitor of CDK5.

In certain embodiments, the compound has structural formula (I):

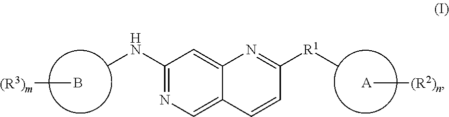

or a pharmaceutically acceptable salt thereof, wherein:

ring A is a monocyclic or bicyclic cycloalkyl or a monocyclic or bicyclic saturated heterocyclyl;

ring B is monocyclic or bicyclic aryl, a monocyclic or bicyclic heteroaryl, or a monocyclic or bicyclic heterocyclyl;

R¹ is —N(R⁵)—, —C(O)—, —S—, —S(O)—, —S(O)₂—, —[C(R⁴)₂]₁₋₂—, —[C(R⁴)₂]₀₋₁—CH═, —N(R⁵)—S(O)₂—, —S(O)₂—N(R⁵), —C(R⁴)₂—N(R⁵)—, —N(R⁵)—C(R⁴)₂—, —C(R⁴)₂—S(O)₂—, —C(═N—OH)—, —C(═N—O—C₁-C₄ alkyl)-, or —S(O)₂—C(R⁴)₂—;

each R² is independently halo, —OH, —C₁-C₆ alkyl, —C₁-C₆ haloalkyl, —C₁-C₆ hydroxyalkyl, —(C₀-C₄ alkylene) C(O)—OH, —(C₀-C₄ alkylene)-C(O)—O—C₁-C₄ alkyl, —(C₀-C₄ alkylene)-O—C₁-C₄ alkyl, —(C₀-C₄ alkylene)-O—C₁-C₄ hydroxyalkyl, —(C₀-C₄ alkylene)-C(O)—N(R⁶)₂, —(C₀-C₄ alkylene)-N(R⁶)₂, or —(C₀-C₄ alkylene)-saturated heterocyclyl, wherein the saturated heterocyclyl is optionally substituted with halo, —OH, or —CH₃;

each R³ is independently halo; —CN; —OH; —N(R⁶)₂; —C₁-C₄ alkyl; —O—C₁-C₄ alkyl; —O—C₁—C₄ alkylene-C(O)—N(R⁶)₂; —C(O)—O—C₁-C₄ alkyl; —C(O)—N(R⁶)₂; —S(O)₂—N(R⁶)₂; —S(O)₂-C₁-C₄ alkyl; C₂-C₄ alkynyl optionally substituted with one or more —OH; 1,2,4-triazol-1-ylmethyl; morpholinylmethyl; cyclopropyl; ═O; —CH₂CH₂—C(O)—O—CHs; —N(R⁶)—S(O)₂—CHs; an optionally substituted aryl; an optionally substituted heteroaryl; or an optionally substituted heterocyclyl, wherein any alkyl portion of R³ is optionally substituted with one or more of halo, —CN, or —N(R⁶)₂, or —OH;

each R⁴ is independently hydrogen, halo, —OH, —CN, —N(R⁶)₂, —C₁-C₄ alkyl optionally substituted with one or more of —OH, halo, —CN, or —N(R⁶)₂; or O—C₁-C₄ alkyl optionally substituted with one or more of —OH, halo, —CN, or —N(R⁶)₂;

or one R⁴ is taken together with a ring carbon atom in ring A to form a cycloalkyl or heterocyclyl ring that is spirofused, fused or bridged to ring A;

or two R⁴ bound to the same carbon atom are taken together to form ═CH₂—(C₀-C₃ alkyl), a C₃-C₆ cycloalkyl, or a C₄-C₇ heterocyclyl;

R⁵ is hydrogen; C₁-C₄ alkyl optionally substituted with one or more of —CN, —OH, —COOH, C(O)—O—C₁-C₄ alkyl, or pyrazolyl; —S(O)₂-C₁-C₄ alkyl; —C(O)C(O)OH; —COOH; or —C(O)—O—C₁-C₄ alkyl;

or R⁵ is taken together with a ring carbon atom in ring A to form a heterocyclyl ring that is spirofused, fused or bridged to ring A;

each R⁶ is independently hydrogen or —C₁-C₄ alkyl;

m is 0, 1, 2, 3, 4, 5, or 6;

n is 0, 1, 2, 3, 4, 5, or 6; and

“ [symbol] ” represents a single bond or a double bond.

In some embodiments of Formula I, each R³ is independently halo; —CN; —OH; —N(R⁶)₂; —C₁-C₄ alkyl; —O—C₁-C₄ alkyl; —O—C₁-C₄ alkylene-C(O)—N(R⁶)₂; —C(O)—O—C₁-C₄ alkyl; —C(O)—N(R⁶)₂; —S(O)₂—N(R⁶)₂; —S(O)₂-C₁-C₄ alkyl; an optionally substituted aryl; an optionally substituted heteroaryl; or an optionally substituted heterocyclyl, wherein any alkyl portion of R³ is optionally substituted with one or more of halo, —CN, or —N(R⁶)₂, or —OH.

In certain embodiments, ring B is phenyl, —C(O)-phenyl, 1,3,4-thiadiazol-2-yl, imidazo[1,2-b]pyridazin-3-yl, isoxazol-3-yl, 1,3-dihydroisobenzofuran-5-yl, 2H-chromen-6-yl, 1,2,3,4-tetrahydroisoquinolin-6-yl, 1,2,3,4-tetrahydroisoquinolin-7-yl, isoindolin-5-yl, 1,2-dihydropyridin-3-yl, 1,2-dihydropyridin-5-yl, pyridinyl or pyrimidinyl.

In certain embodiments, at least one R³ is fluoro, chloro, —OH, ═O, —CH₃, —CH₂CH₃, —C(CH₃)₃, —CH(CH₃)₂, —CN, —CH₂CH₂—C(O)—O—CHs, —C(O)—O—CH₂CH₃, —OCH₃, —O—CH₂CH₂—C(O)—N(R⁶)₂, —N(R⁶)₂, —CH₂—N(R⁶)₂, —S(O)₂—N(R⁶)₂, —N(R⁶)—S(O)₂—CH₃, —S(O)₂CH₃, —C(O)—N(R⁶)₂, —C((CH₃)₂)—OH, —C≡C—C((CH₃)₂)—OH, or —CH₂CN.

In certain embodiments, at least one R³ is 1,2,4-triazol-1-yl, 1,2,4-triazol-1-ylmethyl, 1,2,3,4-tetrazol-1-yl, 1,2,3,4-tetrazol-5-yl, 1,2,4-oxadiazol-3-yl, 1,2-dihydropyridin-6-yl, 1,2-dihydropyridin-3-yl, 1,2-dihydropyridin-5-yl, 1,2-dihydropyridin-1-yl, 4,5-dihydro-1,2,4-oxadiazol-3-yl, isothiazolidin-2-yl, pyrazolyl, pyrazin-2-yl, pyridin-2-yl, pyridin-3-yl, pyridin-4-yl, pyrimindin-4-yl, pyrrolidin-1-yl, morpholin-4-yl, morpholin-4-ylmethyl, thiomorpholin-4-yl, piperidin-1-yl, piperazin-1-yl, tetrahydropyran-4-yl, oxazolidin-3-yl, imidazolidin-1-yl, cyclopropyl, or phenyl, wherein the at least one R³ is optionally and independently substituted with up to 3 substituents independently selected from halo, ═O, —OH, CN, C₁-C₄ alkyl, C₁-C₄ hydroxyalkyl, C₁-C₄ haloalkyl, —COOH, —C(O)—N(R⁶)₂, —(C₀-C₄ alkylene)-C(O)—O—C₁-C₄ alkyl, or —O—C₁-C₄ alkyl.

In certain embodiments, the portion of the compound represented by

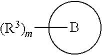

is: 1,3-dihydroisobenzofuran-5-yl, 1-fluoro-2-methylisoindolin-6-yl, 1-oxo-1,2,3,4-tetrahydroisoquinolin-6-yl, 1-oxo-1,2,3,4-tetrahydroisoquinolin-7-yl, 2-(1-hydroxy-1-methylethan-1-yl)pyridin-5-yl, 2-(morpholin-4-yl)phenyl, 2-fluoro-4-(1,2,4-oxadiazol-3-yl)phenyl, 2-fluoro-4-(1,2,4-triazol-1-ylmethyl)phenyl, 2-fluoro-4-(1-ethyl-2-oxo-1,2-dihydropyridin-3-yl)phenyl, 2-fluoro-4-(1-methyl-2-oxo-1,2-dihydropyridin-3-yl)phenyl, 2-fluoro-4-(1-methyl-2-oxo-1,2-dihydropyridin-5-yl)phenyl, 2-fluoro-4-(1-methyl-2-oxo-1,2-dihydropyridin-6-yl)phenyl, 2-fluoro-4-(2-carbamylphenyl)phenyl, 2-fluoro-4-(2-cyanophenyl)phenyl, 2-fluoro-4-(2-ethoxycarbonylphenyl)phenyl, 2-fluoro-4-(2-methoxypyridin-3-yl)phenyl, 2-fluoro-4-(2-methoxypyridin-4-yl)phenyl, 2-fluoro-4-(2-methoxypyridin-5-yl)phenyl, 2-fluoro-4-(2-methoxypyridin-6-yl)phenyl, 2-fluoro-4-(2-oxo-1,2-dihydropyridin-1-yl)phenyl, 2-fluoro-4-(2-oxo-1,2-dihydropyridin-3-yl)phenyl, 2-fluoro-4-(2-oxo-1,2-dihydropyridin-5-yl)phenyl, 2-fluoro-4-(2-oxo-1,2-dihydropyridin-6-yl)phenyl, 2-fluoro-4-(2-oxo-3-methylimidazolidin-1yl)phenyl, 2-fluoro-4-(3-(1-hydroxy-1-methylethan-1-yl)pyrazol-1-yl)phenyl, 2-fluoro-4-(3-carbamylphenyl)phenyl, 2-fluoro-4-(3-carbamylpyrazol-1-yl)phenyl, 2-fluoro-4-(3-carboxyphenyl)phenyl, 2-fluoro-4-(3-carboxypyrazol-1-yl)phenyl, 2-fluoro-4-(3-cyanophenyl)phenyl, 2-fluoro-4-(3-cyanopyrazol-1-yl)phenyl, 2-fluoro-4-(3-ethoxycarbonylphenyl)phenyl, 2-fluoro-4-(3-fluorophenyl)phenyl, 2-fluoro-4-(3-hydroxymethylpyrazol-1-yl)phenyl, 2-fluoro-4-(3-methoxycarbonylpyrazol-1-yl)phenyl, 2-fluoro-4-(3-methoxyphenyl)phenyl, 2-fluoro-4-(3-methoxypyrazin-2-yl)phenyl, 2-fluoro-4-(3-methylcarbamylpyrazol-1-yl)phenyl, 2-fluoro-4-(3-methylphenyl)phenyl, 2-fluoro-4-(3-N,N-dimethylcarbamylpyrazol-1-yl)phenyl, 2-fluoro-4-(4-carbamylphenyl)phenyl, 2-fluoro-4-(4-carboxypyrazol-1-yl)phenyl, 2-fluoro-4-(4-cyanophenyl)phenyl, 2-fluoro-4-(4-cyanopyrazol-1-yl)phenyl, 2-fluoro-4-(4-ethoxycarbonylphenyl)phenyl, 2-fluoro-4-(4-fluorophenyl)phenyl, 2-fluoro-4-(4-methoxycarbonylpyrazol-1-yl)phenyl, 2-fluoro-4-(4-methoxyphenyl)phenyl, 2-fluoro-4-(4-methylphenyl)phenyl, 2-fluoro-4-(5-cyanopyridin-2-yl)phenyl, 2-fluoro-4-(5-hydroxymethylpyrazol-1-yl)phenyl, 2-fluoro-4-(5-oxo-4,5-dihydro-1,2,4-oxadiazol-3-yl)phenyl, 2-fluoro-4-(morpholin-4-ylmethyl)phenyl, 2-fluoro-4-(pyrazol-1-yl)phenyl, 2-fluoro-4-(pyrazol-3-yl)phenyl, 2-fluoro-4-(pyridin-3-yl)phenyl, 2-fluoro-4-(pyridin-4-yl)phenyl, 2-fluoro-4-(pyrimidin-5-yl)phenyl, 2-fluoro-4-methylphenyl, 2-fluoro-5-(1-methyl-2-oxo-1,2-dihydropyridin-3-yl)phenyl, 2-fluoro-5-(2-oxopyrrolidin-1-yl)phenyl, 2-fluoro-5-(morpholin-4-yl)phenyl, 2-fluoro-5-ethylphenyl, 2-fluorophenyl, 2-hydroxypyridin-3-yl, 2-methyl-4-(2-carbamylethoxy)phenyl, 2-methyl-4-(2-oxopyrrolindin-1-yl)phenyl, 2-methyl-4-isopropylcarbamylphenyl, 2-methylphenyl, 2-oxo-1,2-dihydropyridin-4-yl, 2-oxo-1,2-dihydropyridin-5-yl, 2-oxo-2H-chromen-6-yl, 3-(1,2,3,4-tetrazol-1-yl)phenyl, 3-(2-oxoimidazolidin-1-yl)phenyl, 3-(2-oxo-oxazolidin-3-yl)phenyl, 3-(2-oxopyrrolidin-1-yl)phenyl, 3-(3-hydroxy-3-methylbutan-1-yn-1-yl)phenyl, 3-(4-methylpiperazin-1-yl)phenyl, 3-(aminosulfonyl)phenyl, 3-(cyanomethyl)phenyl, 3-(ethoxycarbonyl)phenyl, 3-(methylsulfonyl)phenyl, 3-(morpholin-4-yl)phenyl, 3-(morpholin-4-ylmethyl)phenyl, 3,5-dimethylphenyl, 3-aminophenylcarbonyl, 3-carbamylphenyl, 3-cyanophenyl, 3-cyclopropylphenyl, 3-ethylphenyl, 3-methoxy-4-methylsulfonylaminophenyl, 3-methylphenyl, 4-(1,1-dioxoisothiazolidin-2-yl)phenyl, 4-(1,1-dioxothiomorpholin-4-yl)phenyl, 4-(1,2,3,4-tetrazol-5-yl)phenyl, 4-(1,2,4-triazol-1-yl)phenyl, 4-(2-methoxypyrimdin-4-yl)phenyl, 4-(2-oxo-oxazolidin-3-yl)phenyl, 4-(3-oxomorpholin-4-yl)phenyl, 4-(3-oxopiperazin-1-yl)phenyl, 4-(4-hydroxypiperidin-1-yl)phenyl, 4-(4-methylpiperazin-1-yl)phenyl, 4-(4-methylpiperidin-1-yl)phenyl, 4-(5-oxo-4,5-dihydro-1,2,4-oxadiazol-3-yl)phenyl, 4-(morpholin-4-yl)phenyl, 4-(morpholin-4-ylmethyl)phenyl, 4-(N,N-dimethylaminomethyl)phenyl, 4-(N,N-dimethylaminosulfonyl)phenyl, 4-(pyrrolidin-1-yl)phenyl, 4-(tetrahydropyran-4-yl)phenyl, 4-cyanomethylphenyl, 4-dimethylaminophenyl, 4-isopropylphenyl, 4-methylcarbamylphenyl, 4-methylphenyl, 4-methylsulfonylphenyl, 4-t-butylphenyl, 5-(2-methoxycarbonylethan-1-yl)-1,3,4-thiadiazol-2-yl, 5-methoxypyridin-3-yl, 7-chloroimidazo[1,2-b]pyridazin-3-yl, phenyl, or pyrimidin-5-yl.

In certain embodiments, ring A is piperidinyl, piperidinylidene, piperazinyl, pyrrolidinyl, azetidinyl, cyclohexyl, cyclopentyl, cyclobutyl, azabicyclo[3.3.1]nonanyl, or azabicyclo[2.2.1]heptanyl.

In certain embodiments, each R² is independently —F, —OH, —CH₃, —CH₂CH₃, —CH₂CF₃, —CH₂CH₂OH, —CH₂CH(OH)CH₂OH, —CH(CH₃)₂, —CH(CH₃)—COOH, —COOH, —NH₂, —NH(CH₃), —N(CH₃)₂—CH₂C(O)NH₂, or oxetan-3-ylmethyl.

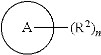

In certain embodiments, the portion of the compound represented by is: 1-(2,2,2-trifluoroethyl)piperidin-4-yl, 1-(2-hydroxyethyl)piperidin-4-yl, 1-(2,3-dihydroxypropyl)piperidin-4-yl, 1-(carbamylmethyl)piperidin-4-yl, 1-(oxetan-3-ylmethyl)piperidin-4-yl, 1,3-dimethypiperidin-4-yl, 1,4-dimethylpiperidin-4-yl, 1-ethylpiperidin-4-yl, 1-isopropylpiperidin-4-yl, 1-methyl-1-oxopiperidin-4-yl, 1-methyl-3,3-difluoropiperidin-4-yl, 1-methyl-4-hydroxypiperidin-4-yl, 1-methylpiperidin-4-yl, 1-methylpiperidin-4-ylidene, 1-methylpyrrolidin-3-yl, 2-azabicyclo[2.2.1]heptan-5-yl, 2-methylpiperidin-4-yl, 3,3-difluoropiperidin-4-yl, 3-aminocyclobutyl, 3-aminopyrrolidin-1-yl, 3-aminopiperidin-1-yl, 3-carboxypiperidin-4-yl, 3-methylpiperidin-4-yl, 4-(dimethylamino)cyclohexyl, 4-(methylamino)cyclohexyl, 4-amino-4-methylcyclohexyl, 4-aminocyclohexyl, 4-hydroxycyclohexyl, 4-hydroxypiperidin-4-yl, 4-methylpiperazin-1-yl, 9-azabicyclo[3.3.1]nonan-3-yl, azetidin-3-yl, piperazin-1-yl, piperidin-4-yl, or piperidin-4-ylidene.

In certain embodiments, R¹ is —N(CH₃)—, —NH—, —N(CH₂CH₂OH)—, —N(CH₂COOH)—, —N(CH₂CH₂COOH)—, —N(S(O)₂CH₃)—, —N(C(O)C(O)OH)—, —C(O)—, —S—, —S(O)—, —S(O)₂—, —C(CH₃)(OH)—, —C(CH₃)(F)—, —C(CH₂CH₃)(OH)—, —C(CF₃)(OH)—, —CH(CH₃)—, —CH(CH₂CH₃)—, —CH(OH)—, —CH(CH₂OH)—, —CH(═CH₂)—, —C(═N—OH), —C(═N—OCH₃), —CF₂—, —CHF—, —CH(OCH₃)—, —CH═, —CH₂—, —CH(NH₂)—, —CH(NHCH₃)—, —NH—S(O)₂—, —N(CH₂CN)—, —S(O)₂—NH—, —N(CH₂COOCH₃)—, —CH₂—S(O)₂—, —N(CH(CH₃)COOH)—, pyrazol-4-ylmethylaminylene, cyclopropan-1,1-diyl, and oxetan-2,2-diyl.

In certain embodiments, the compound has structural formula (Ia):

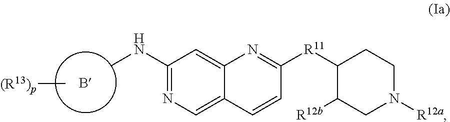

or a pharmaceutically acceptable salt thereof, wherein:

ring B′ is phenyl, pyridin-3-yl, or 1,3-dihydroisobenzofuran-5-yl;

R¹¹ is —S—, —S(O)₂—, —CF₂—, —C(F)(CH₃)—, —C(OH)(CH₃)—, —CH(CH₃)—, or —C(O)—;

R^{12a} is hydrogen, —CH₃, —CH₂CH₂OH, or oxetan-3-ylmethyl;

R^{12b} is hydrogen or —CH₃;

each R¹³, if present, is independently fluoro; C₁-C₄ alkyl optionally substituted with one or more of —CN and —OH; C₂-C₄ alkynyl optionally substituted with one or more —OH; —C(O)N(R⁶)₂; —C(O)O—C₁-C₄ alkyl; —N(R⁶)₂; —S(O)₂N(R⁶)₂; —SO₂-C₁-C₄ alkyl; phenyl optionally substituted one or more of fluoro, —CN, —C(O)N(R⁶), —COOH, —O—C₁-C₄ alkyl, and C₁-C₄ hydroxyalkyl; pyridinyl optionally substituted with one or more O—C₁-C₄ alkyl; pyrazolyl optionally substituted with one or more of —COOH, C₁-C₄ hydroxyalkyl, —C(O)O—C₁-C₄ alkyl; pyrimidinyl optionally substituted with O—C₁-C₄ alkyl; oxo-substituted 1,2-dihydropyridinyl; oxo-substituted pyrazolidinyl optionally further substituted with C₁-C₄ alkyl; oxo-substituted oxazolidinyl; oxo-substituted pyrrolidinyl; oxo-substituted thiazolidinyl; oxo-substituted thiomorpholinyl; morpholinyl; or cyclopropyl;

each R⁶ is independently hydrogen or C₁— C₄ alkyl; and

p is 0, 1 or 2.

In certain embodiments, p is 2, and one R¹³ is fluoro.

In certain embodiments, ring B is phenyl.

In certain embodiments, each R¹³ is independently, fluoro, —CH₃, —CH₂CH₃, —CH₂CN, —CH(CH₃)₂, —C═C—C(CH₃)₂OH, —C(OH)(CH₃)CH₃, —C(CH₃)₃, —C(O)NH₂, —C(O)OCH₂CH₃, —N(CH₃)₂, —S(O)₂NH₂, —SO₂CH₃,1,1-dioxothiazolidin-2-yl, 1,1-dioxothiomorpholin-4-yl, 2-cyanophenyl, 2-methoxypyridin-4-yl, 2-methoxypyridin-5-yl, 2-methoxypyrimidin-4-yl, 2-oxo-1,2-dihydropyridin-6-yl, 2-oxo-1,2-dihydropyridin-3-yl, 2-oxo-3-methylpyrazolidin-1-yl, 2-oxooxazol-3-yl, 2-oxopyrrolidin-1-yl, 3-carbamylphenyl, 3-carboxyphenyl, 3-carboxypyrazol-1-yl, 3-cyanophenyl, 3-fluorophenyl, 3-hydroxymethylpyrazol-1-yl, 3-methoxyphenyl, 4-carboxypyrazol-1-yl, 4-cyanophenyl, 4-methoxycarbonylpyrazol-1-yl, 4-methoxyphenyl, morpholin-4-yl, pyrazol-1-yl, pyrazol-3-yl, pyridin-3-yl, pyrimidin-5-yl, or cyclopropyl.

In certain embodiments, the compound has structural formula (Ib):

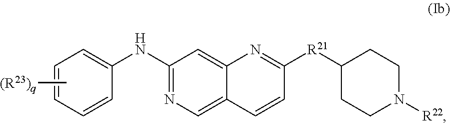

or a pharmaceutically acceptable salt thereof, wherein:

R²¹ is —CH(CH₃)—, —CH(OH)—, —C(CH₃)(OH)—, C(═CH₂)—, N(CH₂C(O)OH)—, —S—, or —S(O)₂—;

R²² is hydrogen, —CH₃, —CH₂CH₃, —CH₂CH₂OH, or azetidin-3-ylmethyl;

each R²³ is independently fluoro; C₁-C₄ alkyl; C₂-C₄ alkynyl optionally substituted with hydroxy; —N(R⁶)₂; —O—C₁-C₄ alkylene-C(O)—N(R⁶)₂; phenyl optionally substituted with one or more of halo, —CN, C₁-C₄ alkyl, —O—C₁-C₄ alkyl, —C(O)N(R⁶)₂, and —C(O)—C₁-C₄ alkyl; pyridinyl optionally substituted with —O—C₁-C₄ alkyl; pyrazolyl optionally substituted with one or more of —CN, —C₁-C₄ alkyl, —C₁-C₄ hydroxyalkyl, —C(O)N(R⁶)₂, —COOH, and —C(O)—O—C₁-C₄ alkyl; oxo-substituted oxadiazolyl; morpholinyl; morpholinylmethyl; tetrahydropyranyl; pyrrolidinyl; pyrimidinyl; tetrazolyl; piperidinyl optionally substituted with C₁-C₄ alkyl; or cyclopropyl; and

q is 1 or 2.

In certain embodiments, q is 2; and one R²³ is —CH₃ or fluoro.

In certain embodiments, each R²³ is independently fluoro, —CH₃, —CH₂CH₃, —CH(CH₃)₂, C≡C—C((CH₃)₂)OH, —N(CH₃)₂, —OCH₂CH₂C(O)NH₂, 1,2,3,4-tetrazol-5-yl, 2-methoxypyridin-3-yl, 2-methoxypyridin-4-yl, 2-methoxypyridin-5-yl, 2-methoxypyridin-6-yl, 3-(N,N-dimethylcarbamyl)pyrazol-1-yl, 3-carbamylphenyl, 3-carbamylpyrazol-1-yl, 3-carboxypyrazol-1-yl, 3-cyanophenyl, 3-cyanopyrazol-1-yl, 3-ethoxycarbonylphenyl, 3-fluorophenyl, 3-hydroxymethylpyrazol-1-yl, 3-methoxycarbonylpyrazol-1-yl, 3-methoxyphenyl, 3-methylphenyl, 4-carbamylphenyl, 4-cyanophenyl, 4-ethoxycarbonylphenyl, 4-fluorophenyl, 4-methoxycarbonylpyrazol-1-yl, 4-methoxyphenyl, 4-methylphenyl, 4-methylpiperidin-1-yl, 5-oxo-1,2,4-oxadiazol-3-yl, cyclopropyl, fluoro, morpholin-4-yl, morpholin-4-ylmethyl, pyrazol-1-yl, pyridin-3-yl, pyridin-4-yl, pyrimidin-5-yl, pyrrolidin-1-yl, or tetrahydropyran-4-yl.

In certain embodiments, the compound is selected from any one of the compounds 100-315 in Table 1, or a pharmaceutically acceptable salt thereof.

In certain embodiments, the compounds of the invention may be racemic. In certain embodiments, the compounds of the invention may be enriched in one enantiomer. For example, a compound of the invention may have greater than 30% ee, 40% ee, 50% ee, 60% ee, 70% ee, 80% ee, 90% ee, or even 95% or greater ee.

The compounds of the invention have more than one stereocenter. Accordingly, the compounds of the invention may be enriched in one or more diastereomers. For example, a compound of the invention may have greater than 30% de, 40% de, 50% de, 60% de, 70% de, 80% de, 90% de, or even 95% or greater de. In certain embodiments, the compounds of the invention have substantially one isomeric configuration at one or more stereogenic centers, and have multiple isomeric configurations at the remaining stereogenic centers.

In certain embodiments, the enantiomeric excess of the stereocenter is at least 40% ee, 50% ee, 60% ee, 70% ee, 80% ee, 90% ee, 92% ee, 94% ee, 95% ee, 96% ee, 98% ee or greater ee.

As used herein, single bonds drawn without stereochemistry do not indicate the stereochemistry of the compound.

As used herein, hashed or bolded non-wedge bonds indicate relative, but not absolute, stereochemical configuration (e.g., do not distinguish between enantiomers of a given diastereomer).

As used herein, hashed or bolded wedge bonds indicate absolute stereochemical configuration.

In some embodiments, the invention relates to pharmaceutical composition comprising a compound of the invention and a pharmaceutically acceptable carrier. In certain embodiments, a therapeutic preparation or pharmaceutical composition of the compound of the invention may be enriched to provide predominantly one enantiomer of a compound. An enantiomerically enriched mixture may comprise, for example, at least 60 mol percent of one enantiomer, or more preferably at least 75, 90, 95, or even 99 mol percent. In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

In certain embodiments, a therapeutic preparation or pharmaceutical composition may be enriched to provide predominantly one diastereomer of the compound of the invention. A diastereomerically enriched mixture may comprise, for example, at least 60 mol percent of one diastereomer, or more preferably at least 75, 90, 95, or even 99 mol percent.

### Pharmaceutical Compositions

The compositions and methods of the present invention may be utilized to treat a subject in need thereof. In certain embodiments, the subject is a mammal such as a human, or a non-human mammal. When administered to subject, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

“Pharmaceutically acceptable salt” is used herein to refer to an acid addition salt or a basic addition salt which is suitable for or compatible with the treatment of patients.

The term “pharmaceutically acceptable acid addition salt” as used herein means any non-toxic organic or inorganic salt of the disclosed compounds. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, bitartaric, citric, ascorbic, maleic, benzoic, phenylacetic, cinnamic, salicylic, and sulfosalicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds dislcosed herein are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds dislcosed herein for laboratory use, or for subsequent conversion to a pharmaceutically acceptable acid addition salt.

The term “pharmaceutically acceptable basic addition salt” as used herein means any non-toxic organic or inorganic base addition salt of any acid compounds disclosed herein. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, intraocular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, about 0.1 to about 99.5% (more preferably, about 0.5 to about 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the subject's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In certain embodiments, the active compound will be administered once daily.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

In certain embodiments, conjoint administration of compounds of the invention with one or more additional therapeutic agent(s) provides improved efficacy relative to each individual administration of the compound of the invention or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s).

This invention includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Methods of Treatment

The compounds and compositions described here may be used to treat a disease or condition characterized by aberrant CDK5 overactivity, such as a disease or condition of the kidney or a ciliopathy. Administration of CDK5 inhibitors will show benefits in therapeutic indications associated with upregulation of CDK5 (i.e., increased levels of CDK5 protein in diseased tissue compared to healthy tissue).

In some embodiments, the disease or condition is a disease or condition of the kidney. In some embodiments, the kidney disease or condition is a cystic kidney disease, renal fibrosis, diabetic nephropathy, a parenchymal renal disease, or decreased renal function. In some embodiments, the kidney disease or condition is chronic kidney disease, polycystic kidney disease, autosomal dominant polycystic kidney disease, autosomal recessive polycystic kidney disease, or nephronophthisis-medullary cystic kidney disease. In some embodiments, the disease is polycystic kidney disease.

In some embodiments, the disease or condition is a ciliopathy. In some embodiments, the ciliopathy is a neurodegenerative disease, a liver disease, inflammation, a cancer, or a tumor. In some embodiments, the neurodegenerative disease is Alzheimer's disease or Parkinson's disease. In some embodiments, the liver disease is polycystic liver disease.

Kidney Disease

Kidney diseases and conditions include, but are not limited to, kidney failure (also known as end stage kidney disease or ESRD), kidney stones, polycystic kidney disease, cystic kidney disease, renal fibrosis, diabetic nephropathy, a parenchymal renal disease, decreased renal function, chronic kidney disease, polycystic kidney disease, autosomal dominant polycystic kidney disease, autosomal recessive polycystic kidney disease, and nephronophthisis-medullary cystic kidney disease. Major causes of kidney diseases in the United States include diabetes, high blood pressure, and glomerulonephritis, a disease that damages the kidneys' filtering units, the glomeruli. (https://www.kidney.org/atoz/content/kidneydiscauses).

Cysde Kidney Disease

Cystic kidney disease refers to a wide range of hereditary, developmental, and acquired conditions. With the inclusion of neoplasms with cystic changes, over 40 classifications and subtypes have been identified. Depending on the disease classification, the presentation of disease may be from birth, or much later into adult life. Cystic disease may involve one or both kidneys and may or may not occur in the presence of other anomalies. A higher incidence of cystic kidney disease is found in the male population and prevalence increases with age. Renal cysts have been reported in more than 50% of patients over the age of 50. Typically, cysts grow up to 2.88 mm annually and cause related pain and/or hemorrhage.

Of the cystic kidney diseases, the most common is Polycystic kidney disease; having two prevalent sub-types: autosomal recessive and autosomal dominant polycystic kidney disease. Autosomal Recessive Polycystic Kidney Disease (ARPKD) is primarily diagnosed in infants and young children. Autosomal dominant polycystic kidney disease (ADPKD) is most often diagnosed in adulthood.

Renal Fibrosis

Fibrotic disorders are commonplace, take many forms and can be life-threatening. No better example of this exists than the progressive fibrosis that accompanies all chronic renal disease. Renal fibrosis is a direct consequence of the kidney's limited capacity to regenerate after injury. Renal scarring results in a progressive loss of renal function, ultimately leading to end-stage renal failure and a requirement for dialysis or kidney transplantation. [Hewitson: Fibrosis in the kidney: is a problem shared a problem halved? Fibrogenesis & Tissue Repair 2012 5 (Suppl 1):S14].

Parenchymal Renal Disease

The renal parenchyma is the functional part of the kidney that includes the renal cortex (the outermost part of the kidney) and the renal medulla. The renal cortex contains the approximately 1 million nephrons (these have glomeruli which are the primary filterer of blood passing through the kidney, and renal tubules which modify the fluid to produce the appropriate amount/content of urine). The renal medulla consists primarily of tubules/ducts which are the beginning of the collecting system that allows the urine to flow onwards to being excreted. Renal parenchyma disease describes medical conditions which damage these parts of the kidney. These diseases may be congenital, hereditary or acquired. Causes vary and include genetic conditions like polycystic kidneys, hereditary conditions passed on from parents, bacterial and viral infections, kidney stones, high blood pressure, diabetes, autoimmune diseases like lupus nephritis or nephritis associated with purpura, medications and others. Common signs include swelling of hands/feet/eyes (edema), high blood pressure, anemia, bone changes, blood in the urine, abdominal swelling, Common symptoms include loss of appetite, itching, nausea and vomiting, fatigue, joint pain, frequent night urination and dizziness. [https://www.nicklauschildrens.org/conditions/renal-parenchyma-diseases]

Chronic Kidney Disease

Chronic kidney disease, also called chronic kidney failure, describes the gradual loss of kidney function. When chronic kidney disease reaches an advanced stage, dangerous levels of fluid, electrolytes and wastes can build up in the body. Chronic kidney disease may not become apparent until kidney function is significantly impaired. Treatment for chronic kidney disease focuses on slowing the progression of the kidney damage, usually by controlling the underlying cause. Chronic kidney disease can progress to end-stage kidney failure, which is fatal without artificial filtering (dialysis) or a kidney transplant. Chronic kidney disease occurs when a disease or condition impairs kidney function, causing kidney damage to worsen over several months or years. Diseases and conditions that cause chronic kidney disease include, but are not limited to, diabetes, high blood pressure, glomerulonephritis, interstitial nephritis, polycystic kidney disease, prolonged obstruction of the urinary tract (e.g., from conditions such as enlarged prostate, kidney stones, and some cancers), vesicoureteral reflux, and recurrent kidney infection, also called pyelonephritis, [https://www.mayoclinic.org/diseases-conditions/chronic-kidney-disease/symptoms-causes/syc-20354521]

Nephronophthisis-Medullary Cystic Kidney Disease

Medullary cystic kidney disease (MCKD) and nephronophthisis (NPH) refer to 2 inherited diseases with similar renal morphology characterized by bilateral small corticomedullary cysts in kidneys of normal or reduced size and tubulointerstitial sclerosis leading to end-stage renal disease (ESR)). These disorders have traditionally been considered as parts of a complex (the NPH complex) because they share many of the clinical and histopathological features. The major differences are in the modes of inheritance, the age of onset of ESRD, and the extrarenal manifestations. [https://emedicine.medscape.com/article/982359-overview].

Nephronophthisis is a genetic disorder of the kidneys which affects children. It is classified as a medullary cystic kidney disease. The disorder is inherited in an autosomal recessive fashion and, although rare, is the most common genetic cause of childhood kidney failure. It is a form of celiopathy. Its incidence has been estimated to be 0.9 cases per million people in the United States, and 1 in 50,000 births in Canada. Infantile, juvenile, and adolescent forms of nephronophthisis have been identified. Although the range of characterizations is broad, people affected by nephronophthisis typically present with polyuria (production of a large volume of urine), polydipsia (excessive liquid intake), and after several months to years, end-stage kidney disease, a condition necessitating either dialysis or a kidney transplant in order to survive. Some individuals that suffer from nephronophthisis also have so-called “extra-renal symptoms” which can include tapetoretinal degeneration, liver problems, ocularmotor apraxia, and cone-shaped epiphysis (Saldino-Mainzer syndrome). Mechanism of nephronophthisis indicates that all proteins mutated in cystic kidney diseases express themselves in primary cilia, NPHP gene mutations cause defects in signaling resulting in flaws of planar cell polarity. The ciliary theory indicates that multiple organs are involved in NPHP (retinal degeneration, cerebellar hypoplasia, liver fibrosis, and intellectual disability).

Medullary cystic kidney disease (MCKD) is an autosomal dominant kidney disorder characterized by tubulointerstitial sclerosis leading to end-stage renal disease. Because the presence of cysts is neither an early nor a typical diagnostic feature of the disease, and because at least 4 different gene mutations may give rise to the condition, the name autosomal dominant tubulointerstitial kidney disease (ADTKD) has been proposed, to be appended with the underlying genetic variant for a particular individual. Importantly, if cysts are found in the medullary collecting ducts they can result in a shrunken kidney, unlike that of polycystic kidney disease. There are two known forms of medullary cystic kidney disease, mucin-1 kidney disease 1 (MKD1) and mucin-2 kidney disease/uromodulin kidney disease (MM 2). A third form of the disease occurs due to mutations in the gene encoding renin (ADTKD-REN), and has formerly been known as familial juvenile hyperuricemic nephropathy type 2. In terms of the signs/symptoms of medullary cystic kidney disease, the disease is not easy to diagnose and is uncommon. In this condition, loss of kidney function occurs slowly over time, however the following signs/symptoms could be observed in an affected individual: Polydipsia, Enuresis, Weakness, lack of appetite, Pruritus, Bone pain, Pallor, Nausea. Some individuals with this disease develop gout, which if untreated, becomes chronic and affects the joints most of the time, instead of intermittently.

Polycystic Kidney Disease

Polycystic kidney disease (PKD) is a genetic disorder in which the renal tubules become structurally abnormal, resulting in the development and growth of multiple cysts within the kidney. These cysts may begin to develop in utero, in infancy, in childhood, or in adulthood. Cysts are non-functioning tubules filled with fluid pumped into them, which range in size from microscopic to enormous, crushing adjacent normal tubules and eventually rendering them non-functional as well. PKD is one of the most common hereditary diseases in the United States, affecting more than 600,000 people. It is the cause of nearly 10% of all end-stage renal disease.

Causes of Polycystic Kidney Disease

PKD is caused by abnormal genes which produce a specific abnormal protein; this protein has an adverse effect on tubule development. PKD is a general term for two types, each having their own pathology and genetic cause: autosomal dominant polycystic kidney disease (ADPKD) and autosomal recessive polycystic kidney disease (ARPKD). The abnormal gene exists in all cells in the body; as a result, cysts may occur in the liver, seminal vesicles, and pancreas. This genetic defect can also cause aortic root aneurysms, and aneurysms in the circle of Willis cerebral arteries, which if they rupture, can cause a subarachnoid hemorrhage.

Diagnosis may be suspected from one, some, or all of the following: new onset flank pain or red urine; a positive family history; palpation of enlarged kidneys on physical exam; an incidental finding on abdominal sonogram; or an incidental finding of abnormal kidney function on routine lab work (BUN, serum creatinine, or eGFR). Polycystic kidney disease can be ascertained via a CT scan of abdomen, as well as, an MRI and ultrasound of the same area. A physical exam/test can reveal enlarged liver, heart murmurs and elevated blood pressure.

Complications include hypertension due to the activation of the renin-angiotensin-aldosterone system (RAAS), frequent cyst infections, urinary bleeding, and declining renal function. Hypertension is treated with angiotensin converting enzyme inhibitors (ACEIs) or angiotensin receptor blockers (ARBs). Infections are treated with antibiotics. Declining renal function is treated with renal replacement therapy (RRT): dialysis and/or transplantation. Management from the time of the suspected or definitive diagnosis is by a board-certified nephrologist. There is no FDA-approved treatment. However, it has been shown that mild to moderate dietary restrictions slow the progression of autosomal dominant polycystic kidney disease (ADPKD). If and when the disease progresses enough in a given case, the nephrologist or other practitioner and the patient will have to decide what form of renal replacement therapy will be used to treat end-stage kidney disease (kidney failure, typically stage 4 or 5 of chronic kidney disease).

Ciliopathy

A ciliopathy is a genetic disorder of the cellular cilia or the cilia anchoring structures, the basal bodies, or of ciliary function. Primary cilia are important in guiding the process of development, so abnormal ciliary function while an embryo is developing can lead to a set of malformations that can occur regardless of the particular genetic problem. The similarity of the clinical features of these developmental disorders means that they form a recognizable cluster of syndromes, loosely attributed to abnormal ciliary function and hence called ciliopathies. Regardless of the actual cause, it is clustering of a set of characteristic features which define whether a syndrome is a ciliopathy.

Polycystic Liver Disease

Polycystic liver disease (PLD) usually describes the presence of multiple cysts scattered throughout normal liver tissue. PLD is commonly seen in association with autosomal-dominant polycystic kidney disease, with a prevalence of 1 in 400 to 1000, and accounts for 8-10% of all cases of end stage renal disease. The much rarer autosomal-dominant polycystic liver disease will progress without any kidney involvement. Associations with PRKCSH and SEC63 have been described. Polycystic liver disease comes in two forms as autosomal dominant polycystic kidney disease (with kidney cysts) and autosomal dominant polycystic liver disease (liver cysts only). Most patients with PLD are asymptomatic with simple cysts found following routine investigations. After confirming the presence of cysts in the liver, laboratory tests may be ordered to check for liver function including bilirubin, alkaline phosphatase, alanine aminotransferase, and prothrombin time. Patients with PLD often have an enlarged liver which will compress adjacent organs, leading to nausea, respiratory issues, and limited physical ability. Classification of the progression of the disease takes into consideration the amount of remaining liver parenchyma compared to the amount and size of cysts. Many patients are asymptomatic and thus are not candidates for surgery. For patients with pain or complications from the cysts, the goal of treatment is to reduce the size of cysts while protecting the functioning liver parenchyma, Cysts may be removed surgically or by using aspiration sclerotherapy.

Alzheimer's Disease

Alzheimer's disease (AD) is a chronic neurodegenerative disease that usually starts slowly and gradually worsens over time. It is the cause of 60-70% of cases of dementia. The most common early symptom is difficulty in remembering recent events. As the disease advances, symptoms can include problems with language, disorientation (including easily getting lost), mood swings, loss of motivation, not managing self-care, and behavioural issues. As a person's condition declines, they often withdraw from family and society. Gradually, bodily functions are lost, ultimately leading to death. Although the speed of progression can vary, the typical life expectancy following diagnosis is three to nine years. The cause of Alzheimer's disease is poorly understood. About 70% of the risk is believed to be inherited from a person's parents with many genes usually involved. Other risk factors include a history of head injuries, depression, and hypertension. The disease process is associated with plaques and neurofibrillary tangles in the brain. A probable diagnosis is based on the history of the illness and cognitive testing with medical imaging and blood tests to rule out other possible causes. Initial symptoms are often mistaken for normal ageing. Examination of brain tissue is needed for a definite diagnosis. Mental and physical exercise, and avoiding obesity may decrease the risk of AD; however, evidence to support these recommendations is weak. There are no medications or supplements that have been shown to decrease risk. No treatments stop or reverse its progression, though some may temporarily improve symptoms. In 2015, there were approximately 29.8 million people worldwide with D. It most often begins in people over 65 years of age, although 4-5% of cases are early-onset Alzheimer's. It affects about 6% of people 65 years and older.

Parkinson's Disease

Parkinson's disease (PD) is a long-term degenerative disorder of the central nervous system that mainly affects the motor system. As the disease worsens, non-motor symptoms become more common. The symptoms usually emerge slowly. Early in the disease, the most obvious symptoms are shaking, rigidity, slowness of movement, and difficulty with walking. Thinking and behavioral problems may also occur. Dementia becomes common in the advanced stages of the disease. Depression and anxiety are also common, occurring in more than a third of people with PD. Other symptoms include sensory, sleep, and emotional problems. The main motor symptoms are collectively called “parkinsonism”, or a “parkinsonian syndrome”. The cause of Parkinson's disease is believed to involve both genetic and environmental factors. Those with a family member affected are more likely to get the disease themselves. There is also an increased risk in people exposed to certain pesticides and among those who have had prior head injuries, while there is a reduced risk in tobacco smokers and those who drink coffee or tea. The motor symptoms of the disease result from the death of cells in the substantia nigra, a region of the midbrain. This results in not enough dopamine in this region of the brain. The cause of this cell death is poorly understood, but it involves the build-up of proteins into Lewy bodies in the neurons. Diagnosis of typical cases is mainly based on symptoms, with tests such as neuroimaging used to rule out other diseases. In 2015, PD affected 6.2 million people and resulted in about 117,400 deaths globally. Parkinson's disease typically occurs in people over the age of 60, of whom about one percent are affected. The average life expectancy following diagnosis is between 7 and 15 years.

Proteinuria

Proteinuria is a pathological condition wherein protein is present in the urine. Albuminuria is a type of proteinuria. Microalbuminuria occurs when the kidney leaks small amounts of albumin into the urine. In a properly functioning body, albumin is not normally present in urine because it is retained in the bloodstream by the kidneys. Microalbuminuria is diagnosed either from a 24-hour urine collection (20 to 200 μg/min) or, more commonly, from elevated concentrations (30 to 300 mg/L) on at least two occasions. Microalbuminuria can be a forerunner of diabetic nephropathy. An albumin level above these values is called macroalbuminuria. Subjects with certain conditions, e.g., diabetic nephropathy, can progress from microalbuminuria to macroalbuminuria and reach a nephrotic range (>3.5 g/24 hours) as kidney disease reaches advanced stages.

Causes of Proteinuria

Proteinuria can be associated with a number of conditions, including focal segmental glomerulosclerosis, IgA nephropathy, diabetic nephropathy, lupus nephritis, membranoproliferative glomerulonephritis, progressive (crescentic) glomerulonephritis, and membranous glomerulonephritis.

A. Focal Segmental Glomerulosclerosis (FSGS)

Focal Segmental Glomerulosclerosis (FSGS) is a disease that attacks the kidney's filtering system (glomeruli) causing serious scarring. FSGS is one of the many causes of a disease known as Nephrotic Syndrome, which occurs when protein in the blood leaks into the urine (proteinuria). Primary FSGS, when no underlying cause is found, usually presents as nephrotic syndrome. Secondary FSGS, when an underlying cause is identified, usually presents with kidney failure and proteinuria. FSGS can be genetic; there are currently several known genetic causes of the hereditary forms of FSGS.

Very few treatments are available for patients with FSGS. Many patients are treated with steroid regimens, most of which have very harsh side effects. Some patients have shown to respond positively to immunosuppressive drugs as well as blood pressure drugs which have shown to lower the level of protein in the urine. To date, there is no commonly accepted effective treatment or cure and there are no FDA approved drugs to treat FSGS. Therefore, more effective methods to reduce or inhibit proteinuria are desirable.

B. IgA Nephropathy

IgA nephropathy (also known as IgA nephritis, IgAN, Berger's disease, and synpharyngitic glomerulonephritis) is a form of glomerulonephritis (inflammation of the glomeruli of the kidney). IgA nephropathy is the most common glomerulonephritis throughout the world. Primary IgA nephropathy is characterized by deposition of the IgA antibody in the glomerulus. There are other diseases associated with glomerular IgA deposits, the most common being Henoch-Sthönlein purpura (HSP), which is considered by many to be a systemic form of IgA nephropathy. Henoch-Schönlein purpura presents with a characteristic purpuric skin rash, arthritis, and abdominal pain and occurs more commonly in young adults (16-35 yrs old). HSP is associated with a more benign prognosis than IgA nephropathy. In IgA nephropathy there is a slow progression to chronic renal failure in 25-30% of cases during a period of 20 years.

C. Diabetic Nephropathy

Diabetic nephropathy, also known as Kimmelstiel-Wilson syndrome and intercapillary glomerulonephritis, is a progressive kidney disease caused by angiopathy of capillaries in the kidney glomeruli. It is characterized by nephrotic syndrome and diffuse glomerulosclerosis. It is due to longstanding diabetes mellitus and is a prime cause for dialysis. The earliest detectable change in the course of diabetic nephropathy is a thickening in the glomerulus. At this stage, the kidney may start allowing more serum albumin than normal in the urine. As diabetic nephropathy progresses, increasing numbers of glomeruli are destroyed by nodular glomerulosclerosis and the amount of albumin excreted in the urine increases.

D. Lupus Nephritis

Lupus nephritis is a kidney disorder that is a complication of systemic lupus erythematosus. Lupus nephritis occurs when antibodies and complement build up in the kidneys, causing inflammation. It often causes proteinuria and may progress rapidly to renal failure. Nitrogen waste products build up in the bloodstream. Systemic lupus erythematosus causes various disorders of the internal structures of the kidney, including interstitial nephritis. Lupus nephritis affects approximately 3 out of 10,000 people.

E. Membranoproliferative Glomerulonephritis I/II/III

Membranoproliferative glomerulonephritis is a type of glomerulonephritis caused by deposits in the kidney glomerular mesangium and basement membrane thickening, activating complement and damaging the glomeruli. There are three types of membranoproliferative glomerulonephritis. Type I is caused by immune complexes depositing in the kidney and is believed to be associated with the classical complement pathway. Type II is similar to Type I, however, it is believed to be associated with the alternative complement pathway. Type III is very rare and it is characterized by a mixture of subepithelial deposits and the typical pathological findings of Type I disease.

There are two major types of MPGN, which are based upon immunofluorescence microscopy: immune complex-mediated and complement-mediated. Hypocomplementemia is common in all types of MPGN. In immune complex-mediated MPGN, complement activation occurs via the classic pathway and is typically manifested by a normal or mildly decreased serum C3 concentration and a low serum C4 concentration. In complement-mediated MPGN, there are usually low serum C3 and normal C4 levels due to activation of the alternate pathway. However, complement-mediated MPGN is not excluded by a normal serum C3 concentration, and it is not unusual to find a normal C3 concentration in adults with dense deposit disease (DDD) or C3 glomerulonephritis (C3GN).

C3 glomerulonephritis (C3GN) shows a glomerulonephritis on light microscopy (LM), bright C3 staining and the absence of Clq, C4 and immunoglobulins (Ig) on immunofluorescence microscopy (if), and mesangial and/or subendothelial electron dense deposits on electron microscopy (EM). Occasional intramembranous and subepithelial deposits are also frequently present. The term ‘C3 glomerulopathy’ is often used to include C3GN and Dense Deposit Disease (DDD), both of which result from dysregulation of the alternative pathway (AP) of complement. C3GN and DDD may be difficult to distinguish from each other on LM and IF studies. However. EM shows mesangial and/or subendothelial, intramembranous and subepithelial deposits in C3GN, while dense osmiophilic deposits are present along the glomerular basement membranes (GBM) and in the mesangium in DDD. Both C3GN and DDD are distinguished from immune-complex mediated glomerulonephritis by the lack of immunoglobulin staining on IF. (Sethi et al., Kidney Int. (2012) 82(4):465-473).

R: Progressive (Crescentic) Glomerulonephritis

Progressive (crescentic) glomerulonephritis (PG) is a syndrome of the kidney that, if left untreated, rapidly progresses into acute renal failure and death within months. In 50% of cases, PG is associated with an underlying disease such as Goodpasture's syndrome, systemic lupus erythematosus, or Wegener granulomatosis; the remaining cases are idiopathic. Regardless of the underlying cause, PG involves severe injury to the kidney's glomeruli, with many of the glomeruli containing characteristic crescent-shaped scars. Patients with PG have hematuria, proteinuria, and occasionally, hypertension and edema. The clinical picture is consistent with nephritic syndrome, although the degree of proteinuria may occasionally exceed 3 g/24 hours, a range associated with nephrotic syndrome. Untreated disease may progress to decreased urinary volume (oliguria), which is associated with poor kidney function.

G. Membranous Glomerulonephritis

Membranous glomerulonephritis (MGN) is a slowly progressive disease of the kidney affecting mostly patients between ages of 30 and 50 years, usually Caucasian. It can develop into nephrotic syndrome. MGN is caused by circulating immune complex. Current research indicates that the majority of the immune complexes are formed via binding of antibodies to antigens in situ to the glomerular basement membrane. The said antigens may be endogenous to the basement membrane, or deposited from systemic circulation.

H. Alport Syndrome

Alport syndrome is a genetic disorder affecting around 1 in 5,000-10,000 children, characterized by glomerulonephritis, end-stage kidney disease, and hearing loss. Alport syndrome can also affect the eyes, though the changes do not usually affect sight, except when changes to the lens occur in later life. Blood in urine is universal. Proteinuria is a feature as kidney disease progresses.

I. Hypertensive Kidney Disease

Hypertensive kidney disease (Hypertensive nephrosclerosis (HN or HNS) or hypertensive nephropathy (HN)) is a medical condition referring to damage to the kidney due to chronic high blood pressure. HN can be divided into two types: benign and malignant. Benign nephrosclerosis is common in individuals over the age of 60 while malignant nephrosclerosis is uncommon and affects 1-5% of individuals with high blood pressure, that have diastolic blood pressure passing 130 mm Hg. Signs and symptoms of chronic kidney disease, including loss of appetite, nausea, vomiting, itching, sleepiness or confusion, weight loss, and an unpleasant taste in the mouth, may develop. Chronic high blood pressure causes damages to kidney tissue this includes the small blood vessels, glomeruli, kidney tubules and interstitial tissues. The tissue hardens and thickens which is known as nephrosclerosis. The narrowing of the blood vessels means less blood is going to the tissue and so less oxygen is reaching the tissue resulting in tissue death (ischemia).

J. Nephrotic Syndrome

Nephrotic syndrome is a collection of symptoms due to kidney damage. This includes protein in the urine, low blood albumin levels, high blood lipids, and significant swelling. Other symptoms may include weight gain, feeling tired, and foamy urine. Complications may include blood clots, infections, and high blood pressure. Causes include a number of kidney diseases such as focal segmental glomerulosclerosis, membranous nephropathy, and minimal change disease. It may also occur as a complication of diabetes or lupus. The underlying mechanism typically involves damage to the glomeruli of the kidney. Diagnosis is typically based on urine testing and sometimes a kidney biopsy. It differs from nephritic syndrome in that there are no red blood cells in the urine. Nephrotic syndrome is characterized by large amounts of proteinuria (>3.5 g per 1.73 m2 body surface area per day, or >40 mg per square meter body surface area per hour in children), hypoalbuminemia (<2.5 g/dl), hyperlipidaemia, and edema that begins in the face. Lipiduria (lipids in urine) can also occur, but is not essential for the diagnosis of nephrotic syndrome, Hyponatremia also occur with a low fractional sodium excretion. Genetic forms of nephrotic syndrome are typically resistant to steroid and other immunosuppressive treatment. Goals of therapy are to control urinary protein loss and swelling, provide good nutrition to allow the child to grow, and prevent complications. Early and aggressive treatment are used to control the disorder.

K. Minimal Change Disease

Minimal change disease (also known as MCD, minimal change glomerulopathy, and nil disease, among others) is a disease affecting the kidneys which causes a nephrotic syndrome. The clinical signs of minimal change disease are proteinuria (abnormal excretion of proteins, mainly albumin, into the urine), edema (swelling of soft tissues as a consequence of water retention), weight gain, and hypoalbuminaemia (low serum albumin). These signs are referred to collectively as nephrotic syndrome. The first clinical sign of minimal change disease is usually edema with an associated increase in weight. The swelling may be mild but patients can present with edema in the lower half of the body, periorbital edema, swelling in the scrotal/labial area and anasarca in more severe cases. In older adults, patients may also present with acute kidney injury (20-25% of affected adults) and high blood pressure. Due to the disease process, patients with minimal change disease are also at risk of blood clots and infections.

L. Membranous Nephropathy

Membranous nephropathy refers to the deposition of immune complexes on the glomerular basement membrane ((IBM) with GBM thickening. The cause is usually unknown (idiopathic), although secondary causes include drugs, infections, autoimmune disorders, and cancer. Manifestations include insidious onset of edema and heavy proteinuria with benign urinary sediment, normal renal function, and normal or elevated blood pressure. Membranous nephropathy is diagnosed by renal biopsy. Spontaneous remission is common. Treatment of patients at high risk of progression is usually with corticosteroids and cyclophosphamide or chlorambucil.

M. Postinfectious Glomerulonephritis

Acute proliferative glomerulonephritis is a disorder of the glomeruli (glomerulonephritis), or small blood vessels in the kidneys. It is a common complication of bacterial infections, typically skin infection by Streptococcus bacteria types 12, 4 and 1 (impetigo) but also after streptococcal pharyngitis, for which it is also known as postinfectious or poststreptococcal glomerulonephritis. It can be a risk factor for future albuminuria. In adults, the signs and symptoms of infection may still be present at the time when the kidney problems develop, and the terms infection-related glomerulonephritis or bacterial infection-related glomerulonephritis are also used. Acute glomerulonephritis resulted in 19,000 deaths in 2013 down from 24,000 deaths in 1990 worldwide. Acute proliferative glomerulonephritis (post-streptococcal glomerulonephritisis) is caused by an infection with streptococcus bacteria, usually three weeks after infection, usually of the pharynx or the skin, given the time required to raise antibodies and complement proteins. The infection causes blood vessels in the kidneys to develop inflammation, this hampers the renal organs ability to filter urine [citation needed] Acute proliferative glomerulonephritis most commonly occurs in children,

N. Thin Basement Membrane Disease

Thin basement membrane disease (TBMD, also known as benign familial hematuria and thin basement membrane nephropathy or TBMN) is, along with IgA nephropathy, the most common cause of hematuria without other symptoms. The only abnormal finding in this disease is a thinning of the basement membrane of the glomeruli in the kidneys. Its importance lies in the fact that it has a benign prognosis, with patients maintaining a normal kidney function throughout their lives. Most patients with thin basement membrane disease are incidentally discovered to have microscopic hematuria on urinalysis. The blood pressure, kidney function, and the urinary protein excretion are usually normal. Mild proteinuria (less than 1.5 g/day) and hypertension are seen in a small minority of patients. Frank hematuria and loin pain should prompt a search for another cause, such as kidney stones or loin pain-hematuria syndrome. Also, there are no systemic manifestations, so presence of hearing impairment or visual impairment should prompt a search for hereditary nephritis such as Alport syndrome, Some individuals with TBMD are thought to be carriers for genes that cause Alport syndrome.

O. Mesangial Proliferative Glomerulonephritis

Mesangial proliferative glomerulonephritis is a form of glomerulonephritis associated primarily with the mesangium. There is some evidence that interleukin-10 may inhibit it in an animal model. [2] It is classified as type H lupus nephritis by the World Health Organization (WHO). Mesangial cells in the renal glomerulus use endocytosis to take up and degrade circulating immunoglobulin. This normal process stimulates mesangial cell proliferation and matrix deposition. Therefore, during times of elevated circulating immunoglobulin (i.e. lupus and IgA nephropathy) one would expect to see an increased number of mesangial cells and matrix in the glomerulus. This is characteristic of nephritic syndromes.

P. Amyloidosis (Primary)

Amyloidosis is a group of diseases in which abnormal protein, known as amyloid fibrils, builds up in tissue. [4] Symptoms depend on the type and are often variable. [2] They may include diarrhea, weight loss, feeling tired, enlargement of the tongue, bleeding, numbness, feeling faint with standing, swelling of the legs, or enlargement of the spleen. [2] There are about 30 different types of amyloidosis, each due to a specific protein misfolding. [5] Some are genetic while others are acquired. [3] They are grouped into localized and systemic forms. [2] The four most common types of systemic disease are light chain (AL), inflammation (AA), dialysis (Aβ2M), and hereditary and old age (ATTR). Primary amyloidosis refers to amyloidosis in which no associated clinical condition is identified.

Q. clq Nephropathy

Clq nephropathy is a rare glomerular disease with characteristic mesangial CIq deposition noted on immunofluorescence microscopy. It is histologically defined and poorly understood. Light microscopic features are heterogeneous and comprise minimal change disease (MCD), focal segmental glomerulosclerosis (FSGS), and proliferative glomerulonephritis. Clinical presentation is also diverse, and ranges from asymptomatic hematuria or proteinuria to frank nephritic or nephrotic syndrome in both children and adults. Hypertension and renal insufficiency at the time of diagnosis are common findings. Optimal treatment is not clear and is usually guided by the underlying light microscopic lesion. Corticosteroids are the mainstay of treatment, with immunosuppressive agents reserved for steroid resistant cases. The presence of nephrotic syndrome and FSGS appear to predict adverse outcomes as opposed to favorable outcomes in those with MCD. (Devasahayam, et al., “Clq Nephropathy: The Unique Underrecognized Pathological Entity,” Analytical Cellular Pathology, vol. 2015, Article ID 490413, 5 pages, 2015. https://doi.org/10.1155/2015/490413.)

R. Anti-GBM Disease

Anti-glomerular basement membrane (GBM) disease; also known as Goodpasture's disease, is a rare condition that causes inflammation of the small blood vessels in the kidneys and lungs. The antiglomerular basement membrane (GBM) antibodies primarily attack the kidneys and lungs, although, generalized symptoms like malaise, weight loss, fatigue, fever, and chills are also common, as are joint aches and pains. 60 to 80% of those with the condition experience both lung and kidney involvement; 20-40% have kidney involvement alone, and less than 10% have lung involvement alone. Lung symptoms usually antedate kidney symptoms and usually include: coughing up blood, chest pain (in less than 50% of cases overall), cough, and shortness of breath. Kidney symptoms usually include blood in the urine, protein in the urine, unexplained swelling of limbs or face, high amounts of urea in the blood, and high blood pressure. GPS causes the abnormal production of anti-GBM antibodies, by the plasma cells of the blood. The anti-GBM antibodies attack the alveoli and glomeruli basement membranes. These antibodies bind their reactive epitopes to the basement membranes and activate the complement cascade, leading to the death of tagged cells. T cells are also implicated. It is generally considered a type II hypersensitivity reaction.

Measurement of Urine Protein Levels

Protein levels in urine can be measured using methods known in the art. Until recently, an accurate protein measurement required a 24-hour urine collection. In a 24-hour collection, the patient urinates into a container, which is kept refrigerated between trips to the bathroom. The patient is instructed to begin collecting urine after the first trip to the bathroom in the morning. Every drop of urine for the rest of the day is to be collected in the container. The next morning, the patient adds the first urination after waking and the collection is complete.

More recently, researchers have found that a single urine sample can provide the needed information. In the newer technique, the amount of albumin in the urine sample is compared with the amount of creatinine, a waste product of normal muscle breakdown. The measurement is called a urine albumin-to-creatinine ratio (UACR). A urine sample containing more than 30 milligrams of albumin for each gram of creatinine (30 mg/g) is a warning that there may be a problem. If the laboratory test exceeds 30 mg/g, another UACR test should be performed 1 to 2 weeks later. If the second test also shows high levels of protein, the person has persistent proteinuria, a sign of declining kidney function, and should have additional tests to evaluate kidney function.

Tests that measure the amount of creatinine in the blood will also show whether a subject's kidneys are removing wastes efficiently. Too much creatinine in the blood is a sign that a person has kidney damage. A physician can use the creatinine measurement to estimate how efficiently the kidneys are filtering the blood. This calculation is called the estimated glomerular filtration rate, or eGFR. Chronic kidney disease is present when the eGFR is less than 60 milliliters per minute (mL/min).

CDK5

Cyclin-dependent kinases (CDKs) are the family of protein kinases first discovered for their role in regulating the cell cycle. They are also involved in regulating transcription, mRNA processing, and the differentiation of nerve cells. They are present in all known eukaryotes, and their regulatory function in the cell cycle has been evolutionarily conserved.

Recently CDK5 has emerged as an essential kinase in sensory pathways. CDK5 is required for proper development of the brain and, to be activated, CDK5 must associate with CDK5R1 or CDK5R2. Cdk5 is involved in the processes of neuronal maturation and migration, phosphorylating the key intracellular adaptor of the reelin signaling chain. Dysregulation of this enzyme has been implicated in several neurodegenerative diseases including Alzheimer's. It is also involved in invasive cancers, apparently by reducing the activity of the actin regulatory protein caldesmon. Recent data also suggest a role for CDK5 as a regulator of differentiation, proliferation, and morphology in podocytes, which are highly specialized and terminally differentiated glomerular cells that play a vital role in renal physiology, including the prevention of proteinuria (Griffin et al., Am J Pathol. (2004) 165(4):1175-1185). CDK5 has also been demonstrated to play a role in other non-neuronal tissues (Dhavan R and Tsai L H, Nat Rev Mol Cell Biol. (2001) 2:749-759).

Accordingly, in certain embodiments, the invention provides methods for treating, or the reducing risk of developing, a disease or condition characterized by aberrant CDK5 overactivity comprising the step of administering to a subject in need thereof a therapeutically effective amount of a compound of the invention (e.g., a compound having structural formula (I)) or a pharmaceutical composition comprising said compound.

In some embodiments, the disease is or condition is a disease or condition of the kidney. In some embodiments, the disease is polycystic kidney disease.

Subjects to be Treated

In one aspect of the invention, a subject is selected on the basis that they have, or are at risk of developing, a disease or condition characterized by aberrant CDK5 overactivity, such as a disease or condition of the kidney, such as polycystic kidney disease.

Subjects that have, or are at risk of developing, a disease or condition of the kidney include those with diabetes, hypertension, or certain family backgrounds. In the United States, diabetes is the leading cause of end-stage renal disease (ESRD). In both type I and type 2 diabetes, albumin in the urine is one of the first signs of deteriorating kidney function. As kidney function declines, the amount of albumin in the urine increases. Another risk factor for developing kidney diseases is hypertension. Proteinuria in a person with high blood pressure is an indicator of declining kidney function. If the hypertension is not controlled, the person can progress to full kidney failure. African Americans are more likely than Caucasians to have high blood pressure and to develop kidney problems from it, even when their blood pressure is only mildly elevated. Other groups at risk for proteinuria are American Indians, Hispanics/Latinos, Pacific Islander Americans, older adults, and overweight subjects.

In one aspect of the invention, a subject is selected on the basis that they have, or are at risk of developing a disease or condition of the kidney. A subject that has, or is at risk of developing, a disease or condition of the kidney is one having one or more symptoms of the condition. Symptoms of proteinuria are known to those of skill in the art and include, without limitation, large amounts of protein in the urine, which may cause it to look foamy in the toilet. Loss of large amounts of protein may result in edema, where swelling in the hands, feet, abdomen, or face may occur. These are signs of large protein loss and indicate that kidney disease has progressed. Laboratory testing is the only way to find out whether protein is in a subject's urine before extensive kidney damage occurs.

The methods are effective for a variety of subjects including mammals, e.g., humans and other animals, such as laboratory animals, e.g., mice, rats, rabbits, or monkeys, or domesticated and farm animals, e.g., cats, dogs, goats, sheep, pigs, cows, or horses. In some embodiments, the subject is a mammal. In some embodiments, the subject is a human.

### EXAMPLES

The invention is further described in the following examples, which do not limit the scope of the invention described in the claims.

Reversed-phase HPLC purifications (“Prep-HPLC”) were performed on Waters C₁₈ columns, using gradient elution with mixtures of water and acetonitrile using either formic acid or ammonium bicarbonate as modifier.

### Example 1. Preparation of Intermediates

The following chemical intermediates were synthesized and are useful in the production of various compounds of the invention. It will be readily apparent to those of skill in the art that certain of the intermediates described in this Example, as well as in the compound synthesis examples that follow are also compounds within the scope of the invention.

### A. 7-chloro-1,6-naphthyridin-2-ol

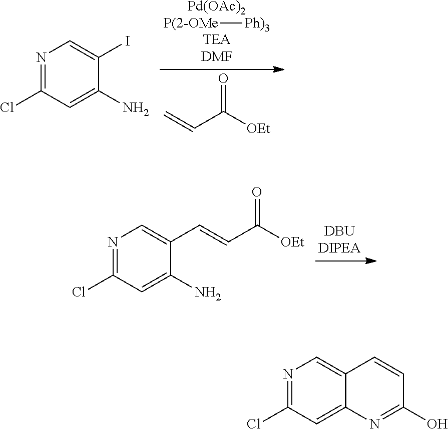

Ethyl (2E)-3-(4-amino-6-chloropyridin-3-yl)prop-2-enoate. To a stirred mixture of 2-chloro-5-iodopyridin-4-amine (350 g, 1375 mmol, 1 equiv.) and tris(2-methoxyphenyl)phosphine (8.37 g, 27.5 mmol, 0.02 equiv.) in DMF (1.5 L) were added TEA (167 g, 1651 mmol, 1.2 equiv.), Pd(OAc)₂ (9.26 g, 41.3 mmol, 0.03 equiv.) and ethyl prop-2-enoate (330.5 g, 3301 mmol, 2.4 equiv.) dropwise at room temperature under nitrogen atmosphere. The resulting mixture was stirred for 2 hours at 100° C. under nitrogen atmosphere. The reaction was monitored by LCMS. The reaction was quenched by the addition of water (9000 mL) at room temperature. The precipitated solids were collected by filtration, and washed with EtOAc (2×1000 mL). The resulting mixture was concentrated under reduced pressure to afford ethyl (2E)-3-(4-amino-6-chloropyridin-3-yl)prop-2-enoate (286 g, 92%) as a brown solid. 7-chloro-1,6-naphthyridin-2-ol. To a solution of ethyl (2E)-3-(4-amino-6-chloropyridin-3-yl)prop-2-enoate (120 g, 1 equiv.) in DIEA (2400 mL) was added DBU (161.2 g, 2 equiv.) at ambient temperature. The resulting mixture was stirred for 32 hours at 120° C. The desired product could be detected by LCMS. The mixture was allowed to cool down to room temperature. The reaction mixture was concentrated under vacuum, the residue was poured into ice/water and filtered, then collected the filter cake to afford 7-chloro-1,6-naphthyridin-2-ol (80 g, 84%) as a light brown solid. ¹H NMR (400 MHz, DMSO-d6) δ 12.11 (s, 1H), 8.70 (s, 1H), 8.01 (d, J=9.6 Hz, 1H), 7.21 (s, 1H), 6.61 (d, J=9.6 Hz, 1H)

### B. 2,7-dichloro-1,6-naphthyridine

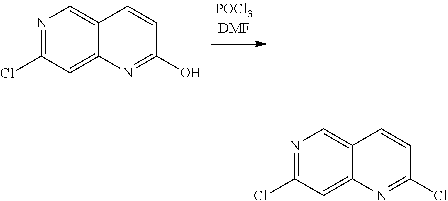 [SMILES: Oc1ccc2cnc(Cl)cc2n1 ; Clc1cc2nc(Cl)ccc2cn1]

To a stirred solution of 7-chloro-1,6-naphthyridin-2-ol (16 g, 89 mmol, 1 equiv.) in phosphorus oxychloride (50 mL) was added DMF (0.1 mL) in portions at room temperature under nitrogen atmosphere. The resulting mixture was stirred for 8 hours at 110° C. under nitrogen atmosphere. The reaction was monitored by LCMS. The resulting mixture was concentrated under reduced pressure. The resulting mixture was diluted with water (250 mL) and extracted with DCM (2×250 mL). The combined organic layers were washed with brine (1×200 mL) and dried over anhydrous Na₂SO₄. After filtration, the filtrate was concentrated under reduced pressure. The residue was purified by silica gel column chromatography, eluting with PE/EtOAc (10:1 to 3:1) to afford 2,7-dichloro-1,6-naphthyridine (10.06 g, 57%) as a white solid. ¹H NMR (400 MHz, Chloroform-d) δ 9.10 (s, 1H), 8.26 (d, J=8.6 Hz, 1H), 7.92 (s, 1H), 7.53 (d, J=8.6 Hz, 1H)

### C. 2-bromo-7-chloro-1,6-naphthyridine

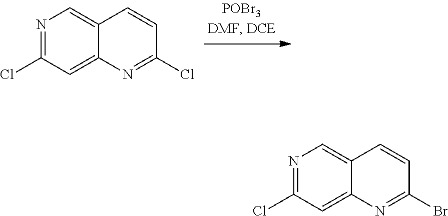 [SMILES: Clc1cc2nc(Cl)ccc2cn1 ; Clc1cc2nc(Br)ccc2cn1]

To a stirred solution of 7-chloro-1,6-naphthyridin-2-ol (10 g, 1 equiv.) in DCE (100 mL) was added POBr₃ (100 g) and DMF (405 mL, 5.54 mmol, 0.1 equiv.) at room temperature. The resulting mixture was stirred for 16 hours at 80° C. The reaction was monitored by LCMS. The mixture was allowed to cool down to room temperature. The resulting mixture was diluted with DCM (400 mL), poured into ice water and extracted with DCM (2×400 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EtOAc (10:1 to 1:1) to afford 2-bromo-7-chloro-1,6-naphthyridine (6 g, 45%) as an off-white solid. ¹H NMR (400 MHz, DMSO-d6) δ 9.34 (s, 1H), 8.63-8.51 (m, 1H), 8.06 (s, 1H), 7.91 (d, J=8.6 Hz, 1H)

### D. 7-chloro-1,6-naphthyridine-2-thiol

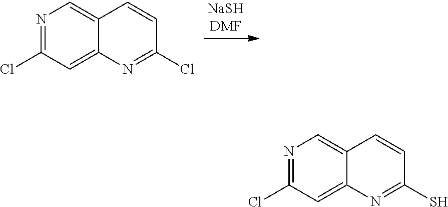 [SMILES: Clc1cc2nc(Cl)ccc2cn1 ; Sc1ccc2cnc(Cl)cc2n1]

To a stirred solution of 2,7-dichloro-1,6-naphthyridine (1000 mg, 5.024 mmol, 1 equiv.) in DMF (20 mL) was added NaSH (1408.41 mg, 25.122 mmol, 5 equiv.) in portions at under nitrogen atmosphere. The resulting mixture was stirred for 16 hours at 110° C. under nitrogen atmosphere. The reaction was monitored by LCMS. The mixture was allowed to cool down to room temperature. The resulting mixture was filtered, the filter cake was washed with ACN (3×10 mL). The filtrate was concentrated under reduced pressure. The residue was purified by reverse phase flash chromatography with the following conditions: Column: Spherical C18, 20-40 um, 330 g; Mobile Phase A: Water (plus 5 mM NH₄NO₃); Mobile Phase B: ACN; Flow rate: 80 mL/min; Gradient: 5%-5% B, 10 min, 25% B-60% B gradient in 25 min; Detector: 245 nm and the desired product were collected at 33% B. Concentrated under reduced pressure to afford 7-chloro-1,6-naphthyridine-2-thiol (650 mg, 66%) as an orange solid. ¹H NMR (400 MHz, DMSO-d6) δ 13.85-13.82 (brs, 1H), 8.85 (s, 1H), 7.90 (d, J=9.2 Hz, 1H), 7.44 (s, 1H), 7.31 (d, J=9.2 Hz, 1H).

### E. Tert-butyl 4-(7-chloro-1,6-naphthyridine-2-carbonyl)piperidine-1-carboxylate

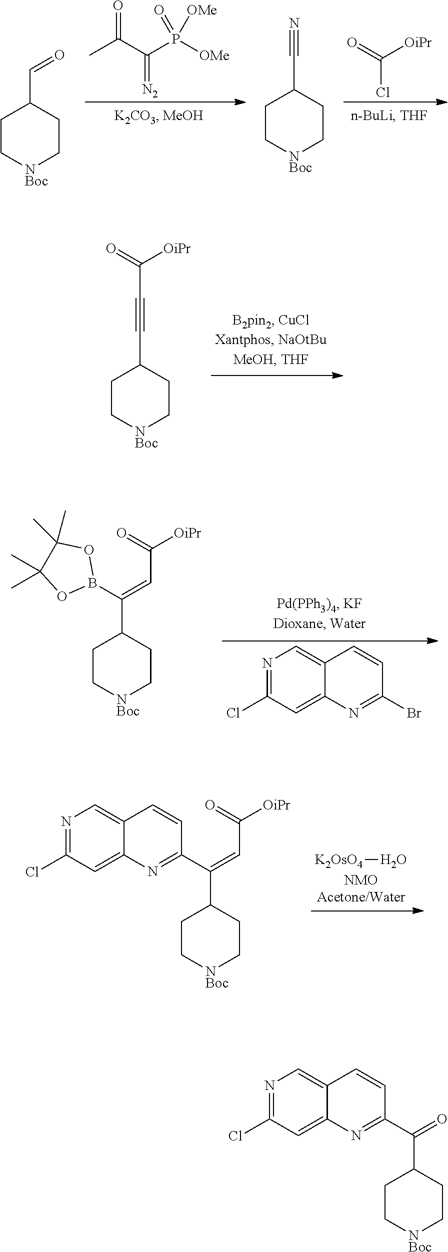

Tert-butyl 4-ethynylpiperidine-1-carboxylate. To a stirred solution of tert-butyl 4-formylpiperidine-1-carboxylate (100 g, 469 mmol, 1 equiv.) and dimethyl-1-diazo-2-oxopropylphosphonate (99.08 g, 515.8 mmol, 1.1 equiv.) in MeOH (1000 mL) was added K₂CO₃ (97.20 g, 703 mmol, 1.5 equiv.) in portions at room temperature under nitrogen atmosphere. The resulting mixture was stirred for 16 hours at room temperature under nitrogen atmosphere. The reaction was monitored by TLC. The resulting mixture was filtered, the filter cake was washed with ethanol (2×150 mL). The filtrate was concentrated under reduced pressure. The residue was purified by silica gel column chromatography, eluted with PE/EtOAc (100:1 to 20:1) to afford tert-butyl 4-ethynylpiperidine-1-carboxylate (97 g, 98%) as a white solid.

Tert-butyl 4-(3-isopropoxy-3-oxoprop-1-yn-1-yl)piperidine-1-carboxylate. To a stirred solution of tert-butyl 4-ethynylpiperidine-1-carboxylate (100 g, 477.808 mmol, 1 equiv.) in THF (1200 mL) was added n-BuLi in hexanes (49.51 mL, 772.9 mmol, 1.1 equiv.) in portions at −78° C. under nitrogen atmosphere. The resulting mixture was stirred for 1 hour at −78° C. under nitrogen atmosphere. The mixture was charged dropwise isopropyl chloroformate (64.41 g, 525.589 mmol, 1.1 equiv.) in THF (100 mL) then stirred for 3 hours at −78° C. under nitrogen atmosphere. The reaction was monitored by TLC. The reaction was quenched with sat. NH₄C₁ (aq.) at −78° C. The resulting mixture was extracted with EtOAc (3×400 mL). The combined organic layers were washed with brine (1×500 mL), dried over anhydrous Na₂SO₄. After filtration, the filtrate was concentrated under reduced pressure. The residue was purified by silica gel column chromatography, eluted with PE/EtOAc (50:1 to 30:1) to afford tert-butyl 4-(3-isopropoxy-3-oxoprop-1-yn-1-yl)piperidine-1-carboxylate (120 g, 85%) as a white solid.

Tert-butyl 4-[3-isopropoxy-3-oxo-1-(4,4,5,5-tetramethyl-1,3,2-dioxaborolan-2-yl)prop-1-en-1-yl]piperidine-1-carboxylate. To a stirred solution of tert-butyl 4-(3-isopropoxy-3-oxoprop-1-yn-1-yl)piperidine-1-carboxylate (120 g, 406 mmol, 1 equiv.), CuCl (1206.57 mg, 12.188 mmol, 0.03 equiv.) and XantPhos (7052.04 mg, 12.188 mmol, 0.03 equiv.) in THF (1200 mL) was added t-BuONa (2340.04 mg, 24.375 mmol, 0.06 equiv.) in portions at room temperature under nitrogen atmosphere. The resulting mixture was stirred for 0.5 hours at room temperature under nitrogen atmosphere. To the mixture was added bis(pinacolato)diboron (134.11 g, 528.135 mmol, 1.3 equiv.) and MeOH (26034.54 mg, 812.515 mmol, 2 equiv.). The resulting mixture was stirred for 16 hours at room temperature under nitrogen atmosphere. The reaction was monitored by TLC. The reaction was quenched with sat. NH₄C₁ (aq.) at room temperature. The resulting mixture was extracted with EtOAc (2×1500 mL). The combined organic layers were washed with brine (1×1500 mL), dried over anhydrous Na₂SO₄. After filtration, the filtrate was concentrated under reduced pressure. The residue was purified by silica gel column chromatography, eluted with PE/EtOAc (30:1 to 20:1) to afford tert-butyl 4-[3-isopropoxy-3-oxo-1-(4,4,5,5-tetramethyl-1,3,2-dioxaborolan-2-yl)prop-1-en-1-yl]piperidine-1-carboxylate (170 g, 99%) as a white solid.

Tert-butyl 4-[1-(7-chloro-1,6-naphthyridin-2-yl)-3-isopropoxy-3-oxoprop-1-en-1-yl]piperidine-1-carboxylate. tert-butyl 4-[3-isopropoxy-3-oxo-1-(4,4,5,5-tetramethyl-1,3,2-dioxaborolan-2-yl)prop-1-en-1-yl]piperidine-1-carboxylate (75 g, 1.50 equiv.), 2-bromo-7-chloro-1,6-naphthyridine (45 g, 1 equiv.) and KF (11.91 g, 10 equiv.) in 1,4-dioxane (1000 mL) and H₂O (200 mL), was added Pd(PPh3)₄ (7.1 g, 0.30 equiv.) at room temperature under argon atmosphere. The resulting mixture was stirred for 75° C. under argon atmosphere for 24 hours. The reaction was quenched by the addition of brine (600 mL) The aqueous layer was extracted with EtOAc (3×600 mL). The collect organic layer was washed with brine (3×500 mL). The was dried anhydrous Na₂SO₄ and concentrated under reduced pressure. The residue was purified by silica gel column chromatography, eluted with PE: EtOAc (20:1-5:1) to afford tert-butyl 4-[1-(7-chloro-1,6-naphthyridin-2-yl)-3-isopropoxy-3-oxoprop-1-en-1-yl]piperidine-1-carboxylate (80 g, 71%) as a yellow solid.

Tert-butyl 4-(7-chloro-1,6-naphthyridine-2-carbonyl)piperidine-1-carboxylate (Intermediate E). To a stirred solution of tert-butyl 4-[1-(7-chloro-1,6-naphthyridin-2-yl)-3-isopropoxy-3-oxoprop-1-en-1-yl]piperidine-1-carboxylate (60.0 g, 130 mmol) in acetone (1.20 L) and water (0.40 L) were added potassium osmate(VI) dehydrate (14.4 g, 39.1 mmol) and N-methylmorpholine-N-oxide (91.7 g, 782 mmol) at ambient temperature. The mixture was stirred at ambient temperature for 36 h. The reaction mixture was quenched by aqueous sodium thiosulfate (300 mL, saturated) at 0° C. and extracted with ethyl acetate (3×500 mL). The combined organic fractions was washed with brine (3×300 mL), dried with anhydrous sodium sulfate and filtered. The filtrate was concentrated under reduced pressure. The residue was purified by silica gel column chromatography, eluted with 1-20% of ethyl acetate in petroleum ether to afford the title compound (36.0 g, 73%) as a yellow solid. ¹H NMR (400 MHz, DMSO-d6) δ 7.82 (d, J=8.2 Hz, 2H), 7.51 (d, J=8.1 Hz, 2H), 4.13 (s, 2H), 2.44 (s, 3H), 1.37-1.29 (m, 2H), 1.13-1.05 (m, 2H).

### F. 7-chloro-2-(1-methylpiperidine-4-carbonyl)-1,6-naphthyridine

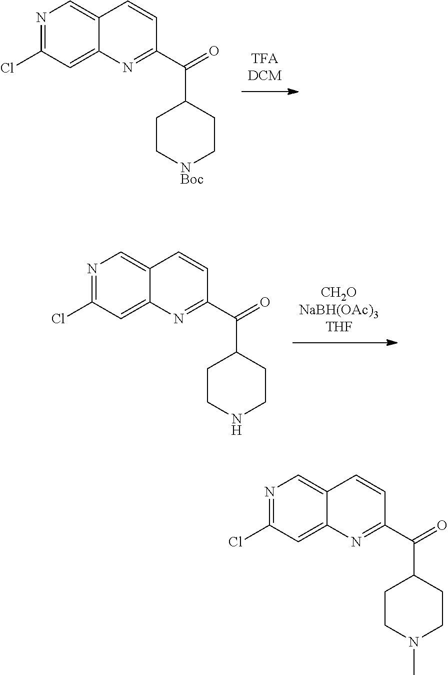 [SMILES: CC(C)(C)OC(=O)N1CCC(C(=O)c2ccc3cnc(Cl)cc3n2)CC1 ; O=C(c1ccc2cnc(Cl)cc2n1)C1CCNCC1 ; CN1CCC(C(=O)c2ccc3cnc(Cl)cc3n2)CC1]

7-chloro-2-(piperidine-4-carbonyl)-1,6-naphthyridine. To a stirred solution of tert-butyl 4-(7-chloro-1,6-naphthyridine-2-carbonyl)piperidine-1-carboxylate (3750 mg, 10 mmol, 1 equiv.) in DCM (100 mL) was added TFA (100 mL) in portions at 0° C. The resulting mixture was stirred for 1 hour at 0° C. The reaction was monitored by LCMS. The resulting mixture was extracted with DCM (3×10 mL). The combined organic layers were washed with DCM (3×10 mL) dried over anhydrous Na₂SO₄. After filtration, the filtrate was concentrated under reduced pressure to afford 7-chloro-2-(piperidine-4-carbonyl)-1,6-naphthyridine (2600 mg, 95%) as a brown solid.

7-chloro-2-(1-methylpiperidine-4-carbonyl)-1,6-naphthyridine (Intermediate F). To a stirred solution of 7-chloro-2-(piperidine-4-carbonyl)-1,6-naphthyridine (140 mg, 0.508 mmol, 1 equiv.) and NaBH(OAc)₃ (161.41 mg, 0.762 mmol, 1.50 equiv.) in THF (3 mL) was added HCHO (30.49 mg, 1.015 mmol, 2 equiv.) in portions at room temperature. The resulting mixture was stirred for 1 hour at room temperature. The reaction was monitored by LCMS. The resulting mixture was concentrated under reduced pressure. The residue was purified by silica gel column chromatography, eluted with DCM/MeOH (10:1) to afford 7-chloro-2-(1-methylpiperidine-4-carbonyl)-1,6-naphthyridine (130 mg, 88) as a brown yellow solid. 1H NMR (400 MHz, DMSO-d6) δ 9.48 (s, 1H), 8.87 (d, J=8.6 Hz, 1H), 8.21 (d, J=8.4 Hz, 1H), 8.19 (s, 1H), 4.13-4.07 (m, 1H), 3.45-3.42 (m, 2H), 3.20-3.16 (m, 2H), 2.77 (s, 3H), 2.20-2.16 (m, 2H), 1.86-1.82 (m, 2H).

### G. 2-fluoro-4-(pyrazol-1-yl)aniline

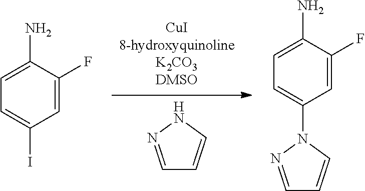 [SMILES: Nc1ccc(I)cc1F ; c1cn[nH]c1 ; Nc1ccc(-n2cccn2)cc1F]

To a stirred mixture of 2-fluoro-4-iodoaniline (10 g, 42.191 mmol, 1 equiv.) and pyrazole (4.31 g, 63.3 mmol, 1.50 equiv.) in DMSO (100 mL) were added 8-hydroxyquinoline (0.92 g, 6.3 mmol, 0.15 equiv.), K₂CO₃ (8.75 g, 63.3 mmol, 1.50 equiv.) and CuI (1.21 g, 6.35 mmol, 0.15 equiv.) in portions at room temperature. The resulting mixture was stirred for 16 hours at 120° C. under nitrogen atmosphere. The reaction was monitored by LCMS. The resulting mixture was concentrated under reduced pressure. The mixture was allowed to cool down to room temperature. The resulting mixture were washed with hartshorn (3×200 mL). The resulting mixture was extracted with EtOAc (4×300 mL). The combined organic layers were washed with brine (2×300 mL), dried over anhydrous Na₂SO₄. After filtration, the filtrate was concentrated under reduced pressure. The residue was purified by silica gel column chromatography, eluted with PE/EtOAc (20:1 to 4:1) to afford 2-fluoro-4-(pyrazol-1-yl)aniline (7.0 g, 94%) as a red oil.

1H NMR (400 MHz, DMSO-d6) δ 8.30 (d, J=2.4 Hz, 1H), 7.64 (d, J=1.8 Hz, 1H), 7.50 (dd, J=12.6, 2.5 Hz, 1H), 7.37-7.35 (m, 1H), 6.86-6.82 (m, 1H), 6.47-6.45 (m, 1H), 5.27-5.25 (brs, 2H).

Preparation of intermediates shown in the table below was achieved by the methods and protocols described for the synthesis of intermediate G, starting with the appropriate materials.

| | | Compound | |
|---|---|---|---|
| Intermediate | Structure | Name | NMR |
| H | 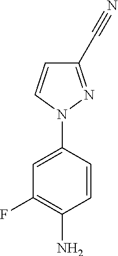 [SMILES: N#Cc1ccn(-c2ccc(N)c(F)c2)n1] | 1-(4-amino-3- fluorophenyl)- 1H-pyrazole-3- carbonitrile | 1H NMR (400 MHz, CD₃OD) δ 8.25 (d, J = 1.2 Hz, 1H), 7.46 (d, J = 12.0 Hz, 1H), 7.38-7.31 (m, 1H), 6.94 (d, J = 1.2 Hz, 1H), 6.81-6.76 (m, 1H). |
| I | 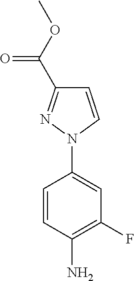 [SMILES: COC(=O)c1ccn(-c2ccc(N)c(F)c2)n1] | methyl 1-(4- amino-3- fluorophenyl)- 1H-pyrazole-3- carboxylate | 1H NMR (400 MHz, DMSO-d6) δ 8.43 (d, J = 2.5 Hz, 1H), 7.57 (dd, J = 12.4, 2.5 Hz, 1H), 7.46- 7.39 (m, 1H), 6.95 (d, J = 2.5 Hz, 1H), 6.92-6.83 (m, 1H), 5.44-5.52 (brs, 2H), 3.84 (s, 3H). |
| J | 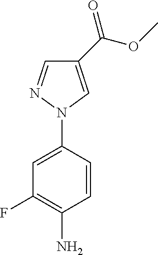 [SMILES: COC(=O)c1cnn(-c2ccc(N)c(F)c2)c1] | methyl 1-(4- amino-3- fluoropheny))- 1H-pyrazole-4- carboxylate | 1H NMR (400 MHz, DMSO-d6) δ 8.91 (s, 1H), 8.07 (s, 1H), 7.61 (dd, J = 12.5, 2.5 Hz, 1H), 7.49- 7.43 (m, 1H), 6.88-6.85 (m, 1H), 5.43-5.39 (brs, 2H), 3.80 (s, 3H). |
| K | 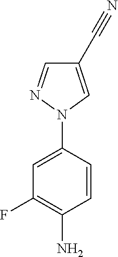 [SMILES: N#Cc1cnn(-c2ccc(N)c(F)c2)c1] | 1-(4-amino-3- fluoropheny))- 1H-pyrazole-4- carbonitrile | 1H NMR (400 MHz, DMSO-d6) δ 9.11 (s, 1H), 8.27 (s, 1H), 7.54 (dd, J = 12.3, 2.5 Hz, 1H), 7.39- 7.36 (m, 1H), 6.89-6.86 (m, 1H), 5.50-5.46 (brs, 2H). |
| L | 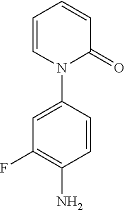 [SMILES: Nc1ccc(-n2ccccc2=O)cc1F] | 1-(4-amino-3- fluorophenyl) pyridin-2(1H)-one | 1H NMR (400 MHz, DMSO-d6) δ 7.57 (dd, J = 6.9, 2.1 Hz, 1H), 7.49-7.45 (m, 1H), 7.11 (dd, J = 12.1, 2.3 Hz, 1H), 6.89-6.82 (m, 2H), 6.47-6.40 (m, 1H), 6.29-6.23 (m, 1H), 5.43-5.40 (brs, 2H). |
| M | 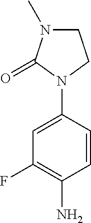 | 1-(4-amino-3- fluoropheny1)-3- methylimidazolidin- 2-one | 1H NMR (400 MHz, DMSO-d6) δ 7.38 (dd, J = 14.1, 2.4 Hz, 1H), 6.95-6.91 (m, 1H), 6.75-6.70 (m, 1H), 4.82-4.49 (brs, 2H), 3.72- 3.63 (m, 2H), 3.42-3.33 (m, 2H), 2.73 (s, 3H). |

### N. [1-(4-amino-3-fluorophenyl)pyrazol-3-yl]methanol

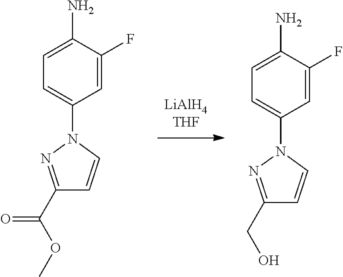 [SMILES: COC(=O)c1ccn(-c2ccc(N)c(F)c2)n1 ; Nc1ccc(-n2ccc(CO)n2)cc1F]

To a stirred solution of methyl 1-(4-amino-3-fluorophenyl)pyrazole-3-carboxylate (9 g, 38.3 mmol, 1 equiv.) in THF (50 mL) was added LiAlH₄ (1.74 g, 45.9 mmol, 1.2 equiv.) in portions at 0° C. under nitrogen atmosphere. The resulting mixture was stirred for 1 hour at room temperature under nitrogen atmosphere. The reaction was quenched with water (1.7 mL) at room temperature, added 15% NaOH (aq.) (1.7 mL) and water (5.1 mL). The resulting mixture was filtered, the filter cake was washed with EtOAc (3×50 mL). The filtrate was concentrated under reduced pressure. The residue was purified by silica gel column chromatography, eluted with DCM/MeOH (50:1 to 10:1) to afford [1-(4-amino-3-fluorophenyl)pyrazol-3-yl]methanol (7 g, 88%) as a white solid. 1H NMR (400 MHz, DMSO-d6) δ 8.20 (d, J=2.4 Hz, 1H), 7.46 (dd, J=12.7, 2.4 Hz, 1H), 7.38-7.27 (m, 1H), 6.86-6.81 (m, 1H), 6.40 (d, J=2.4 Hz, 1H), 5.20-5.19 (brs, 2H), 5.09 (t, J=5.8 Hz, 1H), 4.47 (d, J=5.8 Hz, 2H).

Preparation of intermediates shown in the table below follows the methods and protocols as described for the synthesis of intermediate N, starting with the side product formed in the synthesis of intermediate I:

| Intermediate | Structure | Compound Name | NMR |
|---|---|---|---|
| O | 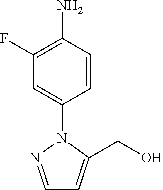 [SMILES: Nc1ccc(-n2nccc2CO)cc1F] | (1-(4-amino-3- fluorophenyl)-1H- pyrazol-5- yl)methanol | 1H NMR (400 MHz, DMSO-d6) δ 7.53 (d, J = 1.8 Hz, 1H), 7.28 (dd, J = 12.4, 2.4 Hz, 1H), 7.15-7.09 (m, 1H), 6.86-6.82 (m, 1H), 6.38 (d, J = 1.8 Hz, 1H), 5.39-5.36 (m, 3H), 4.44 (d, J = 5.4 Hz, 2H). |

### P. 2-(1-(4-amino-3-fluorophenyl)-1H-pyrazol-3-yl)propan-2-ol

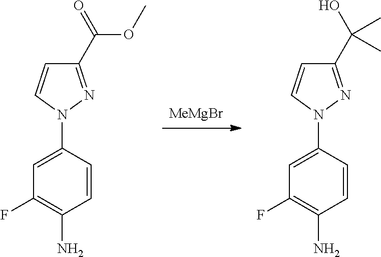 [SMILES: "MeMgBr" ; COC(=O)c1ccn(-c2ccc(N)c(F)c2)n1 ; CC(C)(O)c1ccn(-c2ccc(N)c(F)c2)n1]

To a stirred mixture of methyl 1-(4-amino-3-fluorophenyl)pyrazole-3-carboxylate (300 mg, 1.275 mmol, 1 equiv.) in THF (5 mL) was added bromo(methyl)magnesium (3.40 mL, 10.200 mmol, 8 equiv, 3 M in ether) dropwise at −78° C. under nitrogen atmosphere. The resulting mixture was stirred for 4 hours at room temperature under nitrogen atmosphere. The reaction was quenched with sat. NH₄C₁ (aq.) (5 mL) at 0° C. The resulting mixture was diluted with water (20 mL) and extracted with EtOAc (3×30 mL). The combined organic layers were washed with brine (3×30 mL), dried over anhydrous Na₂SO₄. After filtration, the filtrate was concentrated under reduced pressure. The residue was purified by Prep-TLC (PE/EtOAc 2:1) to afford 2-[1-(4-amino-3-fluorophenyl)pyrazol-3-yl]propan-2-ol (100 mg, 33%) as an off-white solid. 1H NMR (400 MHz, DMSO-d6) δ 8.14 (d, J=2.4 Hz, 1H), 7.57 (dd, J=11.5, 3.0 Hz, 1H), 7.35-7.29 (m, 1H), 6.93-6.78 (m, 1H), 6.40 (d, J=2.4 Hz, 1H), 5.21-5.17 (brs, 2H), 4.96-4.93 (brs, 1H), 1.46 (s, 6H).

### Q. 2-fluoro-4-(morpholin-4-ylmethyl)aniline

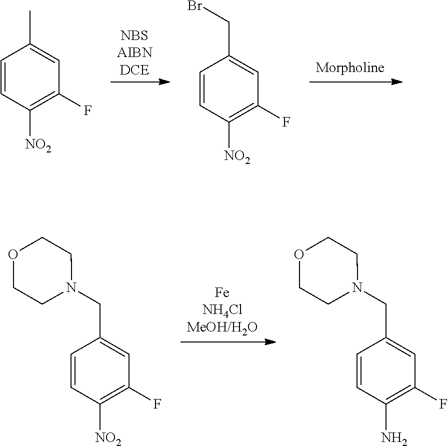

4-(bromomethyl)-2-fluoro-1-nitrobenzene. To a stirred solution of 2-fluoro-4-methyl-1-nitrobenzene (5 g, 32.231 mmol, 1 equiv.) in DCE (41.50 g, 13.13 equiv.) was added NBS (100 mg, 2 equiv.) and AIBN (0.64 g, 3.868 mmol, 0.12 equiv.) in portions at 80° C. under nitrogen atmosphere. The crude product was used as-is.

4-[(3-fluoro-4-nitrophenyl)methyl]morpholine. To a stirred solution of 4-(bromomethyl)-2-fluoro-1-nitrobenzene (7 g, 29.911 mmol, 1 equiv.) and morpholine (7.82 g, 89.734 mmol, 3 equiv.) in DCE (41.5 g) at room temperature under nitrogen atmosphere. Desired product could be detected by LCMS. The residue was acidified/basified/neutralized to pH 6 with HCl (aq.). The residue was purified by reverse phase flash chromatography with the following conditions: Column: Spherical C18, 20-40 um, 330 g; Mobile Phase A: Water (plus 5 mM FA); Mobile Phase B: ACN; Flow rate: 80 mL/min; Gradient: 5%-5% B, 10 min, 33% B-45% B gradient in 20 min; Detector: 220 nm. The fractions containing the desired product were collected at 40% B and concentrated under reduced pressure to afford 4-[(3-fluoro-4-nitrophenyl)methyl]morpholine (1.2 g, 17%) as a yellow solid.

2-fluoro-4-(morpholin-4-ylmethyl)aniline (Intermediate Q). To a stirred mixture of 4-[(3-fluoro-4-nitrophenyl)methyl]morpholine (0.40 g, 1.67 mmol) and ferrous powder (0.65 g, 11.7 mmol) in methanol (12.0 mL) and water (1.20 mL) was added ammonium chloride (0.89 g, 16.7 mmol) at ambient temperature. The reaction mixture was stirred at 65° C. for 0.5 hours. After cooling down to amibient temperature, the mixture was concentrated under reduced pressure and the residue was purified by silica gel column chromatography, eluting with 1-8% of methanol in dichloromethane to afford the title compound (0.28 g, 80%) as a yellow solid. 1H NMR (400 MHz, DMSO-d6) δ 6.97-6.93 (m, 1H), 6.86-6.83 (m, 1H), 6.75-6.70 (m, 1H), 5.13-5.09 (brs, 2H), 3.63-3.59 (m, 4H), 2.46-2.40 (m, 4H).

### R. 6-(4-amino-3-fluorophenyl)-1-methylpyridin-2-one

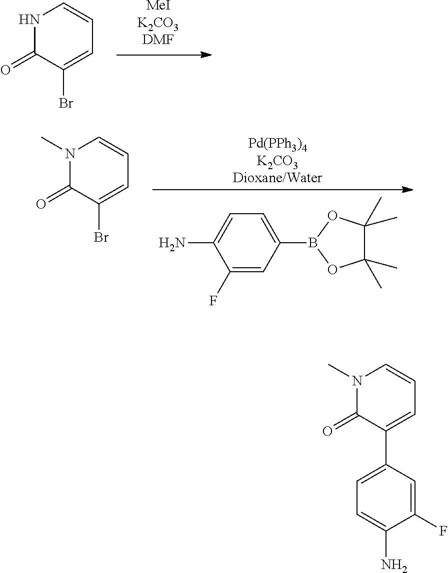

3-bromo-1-methylpyridin-2-one. To a stirred mixture of 3-bromo-1H-pyridin-2-one (10 g, 57.472 mmol, 1 equiv.) and K₂CO₃ (11.91 g, 86.176 mmol, 1.50 equiv.) in DMF (100 mL) was added CH₃I (12.24 g, 86.208 mmol, 1.5 equiv.) dropwise at room temperature. The resulting mixture was stirred for 2 hours at 60° C. The reaction was monitored by LCMS. The resulting mixture was extracted with EtOAc (8×500 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EtOAc (4/1 to 1/1) to afford 3-bromo-1-methylpyridin-2-one (8 g, 74) as a yellow oil. 6-(4-amino-3-fluorophenyl)-1-methylpyridin-2-one (Intermediate R). To a stirred mixture of 2-fluoro-4-(4,4,5,5-tetramethyl-1,3,2-dioxaborolan-2-yl)aniline (1.27 g, 05 mmol, 1 equiv.) and

6-bromo-1-methylpyridin-2-one (1.51 g, 08 mmol, 1.50 equiv.) in 1,4-dioxane (90 mL) and H₂O (30 mL) were added Pd(PPh₃)₄ (0.37 g, 00 mmol, 0.06 equiv.) and K₂CO₃ (1.48 g, 0.011 mmol, 2 equiv.) in portions under nitrogen atmosphere. The resulting mixture was stirred for 2 hours at 80° C. under nitrogen atmosphere. The reaction was monitored by LCMS. The mixture was allowed to cool down to room temperature. The resulting mixture was diluted with water (500 mL). The resulting mixture was extracted with EtOAc (3×150 mL). The combined organic layers were washed with brine (2×100 mL), dried over anhydrous Na₂SO₄. After filtration, the filtrate was concentrated under reduced pressure. The residue was purified by silica gel column chromatography, eluted with DCM/MeOH (50:1 to 20:1) to afford 6-(4-amino-3-fluorophenyl)-1-methylpyridin-2-one (856.8 mg, 73%) as a light yellow solid. 41 NMR (400 MHz, DMSO-d6) δ 7.70-7.55 (m, 1H), 7.57-7.53 (m, 1H), 7.55-7.47 (m, 1H), 7.30-7.27 (m, 1H), 6.79-6.76 (m, 1H), 6.29-6.25 (m, 1H), 5.27-5.23 (brs, 2H), 3.49 (s, 3H).

Preparation of intermediates shown in the table below follows the methods and protocols as described for the synthesis of intermediate R, starting with the appropriate materials.

| Intermediate | Structure | Compound Name | NMR |
|---|---|---|---|
| S | 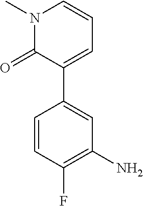 [SMILES: Cn1cccc(-c2ccc(F)c(N)c2)c1=O] | 3-(3-amino-4- fluorophenyl)-1- methylpyridin- 2(1H)-one | 1H NMR (400 MHz, DMSO-d6) δ 7.70 (d, J = 6.7 Hz, 1H), 7.50-7.46 (m, 1H), 7.15 (d, J = 9.0 Hz, 1H), 6.97 (dd, J = 11.4, 8.5 Hz, 1H), 6.80-6.76 (m, IH), 6.30-6.27 (m, 1H), 5.10-5.07 (brs, 2H), 3.49 (s, 3H). |
| T | 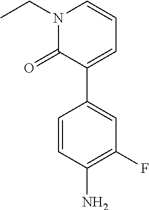 [SMILES: CCn1cccc(-c2ccc(N)c(F)c2)c1=O] | 3-(4-amino-3- fluorophenyl)-1- ethylpyridin- 2(1H)-one | 1H NMR (400 MHz, DMSO-d6) δ 7.64 (d, J = 6.7, 1H), 7.56-7.50 (m, 2H), 7.27 (dd, J = 8.3, 2.0 Hz, 1H), 6.77-6.75 (m, 1H), 6.30-6.27 (m, 1H), 5.28-5.25 (brs, 2H), 3.97 (q, J = 7.1 Hz, 2H), 1.24 (t, J = 7.1 Hz, 3H). |
| U | 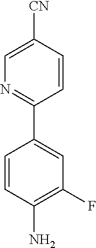 [SMILES: N#Cc1ccc(-c2ccc(N)c(F)c2)nc1] | 6-(4-amino-3- fluorophenyl) nicotinonitrile | 1H NMR (400 MHz, DMSO-d6) δ 8.94 (d, J = 2.1, 1H), 8.22 (dd, J = 8.5, 2.3 Hz, 1H), 8.01 (d, J = 8.5 Hz, 1H), 7.86 (dd, J = 13.3, 2.0 Hz, 1H), 7.83-7.70 (m, 1H), 6.88-6.82 (m, 1H), 5.86-5.82 (brs, 2H). |
| V | 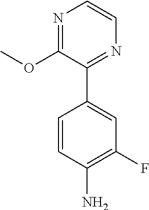 [SMILES: COc1nccnc1-c1ccc(N)c(F)c1] | 2-fluoro-4-(3- methoxypyrazin-2- yl)aniline | 1H NMR (400 MHz, DMSO-d6) δ 8.22 (d, J = 2.4, 1H), 8.06 (d, J = 2.4 Hz, 1H), 7.84-7.75 (m, 2H), 6.86-6.78 (m, 1H), 5.63-5.60 (brs, 2H), 4.00 (s, 3H). |

### W. 5-(4-amino-3-fluorophenyl)-1-methylpyridin-2-one

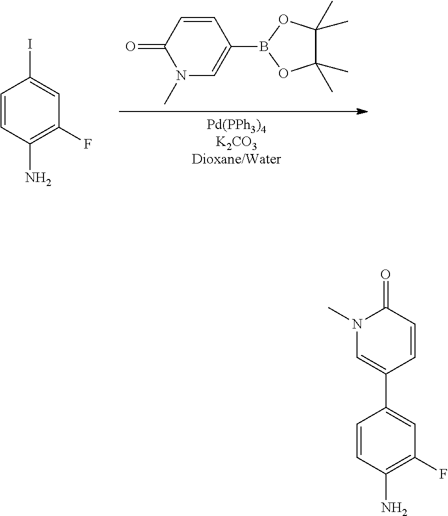 [SMILES: Nc1ccc(I)cc1F ; Cn1cc(B2OC(C)(C)C(C)(C)O2)ccc1=O ; "Pd(PPh3)4K2CO3Dioxane/Water" ; Cn1cc(-c2ccc(N)c(F)c2)ccc1=O]

To a solution of 2-fluoro-4-iodoaniline (500 mg, 2.110 mmol, 1 equiv.) and 1-methyl-5-(4,4,5,5-tetramethyl-1,3,2-dioxaborolan-2-yl)pyridin-2-one (743.91 mg, 3.164 mmol, 1.50 equiv.) in dioxane (10 mL) and H₂O (2 mL) were added K₂CO₃ (728.88 mg, 5.274 mmol, 2.50 equiv.) and Pd(PPh₃)₄ (243.77 mg, 0.211 mmol, 0.10 equiv.). After stirring for 2 hours at 80° C. under nitrogen atmosphere, the resulting mixture was cooled and concentrated under reduced pressure. The residue was purified by silica gel column chromatography, eluted with PE/EtOAc (10:1 to 1:1) to afford 5-(4-amino-3-fluorophenyl)-1-methylpyridin-2-one (400 mg, 87%) as a light yellow solid. 1H NMR (400 MHz, CD₃OD) δ 7.87 (d, J=2.6 Hz, 1H), 7.81 (dd, J=9.3, 2.7 Hz, 1H), 7.19 (dd, J=12.6, 2.1 Hz, 1H), 7.13-7.09 (m, 1H), 6.91-6.87 (m, 1H), 6.62 (d, J=9.3 Hz, 1H), 3.64 (s, 3H).

Preparation of intermediates shown in the table below follows the methods and protocols as described for the synthesis of intermediate W, starting with the appropriate materials.

| Intermediate | Structure | Compound Name | NMR |
|---|---|---|---|
| X | 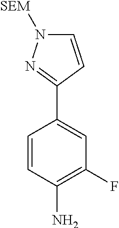 [SMILES: C[Si](C)(C)CCOCn1ccc(-c2ccc(N)c(F)c2)n1] | 2-fluoro-4-(1-((2- (trimethylsilyl) ethoxy)methyl)- 1H-pyrazol-3- yl)aniline | 1H NMR (400 MHz, DMSO-d6) δ 7.50 (d, J = 1.8 Hz, 1H), 7.30 (dd, J = 12.7, 2.0 Hz, 1H), 7.18-7.12 (m, 1H), 6.87-6.81 (m, 1H), 6.40 (d, J = 1.8 Hz, 1H), 5.49-5.45 (brs, 2H), 5.37 (s, 2H), 3.64 (t, J = 8.5 Hz, 2H), 0.86 (t, J = 8.5 Hz, 2H), −0.04 (s, 9H). |
| Y | 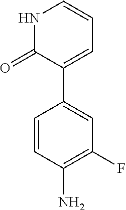 [SMILES: Nc1ccc(-c2ccc[nH]c2=O)cc1F] | 3-(4-amino-3- fluorophenyl) pyridin-2(1H)- one | 1H NMR (400 MHz, DMSO-d6) δ 7.60-7.56 (m, 2H), 7.38-7.26 (m, 2H), 6.77-6.73 (m, 1H), 6.27-6.21 (m, 1H), 5.27-5.23 (brs, 2H). |
| Z | 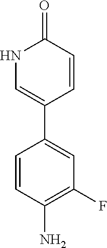 [SMILES: Nc1ccc(-c2ccc(=O)[nH]c2)cc1F] | 5-(4-amino-3- fluorophenyl) pyridin-2(1H)- one | 1H NMR (400 MHz, DMSO-d6) δ 11.70-11.67 (brs, 1H), 7.74 (dd, J = 7.4, 2.8 Hz, 1H), 7.56 (d, J = 2.8 Hz, 1H), 7.24 (dd, J = 13.1, 2.1 Hz, 1H), 7.10-7.06 (m, 1H), 6.80-6.77 (m, 1H), 6.37 (d, J = 9.5 Hz, 1H), 5.20- 5.16 (brs, 2H). |
| AA | 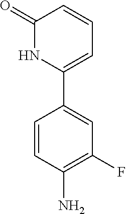 [SMILES: Nc1ccc(-c2cccc(=O)[nH]2)cc1F] | 6-(4-amino-3- fluorophenyl) pyridin-2(1H)- one | 1H NMR (400 MHz, DMSO-d6) δ 11.36-11.32 (brs, 1H), 7.50-7.45 (m, 2H), 7.37 (dd, J = 8.4, 2.2 Hz, 1H), 6.82-6.79 (m, 1H), 6.54 (d, J = 7.1 Hz, 1H), 6.23 (d, J = 8.9 Hz, 1H), 5.62-5.58 (brs, 2H). |

### AB. 1-(3-amino-4-fluorophenyl)pyrrolidin-2-one (Intermediate AB)

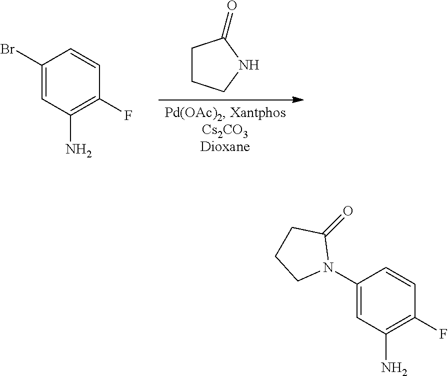

To a stirred mixture of 5-bromo-2-fluoroaniline (800 mg, 4.210 mmol, 1 equiv.) and pyrrolidone (394.14 mg, 4.631 mmol, 1.10 equiv.) in 1,4-dioxane (30 mL) were added Pd(OAc)₂ (141.78 mg, 0.632 mmol, 0.15 equiv.), XantPhos (730.83 mg, 1.263 mmol, 0.30 equiv.) and Cs₂CO₃ (2.74 mg, 8.41 mmol, 2 equiv.) in portions at rt under nitrogen atmosphere. The resulting mixture was stirred for 16 hours at 100° C. under nitrogen atmosphere. The reaction was monitored by LCMS. The mixture was allowed to cool down to rt. The resulting mixture was diluted with DCM/MeOH=10/1 (150 mL). The precipitated solids were collected by filtration. The resulting mixture was concentrated under vacuum. The residue was purified by reverse phase flash chromatography with the following conditions: Column: Spherical C18, 20-40 um, 120 g; Mobile Phase A: Water (plus 5 mM NH₄HCO₃); Mobile Phase B: ACN; Flow rate: 40 mL/min; Gradient: 5%-5% B, 10 min, 20% B-50% B gradient in 30 min; Detector: 220 nm. The fractions containing the desired product were collected at 34% B and concentrated under reduced pressure to afford 1-(3-amino-4-fluorophenyl)pyrrolidin-2-one (315 mg, 38%) as a white solid. 1H NMR (400 MHz, DMSO-d6) δ 7.20-7.17 (m, 1H), 6.97-6.92 (m, 1H), 6.71-6.68 (m, 1H), 5.19-5.15 (brs, 2H), 3.73 (t, J=7.0 Hz, 2H), 2.45 (t, J=8.1 Hz, 2H), 2.10-1.96 (m, 2H). Preparation of intermediate AC shown in the table below follows the methods and protocols as described for the synthesis of intermediate AB, starting with the appropriate materials.

| | | | |
|---|---|---|---|
| | | Compound | |
| Compound | Structure | Name | NMR |
| AC | 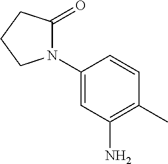 | 1-(3-amino-4- methylphenyl) pyrrolidin-2- one | |

### AD. 2-fluoro-5-(morpholin-4-yl)aniline

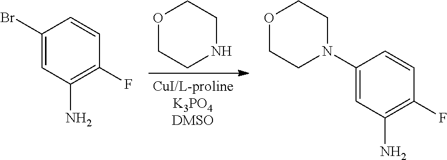

To a stirred mixture of 5-bromo-2-fluoroaniline (1 g, 5.263 mmol, 1 equiv.) and morpholine (550.20 mg, 6.315 mmol, 1.20 equiv.) in DMSO (10 mL) were added K₃PO₄ (3.35 g, 15.782 mmol, 3 equiv.), L-proline (363.54 mg, 3.158 mmol, 0.60 equiv.) and CuI (300.69 mg, 1.579 mmol, 0.30 equiv.) dropwise at room temperature. The resulting mixture was stirred for 2 hours at 120° C. under nitrogen atmosphere. The reaction was monitored by LCMS. The mixture was allowed to cool down to room temperature. The resulting mixture was concentrated under reduced pressure. The resulting mixture was extracted with EtOAc (3×100 mL). The combined organic layers were washed with brine (2×100 mL), dried over anhydrous Na₂SO₄. After filtration, the filtrate was concentrated under reduced pressure. The residue product was purified by reverse phase flash with the following conditions (column, C_{18, 330} g; mobile phase: A:Water/0.05% NH₄HCO₃, Mobile Phase B:ACN; Flow rate:80 mL/min; Gradient: 25% B to 60% B in 25 min; Detector, 220 nm, Monitor, 254 nm, the desired product were collected at 33% B)) to afford 2-fluoro-5-(morpholin-4-yl)aniline (190 mg, 18%) as a light brown solid. 1H NMR (400 MHz, DMSO-d6) δ 6.85-6.88 (m, 1H), 6.37-6.35 (m, 1H), 6.10-6.07 (m, 1H), 4.99-4.96 (brs, 2H), 3.73-3.70 (m, 4H), 2.97-2.93 (m, 4H).

### AE. 3-(4-amino-3-fluorophenyl)-4H-1,2,4-oxadiazol-5-one

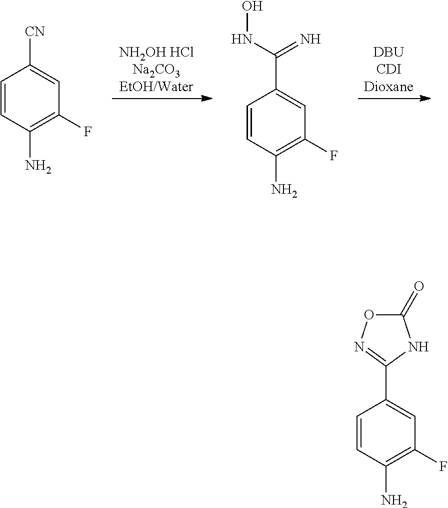 [SMILES: N#Cc1ccc(N)c(F)c1 ; "NH2OH HClNa2CO3EtOH/Water" ; N=C(NO)c1ccc(N)c(F)c1 ; "DBUCDIDioxane" ; Nc1ccc(-c2noc(=O)[nH]2)cc1F]

4-amino-3-fluoro-N-hydroxybenzenecarboximidamide. To a stirred mixture of 4-amino-3-fluorobenzonitrile (1 g, 7.346 mmol, 1 equiv.) and Na₂CO₃ (4.28 g, 40.403 mmol, 5.50 equiv.) in ethyl alcohol (20 mL) and H₂O (5 mL) was added hydroxylamine hydrochloride (2.55 g, 36.730 mmol, 5 equiv.) in portions at room temperature under nitrogen atmosphere. The resulting mixture was stirred for 16 hours at 70° C. under nitrogen atmosphere. Desired product could be detected by LCMS. The mixture was allowed to cool down to room temperature. Used product crude in next step. 3-(4-amino-3-fluorophenyl)-4H-1,2,4-oxadiazol-5-one (Intermediate AE). To a stirred mixture of 4-amino-3-fluoro-N-hydroxybenzenecarboximidamide (2.60 g, 15.370 mmol, 1 equiv.) and DBU (2.60 g, 17.079 mmol, 1.11 equiv.) in 1,4-dioxane (50 mL) was added CDI (3.74 g, 23.055 mmol, 1.50 equiv.) in portions at room temperature under nitrogen atmosphere. The resulting mixture was stirred for 16 hours at 110° C. under nitrogen atmosphere. The resulting mixture was concentrated under reduced pressure. The residue product was purified by reverse phase flash with the following conditions (Column: Spherical C18, 20˜40 um, 80 g; Mobile Phase A: Water (plus 0.05% FA); Mobile Phase B: ACN; Flow rate: 80 mL/min; Gradient of B: 5%, 6 min; 5%-25%, 15 min; 25%-45%, 15 min; 45%-95%, 15 min, Detector: 220 nm. The fractions containing the desired product were collected at 30% B and concentrated under reduced pressure to afford 3-(4-amino-3-fluorophenyl)-4H-1,2,4-oxadiazol-5-one (730 mg, 24) as a light brown solid. 1H NMR (400 MHz, DMSO-d6) δ 7.57-7.26 (m, 2H), 6.87-6.83 (m, 1H), 5.99-5.96 (brs, 2H).

### AF. 2-fluoro-4-(1,2,4-oxadiazol-3-yl)aniline

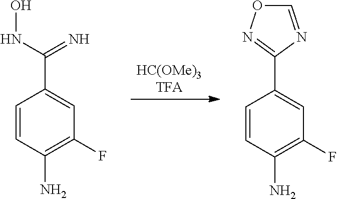 [SMILES: "HC(OMe)3TFA" ; N=C(NO)c1ccc(N)c(F)c1 ; Nc1ccc(-c2ncon2)cc1F]

To a stirred solution of 4-amino-3-fluoro-N-hydroxybenzenecarboximidamide (Crude product from step 1 of synthesis of Intermediate AE, 500 mg, 2.956 mmol, 1 equiv.) in trimethyl orthoformate (20 mL) was added TFA (1 mL, 13.463 mmol, 4.55 equiv.) in portions at room temperature under nitrogen atmosphere. The resulting mixture was stirred for 30 min at room temperature, then was stirred for 1 hour at 60° C. under nitrogen atmosphere. The reaction was monitored by LCMS. Desired product could be detected by LCMS. The resulting mixture was concentrated under reduced pressure. The residue was purified by silica gel column chromatography, eluted with PE/EtOAc (4:1) to afford 2-fluoro-4-(1,2,4-oxadiazol-3-yl)aniline (220 mg, 42%) as a light yellow solid. 1H NMR (400 MHz, DMSO-d6) δ 9.56 (s, 1H), 7.62-7.53 (m, 2H), 6.90-6.86 (m, 1H), 5.89-5.85 (brs, 2H).

### Example 2. Preparation of Compound 100

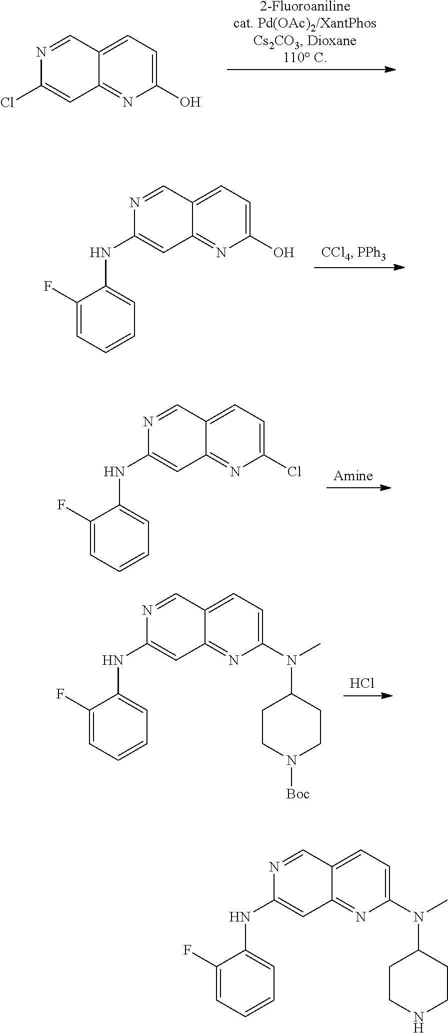

7-[(2-fluorophenyl)amino]-1,6-naphthyridin-2-ol. Into a 100 mL round-bottom flask were added 7-chloro-1,6-naphthyridin-2-ol (3 g, 16.612 mmol, 1 equiv.), 2-fluoroaniline (2.03 g, 18.273 mmol, 1.10 equiv.), Pd(OAc)₂ (0.37 g, 1.661 mmol, 0.10 equiv.), XantPhos (1.92 g, 3.322 mmol, 0.20 equiv.), and Cs₂CO₃ (16.24 g, 49.837 mmol, 3 equiv.) in dioxane (40 mL) at room temperature. The resulting mixture was stirred for 16 hours at 110° C. under nitrogen atmosphere. The reaction was monitored by LCMS. The mixture was allowed to cool down to room temperature. The resulting mixture was extracted with EtOAc (3×400 mL). The combined organic layers were washed with water (3×100 mL), dried over anhydrous Na₂SO₄. After filtration, the filtrate was concentrated under reduced pressure. The residue was purified by silica gel column chromatography, eluted with DCM/MeOH (12:1) to afford 7-[(2-fluorophenyl)amino]-1,6-naphthyridin-2-ol (3.2 g, 75%) as a brown solid.

2-chloro-N-(2-fluorophenyl)-1,6-naphthyridin-7-amine. Into a 100 mL round-bottom flask were added 7-[(2-fluorophenyl)amino]-1,6-naphthyridin-2-ol (3.20 g, 12.537 mmol, 1 equiv.), PPh₃ (9.86 g, 37.610 mmol, 3 equiv.) and CCl₄ (5.79 g, 37.610 mmol, 3 equiv.) in DCE (40 mL) at room temperature. The resulting mixture was stirred for 16 hours at 80° C. The reaction was monitored by LCMS. The mixture was allowed to cool down to room temperature. The reaction was quenched with NaHCO₃ at room temperature. The resulting mixture was extracted with DCM (3×100 mL). The combined organic layers were washed with water (3×50 mL), dried over anhydrous Na₂SO₄. After filtration, the filtrate was concentrated under reduced pressure. The residue was purified by silica gel column chromatography, eluted with hexane/EtOAc (5:1) to afford 2-chloro-N-(2-fluorophenyl)-1,6-naphthyridin-7-amine (1.2 g, 35%) as a yellow solid.

Tert-butyl 4-([7-[(2-fluorophenyl)amino]-1,6-naphthyridin-2-yl](methyl)amino)piperidine-1-carboxylate. Into a 25 mL sealed tube were added 2-chloro-N-(2-fluorophenyl)-1,6-naphthyridin-7-amine (100 mg, 0.365 mmol, 1 equiv.) and tert-butyl 4-(methylamino)piperidine-1-carboxylate (86.13 mg, 0.402 mmol, 1.10 equiv.) at room temperature. The resulting mixture was stirred for 1 hour at 100° C. under nitrogen atmosphere. The reaction was monitored by LCMS. The mixture was allowed to cool down to room temperature. This resulted in tert-butyl 4-([7-[(2-fluorophenyl)amino]-1,6-naphthyridin-2-yl](methyl)amino)piperidine-1-carboxylate (80 mg, 51%) as a yellow solid. The crude product was used in the next step directly without further purification.

N7-(2-fluorophenyl)-N2-methyl-N2-(piperidin-4-yl)-1,6-naphthyridine-2,7-diamine (Compound 100). To a stirred solution of tert-butyl 4-([7-[(2-fluorophenyl)amino]-1,6-naphthyridin-2-yl](methyl)amino)piperidine-1-carboxylate (80 mg, 0.177 mmol, 1 equiv.) in DCM (5 mL) was added HCl (gas) in 1,4-dioxane (3 mL, 98.736 mmol, 557.30 equiv.) dropwise at room temperature. The resulting mixture was stirred for 1 hour at room temperature. The reaction was monitored by TLC. The reaction was quenched with NaHCO₃ at room temperature. The resulting mixture was extracted with DCM (3×30 mL). The combined organic layers were washed with water (3×15 mL), dried over anhydrous Na₂SO₄. After filtration, the filtrate was concentrated under reduced pressure. The residue was purified by reverse phase flash chromatography with the following conditions: Column: Spherical C18, 20-40 um, 120 g; Mobile Phase A: Water (plus 3.2 g NH₄HCO₃); Mobile Phase B: ACN; Flow rate: 90 mL/min; Gradient: 5%-5% B, 10 min, 30% B-50% B gradient in 20 min; Detector: 254 nm. The fractions containing the desired product were collected at 45% B and concentrated under reduced pressure to afford N7-(2-fluorophenyl)-N2-methyl-N2-(piperidin-4-yl)-1,6-naphthyridine-2,7-diamine (39.1 mg) as an off-white solid.

### Example 3. Preparation of Compounds 101 and 112

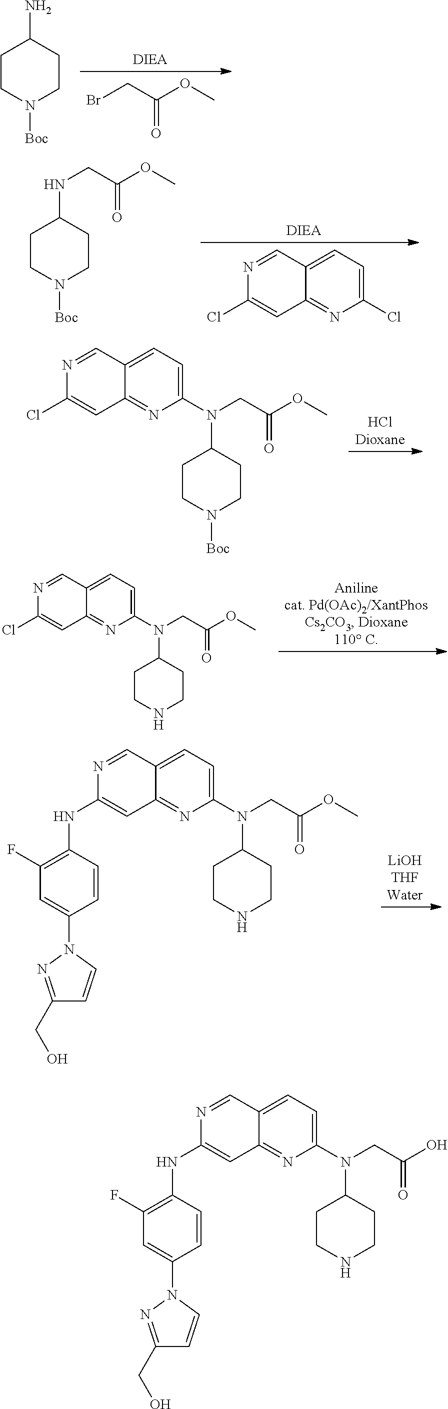

Tert-butyl 4-[(2-methoxy-2-oxoethyl)amino]piperidine-1-carboxylate. To a stirred mixture of tert-butyl 4-aminopiperidine-1-carboxylate (10 g, 49.930 mmol, 1 equiv.) and methyl 2-bromoacetate (6.11 g, 39.941 mmol, 0.80 equiv.) was added DIEA (19.36 g, 149.795 mmol, 3 equiv.) in portions at rt under nitrogen atmosphere. The resulting mixture was stirred for 16 hours at 100° C. under nitrogen atmosphere. The reaction was monitored by TLC. The mixture was allowed to cool down to rt. The residue was purified by silica gel column chromatography, eluted with PE/EtOAc (5/1 to 1/1) to afford tert-butyl 4-[(2-methoxy-2-oxoethyl)amino]piperidine-1-carboxylate (4 g, 29%) as a light yellow oil.

Tert-butyl 4-[(7-chloro-1,6-naphthyridin-2-yl) (2-methoxy-2-oxoethyl)amino]piperidine-1-carboxylate. To a stirred mixture of 2,7-dichloro-1,6-naphthyridine (1.12 g, 5.627 mmol, 0.90 equiv.) and tert-butyl 4-[(2-methoxy-2-oxoethyl)amino]piperidine-1-carboxylate (1.70 g, 6.242 mmol, 1 equiv.) was added DIEA (2.42 g, 18.724 mmol, 3 equiv.) in portions at rt under nitrogen atmosphere. The resulting mixture was stirred for 2 hours at 110° C. under nitrogen atmosphere. The reaction was monitored by LCMS. The residue was purified by reverse phase flash chromatography with the following conditions: Column: Spherical C18, 20-40 um, 330 g; Mobile Phase A: Water (plus 5 mM NH₄HCO₃); Mobile Phase B: ACN; Flow rate: 80 mL/min; Gradient: 5%-5% B, 10 min, 50% B-75% B gradient in 20 min; Detector: 220 nm. The fractions containing the desired product were collected at 70% B and concentrated under reduced pressure to afford tert-butyl 4-[(7-chloro-1,6-naphthyridin-2-yl) (2-methoxy-2-oxoethyl)amino]piperidine-1-carboxylate (800 mg, 29%) as a yellow solid.

Methyl 2-[(7-chloro-1,6-naphthyridin-2-yl) (piperidin-4-yl)amino]acetate. To a stirred solution of tert-butyl 4-[(7-chloro-1,6-naphthyridin-2-yl) (2-methoxy-2-oxoethyl)amino]piperidine-1-carboxylate (800 mg) in MeOH (20 mL) was added HCl (gas) in 1,4-dioxane (20 mL) in portions at rt under nitrogen atmosphere. The resulting mixture was stirred for 2 hours at rt under nitrogen atmosphere. The resulting mixture was concentrated under vacuum. The reaction was monitored by LCMS. This resulted in methyl 2-[(7-chloro-1,6-naphthyridin-2-yl) (piperidin-4-yl)amino]acetate (600 mg) as a yellow solid.

Methyl 2-[[7-([2-fluoro-4-[3-(hydroxymethyl)pyrazol-1-yl]phenyl]amino)-1,6-naphthyridin-2-yl](piperidin-4-yl)amino]acetate (Compound 112). To a stirred mixture of methyl 2-[(7-chloro-1,6-naphthyridin-2-yl) (piperidin-4-yl)amino]acetate (500 mg, 1.493 mmol, 1 equiv.) and [1-(4-amino-3-fluorophenyl)pyrazol-3-yl]methanol (340.40 mg, 1.643 mmol, 1.10 equiv.) in 1,4-dioxane (20 mL) were added Pd(OAc)₂ (50.29 mg, 0.224 mmol, 0.15 equiv.), XantPhos (259.24 mg, 0.448 mmol, 0.30 equiv.) and Cs₂CO₃ (973.18 mg, 2.987 mmol, 2 equiv.) in portions at rt under nitrogen atmosphere. The resulting mixture was stirred for 2 hours at 100° C. under nitrogen atmosphere. The reaction was monitored by LCMS. The mixture was allowed to cool down to rt. The resulting mixture was extracted with EtOAc (3×500 mL). The combined organic layers were washed with brine (2×300 mL), dried over anhydrous Na₂SO₄. After filtration, the filtrate was concentrated under reduced pressure. The residue was purified by reverse phase flash chromatography with the following conditions: Column: Spherical C18, 20-40 um, 330 g; Mobile Phase A: Water (plus 5 mM NH₄HCO₃); Mobile Phase B: ACN; Flow rate: 80 mL/min; Gradient: 5%-5% B, 10 min, 50% B-70% B gradient in 20 min; Detector: 220 nm. The fractions containing the desired product were collected at 65% B and concentrated under reduced pressure to afford methyl 2-[[7-([2-fluoro-4-[3-(hydroxymethyl)pyrazol-1-yl]phenyl]amino)-1,6-naphthyridin-2-yl](piperidin-4-yl)amino]acetate (200 mg, 26%) as a yellow solid.

[[7-([2-fluoro-4-[3-(hydroxymethyl)pyrazol-1-yl]phenyl]amino)-1,6-naphthyridin-2-yl](piperidin-4-yl)amino]acetic acid (Compound 101). To a stirred solution of methyl 2-[[7-([2-fluoro-4-[3-(hydroxymethyl)pyrazol-1-yl]phenyl]amino)-1,6-naphthyridin-2-yl](piperidin-4-yl)amino]acetate (10 mg, 0.020 mmol, 1 equiv.) in THF and H₂O was added LiOH (1.42 mg, 0.059 mmol, 3 equiv.) at room temperature. The resulting mixture was stirred for 1 hour at room temperature. The reaction was monitored by TLC. The mixture/residue was acidified to pH 4 with citric acid. The resulting mixture was concentrated under reduced pressure. The crude product (10 mg) was purified by Prep-HPLC to afford [[7-([2-fluoro-4-[3-(hydroxymethyl)pyrazol-1-yl]phenyl]amino)-1,6-naphthyridin-2-yl](piperidin-4-yl)amino]acetic acid (2 mg, 21%) as a light yellow solid.

Compounds 111, 120, 125, 126, 176 and 178 were synthesized by the methods and protocols as described for the synthesis of Compound 101, starting with the appropriate materials.

### Example 4. Preparation of Compound 119

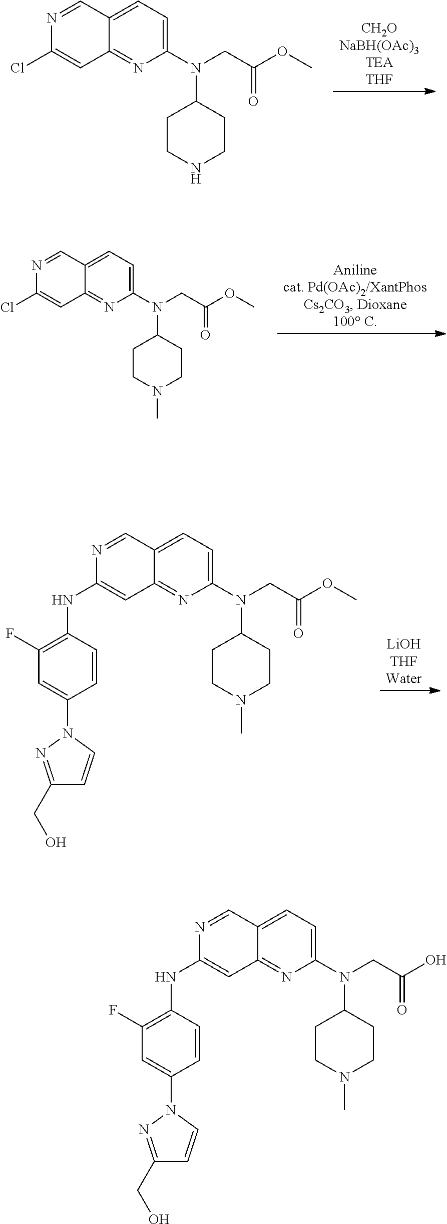

Methyl 2-[(7-chloro-1,6-naphthyridin-2-yl) (1-methylpiperidin-4-yl)amino]acetate. To a stirred mixture of methyl 2-[(7-chloro-1,6-naphthyridin-2-yl) (piperidin-4-yl)amino]acetate (Step 3 from synthesis of Compound 101, 120 mg, 0.358 mmol, 1 equiv.) and HCHO (16.14 mg, 0.538 mmol, 1.50 equiv.) in THF (15 mL) were added TEA (72.54 mg, 0.717 mmol, 2 equiv.) and NaBH(OAc)₃ (113.95 mg, 0.538 mmol, 1.50 equiv.) in portions at 0° C. under nitrogen atmosphere. The resulting mixture was stirred for 1 hour at rt under nitrogen atmosphere. The reaction was monitored by LCMS. The resulting mixture was concentrated under reduced pressure. The residue was purified by reverse phase flash chromatography with the following conditions: Column: Spherical C18, 20-40 um, 120 g; Mobile Phase A: Water (plus 5 mM NH₄HCO₃); Mobile Phase B: ACN; Flow rate: 40 mL/min; Gradient: 5%-5% B, 10 min, 40% B-55% B gradient in 15 min; Detector: 220 nm. The fractions containing the desired product were collected at 45% B and concentrated under reduced pressure to afford methyl 2-[(7-chloro-1,6-naphthyridin-2-yl) (1-methylpiperidin-4-yl)amino]acetate (90 mg, 72%) as a yellow solid.

Methyl 2-[[7-([2-fluoro-4-[3-(hydroxymethyl)pyrazol-1-yl]phenyl]amino)-1,6-naphthyridin-2-yl](1-methylpiperidin-4-yl)amino]acetate. To a stirred mixture of methyl 2-[(7-chloro-1,6-naphthyridin-2-yl) (1-methylpiperidin-4-yl)amino]acetate (100 mg, 0.287 mmol, 1 equiv.) and [1-(4-amino-3-fluorophenyl)pyrazol-3-yl]methanol (65.34 mg, 0.315 mmol, 1.10 equiv.) in 1,4-dioxane (10 mL) were added Pd(OAc)₂ (9.65 mg, 0.043 mmol, 0.15 equiv.), XantPhos (49.76 mg, 0.086 mmol, 0.30 equiv.) and Cs₂CO₃ (186.81 mg, 0.573 mmol, 2 equiv.) in portions at rt under nitrogen atmosphere. The resulting mixture was stirred for 2 hours at 100° C. under nitrogen atmosphere. The reaction was monitored by LCMS. The mixture was allowed to cool down to rt. The resulting mixture was extracted with EtOAc (3×200 mL). The combined organic layers were washed with brine (2×100 mL), dried over anhydrous Na₂SO₄. After filtration, the filtrate was concentrated under reduced pressure. The residue was purified by reverse phase flash chromatography with the following conditions: Column: Spherical C18, 20-40 um, 120 g; Mobile Phase A: Water (plus 5 mM NH₄HCO₃); Mobile Phase B: ACN; Flow rate: 40 mL/min; Gradient: 5%-5% B, 10 min, 60% B-95% B gradient in 20 min; Detector: 220 nm. The fractions containing the desired product were collected at 90% B and concentrated under reduced pressure to afford methyl 2-[[7-([2-fluoro-4-[3-(hydroxymethyl)pyrazol-1-yl]phenyl]amino)-1,6-naphthyridin-2-yl](1-methylpiperidin-4-yl)amino]acetate (50 mg, 34%) as a yellow solid.

[[7-([2-fluoro-4-[3-(hydroxymethyl)pyrazol-1-yl]phenyl]amino)-1,6-naphthyridin-2-yl](1-methylpiperidin-4-yl)amino]acetic acid (Compound 119). To a stirred solution of methyl 2-[[7-([2-fluoro-4-[3-(hydroxymethyl)pyrazol-1-yl]phenyl]amino)-1,6-naphthyridin-2-yl](1-methylpiperidin-4-yl)amino]acetate (200 mg, 0.385 mmol, 1 equiv.) in THF (25 mL) and water (5 mL) was added LiOH (46.09 mg, 1.925 mmol, 5 equiv.) dropwise at rt. The reaction mixture was stirred for 2 hours at rt. The reaction was monitored by LCMS. The resulting mixture was concentrated under reduced pressure. The residue was purified by Prep-HPLC to afford formic acid; [[7-([2-fluoro-4-[3-(hydroxymethyl)pyrazol-1-yl]phenyl]amino)-1,6-naphthyridin-2-yl](1-methylpiperidin-4-yl)amino]acetic acid (150.4 mg, 71%) as a light green solid.

Compounds 132 and 170 were synthesized following the methods and protocols as described for the synthesis of Compound 119, starting with the appropriate materials.

### Example 5. Preparation of Compound 144

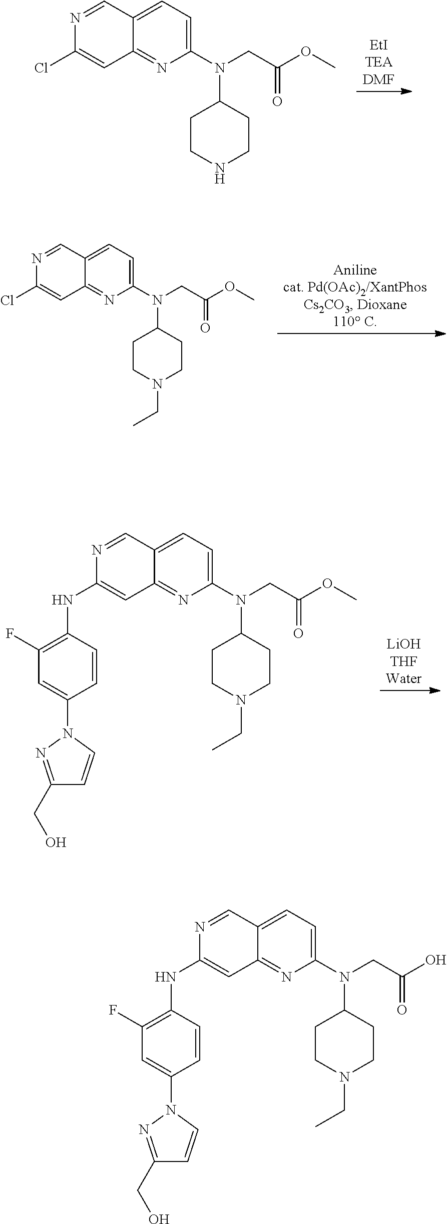

Methyl 2-[(7-chloro-1,6-naphthyridin-2-yl) (1-ethylpiperidin-4-yl)amino]acetate. To a stirred mixture of methyl 2-[(7-chloro-1,6-naphthyridin-2-yl) (piperidin-4-yl)amino]acetate (Step 3 from synthesis of Compound 101, 300 mg, 0.896 mmol, 1 equiv.) and TEA (272.02 mg, 2.688 mmol, 3 equiv.) in DMF (10 mL) was added ethyl iodide (139.75 mg, 0.896 mmol, 1 equiv.) in portions at rt under nitrogen atmosphere. The resulting mixture was stirred for 2 hours at rt under nitrogen atmosphere. The reaction was monitored by LCMS. The residue was purified by reverse phase flash chromatography with the following conditions: Column: Spherical C18, 20-40 um, 330 g; Mobile Phase A: Water (plus 5 mM NH₄HCO₃); Mobile Phase B: ACN; Flow rate: 80 mL/min; Gradient: 5%-5% B, 10 min, 30% B-55% B gradient in 20 min; Detector: 220 nm. The fractions containing the desired product were collected at 48% B and concentrated under reduced pressure to afford methyl 2-[(7-chloro-1,6-naphthyridin-2-yl) (1-ethylpiperidin-4-yl)amino]acetate (180 mg, 55%) as a yellow solid.

Methyl 2-[(1-ethylpiperidin-4-yl)[7-([2-fluoro-4-[3-(hydroxymethyl)pyrazol-1-yl]phenyl]amino)-1,6-naphthyridin-2-yl]amino]acetate. To a stirred mixture of methyl 2-[(7-chloro-1,6-naphthyridin-2-yl) (1-ethylpiperidin-4-yl)amino]acetate (180 mg, 0.496 mmol, 1 equiv.) and [1-(4-amino-3-fluorophenyl)pyrazol-3-yl]methanol (113.07 mg, 0.546 mmol, 1.10 equiv.) in 1,4-dioxane (10 mL) were added Pd(OAc)₂ (16.71 mg, 0.074 mmol, 0.15 equiv.), XantPhos (86.11 mg, 0.149 mmol, 0.30 equiv.) and Cs₂CO₃ (323.25 mg, 0.992 mmol, 2 equiv.) in portions at rt under nitrogen atmosphere. The resulting mixture was stirred for 2 hours at 110° C. under nitrogen atmosphere. The reaction was monitored by LCMS. The mixture was allowed to cool down to rt. The resulting mixture was extracted with EtOAc (3×300 mL). The combined organic layers were washed with brine (2×100 mL), dried over anhydrous Na₂SO₄. After filtration, the filtrate was concentrated under reduced pressure. The residue was purified by reverse phase flash chromatography with the following conditions: Column: Spherical C18, 20-40 um, 330 g; Mobile Phase A: Water (plus 5 mM NH₄HCO₃); Mobile Phase B: ACN; Flow rate: 80 mL/min; Gradient: 5%-5% B, 10 min, 30% B-60% B gradient in 25 min; Detector: 220 nm. The fractions containing the desired product were collected at 55% B and concentrated under reduced pressure to afford methyl 2-[(1-ethylpiperidin-4-yl)[7-([2-fluoro-4-[3-(hydroxymethyl)pyrazol-1-yl]phenyl]amino)-1,6-naphthyridin-2-yl]amino]acetate (200 mg, 76%) as a yellow solid.

[(1-ethylpiperidin-4-yl)[7-([2-fluoro-4-[3-(hydroxymethyl)pyrazol-1-yl]phenyl]amino)-1,6-naphthyridin-2-yl]amino]acetic acid (Compound 144). To a stirred solution of methyl 2-[(1-ethylpiperidin-4-yl)[7-([2-fluoro-4-[3-(hydroxymethyl)pyrazol-1-yl]phenyl]amino)-1,6-naphthyridin-2-yl]amino]acetate (200 mg, 0.375 mmol, 1 equiv.) in THF (25 mL) and water (5 mL) was added LiOH (44.88 mg, 1.874 mmol, 5 equiv.) dropwise at rt. The reaction mixture was stirred for 2 hours at rt. The reaction was monitored by LCMS. The resulting mixture was concentrated under reduced pressure. The residue was purified by Prep-HPLC to afford [(1-ethylpiperidin-4-yl)[7-([2-fluoro-4-[3-(hydroxymethyl)pyrazol-1-yl]phenyl]amino)-1,6-naphthyridin-2-yl]amino]acetic acid; formic acid (111.0 mg, 52%) as a green solid. Compounds 143, 145, and 147 were synthesized following the methods and protocols as described for the synthesis of Compound 144, starting with the appropriate materials.

### Example 6. Preparation of Compound 155

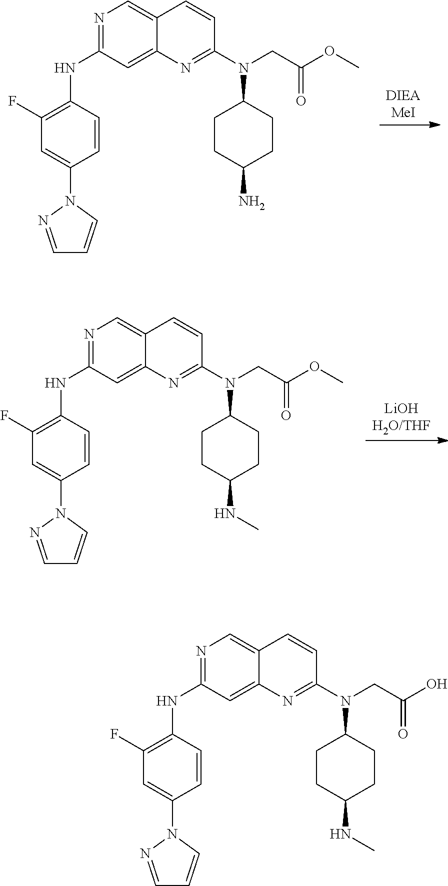 [SMILES: COC(=O)CN(c1ccc2cnc(Nc3ccc(-n4cccn4)cc3F)cc2n1)[C@H]1CC[C@@H](N)CC1 ; "DIEAMeI" ; CN[C@H]1CC[C@@H](N(CC(=O)OC)c2ccc3cnc(Nc4ccc(-n5cccn5)cc4F)cc3n2)CC1 ; "LiOHH2O/THF" ; CN[C@H]1CC[C@@H](N(CC(=O)O)c2ccc3cnc(Nc4ccc(-n5cccn5)cc4F)cc3n2)CC1]

Methyl 2-[(7-[[2-fluoro-4-(pyrazol-1-yl)phenyl]amino]-1,6-naphthyridin-2-yl)[(1s,4s)-4-(methylamino)cyclohexyl]amino]acetate. To a stirred solution of methyl 2-[(7-[[2-fluoro-4-(pyrazol-1-yl)phenyl]amino]-1,6-naphthyridin-2-yl)[(1s,4s)-4-aminocyclohexyl]amino]acetate (Penultimate intermediate from synthesis of Compound 126, 300 mg, 0.613 mmol, 1 equiv.) in CH₃I (217.45 mg, 1.532 mmol, 1.50 equiv.) was added DIEA (396 mg, 3.064 mmol, 3 equiv.) in portions at 0° C. The resulting mixture was stirred for 6 hours at room temperature. The reaction was monitored by LCMS. The resulting mixture was concentrated under reduced pressure. The residue was purified by reverse phase flash with the following conditions (Column: C18, 120 g; Mobile Phase A:Water/0.05% NH₄HCO₃, Mobile Phase B:ACN; Flow rate:40 mL/min; Gradient: 15% B to 45% B in 20 min; Detector, 254 nm and 220 nm, the desired product were collected at 28% B). Concentrated under reduced pressure to afford methyl 2-[(7-[[2-fluoro-4-(pyrazol-1-yl)phenyl]amino]-1,6-naphthyridin-2-yl)[(1s,4s)-4-(methylamino)cyclohexyl]amino]acetate (100 mg, 32%) as an orange solid.

[(7-[[2-fluoro-4-(pyrazol-1-yl)phenyl]amino]-1,6-naphthyridin-2-yl)[(1s,4s)-4-(methylamino)cyclohexyl]amino]acetic acid (Compound 155). To a stirred solution of methyl 2-[(7-[[2-fluoro-4-(pyrazol-1-yl)phenyl]amino]-1,6-naphthyridin-2-yl)[(1s,4s)-4-(methylamino)cyclohexyl]amino]acetate (100 mg, 0.199 mmol, 1 equiv.) in THF (5 mL) and H₂O (1 mL) was added LiOH (14.27 mg, 0.596 mmol, 3 equiv.) in portions at room temperature. The resulting mixture was stirred for 60 min at room temperature. The resulting mixture was concentrated under reduced pressure. The reaction was monitored by LCMS. The resulting mixture was concentrated under reduced pressure. The residue was purified by reverse phase flash chromatography with the following conditions: (Column: XBridge Shield RP18 OBD Column, 5 μm, 19*150 mm). Concentrated under reduced pressure to afford [(7-[[2-fluoro-4-(pyrazol-1-yl)phenyl]amino]-1,6-naphthyridin-2-yl)[(1s,4s)-4-(methylamino)cyclohexyl]amino]acetic acid (1.3 mg, 1%) as a white solid.

### Example 7. Preparation of Compound 182

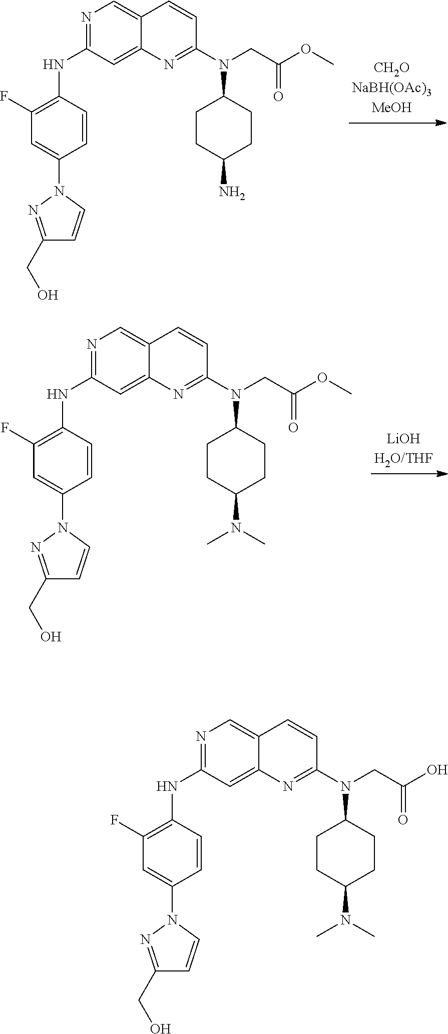 [SMILES: COC(=O)CN(c1ccc2cnc(Nc3ccc(-n4ccc(CO)n4)cc3F)cc2n1)[C@H]1CC[C@@H](N)CC1 ; COC(=O)CN(c1ccc2cnc(Nc3ccc(-n4ccc(CO)n4)cc3F)cc2n1)[C@H]1CC[C@@H](N(C)C)CC1 ; CN(C)[C@H]1CC[C@@H](N(CC(=O)O)c2ccc3cnc(Nc4ccc(-n5ccc(CO)n5)cc4F)cc3n2)CC1]

Methyl 2-[[7-([2-fluoro-4-[3-(hydroxymethyl)pyrazol-1-yl]phenyl]amino)-1,6-naphthyridin-2-yl][(1s,4s)-4-(dimethylamino)cyclohexyl]amino]acetate. To a stirred solution of methyl 2-[[7-([2-fluoro-4-[3-(hydroxymethyl)pyrazol-1-yl]phenyl]amino)-1,6-naphthyridin-2-yl][(1s,4s)-4-aminocyclohexyl]amino]acetate (Penultimate intermediate from synthesis of Compound 126, 320 mg, 0.616 mmol, 1 equiv.) in CH₃OH (5 mL) was added NaBH(OAc)₃ (261.06 mg, 1.232 mmol, 2 equiv.) and HCHO (0.20 mL, 6.571 mmol, 2 equiv.) in portions at 0° C. The resulting mixture was stirred for 6 hours at room temperature. The reaction was monitored by LCMS. The resulting mixture was concentrated under reduced pressure. The residue was purified by reverse phase flash with the following conditions (Column: C18, 120 g; Mobile Phase A:Water/0.05% NH₄HCO₃, Mobile Phase B:ACN; Flow rate:40 mL/min; Gradient: 15% B to 45% B in 20 min; Detector, 254 nm and 220 nm, the desired product were collected at 28% B). Concentrated under reduced pressure to afford methyl 2-[[7-([2-fluoro-4-[3-(hydroxymethyl)pyrazol-1-yl]phenyl]amino)-1,6-naphthyridin-2-yl][(1s,4s)-4-(dimethylamino)cyclohexyl]amino]acetate (100 mg, 30%) as a light green solid.

[[7-([2-fluoro-4-[3-(hydroxymethyl)pyrazol-1-yl]phenyl]amino)-1,6-naphthyridin-2-yl][(1s,4s)-4-(dimethylamino)cyclohexyl]amino]acetic acid (Compound 182). To a stirred solution of methyl 2-[[7-([2-fluoro-4-[3-(hydroxymethyl)pyrazol-1-yl]phenyl]amino)-1,6-naphthyridin-2-yl][(1s,4s)-4-(dimethylamino)cyclohexyl]amino]acetate (100 mg, 0.183 mmol, 1 equiv.) in THF (5 mL) was added LiOH (13.12 mg, 0.548 mmol, 3 equiv.) and H₂O (1 mL) in portions at 0° C. The resulting mixture was stirred for 1 hour at room temperature. The reaction was monitored by LCMS. The resulting mixture was concentrated under reduced pressure. The crude product (mg) was purified by Prep-HPLC to afford [[7-([2-fluoro-4-[3-(hydroxymethyl)pyrazol-1-yl]phenyl]amino)-1,6-naphthyridin-2-yl][(1s,4s)-4-(dimethylamino)cyclohexyl]amino]acetic acid (4.7 mg, 5%) as a light yellow solid. Compound 160 was synthesized following the methods and protocols as described for the synthesis of Compound 182, starting with the appropriate materials.

### Example 8. Preparation of Compound 102

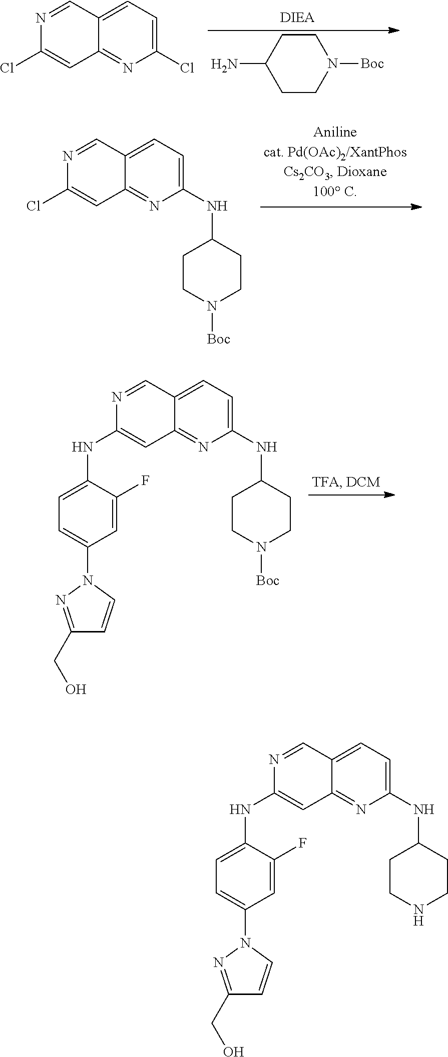

Tert-butyl 4-[(7-chloro-1,6-naphthyridin-2-yl)amino]piperidine-1-carboxylate. 2,7-dichloro-1,6-naphthyridine (2 g, 10.049 mmol, 1 equiv.) were added tert-butyl 4-aminopiperidine-1-carboxylate (2.01 g, 10.036 mmol, 1 equiv.) and DIEA (2.60 g, 20.117 mmol, 2 equiv.) at room temperature. The viscous mixture was heated at 100° C. for 16 hours. The desired product could be detected by LCMS. The reaction mixture was purified by reverse phase flash with the following conditions (Column: C18, 330 g; Mobile Phase A:Water/0.05% NH₄HCO₃, Mobile Phase B:ACN; Flow rate:80 mL/min; Gradient: 40% B to 70% B in 20 min; Detector, 254 nm, Monitor, 220 nm, the desired product were collected at 62% B) to afford tert-butyl 4-[(7-chloro-1,6-naphthyridin-2-yl)amino]piperidine-1-carboxylate (3 g, 82%) as a yellow solid.

Tert-butyl 4-[[7-([2-fluoro-4-[3-(hydroxymethyl)pyrazol-1-yl]phenyl]amino)-1,6-naphthyridin-2-yl]amino]piperidine-1-carboxylate. To a solution of tert-butyl 4-[(7-chloro-1,6-naphthyridin-2-yl)amino]piperidine-1-carboxylate (150 mg, 0.413 mmol, 1 equiv.) in 1,4-dioxane (6 mL) were added [1-(4-amino-3-fluorophenyl)pyrazol-3-yl]methanol (102.79 mg, 0.496 mmol, 1.20 equiv.), XantPhos (47.84 mg, 0.083 mmol, 0.20 equiv.), Cs₂CO₃ (404.06 mg, 1.240 mmol, 3 equiv.) and Pd(OAc)₂ (9.28 mg, 0.041 mmol, 0.10 equiv.) under nitrogen atmosphere. The resulting mixture was stirred for 16 hours at 100° C. The desired product could be detected by LCMS. The mixture was allowed to cool down to room temperature. The mixture was added EA (100 mL) and filtered. The filtrate was concentrated to afford crude product. The crude product was purified by reverse phase flash with the following conditions (Column: C18, 330 g; Mobile Phase A:Water/0.05% NH₄HCO₃, Mobile Phase B:ACN; Flow rate:80 mL/min; Gradient: 40% B to 70% B in 20 min; Detector, 254 nm, Monitor, 220 nm, the desired product were collected at 65% B) to afford tert-butyl 4-[[7-([2-fluoro-4-[3-(hydroxymethyl)pyrazol-1-yl]phenyl]amino)-1,6-naphthyridin-2-yl]amino]piperidine-1-carboxylate (215 mg, 97%) as a white solid.

[1-(3-fluoro-4-[[2-(piperidin-4-ylamino)-1,6-naphthyridin-7-yl]amino]phenyl)pyrazol-3-yl]methanol (Compound 102). To a solution of TFA (3 mL) in DCM (12 mL) was added tert-butyl 4-[[7-([2-fluoro-4-[3-(hydroxymethyl)pyrazol-1-yl]phenyl]amino)-1,6-naphthyridin-2-yl]amino]piperidine-1-carboxylate (210 mg, 0.394 mmol, 1 equiv.) at ambient temperature. Then the mixture was stirred for 2 hours at ambient temperature. The desired product could be detected by LCMS. The resulting mixture was concentrated under reduced pressure. The mixture was basified to pH 8 with NaHCO₃ (aq.) and concentrated under reduced pressure to afford crude product. The crude product was purified by Prep-HPLC to afford [1-(3-fluoro-4-[[2-(piperidin-4-ylamino)-1,6-naphthyridin-7-yl]amino]phenyl)pyrazol-3-yl]methanol (33.2 mg, 19%) as a light green solid.

Compounds 104, 166, 172, 173, and 179 were synthesized following the methods and protocols as described for the synthesis of Compound 102, starting with the appropriate materials.

### Example 9. Preparation of Compounds 103 and 105

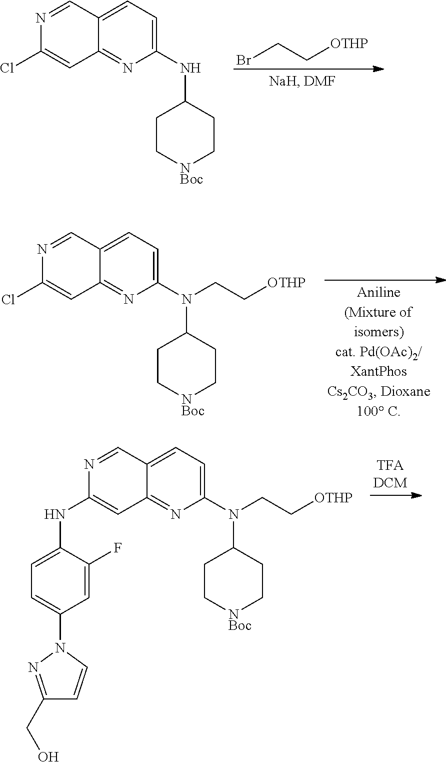

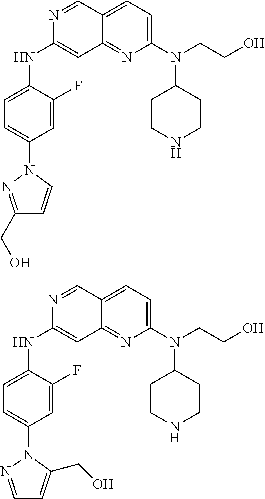 [SMILES: OCCN(c1ccc2cnc(Nc3ccc(-n4ccc(CO)n4)cc3F)cc2n1)C1CCNCC1 ; OCCN(c1ccc2cnc(Nc3ccc(-n4nccc4CO)cc3F)cc2n1)C1CCNCC1]

Tert-butyl 4-[(7-chloro-1,6-naphthyridin-2-yl)[2-(oxan-2-yloxy)ethyl]amino]piperidine-1-carboxylate. To a stirred solution of tert-butyl 4-[(7-chloro-1,6-naphthyridin-2-yl)amino]piperidine-1-carboxylate (from step 1 of synthesis of Compound 102, 100 mg, 0.276 mmol, 1 equiv.) in DMF (5 mL, 64.609 mmol, 234.44 equiv.) in DMF (15 mL, 193.826 mmol, 175.83 equiv.) was added NaH (34.39 mg, 1.433 mmol, 1.3 equiv.) at room temperature under nitrogen atmosphere. The resulting mixture was stirred for 30 min under nitrogen atmosphere. To the above mixture was added 2-(2-bromoethoxy)oxane (345.73 mg, 1.654 mmol, 1.5 equiv.) at room temperature. The resulting mixture was stirred for 16 hours at 80° C. The reaction was monitored by LCMS. The reaction was quenched with sat. NH₄C₁ (aq.). The resulting mixture was extracted with EtOAc (2×50 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EtOAc (20:1 to3:1) to afford tert-butyl 4-[(7-chloro-1,6-naphthyridin-2-yl)[2-(oxan-2-yloxy)ethyl]amino]piperidine-1-carboxylate (350 mg, 65%) as an off-white solid.

Tert-butyl 4-[[7-([2-fluoro-4-[3-(hydroxymethyl)pyrazol-1-yl]phenyl]amino)-1,6-naphthyridin-2-yl][2-(oxan-2-yloxy)ethyl]amino]piperidine-1-carboxylate. To a stirred solution of tert-butyl 4-[(7-chloro-1,6-naphthyridin-2-yl)[2-(oxan-2-yloxy)ethyl]amino]piperidine-1-carboxylate (220 mg, 0.448 mmol, 1 equiv.), [1-(4-amino-3-fluorophenyl)pyrazol-3-yl]methanol (111.40 mg, 0.538 mmol, 1.2 equiv.), XantPhos (51.85 mg, 0.090 mmol, 0.2 equiv.) and Cs₂CO₃ (291.96 mg, 0.896 mmol, 2 equiv.) in 1,4-dioxane (6 mL) was added Pd(OAc)₂ (20.12 mg, 0.090 mmol, 0.2 equiv.) dropwise at room temperature under nitrogen atmosphere. The resulting mixture was stirred for 3 hours at 100° C. under nitrogen atmosphere. The reaction was monitored by LCMS. The resulting mixture was concentrated under vacuum. The residue was purified by reverse phase flash chromatography with the following conditions: Column: Spherical C18, 20-40 um, 330 g; Mobile Phase A: Water (plus 5 mM NH₄CO₃); Mobile Phase B: ACN; Flow rate: 80 mL/min; Gradient: 5%-5% B, 10 min, 5% B-45% B gradient in 20 min; Detector: 220 nm. The fractions containing the desired product were collected at 40% B and concentrated under reduced pressure to afford tert-butyl 4-[[7-([2-fluoro-4-[3-(hydroxymethyl)pyrazol-1-yl]phenyl]amino)-1,6-naphthyridin-2-yl][2-(oxan-2-yloxy)ethyl]amino]piperidine-1-carboxylate (150 mg, 51%) as a white solid

2-[[7-([2-fluoro-4-[5-(hydroxymethyl)pyrazol-1-yl]phenyl]amino)-1,6-naphthyridin-2-yl](piperidin-4-yl)amino]ethanol (Compound 103) and 2-[[7-([2-fluoro-4-[3-(hydroxymethyl)pyrazol-1-yl]phenyl]amino)-1,6-naphthyridin-2-yl](piperidin-4-yl)amino]ethanol (Compound 105). To a stirred solution of tert-butyl 4-[[7-([2-fluoro-4-[3-(hydroxymethyl)pyrazol-1-yl]phenyl]amino)-1,6-naphthyridin-2-yl][2-(oxan-2-yloxy)ethyl]amino]piperidine-1-carboxylate (75 mg, 0.113 mmol, 1 equiv.) in DCM (15 mL, 235.951 mmol, 2081.96 equiv.) was added TFA (45 mL, 605.837 mmol, 5345.73 equiv.) at room temperature under nitrogen atmosphere. The resulting mixture was stirred for 2 hours at room temperature. The reaction was monitored by LCMS. The resulting mixture was concentrated under reduced pressure. The resulting mixture was diluted with ACN (10 mL). The mixture was basified to pH 8 with saturated NaHCO₃ (aq.). The resulting mixture was concentrated under reduced pressure.

The residue was purified by reverse phase flash chromatography with the following conditions: Column: Spherical C18, 20-40 um, 330 g; Mobile Phase A: Water (plus 5 mM NH₄CO₃); Mobile Phase B: ACN; Flow rate: 85 mL/min; Gradient: 5%-5% B, 10 min, 30% B-75% B gradient in 20 min; Detector: 254 nm. The fractions containing the desired product were collected at 38% B and concentrated under reduced pressure to afford desired product (35 mg mixture). The was mixture separated by Chiral-HPLC with the following conditions: Column: CHIRALPAK IG, 2*25 cm, 5 μm; Mobile Phase A:HEX:DCM=3:1 (0.2% IPA)-HPLC, Mobile Phase B:EtOH-HPLC; Flow rate:20 mL/min; Gradient:30 B to 30 B in 25 min; 220/254 nm; The fractions at 17.7 min were collected and concentrated under reduced pressure to afford 2-[[7-([2-fluoro-4-[5-(hydroxymethyl)pyrazol-1-yl]phenyl]amino)-1,6-naphthyridin-2-yl](piperidin-4-yl)amino]ethanol (9.8 mg, 18%) as an off-white solid. The fractions at 22.2 min were collected and concentrated under reduced pressure to afford 2-[[7-([2-fluoro-4-[3-(hydroxymethyl)pyrazol-1-yl]phenyl]amino)-1,6-naphthyridin-2-yl](piperidin-4-yl)amino]ethanol (3.9 mg, 7%) as an off-white solid.

### Example 10. Preparation of Compound 106

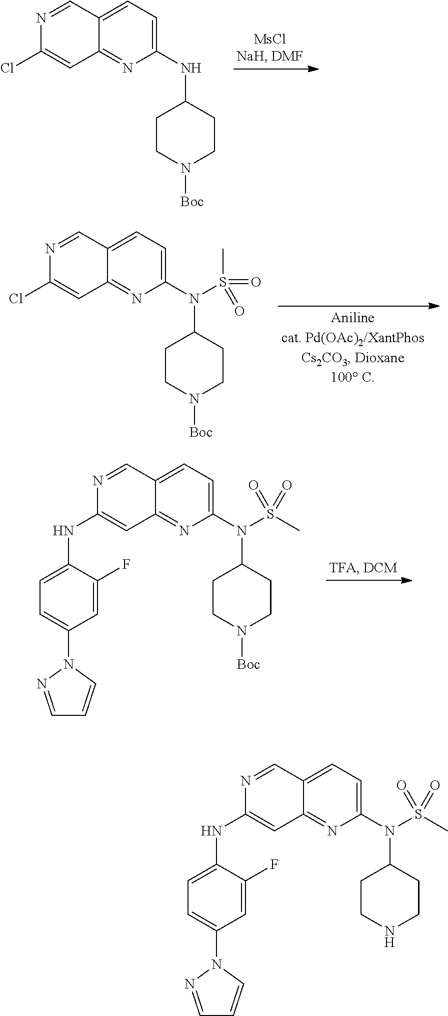

Tert-butyl 4-[N-(7-chloro-1,6-naphthyridin-2-yl)methanesulfonamido]piperidine-1-carboxylate. To a stirred solution of tert-butyl 4-[(7-chloro-1,6-naphthyridin-2-yl)amino]piperidine-1-carboxylate (from step 1 of synthesis of Compound 102, 250 mg, 0.689 mmol, 1 equiv.) in DMF (5 mL) was added NaH (55.11 mg, 1.378 mmol, 2 equiv, 60%) at room temperature. The resulting mixture was stirred for 30 min at room temperature. Then MsCl (236.77 mg, 2.067 mmol, 3 equiv.) was added. The resulting mixture was stirred for 16 hours at room temperature. The reaction was monitored by LCMS. The crude product was purified by reverse phase flash with the following conditions (Column: C18, 330 g; Mobile Phase A:Water/0.05% TFA, Mobile Phase B:ACN; Flow rate:80 mL/min; Gradient: 40% B to 70% B in 20 min; Detector, 220 nm, Monitor, 254 nm, the desired product were collected at 70% B) to afford tert-butyl 4-[N-(7-chloro-1,6-naphthyridin-2-yl)methanesulfonamido]piperidine-1-carboxylate (200 mg, 66%) as a yellow oil.

Tert-butyl 4-[N-(7-[[2-fluoro-4-(pyrazol-1-yl)phenyl]amino]-1,6-naphthyridin-2-yl)methanesulfonamido]piperidine-1-carboxylate. To a stirred mixture of tert-butyl 4-[N-(7-chloro-1,6-naphthyridin-2-yl)methanesulfonamido]piperidine-1-carboxylate (200 mg, 0.454 mmol, 1 equiv.) and 2-fluoro-4-(pyrazol-1-yl)aniline (88.40 mg, 0.499 mmol, 1.10 equiv.) in 1,4-dioxane (10 mL) were added XantPhos (52.49 mg, 0.091 mmol, 0.20 equiv.), Cs₂CO₃ (295.57 mg, 0.907 mmol, 2 equiv.) and Pd(OAc)₂ (10.18 mg, 0.045 mmol, 0.10 equiv.) at room temperature under nitrogen atmosphere. The resulting mixture was stirred for 3 hours at 100° C. under nitrogen atmosphere. The reaction was monitored by LCMS. The resulting mixture was filtered, the filter cake was washed with DCM (3×10 mL). The filtrate was concentrated under reduced pressure. The residue was purified by reverse phase flash with the following conditions (Column: C18, 330 g; Mobile Phase A:Water/0.05% TFA, Mobile Phase B:ACN; Flow rate:80 mL/min; Gradient: 40% B to 70% B in 20 min; Detector, 254 nm, Monitor, 220 nm, the desired product were collected at 69% B) to afford tert-butyl 4-[N-(7-[[2-fluoro-4-(pyrazol-1-yl)phenyl]amino]-1,6-naphthyridin-2-yl)methanesulfonamido]piperidine-1-carboxylate (200 mg, 76%) as a yellow solid.

N-(7-[[2-fluoro-4-(pyrazol-1-yl)phenyl]amino]-1,6-naphthyridin-2-yl)-N-(piperidin-4-yl)methanesulfonamide (Compound 106). To a stirred solution of tert-butyl 4-[N-(7-[[2-fluoro-4-(pyrazol-1-yl)phenyl]amino]-1,6-naphthyridin-2-yl)methanesulfonamido]piperidine-1-carboxylate (50 mg, 0.086 mmol, 1 equiv.) in DCM (4 mL) was added TFA (1 mL, 13.463 mmol, 156.62 equiv.) at room temperature. The resulting mixture was stirred for 2 hours at room temperature. The reaction was monitored by LCMS. The resulting mixture was concentrated under reduced pressure. The residue was basified to pH 8 with saturated NaHCO₃ (aq.). The resulting mixture was concentrated under reduced pressure. The crude product (30 mg) was purified by Prep-HPLC to afford N-(7-[[2-fluoro-4-(pyrazol-1-yl)phenyl]amino]-1,6-naphthyridin-2-yl)-N-(piperidin-4-yl)methanesulfonamide (12.8 mg, 31%) as a yellow solid.

### Example 11. Preparation of Compound 109

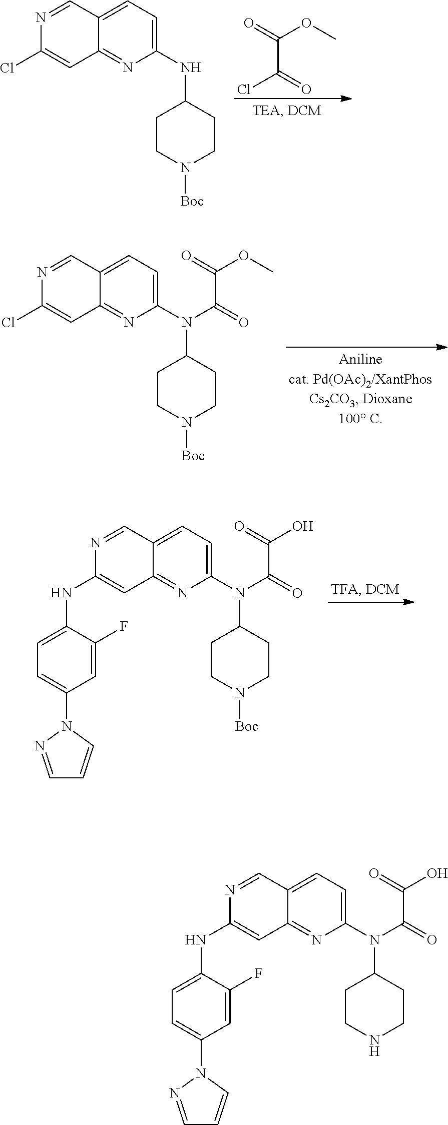

Tert-butyl 4-[N-(7-chloro-1,6-naphthyridin-2-yl)-2-methoxy-2-oxoacetamido]piperidine-1-carboxylate. To a stirred mixture of tert-butyl 4-[(7-chloro-1,6-naphthyridin-2-yl)amino]piperidine-1-carboxylate (from step 1 of synthesis of Compound 102, 50 mg, 0.138 mmol, 1 equiv.) and TEA (27.89 mg, 0.276 mmol, 2 equiv.) in DCM (10 mL) was added methyl oxalochloridate (25.32 mg, 0.207 mmol, 1.50 equiv.) dropwise at 0° C. The resulting mixture was stirred for 16 hours at room temperature. The reaction was monitored by LCMS. The resulting mixture was extracted with DCM (3×100 mL). The combined organic layers were washed with brine (1×100 mL), dried over anhydrous Na₂SO₄. After filtration, the filtrate was concentrated under reduced pressure. The residue was purified by reverse phase flash with the following conditions (Column: C18, 330 g; Mobile Phase A:Water/0.05% TFA, Mobile Phase B:ACN; Flow rate:80 mL/min; Gradient: 40% B to 80% B in 20 min; Detector, 254 nm, Monitor, 220 nm, the desired product were collected at 74% B) to afford tert-butyl 4-[N-(7-chloro-1,6-naphthyridin-2-yl)-2-methoxy-2-oxoacetamido]piperidine-1-carboxylate (450 mg, 91%) as a pink solid.

[[1-(tert-butoxycarbonyl)piperidin-4-yl](7-[[2-fluoro-4-(pyrazol-1-yl)phenyl]amino]-1,6-naphthyridin-2-yl)carbamoyl]formic acid. To a stirred mixture of tert-butyl 4-[N-(7-chloro-1,6-naphthyridin-2-yl)-2-methoxy-2-oxoacetamido]piperidine-1-carboxylate (400 mg, 0.891 mmol, 1 equiv.) and 2-fluoro-4-(pyrazol-1-yl)aniline (189.46 mg, 1.069 mmol, 1.20 equiv.) in 1,4-dioxane (10 mL) were added XantPhos (103.12 mg, 0.178 mmol, 0.20 equiv.), Cs₂CO₃ (580.65 mg, 1.782 mmol, 2 equiv.) and Pd(OAc)₂ (20.01 mg, 0.089 mmol, 0.10 equiv.) at room temperature under nitrogen atmosphere. The resulting mixture was stirred for 5 hours at 100° C. under nitrogen atmosphere. The reaction was monitored by LCMS. The mixture was allowed to cool down to room temperature. The resulting mixture was filtered, the filter cake was washed with EtOAc (3×10 mL). The filtrate was concentrated under reduced pressure. The residue was purified by reverse phase flash with the following conditions (Column: C18, 330 g; Mobile Phase A:Water/0.05% NH₄HCO₃, Mobile Phase B:ACN; Flow rate:80 mL/min; Gradient: 30% B to 70% B in 30 min; Detector, 254 nm, Monitor, 220 nm, the desired product were collected at 57% B) to afford [[1-(tert-butoxycarbonyl)piperidin-4-yl](7-[[2-fluoro-4-(pyrazol-1-yl)phenyl]amino]-1,6-naphthyridin-2-yl)carbamoyl]formic acid (100 mg, 19%) as a yellow solid.

[(7-[[2-fluoro-4-(pyrazol-1-yl)phenyl]amino]-1,6-naphthyridin-2-yl) (piperidin-4-yl)carbamoyl]formic acid (Compound 109). To a stirred solution of [[1-(tert-butoxycarbonyl)piperidin-4-yl](7-[[2-fluoro-4-(pyrazol-1-yl)phenyl]amino]-1,6-naphthyridin-2-yl)carbamoyl]formic acid (100 mg, 1 equiv.) in DCM (4 mL) was added TFA (1 mL) at room temperature. The resulting mixture was stirred for 2 hours at room temperature. The reaction was monitored by LCMS. The mixture was basified to pH 8 with saturated NaHCO₃ (aq.). The resulting mixture was concentrated under reduced pressure. The crude product (50 mg) was purified by Prep-HPLC to afford [(7-[[2-fluoro-4-(pyrazol-1-yl)phenyl]amino]-1,6-naphthyridin-2-yl) (piperidin-4-yl)carbamoyl]formic acid (24.1 mg, 29%) as a yellow solid.

### Example 12. Preparation of Compound 110

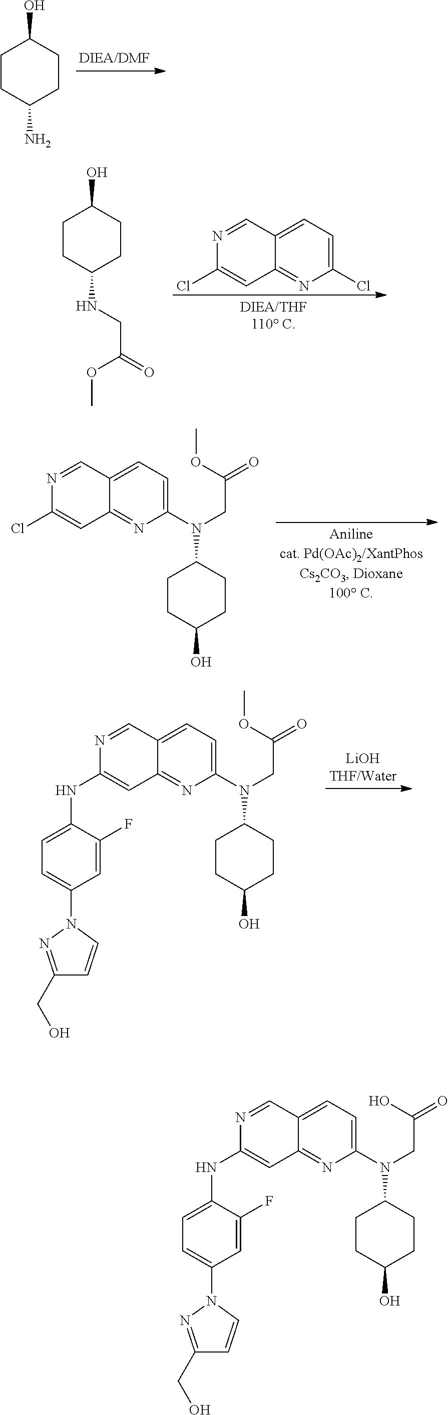 [SMILES: N[C@H]1CC[C@H](O)CC1 ; COC(=O)CN[C@H]1CC[C@H](O)CC1 ; Clc1cc2nc(Cl)ccc2cn1 ; COC(=O)CN(c1ccc2cnc(Cl)cc2n1)[C@H]1CC[C@H](O)CC1 ; COC(=O)CN(c1ccc2cnc(Nc3ccc(-n4ccc(CO)n4)cc3F)cc2n1)[C@H]1CC[C@H](O)CC1 ; O=C(O)CN(c1ccc2cnc(Nc3ccc(-n4ccc(CO)n4)cc3F)cc2n1)[C@H]1CC[C@H](O)CC1]

Methyl 2-[[(1r,4r)-4-hydroxycyclohexyl]amino]acetate. To a stirred mixture of (1r,4r)-4-aminocyclohexan-1-ol (5 g, 43.412 mmol, 1 equiv.) and methyl 2-bromoacetate (6.64 g, 0.043 mmol, 1 equiv.) in DMF (10 mL) was added DIEA (11.22 g, 0.087 mmol, 2 equiv.) at room temperature under nitrogen atmosphere. The resulting mixture was stirred for 16 hours at 110° C. under nitrogen atmosphere. The reaction was monitored by LCMS. The mixture was allowed to cool down to room temperature. The resulting mixture was concentrated under reduced pressure. The residue was purified by silica gel column chromatography, eluted with DCM/MeOH (20:1 to 15:1) to afford methyl 2-[[(1r,4r)-4-hydroxycyclohexyl]amino]acetate (3 g, 37%) as a white solid.

Methyl 2-[(7-chloro-1,6-naphthyridin-2-yl)[(1r,4r)-4-hydroxycyclohexyl]amino]acetate. To a stirred mixture of methyl 2-[[(1r,4r)-4-hydroxycyclohexyl]amino]acetate (2 g, 10.682 mmol, 1 equiv.) and 2,7-dichloro-1,6-naphthyridine (1.06 g, 5.341 mmol, 0.50 equiv.) in THF (2 mL) was added DIEA (1.38 g, 10.678 mmol, 1 equiv.) at room temperature. The resulting mixture was stirred for 16 hours at 110° C. under nitrogen atmosphere. The reaction was monitored by LCMS. The mixture was allowed to cool down to room temperature. The residue was purified by silica gel column chromatography, eluted with PE/EtOAc (20:1 to 5:1) to afford methyl 2-[(7-chloro-1,6-naphthyridin-2-yl)[(1r,4r)-4-hydroxycyclohexyl]amino]acetate (200 mg, 36%) as a yellow oil.

Methyl 2-[[7-([2-fluoro-4-[3-(hydroxymethyl)pyrazol-1-yl]phenyl]amino)-1,6-naphthyridin-2-yl][(1r,4r)-4-hydroxycyclohexyl]amino]acetate. To a stirred mixture of methyl 2-[(7-chloro-1,6-naphthyridin-2-yl)[(1r,4r)-4-hydroxycyclohexyl]amino]acetate (300 mg, 0.858 mmol, 1 equiv.) and [1-(4-amino-3-fluorophenyl)pyrazol-3-yl]methanol (195.47 mg, 0.943 mmol, 1.10 equiv.) in 1,4-dioxane (10 mL) were added XantPhos (148.86 mg, 0.257 mmol, 0.30 equiv.), Cs₂CO₃ (558.84 mg, 1.715 mmol, 2 equiv.) and Pd(OAc)₂ (28.88 mg, 0.129 mmol, 0.15 equiv.) at room temperature under nitrogen atmosphere. The resulting mixture was stirred for 2 hours at 100° C. under nitrogen atmosphere. The reaction was monitored by LCMS. The mixture was allowed to cool down to room temperature. The resulting mixture was filtered, the filter cake was washed with EtOAc (3×10 mL). The filtrate was concentrated under reduced pressure. The residue was purified by reverse phase flash with the following conditions (Column: C18, 330 g; Mobile Phase A:Water/0.05% NH₄HCO₃, Mobile Phase B:ACN; Flow rate:80 mL/min; Gradient: 20% B to 50% B in 20 min; Detector, 254 nm and 220 nm, the desired product were collected at 46% B) to afford methyl 2-[[7-([2-fluoro-4-[3-(hydroxymethyl)pyrazol-1-yl]phenyl]amino)-1,6-naphthyridin-2-yl][(1r,4r)-4-hydroxycyclohexyl]amino]acetate (200 mg, 45%) as a yellow solid.

[[7-([2-fluoro-4-[3-(hydroxymethyl)pyrazol-1-yl]phenyl]amino)-1,6-naphthyridin-2-yl][(1r,4r)-4-hydroxycyclohexyl]amino]acetic acid (Compound 110). To a stirred solution of methyl 2-[[7-([2-fluoro-4-[3-(hydroxymethyl)pyrazol-1-yl]phenyl]amino)-1,6-naphthyridin-2-yl][(1r,4r)-4-hydroxycyclohexyl]amino]acetate (120 mg, 0.231 mmol, 1 equiv.) in THF (10 mL) and H₂O (2 mL) was added LiOH (27.60 mg, 1.153 mmol, 5 equiv.) at room temperature. The resulting mixture was stirred for 16 hours at room temperature. The mixture was acidified to pH 6 with HCl (aq.). The reaction was monitored by LCMS. The resulting mixture was concentrated under reduced pressure. The crude product (100 mg) was purified by Prep-HPLC to afford [[7-([2-fluoro-4-[3-(hydroxymethyl)pyrazol-1-yl]phenyl]amino)-1,6-naphthyridin-2-yl][(1r,4r)-4-hydroxycyclohexyl]amino]acetic acid (71.5 mg, 73%) as a green solid.

### Example 13. Preparation of Compounds 114 and 116

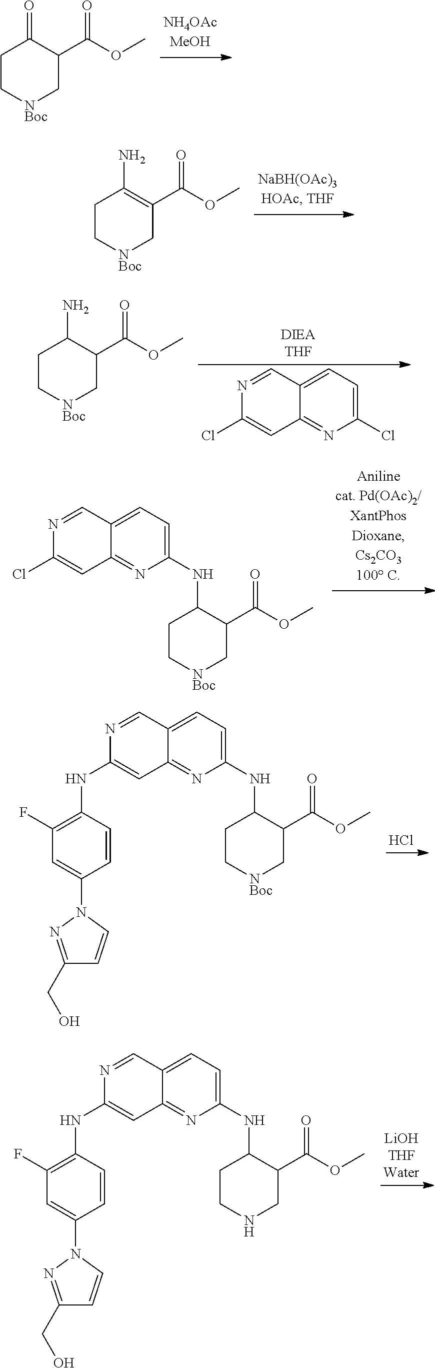

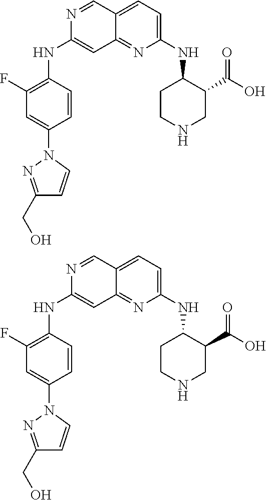 [SMILES: O=C(O)[C@@H]1CNCC[C@H]1Nc1ccc2cnc(Nc3ccc(-n4ccc(CO)n4)cc3F)cc2n1 ; O=C(O)[C@H]1CNCC[C@@H]1Nc1ccc2cnc(Nc3ccc(-n4ccc(CO)n4)cc3F)cc2n1]

1-tert-butyl 3-methyl 4-amino-5,6-dihydro-2H-pyridine-1,3-dicarboxylate. To a stirred solution of 1-tert-butyl 3-methyl 4-oxopiperidine-1,3-dicarboxylate (4 g, 15.547 mmol, 1 equiv.) in MeOH (100 mL) was added NH₄OAc (3.60 g, 46.641 mmol, 3 equiv.) at 0° C. The resulting mixture was stirred for 16 hours at room temperature. The reaction was monitored by TLC. The resulting mixture was extracted with DCM (3×200 mL). The combined organic layers were washed with brine (1×300 mL), dried over anhydrous Na₂SO₄. After filtration, the filtrate was concentrated under reduced pressure. This resulted in 1-tert-butyl 3-methyl 4-amino-5,6-dihydro-2H-pyridine-1,3-dicarboxylate (3.8 g, 95%) as a white solid.

1-tert-butyl 3-methyl 4-aminopiperidine-1,3-dicarboxylate. To a stirred solution of 1-tert-butyl 3-methyl 4-amino-5,6-dihydro-2H-pyridine-1,3-dicarboxylate (3.80 g, 14.826 mmol, 1 equiv.) in THF (35 mL) were added NaBH(OAc)₃ (7.86 g, 37.086 mmol, 2.50 equiv.) and HOAc (10 mL, 174.515 mmol, 11.77 equiv.) at 0° C. The resulting mixture was stirred for 2 hours at room temperature. The reaction was monitored by LCMS. The mixture was basified to pH 8 with saturated NaHCO₃ (aq.). The resulting mixture was extracted with DCM/MeOH (10:1) (3×100 mL). The combined organic layers were washed with brine (1×100 mL), dried over anhydrous Na₂SO₄. After filtration, the filtrate was concentrated under reduced pressure. This resulted in 1-tert-butyl 3-methyl 4-aminopiperidine-1,3-dicarboxylate (1.2 g, 31%) as a white solid.

1-tert-butyl 3-methyl 4-[(7-chloro-1,6-naphthyridin-2-yl)amino]piperidine-1,3-dicarboxylate. To a stirred mixture of 1-tert-butyl 3-methyl 4-aminopiperidine-1,3-dicarboxylate (500 mg, 1.936 mmol, 1 equiv.) and 2,7-dichloro-1,6-naphthyridine (192.62 mg, 0.968 mmol, 0.5 equiv.) in THF (5 mL) was added DIEA (250.16 mg, 1.936 mmol, 1 equiv.) at room temperature. The resulting mixture was stirred for 16 hours at 110° C. The reaction was monitored by LCMS. The mixture was allowed to cool down to room temperature. The crude product was purified by reverse phase flash with the following conditions (Column: C18, 330 g; Mobile Phase A:Water/0.05% TFA, Mobile Phase B:ACN; Flow rate:80 mL/min; Gradient: 20% B to 50% B in 20 min; Detector, 254 nm and 220 nm, the desired product were collected at 50% B) to afford 1-tert-butyl 3-methyl 4-[(7-chloro-1,6-naphthyridin-2-yl)amino]piperidine-1,3-dicarboxylate (500 mg, 61%) as a white solid.

1-tert-butyl 3-methyl 4-[[7-([2-fluoro-4-[3-(hydroxymethyl)pyrazol-1-yl]phenyl]amino)-1,6-naphthyridin-2-yl]amino]piperidine-1,3-dicarboxylate. To a stirred solution of 1-tert-butyl 3-methyl 4-[(7-chloro-1,6-naphthyridin-2-yl)amino]piperidine-1,3-dicarboxylate (0.50 g, 1.19 mmol) and [1-(4-amino-3-fluorophenyl)pyrazol-3-yl]methanol (0.27 g, 1.31 mmol) in 1,4-dioxane (10.0 mL) were added XantPhos (0.21 g, 0.36 mmol), cesium carbonate (0.77 g, 2.38 mmol) and palladium acetate (40.0 mg, 0.18 mmol) at ambient temperature. The reaction mixture was purged with nitrogen for 3 times and stirred under nitrogen atmosphere at 100° C. for 2 hours. The resulting mixture was cooled down to ambient temperature and filtered. The filter cake was washed with ethyl acetate (3×10.0 mL). The filtrate was concentrated under reduced pressure. The residue was purified by reverse phase flash chromatography. The fractions containing the desired product were collected and concentrated under reduced pressure to afford the title compound (0.27 g, 38%) as a yellow solid.

Methyl 4-[[7-([2-fluoro-4-[3-(hydroxymethyl)pyrazol-1-yl]phenyl]amino)-1,6-naphthyridin-2-yl]amino]piperidine-3-carboxylate. To a stirred solution of 1-tert-butyl 3-methyl 4-[[7-([2-fluoro-4-[3-(hydroxymethyl)pyrazol-1-yl]phenyl]amino)-1,6-naphthyridin-2-yl]amino]piperidine-1,3-dicarboxylate (270 mg, 0.456 mmol, 1 equiv.) in MeOH (8 mL) was added HCl (gas) in 1,4-dioxane (2 mL) dropwise at 0° C. The resulting mixture was stirred for 1 hour at room temperature. The reaction was monitored by LCMS. The resulting mixture was concentrated under reduced pressure. The residue was basified to pH 8 with saturated NaHCO₃ (aq.). The resulting mixture was concentrated under reduced pressure. The residue product was purified by reverse phase flash with the following conditions (Column: C18, 330 g; Mobile Phase A:Water/0.05% NH₄HCO₃, Mobile Phase B:ACN; Flow rate:80 mL/min; Gradient: 20% B to 50% B in 20 min; Detector, 254 nm and 220 nm, the desired product were collected at 45% B) to afford methyl 4-[[7-([2-fluoro-4-[3-(hydroxymethyl)pyrazol-1-yl]phenyl]amino)-1,6-naphthyridin-2-yl]amino]piperidine-3-carboxylate (150 mg, 66%) as a white solid. The crude product (150 mg) was purified by Prep-Chiral-HPLC with the following conditions (Column: CHIRALPAK IE, 2*25 cm, 5 μm; Mobile Phase A:MTBE (10 mM NH₃-MEOH)—HPLC, Mobile Phase B:EtOH—HPLC; Flow rate:20 mL/min; Gradient:15 B to 15 B in 20 min; 220/254 nm; RT1:12.224; RT2:14.576) to afford methyl (3S,4S)-4-[[7-([2-fluoro-4-[3-(hydroxymethyl)pyrazol-1-yl]phenyl]amino)-1,6-naphthyridin-2-yl]amino]piperidine-3-carboxylate and methyl (3R,4R)-4-[[7-([2-fluoro-4-[3-(hydroxymethyl)pyrazol-1-yl]phenyl]amino)-1,6-naphthyridin-2-yl]amino]piperidine-3-carboxylate (each of 60 mg).

(3S,4S)-4-[[7-([2-fluoro-4-[3-(hydroxymethyl)pyrazol-1-yl]phenyl]amino)-1,6-naphthyridin-2-yl]amino]piperidine-3-carboxylic acid (Compound 116). To a stirred solution of methyl (3S,4S)-4-[[7-([2-fluoro-4-[3-(hydroxymethyl)pyrazol-1-yl]phenyl]amino)-1,6-naphthyridin-2-yl]amino]piperidine-3-carboxylate (60 mg, 0.122 mmol, 1 equiv.) in THF (2 mL) and H₂O (10 mL) was added LiOH (14.62 mg, 0.610 mmol, 5 equiv.) at room temperature. The resulting mixture was stirred for 2 hours at room temperature. The reaction was monitored by LCMS. The resulting mixture was concentrated under reduced pressure. The crude product (60 mg) was purified by Prep-HPLC to afford (3S,4S)-4-[[7-([2-fluoro-4-[3-(hydroxymethyl)pyrazol-1-yl]phenyl]amino)-1,6-naphthyridin-2-yl]amino]piperidine-3-carboxylic acid (33.8 mg, 57%) as a yellow solid.

(3R,4R)-4-[[7-([2-fluoro-4-[3-(hydroxymethyl)pyrazol-1-yl]phenyl]amino)-1,6-naphthyridin-2-yl]amino]piperidine-3-carboxylic acid (Compound 114). To a stirred solution of methyl (3R,4R)-4-[[7-([2-fluoro-4-[3-(hydroxymethyl)pyrazol-1-yl]phenyl]amino)-1,6-naphthyridin-2-yl]amino]piperidine-3-carboxylate (60 mg, 0.122 mmol, 1 equiv.) in THF (10 mL) and H₂O (2 mL) was added LiOH (14.62 mg, 0.610 mmol, 5 equiv.) at room temperature. The resulting mixture was stirred for 2 hours at room temperature. The reaction was monitored by LCMS. The resulting mixture was concentrated under reduced pressure. The crude product (60 mg) was purified by Prep-HPLC to afford (3R,4R)-4-[[7-([2-fluoro-4-[3-(hydroxymethyl)pyrazol-1-yl]phenyl]amino)-1,6-naphthyridin-2-yl]amino]piperidine-3-carboxylic acid (29.3 mg, 50%) as a yellow solid.

### Example 14. Preparation of Compound 131

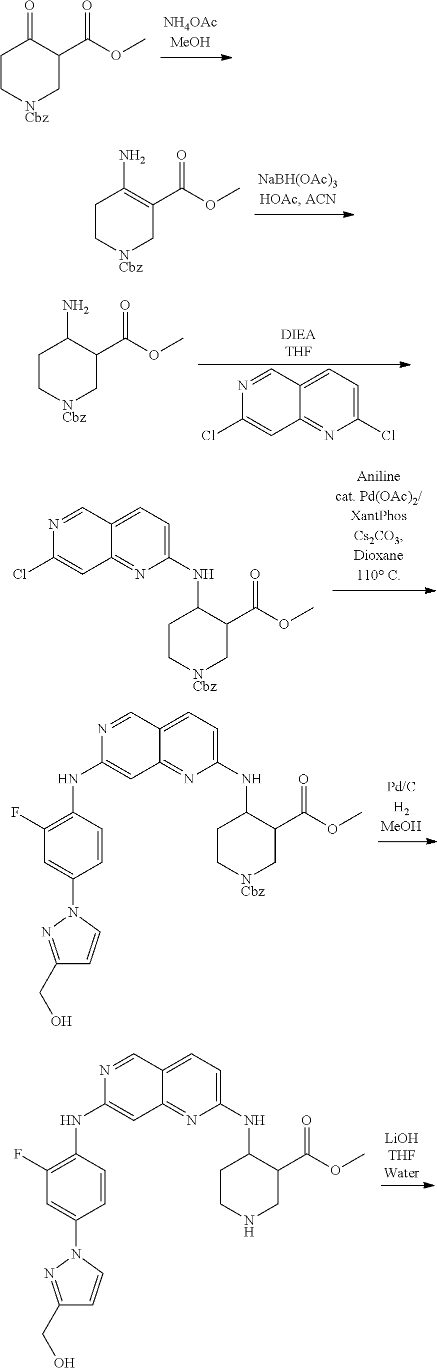

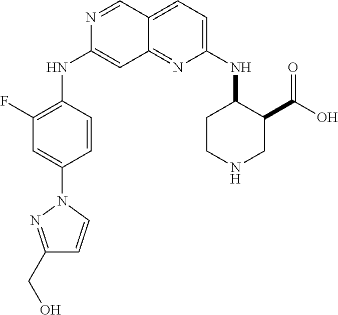

1-benzyl 3-methyl 4-amino-5,6-dihydro-2H-pyridine-1,3-dicarboxylate. To a stirred solution of 1-benzyl 3-methyl 4-oxopiperidine-1,3-dicarboxylate (3 g, 10.299 mmol, 1 equiv.) in MeOH (100 mL) was added NH₄OAc (2.38 g, 30.896 mmol, 3 equiv.) at room temperature. The resulting mixture was stirred for 16 hours at room temperature. The reaction was monitored by LCMS. The resulting mixture was extracted with DCM (3×300 mL). The combined organic layers were washed with brine (1×300 mL), dried over anhydrous Na₂SO₄. After filtration, the filtrate was concentrated under reduced pressure. This resulted in 1-benzyl 3-methyl 4-amino-5,6-dihydro-2H-pyridine-1,3-dicarboxylate (2.8 g, 93%) as a white solid.

1-benzyl 3-methyl 4-aminopiperidine-1,3-dicarboxylate. To a stirred solution of 1-benzyl 3-methyl 4-amino-5,6-dihydro-2H-pyridine-1,3-dicarboxylate (2.80 g, 9.645 mmol, 1 equiv.) in ACN (45 mL) were added NaBH(OAc)₃ (8.18 g, 38.578 mmol, 4 equiv.) and HOAc (30 mL) at 0° C. The resulting mixture was stirred for 16 hours at room temperature. The reaction was monitored by LCMS. The resulting mixture was concentrated under reduced pressure. The residue was purified by reverse phase flash with the following conditions (Column: C18, 330 g; Mobile Phase A:Water/0.05% NH₄HCO₃, Mobile Phase B:ACN; Flow rate:80 mL/min; Gradient: 15% B to 35% B in 20 min; Detector, 220 nm, Monitor, 254 nm, the desired product were collected at 32% B) to afford 1-benzyl 3-methyl 4-aminopiperidine-1,3-dicarboxylate (1.6 g, 56%) as a white solid.

1-benzyl 3-methyl 4-[(7-chloro-1,6-naphthyridin-2-yl)amino]piperidine-1,3-dicarboxylate. To a stirred mixture of 1-benzyl 3-methyl 4-aminopiperidine-1,3-dicarboxylate (800 mg, 2.737 mmol, 1 equiv.) and DIEA (707.37 mg, 5.473 mmol, 2 equiv.) in THF (2 mL) was added 2,7-dichloro-1,6-naphthyridine (653.60 mg, 3.284 mmol, 1.20 equiv.) at room temperature. The resulting mixture was stirred for 16 hours at 110° C. The reaction was monitored by LCMS. The mixture was allowed to cool down to room temperature. The resulting mixture was concentrated under reduced pressure. The residue was purified by reverse phase flash with the following conditions (Column: C18, 330 g; Mobile Phase A:Water/0.05% TFA, Mobile Phase B:ACN; Flow rate:80 mL/min; Gradient: 20% B to 50% B in 20 min; Detector, 254 nm, Monitor, 220 nm, the desired product were collected at 42% B) to afford 1-benzyl 3-methyl 4-[(7-chloro-1,6-naphthyridin-2-yl)amino]piperidine-1,3-dicarboxylate (140 mg, 11%).

1-benzyl 3-methyl 4-[[7-([2-fluoro-4-[3-(hydroxymethyl)pyrazol-1-yl]phenyl]amino)-1,6-naphthyridin-2-yl]amino]piperidine-1,3-dicarboxylate. To a stirred mixture of 1-benzyl 3-methyl 4-[(7-chloro-1,6-naphthyridin-2-yl)amino]piperidine-1,3-dicarboxylate (140 mg, 0.308 mmol, 1 equiv.) and [1-(4-amino-3-fluorophenyl)pyrazol-3-yl]methanol (76.52 mg, 0.369 mmol, 1.20 equiv.) in 1,4-dioxane (4 mL) were added Pd(OAc)₂ (10.36 mg, 0.046 mmol, 0.15 equiv.), XantPhos (53.42 mg, 0.092 mmol, 0.30 equiv.) and Cs₂CO₃ (200.54 mg, 0.616 mmol, 2 equiv.) at room temperature under nitrogen atmosphere. The resulting mixture was stirred for 2 hours at 110° C. under nitrogen atmosphere. The reaction was monitored by LCMS. The mixture was allowed to cool down to room temperature. The resulting mixture was filtered, the filter cake was washed with EtOAc (3×10 mL). The filtrate was concentrated under reduced pressure. The residue was purified by reverse phase flash with the following conditions (Column: C18, 330 g; Mobile Phase A:Water/0.05% TFA, Mobile Phase B:ACN; Flow rate:80 mL/min; Gradient: 30% B to 60% B in 20 min; Detector, 254 nm, Monitor, 220 nm, the desired product were collected at 48% B) to afford 1-benzyl 3-methyl 4-[[7-([2-fluoro-4-[3-(hydroxymethyl)pyrazol-1-yl]phenyl]amino)-1,6-naphthyridin-2-yl]amino]piperidine-1,3-dicarboxylate (110 mg, 57%) as a green solid.

Methyl 4-[[7-([2-fluoro-4-[3-(hydroxymethyl)pyrazol-1-yl]phenyl]amino)-1,6-naphthyridin-2-yl]amino]piperidine-3-carboxylate. To a stirred solution of 1-benzyl 3-methyl 4-[[7-([2-fluoro-4-[3-(hydroxymethyl)pyrazol-1-yl]phenyl]amino)-1,6-naphthyridin-2-yl]amino]piperidine-1,3-dicarboxylate (110 mg, 0.176 mmol, 1 equiv.) in MeOH (10 mL) was added Pd/C (9.36 mg, 0.088 mmol, 0.50 equiv.) at room temperature under hydrogen atmosphere. The resulting mixture was stirred for 2 hours at room temperature under hydrogen atmosphere. The reaction was monitored by LCMS. The resulting mixture was filtered, the filter cake was washed with MeOH (3×20 mL). The filtrate was concentrated under reduced pressure. The crude product was used in the next step directly without further purification.

cis-4-[[7-([2-fluoro-4-[3-(hydroxymethyl)pyrazol-1-yl]phenyl]amino)-1,6-naphthyridin-2-yl]amino]piperidine-3-carboxylic acid (Compound 131). To a stirred solution of methyl 4-[[7-([2-fluoro-4-[3-(hydroxymethyl)pyrazol-1-yl]phenyl]amino)-1,6-naphthyridin-2-yl]amino]piperidine-3-carboxylate (60 mg, 0.122 mmol, 1 equiv.) in THF (5 mL) and H₂O (1 mL) was added LiOH (14.62 mg, 0.610 mmol, 5 equiv.) at room temperature under nitrogen atmosphere. The resulting mixture was stirred for 1 hour at room temperature. The reaction was monitored by LCMS. The resulting mixture was concentrated under reduced pressure. The crude product (30 mg) was purified by Prep-HPLC to afford cis-4-[[7-([2-fluoro-4-[3-(hydroxymethyl)pyrazol-1-yl]phenyl]amino)-1,6-naphthyridin-2-yl]amino]piperidine-3-carboxylic acid (? mg, 12%) as a light yellow solid.

### Example 15. Preparation of Compound 140

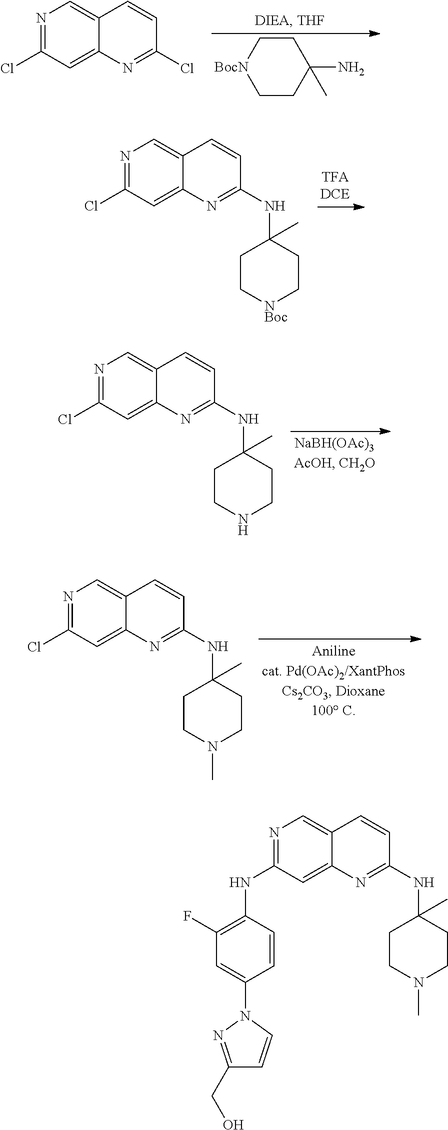 [SMILES: Clc1cc2nc(Cl)ccc2cn1 ; CC1(N)CCN(C(=O)OC(C)(C)C)CC1 ; CC1(Nc2ccc3cnc(Cl)cc3n2)CCN(C(=O)OC(C)(C)C)CC1 ; CC1(Nc2ccc3cnc(Cl)cc3n2)CCNCC1 ; CN1CCC(C)(Nc2ccc3cnc(Cl)cc3n2)CC1 ; CN1CCC(C)(Nc2ccc3cnc(Nc4ccc(-n5ccc(CO)n5)cc4F)cc3n2)CC1]

Tert-butyl 4-[(7-chloro-1,6-naphthyridin-2-yl)amino]-4-methylpiperidine-1-carboxylate. To a stirred mixture of tert-butyl 4-amino-4-methylpiperidine-1-carboxylate (646.06 mg, 3.015 mmol, 1.20 equiv.) and 2,7-dichloro-1,6-naphthyridine (500 mg, 2.512 mmol, 1 equiv.) in THF (2 mL) were added DIEA (974.05 mg, 7.537 mmol, 3 equiv.) in portions at room temperature under nitrogen atmosphere. The resulting mixture was stirred for 16 hours at 110° C. under nitrogen atmosphere. The reaction was monitored by LCMS. The mixture was allowed to cool down to room temperature, and was concentrated under reduced pressure. The residue was purified by reverse phase flash chromatography with the following conditions: Column: Spherical C18, 20-40 um, 330 g; Mobile Phase A: Water (plus 5 mM NH₄NO3); Mobile Phase B: ACN; Flow rate: 80 mL/min; Gradient: 5%-5% B, 10 min, 55% B-95% B gradient in 30 min; Detector: 245 nm. The fractions containing the desired product were collected at 83% B and concentrated under reduced pressure to afford tert-butyl 4-[(7-chloro-1,6-naphthyridin-2-yl)amino]-4-methylpiperidine-1-carboxylate (170 mg, 17%) as a white solid.

7-chloro-N-(4-methylpiperidin-4-yl)-1,6-naphthyridin-2-amine. To a stirred solution of tert-butyl 4-[(7-chloro-1,6-naphthyridin-2-yl)amino]-4-methylpiperidine-1-carboxylate (170 mg, 0.451 mmol, 1 equiv.) in C₁CH₂CH₂C₁ (10 mL) was added TFA (1 mL, 13.463 mmol, 29.85 equiv.) in portions at room temperature. The resulting mixture was stirred for 30 min at room temperature. The reaction was monitored by LCMS. The resulting mixture was concentrated under reduced pressure. The residue was purified by reverse phase flash chromatography with the following conditions: Column: Spherical C18, 20-40 um, 330 g; Mobile Phase A: Water (plus 5 mM NH₄NO₃); Mobile Phase B: ACN; Flow rate: 80 mL/min; Gradient: 5%-5% B, 10 min, 35% B-65% B gradient in 30 min; Detector: 254 nm. The fractions containing the desired product were collected at 59% B and concentrated under reduced pressure to afford as 7-chloro-N-(4-methylpiperidin-4-yl)-1,6-naphthyridin-2-amine (50 mg, 40%) a light brown solid.

7-chloro-N-(1,4-dimethylpiperidin-4-yl)-1,6-naphthyridin-2-amine. To a stirred mixture of 7-chloro-N-(4-methylpiperidin-4-yl)-1,6-naphthyridin-2-amine (50 mg, 0.181 mmol, 1 equiv.) and NaBH(OAc)₃ (76.58 mg, 0.361 mmol, 2 equiv.) in THF (5 mL) were added CH₃COOH (21.70 mg, 0.361 mmol, 2 equiv.) and HCHO (10.85 mg, 0.361 mmol, 2 equiv.) at room temperature under nitrogen atmosphere. The resulting mixture was stirred for 4 hours at 80° C. under nitrogen atmosphere. The reaction was monitored by LCMS. The mixture was allowed to cool down to room temperature. The residue was purified by reverse phase flash chromatography with the following conditions: Column: Spherical C18, 20-40 um, 330 g; Mobile Phase A: Water (plus 5 mM NH₄NO₃); Mobile Phase B: ACN; Flow rate: 80 mL/min; Gradient: 5%-5% B, 10 min, 35% B-75% B gradient in 30 min; Detector: 254 nm. The fractions containing the desired product were collected at 59% B and concentrated under reduced pressure to afford 7-chloro-N-(1,4-dimethylpiperidin-4-yl)-1,6-naphthyridin-2-amine (20 mg, 38%) as a light brown solid.

[1-[4-([2-[(1,4-dimethylpiperidin-4-yl)amino]-1,6-naphthyridin-7-yl]amino)-3-fluorophenyl]pyrazol-3-yl]methanol (Compound 140). To a stirred mixture of 7-chloro-N-(1,4-dimethylpiperidin-4-yl)-1,6-naphthyridin-2-amine (20 mg, 0.069 mmol, 1 equiv.) and [1-(4-amino-3-fluorophenyl)pyrazol-3-yl]methanol (18.53 mg, 0.089 mmol, 1.30 equiv.) in 1,4-dioxane (2 mL) were added XantPhos (11.94 mg, 0.021 mmol, 0.30 equiv.), Cs₂CO₃ (44.82 mg, 0.138 mmol, 2 equiv.) and palladium acetate (3.09 mg, 0.014 mmol) at room temperature under nitrogen atmosphere. The resulting mixture was stirred for 2 hours at 100° C. under nitrogen atmosphere. The reaction was monitored by LCMS. The mixture was allowed to cool down to room temperature. The residue was purified by reverse phase flash chromatography with the following conditions: (Column: Sunfire Prep C₁₈ OBD Column, 10 um, 19*250 mm) to afford [1-[4-([2-[(1,4-dimethylpiperidin-4-yl)amino]-1,6-naphthyridin-7-yl]amino)-3-fluorophenyl]pyrazol-3-yl]methanol (5.1 mg, 14%) as a white solid.

Compounds 313 and 318 were synthesized following the methods and protocols as described for the synthesis of Compound 140, starting with the appropriate materials.

### Example 16. Preparation of Compound 162

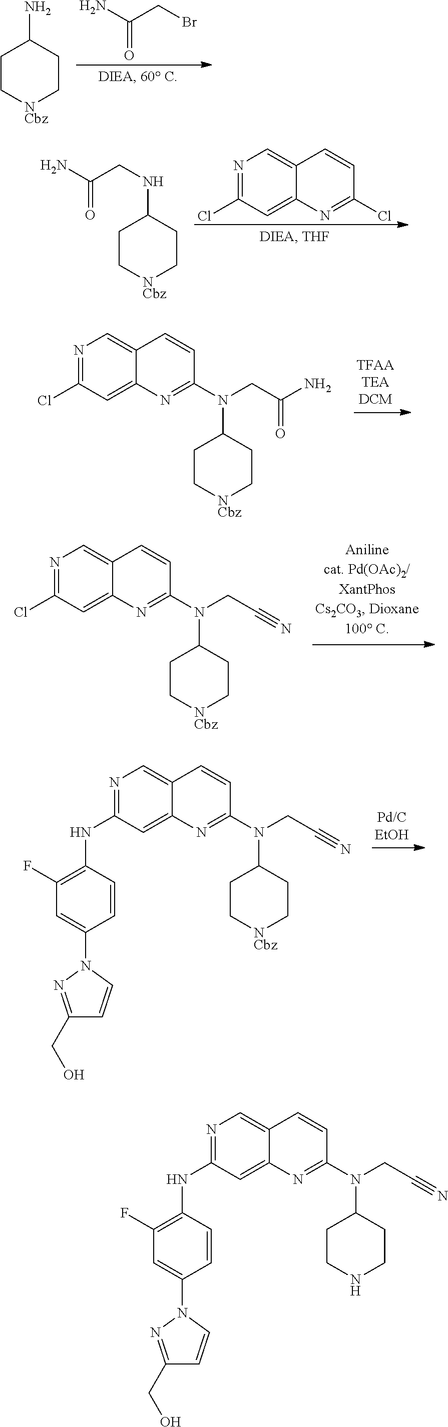

Benzyl 4-[(carbamoylmethyl)amino]piperidine-1-carboxylate: To a stirred solution of benzyl 4-aminopiperidine-1-carboxylate (3.00 g, 12.8 mmol) in N-ethyl-N-isopropylpropan-2-amine (10.0 mL) was added 2-bromoacetamide (1.41 g, 10.2 mmol) at ambient temperature. The reaction mixture was stirred at 60° C. for 16 hours. The resulting mixture was cooled down to ambient temperature and concentrated under reduced pressure. The residue was purified by reverse phase flash chromatography with the following conditions: Column: C₁₈ Column 330 g; Mobile Phase A: Water (plus 5 mM NH₄HCO₃); Mobile Phase B: ACN; Flow rate: 80 mL/min; Gradient: 33% B to 45% B in 20 min; Detector: UV 254/220 nm. The fractions containing the desired product were collected at 40% B and concentrated under reduced pressure to afford the title compound (2.00 g, 53%) as a white solid.

Benzyl 4-[(carbamoylmethyl)(7-chloro-1,6-naphthyridin-2-yl)amino]piperidine-1-carboxylate: To a stirred solution of 2,7-dichloro-1,6-naphthyridine (0.70 g, 3.52 mmol) in N-ethyl-N-isopropylpropan-2-amine (3.00 mL) was added benzyl 4-[(carbamoylmethyl)amino]piperidine-1-carboxylate (1.23 g, 4.22 mmol) at ambient temperature. The reaction mixture was stirred at 100° C. for 48 h. The resulting mixture was cooled down to ambient temperature and concentrated under reduced pressure. The residue was purified by reverse phase flash chromatography with the following conditions: Column: C₁₈ Column 330 g; Mobile Phase A: Water (plus 5 mM NH₄HCO₃); Mobile Phase B: ACN; Flow rate: 80 mL/min; Gradient: 40% B to 70% B in 20 min; Detector: UV 254/220 nm. The fractions containing the desired product were collected at 64% B and concentrated under reduced pressure to afford the title compound (0.70 g, 43%) as a yellow solid.

Benzyl 4-[(7-chloro-1,6-naphthyridin-2-yl)(cyanomethyl)amino]piperidine-1-carboxylate: To a stirred solution of benzyl 4-[(carbamoylmethyl)(7-chloro-1,6-naphthyridin-2-yl)amino]piperidine-1-carboxylate (0.70 g, 1.54 mmol) and trifluoroacetic anhydride (0.65 g, 3.08 mmol) in dichloromethane (20.0 mL) was added triethylamine (0.47 g, 4.63 mmol) at 0° C. The reaction solution was warmed slowly to room temperature and stirred for 2 hours. The resulting mixture was concentrated under reduced pressure. The residue was purified by silica gel column chromatography, eluting with 1-50% of ethyl acetate in petroleum ether to afford the title compound (0.50 g, 74%) as a yellow solid.

Benzyl 4-[(cyanomethyl)[7-([2-fluoro-4-[3-(hydroxymethyl)pyrazol-1-yl]phenyl]amino)-1,6-naphthyridin-2-yl]amino]piperidine-1-carboxylate. To a stirred mixture of benzyl 4-[(7-chloro-1,6-naphthyridin-2-yl)(cyanomethyl)amino]piperidine-1-carboxylate (120 mg, 0.275 mmol, 1 equiv.) and [1-(4-amino-3-fluorophenyl)pyrazol-3-yl]methanol (62.75 mg, 0.303 mmol, 1.10 equiv.) in 1,4-dioxane (10.00 mL) were added Pd(OAc)₂ (9.27 mg, 0.041 mmol, 0.15 equiv.), XantPhos (47.79 mg, 0.083 mmol, 0.30 equiv.) and Cs₂CO₃ (179.39 mg, 0.551 mmol, 2.00 equiv.) in portions at room temperature under nitrogen atmosphere. The resulting mixture was stirred for 2 hours at 100° C. under nitrogen atmosphere. The reaction was monitored by LCMS. The mixture was allowed to cool down to room temperature. The resulting mixture was extracted with EtOAc (3×300 mL). The combined organic layers were washed with brine (2×100 mL), dried over anhydrous Na₂SO₄. After filtration, the filtrate was concentrated under reduced pressure. The residue was purified by reverse phase flash chromatography with the following conditions: Column: Spherical C18, 20-40 um, 120 g; Mobile Phase A: Water (plus 5 mM NH₄HCO₃); Mobile Phase B: ACN; Flow rate: 45 mL/min; Gradient: 5%-5% B, 10 min, 60% B-90% B gradient in 20 min; Detector: 220 nm. The fractions containing the desired product were collected at 85% B and concentrated under reduced pressure to afford benzyl 4-[(cyanomethyl)[7-([2-fluoro-4-[3-(hydroxymethyl)pyrazol-1-yl]phenyl]amino)-1,6-naphthyridin-2-yl]amino]piperidine-1-carboxylate (120 mg, 72%) as a yellow solid.

2-[[7-([2-fluoro-4-[3-(hydroxymethyl)pyrazol-1-yl]phenyl]amino)-1,6-naphthyridin-2-yl](piperidin-4-yl)amino]acetonitrile (Compound 162). To a solution of benzyl 4-[(cyanomethyl)[7-([2-fluoro-4-[3-(hydroxymethyl)pyrazol-1-yl]phenyl]amino)-1,6-naphthyridin-2-yl]amino]piperidine-1-carboxylate (100 mg) in EtOH (40 mL) was added Pd/C (30 mg) under nitrogen atmosphere in a 100 mL round-bottom flask. The mixture was hydrogenated at room temperature for 48 hours under hydrogen atmosphere using a hydrogen balloon, filtered through a Celite pad and concentrated under reduced pressure. The reaction was monitored by LCMS. The residue was purified by Prep-HPLC to afford 2-[[7-([2-fluoro-4-[3-(hydroxymethyl)pyrazol-1-yl]phenyl]amino)-1,6-naphthyridin-2-yl](piperidin-4-yl)amino]acetonitrile (7.8 mg) as a yellow solid.

### Example 17. Preparation of Compounds 287 and 309

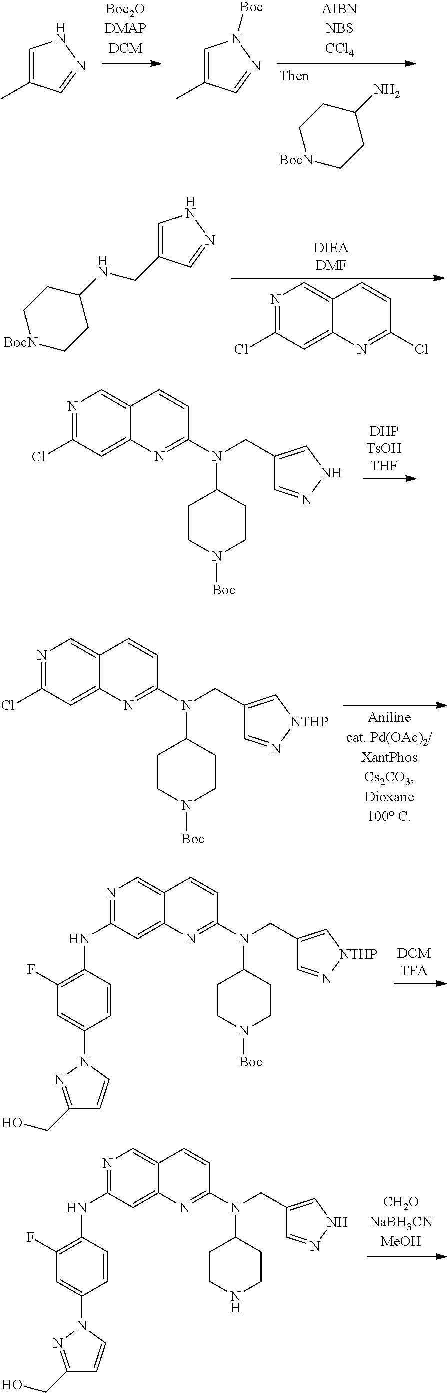

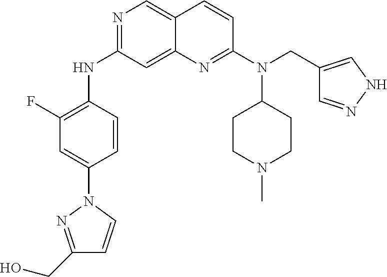 [SMILES: CN1CCC(N(Cc2cn[nH]c2)c2ccc3cnc(Nc4ccc(-n5ccc(CO)n5)cc4F)cc3n2)CC1]

Tert-butyl 4-methylpyrazole-1-carboxylate. To a stirred solution of fomepizole (2.50 g, 30.448 mmol, 1 equiv.) and di-tert-butyl dicarbonate (7.31 g, 33.5 mmol) in DCM (40 mL) was added DMAP (371.98 mg, 3.045 mmol, 0.10 equiv.) at room temperature. The resulting mixture was stirred for 1 hour at room temperature. The resulting mixture was diluted with DCM (100 mL), the resulting mixture was washed with diluted hydrochloric acid (100 mL, 0.5 N), water (100 mL), saturated brine (100 mL), dried over anhydrous sodium sulfate. After filtration, the filtrate was concentrated under reduced pressure to afford tert-butyl 4-methylpyrazole-1-carboxylate (5.20 g, 93%) as a brown oil.

Tert-butyl 4-[(1H-pyrazol-4-ylmethyl)amino]piperidine-1-carboxylate. To a stirred solution of tert-butyl 4-methylpyrazole-1-carboxylate (4.00 g, 21.9 mmol) in carbon tetrachloride (80 mL) were added AIBN (0.36 g, 2.19 mmol) and NBS (4.30 g, 24.1 mmol) at ambient temperature. The reaction mixture was stirred at 70° C. for 16 hours. The resulting mixture was cooled down to ambient temperature. To the above mixture was added tert-butyl 4-aminopiperidine-1-carboxylate (6.59 g, 32.9 mmol) and N-ethyl-N-isopropylpropan-2-amine (8.50 g, 65.9 mmol). The resulting mixture was stirred at ambient temperature for additional 16 h. The resulting mixture was concentrated under reduced pressure. The residue was purified by reverse phase flash chromatography with the following conditions: Column: C₁₈ Column 330 g; Mobile Phase A: Water (plus 0.05% FA); Mobile Phase B: ACN; Flow rate: 45 mL/min; Gradient: 10% B to 35% B in 20 min; Detector: UV 254/220 nm. The fractions containing the desired product were collected at 30% B and concentrated under reduced pressure to afford tert-butyl 4-[(1H-pyrazol-4-ylmethyl)amino]piperidine-1-carboxylate (2.99 g, 36%) as a yellow solid.

Tert-butyl 4-[(7-chloro-1,6-naphthyridin-2-yl) (1H-pyrazol-4-ylmethyl)amino]piperidine-1-carboxylate. A mixture of tert-butyl 4-[(1H-pyrazol-4-ylmethyl)amino]piperidine-1-carboxylate (1 g, 3.567 mmol, 1 equiv.), 2,7-dichloro-1,6-naphthyridine (0.71 g, 3.567 mmol, 1 equiv.) and DIEA (1.38 g, 10.700 mmol, 3 equiv.) in DMF (20 mL) was stirred for 16 hours at ° C. under nitrogen atmosphere. The resulting mixture was purified by reverse phase flash with the following conditions (Column: Spherical C18, 20˜040 um, 330 g; Mobile Phase A: Water (10 mM NH₄HCO₃), Mobile Phase B: ACN; Flow rate: 85 mL/min; Gradient (B %): 5%, 5 min; 5%-35%, 15 min; 35%˜75%, 18 min, 75%-95% 15 min; 95%, 5 min; Detector: 220 nm. The fractions containing desired product were collected and concentrated under reduced pressure to afford tert-butyl 4-[(7-chloro-1,6-naphthyridin-2-yl) (1H-pyrazol-4-ylmethyl)amino]piperidine-1-carboxylate (245 mg, 15%) as a light yellow solid.

Tert-butyl 4-[(7-chloro-1,6-naphthyridin-2-yl)([[1-(oxan-2-yl)pyrazol-4-yl]methyl])amino]piperidine-1-carboxylate. To a stirred solution of tert-butyl 4-[(7-chloro-1,6-naphthyridin-2-yl) (1H-pyrazol-4-ylmethyl)amino]piperidine-1-carboxylate (225 mg, 0.508 mmol, 1 equiv.) and 3,4-dihydro-2H-pyran (0.85 g, 10.2 mmol) in THF (10 mL) was added p-Toluenesulfonic acid (17.49 mg, 0.102 mmol, 0.20 equiv.) at room temperature. The resulting mixture was stirred for 16 hours at 60° C. under nitrogen atmosphere. The resulting mixture was concentrated under reduced pressure. The residue was purified by silica gel column chromatography, eluted with DCM/MeOH (100:1 to 30:1) to afford tert-butyl 4-[(7-chloro-1,6-naphthyridin-2-yl)([[1-(oxan-2-yl)pyrazol-4-yl]methyl])amino]piperidine-1-carboxylate (165 mg, 61%) as a light yellow solid.

Tert-butyl 4-[[7-([2-fluoro-4-[3-(hydroxymethyl)pyrazol-1-yl]phenyl]amino)-1,6-naphthyridin-2-yl]([[1-(oxan-2-yl)pyrazol-4-yl]methyl])amino]piperidine-1-carboxylate. To a stirred solution of tert-butyl 4-[(7-chloro-1,6-naphthyridin-2-yl)([[1-(oxan-2-yl)pyrazol-4-yl]methyl])amino]piperidine-1-carboxylate (50.0 mg, 0.095 mmol) and [1-(4-amino-3-fluorophenyl)pyrazol-3-yl]methanol (21.6 mg, 0.11 mmol) in 1,4-dioxane (3 mL) were added palladium acetate (3.19 mg, 0.014 mmol), XantPhos (16.5 mg, 0.028 mmol) and cesium carbonate (61.8 mg, 0.19 mmol) at ambient temperature. The reaction mixture was purged with nitrogen for 3 times and stirred under nitrogen atmosphere at 100° C. for 2 h. The resulting mixture was cooled down to ambient temperature, diluted with water (20.0 mL) and extracted with dichloromethane (3×20.0 mL). The combined organic layers was washed with brine (3×10.0 mL), dried over anhydrous sodium sulfate and filtered. The filtrate was concentrated under reduced pressure. The residue was purified by prep-TLC (dichloromethane:methanol=15:1) to afford the title compound (50.0 mg, 62% purity on LCMS, 75% yield) as a yellow solid which was used in the next step directly without further purification

[1-[3-fluoro-4-([2-[piperidin-4-yl(1H-pyrazol-4-ylmethyl)amino]-1,6-naphthyridin-7-yl]amino)phenyl]pyrazol-3-yl]methanol (Compound 287). To a stirred solution of tert-butyl[7-([2-fluoro-4-[3-(hydroxymethyl)pyrazol-1-yl]phenyl]amino)-1,6-naphthyridin-2-yl]([[1-(oxan-2-yl)pyrazol-4-yl]methyl)amino]piperidine-1-carboxylate (12 mg) in DCM (4 mL) was added TFA (0.5 mL) dropwise at rt. The reaction mixture was stirred for 2 hours at rt. The reaction was monitored by LCMS. The resulting mixture was concentrated under reduced pressure. The residue was basified to pH=8 with saturated NaHCO₃ (aq.). The resulting mixture was extracted with EtOAc (2×50 mL). The combined organic layers were washed with brine (1×50 mL), dried over anhydrous Na₂SO₄. After filtration, the filtrate was concentrated under reduced pressure. The residue was purified by Prep-HPLC to afford [1-[3-fluoro-4-([2-[piperidin-4-yl(1H-pyrazol-4-ylmethyl)amino]-1,6-naphthyridin-7-yl]amino)phenyl]pyrazol-3-yl]methanol (3.4 mg) as a white solid. [1-[3-fluoro-4-([2-[(1-methylpiperidin-4-yl) (1H-pyrazol-4-ylmethyl)amino]-1,6-naphthyridin-7-yl]amino)phenyl]pyrazol-3-yl]methanol (Compound 309). To a stirred mixture of [1-[3-fluoro-4-([2-[piperidin-4-yl(1H-pyrazol-4-ylmethyl)amino]-1,6-naphthyridin-7-yl]amino)phenyl]pyrazol-3-yl]methanol (4 mg, 08 mmol, 1 equiv.) and HCHO (0.01 mL, 0.333 mmol, 35.06 equiv.) in MeOH (5 mL) was added NaBH₃CN (0.73 mg, 0.012 mmol, 1.49 equiv.) dropwise at 0° C. The reaction mixture was stirred for 2 hours at rt. The reaction was monitored by LCMS. The residue was basified to pH=8 with saturated NaHCO₃ (aq.). The resulting mixture was concentrated under reduced pressure. The residue was purified by Prep-HPLC to afford [1-[3-fluoro-4-([2-[(1-methylpiperidin-4-yl) (1H-pyrazol-4-ylmethyl)amino]-1,6-naphthyridin-7-yl]amino)phenyl]pyrazol-3-yl]methanol (1.4 mg, 34%) as a white solid.

### Example 18. Preparation of Compound 124

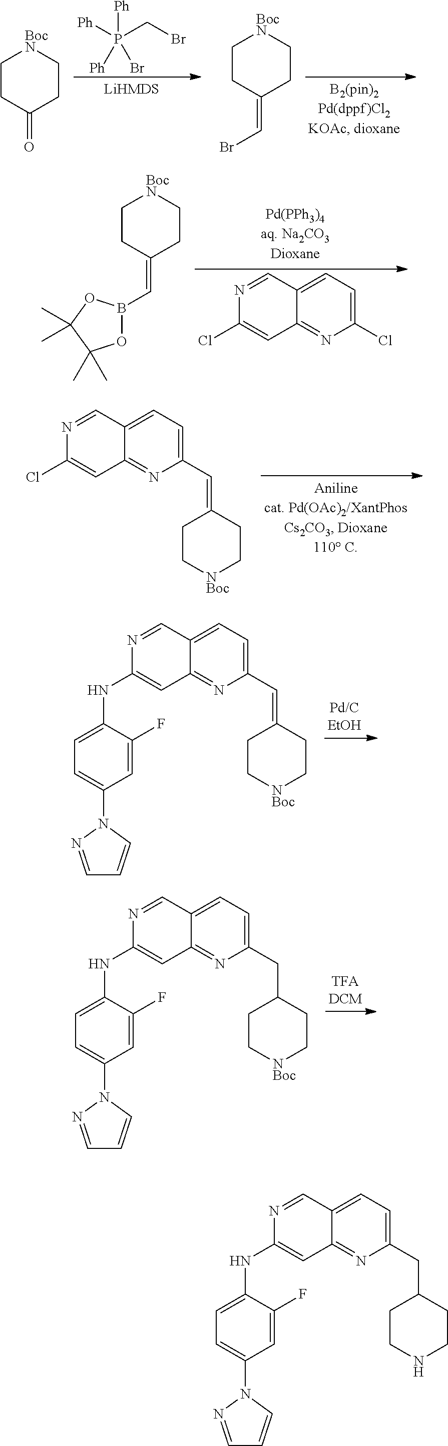

Tert-butyl 4-(bromomethylidene)piperidine-1-carboxylate. To a stirred solution/mixture of (bromomethyl)triphenylphosphanium bromide (15321.66 mg, 35.132 mmol, 1.4 equiv.) in THF (50 mL) was added LiHMDS (7558.01 mg, 45.169 mmol, 1.8 equiv.) dropwise/in portions at −20° C. under nitrogen atmosphere. The resulting mixture was stirred for 1 hour at −20° C. under nitrogen atmosphere. To the above mixture was added tert-butyl 4-oxopiperidine-1-carboxylate (5 g, 25.094 mmol, 1 equiv.) dropwise at −20° C. The resulting mixture was stirred for additional 16 hours at room temperature. The reaction was monitored by LCMS. The reaction was quenched with sat. NH₄C₁ (aq.) at room temperature. The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EtOAc (20:1 to 5:1) to afford tert-butyl 4-(bromomethylidene)piperidine-1-carboxylate (2.5 g, 36%) as an off-white solid.

Tert-butyl 4-[(4,4,5,5-tetramethyl-1,3,2-dioxaborolan-2-yl)methylidene]piperidine-1-carboxylate. To a stirred solution/mixture of tert-butyl 4-(bromomethylidene)piperidine-1-carboxylate (2.50 g, 9.052 mmol, 1 equiv.) and bis(pinacolato)diboron (3.45 g, 13.578 mmol, 1.5 equiv.) in 1,4-dioxane were added Pd(dppf)C₁₂ (0.66 g, 0.905 mmol, 0.10 equiv.) and KOAc (2.67 g, 27.157 mmol, 3 equiv.) at room temperature under nitrogen atmosphere. The resulting mixture was stirred for 16 hours at 80° C. under nitrogen atmosphere. The reaction was quenched with Water at room temperature. The resulting mixture was extracted with EtOAc (3×30 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EtOAc (5:1) to afford tert-butyl 4-[(4,4,5,5-tetramethyl-1,3,2-dioxaborolan-2-yl)methylidene]piperidine-1-carboxylate (1.2 g, 41%) as an off-white solid.

Tert-butyl 4-[(7-chloro-1,6-naphthyridin-2-yl)methylidene]piperidine-1-carboxylate. A mixture of 2,7-dichloro-1,6-naphthyridine (1.98 g, 9.948 mmol, 1 equiv.), tert-butyl 4-[(4,4,5,5-tetramethyl-1,3,2-dioxaborolan-2-yl)methylidene]piperidine-1-carboxylate (6.43 g, 19.896 mmol, 2 equiv.), Pd(PPh₃)₄ (2.30 g, 1.990 mmol, 0.20 equiv.) in dioxane (100 mL) and Na₂CO₃ (2.11 g, 19.896 mmol, 2 equiv.) in H₂O (10 mL, 555.084 mmol, 55.80 equiv.) and this resulting mixture was stirred at 100° C. for 40 hours under N₂ atmosphere. The reaction mixture was cooled down to room temperature and added H₂O (100 mL). The resulting mixture was extracted with EtOAc (3×100 mL). The combined organic layers were washed with brine (2×100 mL), dried over anhydrous Na₂SO₄. After filtration, the filtrate was concentrated under reduced pressure to give the residue, which was purified by silica gel column chromatography, eluted with PE:EtOAc (4:1 to 2:1) to afford tert-butyl 4-[(7-chloro-1,6-naphthyridin-2-yl)methylidene]piperidine-1-carboxylate (2.0 g, 65% purity, 36%) as a light yellow oil.

Tert-butyl 4-[(7-[[2-fluoro-4-(pyrazol-1-yl)phenyl]amino]-1,6-naphthyridin-2-yl)methylidene]piperidine-1-carboxylate. To a stirred solution/mixture of tert-butyl 4-[(7-chloro-1,6-naphthyridin-2-yl)methylidene]piperidine-1-carboxylate (100 mg, 0.278 mmol, 1 equiv.) and 2-fluoro-4-(pyrazol-1-yl)aniline (54.16 mg, 0.306 mmol, 1.10 equiv.) in 1,4-dioxane were added Pd(OAc)₂ (9.36 mg, 0.042 mmol, 0.15 equiv.) and XantPhos (48.24 mg, 0.083 mmol, 0.3 equiv.) and Cs₂CO₃ (181.09 mg, 0.556 mmol, 2 equiv.) at room temperature under nitrogen atmosphere. The resulting mixture was stirred for 2 hours at 110° C. under nitrogen atmosphere. The reaction was quenched with Water at room temperature. The resulting mixture was extracted with EtOAc (3×10 mL). The combined organic layers were washed with brine (2×10 mL), dried over anhydrous Na₂SO₄. After filtration, the filtrate was concentrated under reduced pressure. The residue was purified by silica gel column chromatography, eluted with DCM/MeOH (10:1) to afford tert-butyl 4-[(7-[[2-fluoro-4-(pyrazol-1-yl)phenyl]amino]-1,6-naphthyridin-2-yl)methylidene]piperidine-1-carboxylate (80 mg, 57%) as a light brown solid.

Tert-butyl 4-[(7-[[2-fluoro-4-(pyrazol-1-yl)phenyl]amino]-1,6-naphthyridin-2-yl)methyl]piperidine-1-carboxylate. To a stirred solution/mixture of tert-butyl 4-[(7-[[2-fluoro-4-(pyrazol-1-yl)phenyl]amino]-1,6-naphthyridin-2-yl)methylidene]piperidine-1-carboxylate (80 mg, 0.160 mmol, 1 equiv.) in ethanol was added Pd/C (1.70 mg, 0.016 mmol, 0.1 equiv.) at room temperature under hydrogen atmosphere. The resulting mixture was stirred for 16 hours at room temperature under hydrogen atmosphere. Then the resulting mixture was stirred for 4 hours at 50° C. under hydrogen atmosphere. The resulting mixture was filtered, the filter cake was washed with ethanol (3×10 mL). The filtrate was concentrated under reduced pressure to afford tert-butyl 4-[(7-[[2-fluoro-4-(pyrazol-1-yl)phenyl]amino]-1,6-naphthyridin-2-yl)methyl]piperidine-1-carboxylate (80 mg, crude) as a brown oil.

N-[2-fluoro-4-(pyrazol-1-yl)phenyl]-2-(piperidin-4-ylmethyl)-1,6-naphthyridin-7-amine (Compound 124). To a stirred solution/mixture of tert-butyl 4-[(7-[[2-fluoro-4-(pyrazol-1-yl)phenyl]amino]-1,6-naphthyridin-2-yl)methyl]piperidine-1-carboxylate (80 mg) and in DCM was added TFA (0.50 mL) at room temperature. The resulting mixture was stirred for 4 hours at room temperature. The resulting mixture was concentrated under reduced pressure. The crude product (40 mg) was purified by Prep-HPLC to afford N-[2-fluoro-4-(pyrazol-1-yl)phenyl]-2-(piperidin-4-ylmethyl)-1,6-naphthyridin-7-amine; trifluoroacetic acid (9 mg) as a red solid.

### Example 19. Preparation of Compound 130

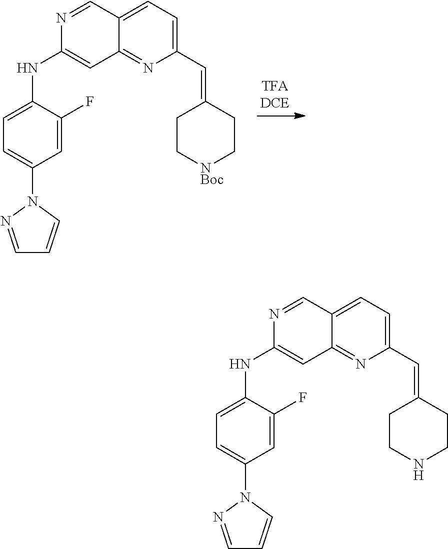 [SMILES: CC(C)(C)OC(=O)N1CCC(=Cc2ccc3cnc(Nc4ccc(-n5cccn5)cc4F)cc3n2)CC1 ; Fc1cc(-n2cccn2)ccc1Nc1cc2nc(C=C3CCNCC3)ccc2cn1]

To a stirred solution of tert-butyl 4-[(7-[[2-fluoro-4-(pyrazol-1-yl)phenyl]amino]-1,6-naphthyridin-2-yl)methylidene]piperidine-1-carboxylate (100 mg, 0.200 mmol, 1 equiv.) in C₁CH₂CH₂C₁ (6 mL) was added TFA (0.60 mL, 8.078 mmol, 40.44 equiv.) in portions at room temperature. The resulting mixture was stirred for 30 min at room temperature. The reaction was monitored by LCMS. The resulting mixture was concentrated under reduced pressure. The residue was purified by Prep-HPLC to afford N-[2-fluoro-4-(pyrazol-1-yl)phenyl]-2-(piperidin-4-ylidenemethyl)-1,6-naphthyridin-7-amine (3.1 mg, 3%) as a yellow solid.

### Example 20. Preparation of Compound 150

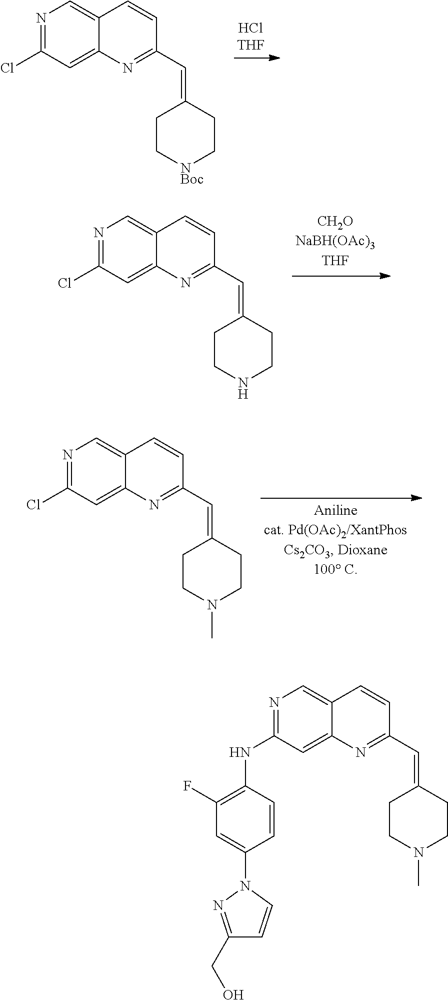 [SMILES: CC(C)(C)OC(=O)N1CCC(=Cc2ccc3cnc(Cl)cc3n2)CC1 ; Clc1cc2nc(C=C3CCNCC3)ccc2cn1 ; CN1CCC(=Cc2ccc3cnc(Cl)cc3n2)CC1 ; CN1CCC(=Cc2ccc3cnc(Nc4ccc(-n5ccc(CO)n5)cc4F)cc3n2)CC1]

7-chloro-2-(piperidin-4-ylidenemethyl)-1,6-naphthyridine. To a stirred solution of tert-butyl 4-[(7-chloro-1,6-naphthyridin-2-yl)methylidene]piperidine-1-carboxylate (Compound 124 step 3, 200 mg) in THF (5 mL) was added HCl (gas) in 1,4-dioxane (5 mL) in portions at room temperature under nitrogen atmosphere. The resulting mixture was stirred for 1 hour at room temperature. The reaction was monitored by LCMS. The resulting mixture was concentrated under reduced pressure. The residue was purified by reverse phase flash chromatography with the following conditions: Column: Spherical C18, 20-40 um, 330 g; Mobile Phase A: Water (0.5% TFA); Mobile Phase B: ACN; Flow rate: 85 mL/min; Gradient: 5%-5% B, 10 min, 33% B—45% B gradient in 20 min; Detector: 254 nm. The fractions containing the desired product were collected at 40% B and concentrated under reduced pressure to afford 7-chloro-2-(piperidin-4-ylidenemethyl)-1,6-naphthyridine (90 mg, 62%) as a yellow solid.

7-chloro-2-[(1-methylpiperidin-4-ylidene)methyl]-1,6-naphthyridine. To a stirred solution of 7-chloro-2-(piperidin-4-ylidenemethyl)-1,6-naphthyridine (300 mg, 1.155 mmol, 1 equiv.) and HCHO (52.02 mg, 1.733 mmol, 1.5 equiv.) in THF (30 mL, 370.290 mmol, 320.60 equiv.) was added NaBH(OAc)₃ (367.19 mg, 1.733 mmol, 1.5 equiv.) in portions at room temperature under nitrogen atmosphere. The resulting mixture was stirred for 1 hour at room temperature. The reaction was monitored by LCMS. The resulting mixture was concentrated under reduced pressure. The residue was purified by reverse phase flash chromatography with the following conditions: Column: Spherical C18, 20-40 um, 330 g; Mobile Phase A: Water (0.5% TFA); Mobile Phase B: ACN; Flow rate: 85 mL/min; Gradient: 5%-5% B, 10 min, 33% B-45% B gradient in 20 min; Detector: 254 nm. The fractions containing the desired product were collected at 40% B and concentrated under reduced pressure to afford 7-chloro-2-[(1-methylpiperidin-4-ylidene)methyl]-1,6-naphthyridine (130 mg, 41%) as a yellow solid.

[1-[3-fluoro-4-([2-[(1-methylpiperidin-4-ylidene)methyl]-1,6-naphthyridin-7-yl]amino)phenyl]pyrazol-3-yl]methanol. To a stirred solution of 7-chloro-2-[(1-methylpiperidin-4-ylidene)methyl]-1,6-naphthyridine (54 mg, 0.197 mmol, 1 equiv.), [1-(4-amino-3-fluorophenyl)pyrazol-3-yl]methanol (44.96 mg, 0.217 mmol, 1.10 equiv.), XantPhos (34.24 mg, 0.059 mmol, 0.3 equiv.) and Cs₂CO₃ (128.54 mg, 0.395 mmol, 2.0 equiv.) in 1,4-dioxane (20 mL) was added Pd(OAc)₂ (6.64 mg, 0.030 mmol, 0.15 equiv.) dropwise at room temperature under nitrogen atmosphere. The resulting mixture was stirred for 2 hours at 100° C. under nitrogen atmosphere. The reaction was monitored by LCMS. The mixture was allowed to cool down to room temperature. The resulting mixture was filtered, the filter cake was washed with ethyl acetate (3×20 mL). The filtrate was concentrated under reduced pressure. The residue was purified by reverse phase flash chromatography with the following conditions: Column: Spherical C18, 20-40 um, 330 g; Mobile Phase A: Water (10 mM NH₄CO₃); Mobile Phase B: ACN; Flow rate: 85 mL/min; Gradient: 5%-5% B, 10 min, 33% B-45% B gradient in 20 min; Detector: 254 nm. The fractions containing the desired product were collected at 40% B and concentrated under reduced pressure to afford [1-[3-fluoro-4-([2-[(1-methylpiperidin-4-ylidene)methyl]-1,6-naphthyridin-7-yl]amino)phenyl]pyrazol-3-yl]methanol (5.8 mg, 6%) as a yellow solid

### Example 21. Preparation of Compounds 209 and 240

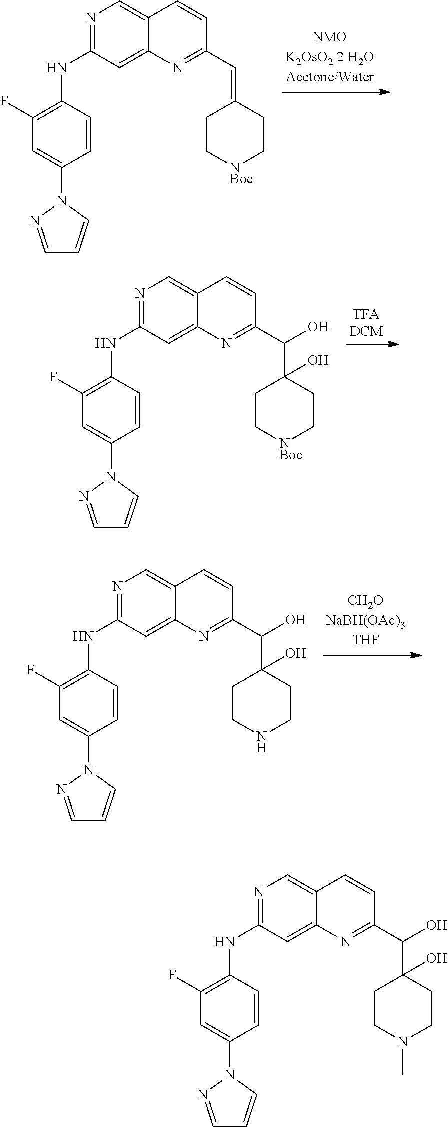

Tert-butyl 4-[(7-[[2-fluoro-4-(pyrazol-1-yl)phenyl]amino]-1,6-naphthyridin-2-yl) (hydroxy)methyl]-4-hydroxypiperidine-1-carboxylate. To a stirred mixture of tert-butyl 4-[(7-[[2-fluoro-4-(pyrazol-1-yl)phenyl]amino]-1,6-naphthyridin-2-yl)methy dene]piperidine-1-carboxylate (Compound 124 step 4, 25 mg, 1 equiv.) and NMO (15 mg, 2.50 equiv.) in acetone (1.50 mL) were added K₂OsO₄.2H₂O (2 mg, 0.10 equiv.) and H₂O (0.15 mL) at room temperature under nitrogen atmosphere. The resulting mixture was stirred for 2 hours at room temperature under nitrogen atmosphere. The reaction was monitored by LCMS. The mixture was allowed to cool down to room temperature. The resulting mixture was filtered, the filter cake was washed with EtOAc (3×10 mL). The filtrate was concentrated under reduced pressure. The residue was purified by Prep-TLC (DCM/MeOH 100:1) to afford tert-butyl 4-[(7-[[2-fluoro-4-(pyrazol-1-yl)phenyl]amino]-1,6-naphthyridin-2-yl) (hydroxy)methyl]-4-hydroxypiperidine-1-carboxylate (26 mg) as a yellow solid.

4-[(7-[[2-fluoro-4-(pyrazol-1-yl)phenyl]amino]-1,6-naphthyridin-2-yl) (hydroxy)methyl]piperidin-4-ol (Compound 209). To a stirred solution of tert-butyl 4-[(7-[[2-fluoro-4-(pyrazol-1-yl)phenyl]amino]-1,6-naphthyridin-2-yl) (hydroxy)methyl]-4-hydroxypiperidine-1-carboxylate (110 mg, 1 equiv.) in DCM (10 mL) was added TFA (1 mL) at 0° C. The resulting mixture was stirred for 1 hour at 0° C. The reaction was monitored by LCMS. The mixture was basified to pH 8 with saturated NaHCO₃ (aq.). The resulting solid was dried in an oven under reduced pressure to afford 4-[(7-[[2-fluoro-4-(pyrazol-1-yl)phenyl]amino]-1,6-naphthyridin-2-yl) (hydroxy)methyl]piperidin-4-ol (6.7 mg) as a yellow solid.

4-[(7-[[2-fluoro-4-(pyrazol-1-yl)phenyl]amino]-1,6-naphthyridin-2-yl) (hydroxy)methyl]-1-methylpiperidin-4-ol (Compound 240). To a stirred mixture of 4-[(7-[[2-fluoro-4-(pyrazol-1-yl)phenyl]amino]-1,6-naphthyridin-2-yl) (hydroxy)methyl]piperidin-4-ol (82 mg, 3.286 mmol, 1 equiv.) and HCHO (27.30 mg, 4.272 mmol, 2 equiv.) in DCM (5 mL) was added NaBH(OAc)₃ (53.40 mg, 0.329 mmol, 1.50 equiv.) at room temperature. The resulting mixture was stirred for 1 hour at room temperature. The reaction was monitored by LCMS. The resulting mixture was concentrated under reduced pressure. The residue was purified by Prep-HPLC to afford 4-[(7-[[2-fluoro-4-(pyrazol-1-yl)phenyl]amino]-1,6-naphthyridin-2-yl) (hydroxy)methyl]-1-methylpiperidin-4-ol (27.3 mg) as a light yellow solid.

### Example 22. Preparation of Compound 164

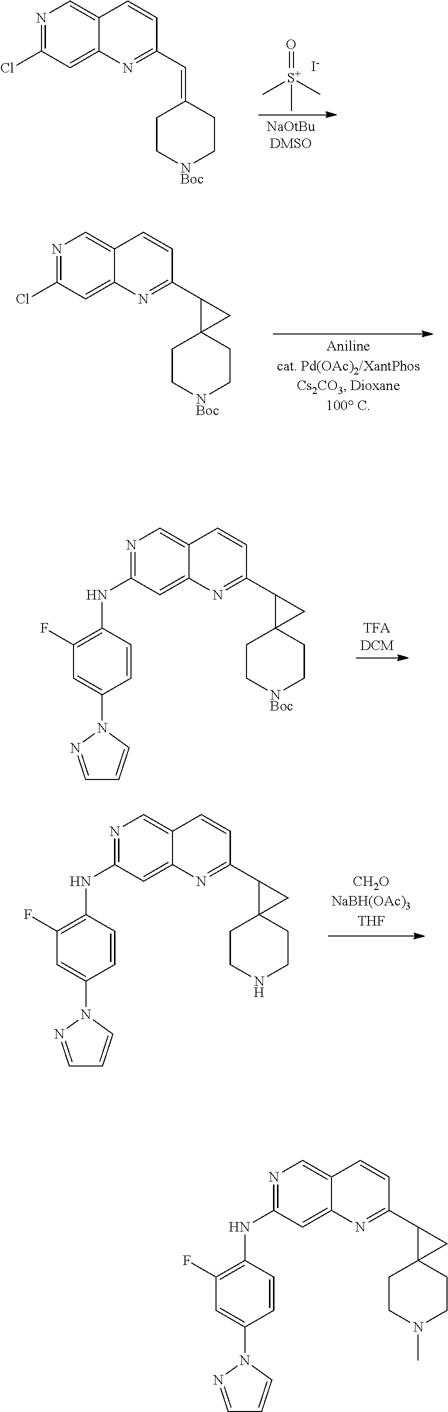

Tert-butyl 1-(7-chloro-1,6-naphthyridin-2-yl)-6-azaspiro[2.5]octane-6-carboxylate. To a solution of tert-butyl 4-[(7-chloro-1,6-naphthyridin-2-yl)methylidene]piperidine-1-carboxylate (Compound 124 step 3, 430 mg, 1.195 mmol, 1 equiv.) in DMSO (40 mL) was added dropwise t-BuONa (172.26 mg, 1.792 mmol, 1.50 equiv.) and trimethyl(oxo)-1\hat{ }[6]-sulfanylium iodide (394.46 mg, 1.792 mmol, 1.50 equiv.) in DMSO (20 mL) at 0° C. under N₂ atmosphere. This resulting mixture was warmed to 70° C. and stirred at 70° C. for 16 hours. The reaction mixture was cooled down to room temperature and added DCM (100 mL) and H₂O (100 mL). The resulting mixture was extracted with DCM (5×100 mL). The combined organic layers were washed with brine (3×100 mL), dried over anhydrous Na₂SO₄. After filtration, the filtrate was concentrated under reduced pressure to give the residue, which was purified by silica gel column chromatography, eluted with PE:EtOAc (4:1) to afford tert-butyl 1-(7-chloro-1,6-naphthyridin-2-yl)-6-azaspiro[2.5]octane-6-carboxylate (288 mg, 64%) as a light yellow semi-solid.

Tert-butyl 1-(7-[[2-fluoro-4-(pyrazol-1-yl)phenyl]amino]-1,6-naphthyridin-2-yl)-6-azaspiro[2.5]octane-6-carboxylate. In a 5 mL microwave vial was charged with tert-butyl 1-(7-chloro-1,6-naphthyridin-2-yl)-6-azaspiro[2.5]octane-6-carboxylate (37.40 mg, 0.100 mmol, 1 equiv.), 2-fluoro-4-(pyrazol-1-yl)aniline (17.72 mg, 0.100 mmol, 1 equiv.), Pd(OAc)₂ (3.37 mg, 0.015 mmol, 0.15 equiv.), XantPhos (17.36 mg, 0.030 mmol, 0.30 equiv.), Cs₂CO₃ (65.18 mg, 0.200 mmol, 2 equiv.) and dioxane (2 mL, 23.608 mmol, 236.01 equiv.) and this resulting mixture was stirred at 100° C. for 2 hours under N₂ atmosphere via sealed tube. Desired product could be detected by LCMS and no work up was performed.

2-[6-azaspiro[2.5]octan-1-yl]-N-[2-fluoro-4-(pyrazol-1-yl)phenyl]-1,6-naphthyridin-7-amine. A mixture of tert-butyl 1-(7-[[2-fluoro-4-(pyrazol-1-yl)phenyl]amino]-1,6-naphthyridin-2-yl)-6-azaspiro[2.5]octane-6-carboxylate (150 mg, 0.291 mmol, 1 equiv.) in DCM (20 mL, 314.601 mmol, 1079.30 equiv.) and TFA (2 mL, 26.926 mmol, 92.38 equiv.) was stirred at room temperature for 1.5 hours. The reaction mixture was basified to pH 9 with sat. NaHCO₃ (aq), then the resulting mixture was extracted with DCM (5×100 mL). The combined organic layers were washed with brine (2×100 mL), dried over anhydrous Na₂SO₄. After filtration, the filtrate was concentrated under reduced pressure to afford 2-[6-azaspiro[2.5]octan-1-yl]-N-[2-fluoro-4-(pyrazol-1-yl)phenyl]-1,6-naphthyridin-7-amine (150 mg, 96%) as a light yellow solid without purification further.

N-[2-fluoro-4-(pyrazol-1-yl)phenyl]-2-[6-methyl-6-azaspiro[2.5]octan-1-yl]-1,6-naphthyridin-7-amine. A mixture of 2-[6-azaspiro[2.5]octan-1-yl]-N-[2-fluoro-4-(pyrazol-1-yl)phenyl]-1,6-naphthyridin-7-amine (150 mg, 0.282 mmol, 1 equiv, 78%), HCHO (34.36 mg, 0.423 mmol, 1.50 equiv, 37%) in THF (10 mL) and NaBH(OAc)₃ (89.74 mg, 0.423 mmol, 1.50 equiv.) was stirred at room temperature for 1 hour. The reaction was quenched by the addition of H₂O (20 mL) at room temperature and the resulting mixture was extracted with DCM (3×100 mL). The combined organic layers were washed with brine (100 mL) and concentrated under reduced pressure to give the residue, which was purified by reverse flash chromatography with the following conditions: Column: XBridge Prep OBD C₁₈ Column, 30×150 mm 5 μm; Mobile Phase A:Water (10 mM NH₄HCO₃), Mobile Phase B:ACN; Flow rate:60 mL/min; Gradient:40 B to 60 B in 7 min; 254/220 nm; RT1: 6.5 min to afford N-[2-fluoro-4-(pyrazol-1-yl)phenyl]-2-[6-methyl-6-azaspiro[2.5]octan-1-yl]-1,6-naphthyridin-7-amine (54.9 mg, 45%) as a light yellow solid.

### Example 23. Preparation of Compounds 141 and 149

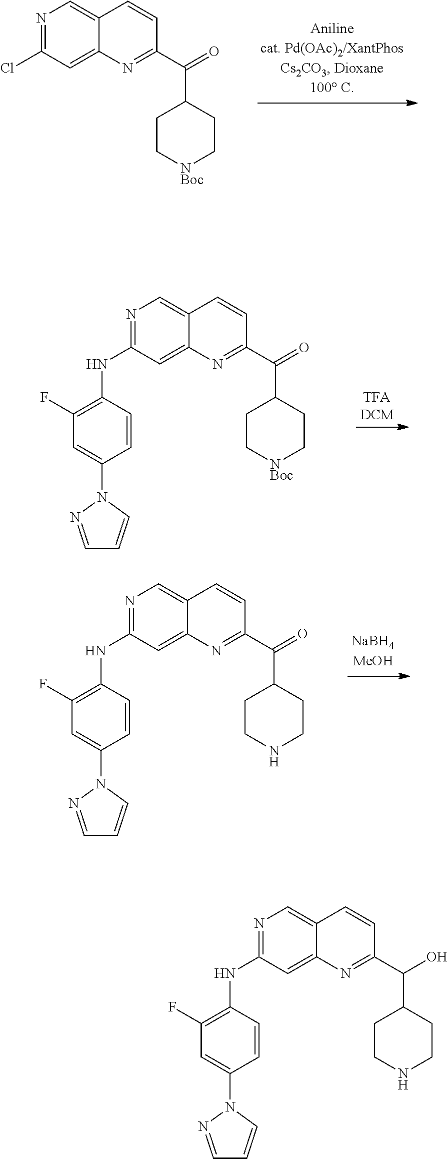 [SMILES: CC(C)(C)OC(=O)N1CCC(C(=O)c2ccc3cnc(Cl)cc3n2)CC1 ; CC(C)(C)OC(=O)N1CCC(C(=O)c2ccc3cnc(Nc4ccc(-n5cccn5)cc4F)cc3n2)CC1 ; O=C(c1ccc2cnc(Nc3ccc(-n4cccn4)cc3F)cc2n1)C1CCNCC1 ; OC(c1ccc2cnc(Nc3ccc(-n4cccn4)cc3F)cc2n1)C1CCNCC1]

Tert-butyl 4-(7-[[2-fluoro-4-(pyrazol-1-yl)phenyl]amino]-1,6-naphthyridine-2-carbonyl)piperidine-1-carboxylate. To a stirred mixture of tert-butyl 4-(7-chloro-1,6-naphthyridine-2-carbonyl)piperidine-1-carboxylate (150 mg, 0.399 mmol, 1 equiv.) and 2-fluoro-4-(pyrazol-1-yl)aniline (84.86 mg, 0.479 mmol, 1.2 equiv.) in dry 1,4-dioxane (15 mL) were added Cs₂CO₃ (260.07 mg, 0.798 mmol, 2 equiv.), Pd(OAc)₂ (13.44 mg, 0.060 mmol, 0.15 equiv.) and Xantphos (69.28 mg, 0.120 mmol, 0.3 equiv.) at room temperature under nitrogen atmosphere. The resulting mixture was stirred for 2 hours at 100° C. under nitrogen atmosphere. The resulting mixture was filtered, the filter cake was washed with the solution of DCM and MeOH (10:1). The filtrate was concentrated under reduced pressure. The residue was purified by Prep-TLC (DCM/MeOH, 40:1) to afford tert-butyl 4-(7-[[2-fluoro-4-(pyrazol-1-yl)phenyl]amino]-1,6-naphthyridine-2-carbonyl)piperidine-1-carboxylate (140 mg, 67%) as a yellow solid.

N-[2-fluoro-4-(pyrazol-1-yl)phenyl]-2-(piperidine-4-carbonyl)-1,6-naphthyridin-7-amine (Compound 141). To a stirred solution of tert-butyl 4-(7-[[2-fluoro-4-(pyrazol-1-yl)phenyl]amino]-1,6-naphthyridine-2-carbonyl)piperidine-1-carboxylate (140 mg) in DCM (18 mg) was added TFA (2 mL) dropwise at 0° C. The resulting mixture was stirred for 2 hours at room temperature. The resulting mixture was concentrated under vacuum. The residue was dissolved in DCM (50 mL) and basified to pH 8 with saturated NaHCO₃ (aq.). The resulting mixture was extracted with DCM (2×50 mL). The combined organic layers dried over anhydrous Na₂SO₄. After filtration, the filtrate was concentrated under reduced pressure. The residue was purified by Prep-TLC (DCM/MeOH 20:1) to afford N-[2-fluoro-4-(pyrazol-1-yl)phenyl]-2-(piperidine-4-carbonyl)-1,6-naphthyridin-7-amine (100 mg) as a yellow solid.

(7-[[2-fluoro-4-(pyrazol-1-yl)phenyl]amino]-1,6-naphthyridin-2-yl) (piperidin-4-yl)methanol (Compound 149). To a stirred solution of N-[2-fluoro-4-(pyrazol-1-yl)phenyl]-2-(piperidine-4-carbonyl)-1,6-naphthyridin-7-amine (100 mg, 0.240 mmol, 1 equiv.) in MeOH (10 mL) was added NaBH₄ (18.17 mg, 0.480 mmol, 2 equiv.) in portions at 0° C. The resulting mixture was stirred for 1 hour at room temperature. The resulting mixture was concentrated under vacuum. The crude product was purified by Prep-HPLC to afford (7-[[2-fluoro-4-(pyrazol-1-yl)phenyl]amino]-1,6-naphthyridin-2-yl) (piperidin-4-yl)methanol (80 mg, 79%) as a light yellow solid.

### Example 24. Preparation of Compounds 247 and 254

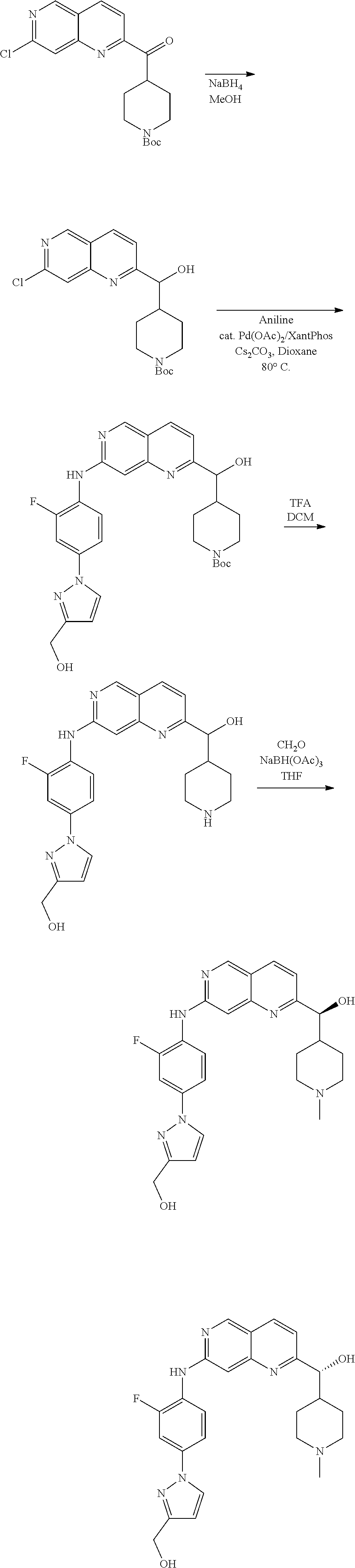 [SMILES: CC(C)(C)OC(=O)N1CCC(C(=O)c2ccc3cnc(Cl)cc3n2)CC1 ; CC(C)(C)OC(=O)N1CCC(C(O)c2ccc3cnc(Cl)cc3n2)CC1 ; CC(C)(C)OC(=O)N1CCC(C(O)c2ccc3cnc(Nc4ccc(-n5ccc(CO)n5)cc4F)cc3n2)CC1 ; OCc1ccn(-c2ccc(Nc3cc4nc(C(O)C5CCNCC5)ccc4cn3)c(F)c2)n1 ; CN1CCC([C@H](O)c2ccc3cnc(Nc4ccc(-n5ccc(CO)n5)cc4F)cc3n2)CC1 ; CN1CCC([C@@H](O)c2ccc3cnc(Nc4ccc(-n5ccc(CO)n5)cc4F)cc3n2)CC1]

Tert-butyl 4-[(7-chloro-1,6-naphthyridin-2-yl) (hydroxy)methyl]piperidine-1-carboxylate. To a stirred solution of tert-butyl 4-(7-chloro-1,6-naphthyridine-2-carbonyl)piperidine-1-carboxylate (200 mg, 0.532 mmol, 1 equiv.) in MeOH (5 mL) was added NaBH₄ (30.20 mg, 0.798 mmol, 1.50 equiv.) in portions at room temperature. The resulting mixture was stirred for 10 min at 0° C. The reaction was monitored by LCMS. The resulting mixture was concentrated under reduced pressure. The residue was purified by reverse phase flash with the following conditions (Column: C18, 120 g; Mobile Phase A:Water/0.05% NH₄HCO₃, Mobile Phase B:ACN; Flow rate:40 mL/min; Gradient: 40% B to 85% B in 20 min; Detector, 254 nm and 220 nm, the desired product were collected at 68% B). Concentrated under reduced pressure to afford tert-butyl 4-[(7-chloro-1,6-naphthyridin-2-yl) (hydroxy)methyl]piperidine-1-carboxylate (130 mg, 64%) as a white solid.

Tert-butyl 4-[[7-([2-fluoro-4-[3-(hydroxymethyl)pyrazol-1-yl]phenyl]amino)-1,6-naphthyridin-2-yl](hydroxy)methyl]piperidine-1-carboxylate. To a stirred mixture of tert-butyl 4-[(7-chloro-1,6-naphthyridin-2-yl) (hydroxy)methyl]piperidine-1-carboxylate (450 mg, 1.191 mmol, 1 equiv.) and [1-(4-amino-3-fluorophenyl)pyrazol-3-yl]methanol (271.44 mg, 1.310 mmol, 1.10 equiv.) in 1,4-dioxane (15 mL) were added Pd(OAc)₂ (40.10 mg, 0.179 mmol, 0.15 equiv.), XantPhos (206.72 mg, 0.357 mmol, 0.30 equiv.) and Cs₂CO₃ (776.03 mg, 2.382 mmol, 2 equiv.) in portions at rt under nitrogen atmosphere. The resulting mixture was stirred for 1 hour at 80° C. under nitrogen atmosphere. The reaction was monitored by LCMS. The mixture was allowed to cool down to rt. The resulting mixture was extracted with EtOAc (3×300 mL). The combined organic layers were washed with brine (2×100 mL), dried over anhydrous Na₂SO₄. After filtration, the filtrate was concentrated under reduced pressure. The residue was purified by reverse phase flash chromatography with the following conditions: Column: Spherical C18, 20-40 um, 330 g; Mobile Phase A: Water (plus 5 mM FA); Mobile Phase B: ACN; Flow rate: 80 mL/min; Gradient: 5%-5% B, 10 min, 30% B-60% B gradient in 25 min; Detector: 220 nm. The fractions containing the desired product were collected at 55% B and concentrated under reduced pressure to afford tert-butyl 4-[[7-([2-fluoro-4-[3-(hydroxymethyl)pyrazol-1-yl]phenyl]amino)-1,6-naphthyridin-2-yl](hydroxy)methyl]piperidine-1-carboxylate (280 mg, 42%) as a yellow solid.

[7-([2-fluoro-4-[3-(hydroxymethyl)pyrazol-1-yl]phenyl]amino)-1,6-naphthyridin-2-yl](piperidin-4-yl)methanol. To a stirred solution of tert-butyl 4-[[7-([2-fluoro-4-[3-(hydroxymethyl)pyrazol-1-yl]phenyl]amino)-1,6-naphthyridin-2-yl](hydroxy)methyl]piperidine-1-carboxylate (280 mg, 1 equiv.) in DCM (10 mL) was added TFA (1 mL) dropwise at 0° C. The reaction mixture was stirred for 0.5 hours at 0° C. The reaction was monitored by LCMS. The resulting mixture was concentrated under reduced pressure. The residue was basified to pH=8 with saturated NaHCO₃ (aq.). The resulting mixture was extracted with EtOAc (3×200 mL). The combined organic layers were washed with brine (1×100 mL), dried over anhydrous Na₂SO₄. After filtration, the filtrate was concentrated under reduced pressure. The residue was purified by reverse phase flash chromatography with the following conditions: Column: Spherical C18, 20-40 um, 120 g Mobile Phase A: Water (plus 5 mM NH₄HCO₃); Mobile Phase B: ACN; Flow rate: 40 mL/min; Gradient: 5%-5% B, 8 min, 45%-70% B gradient in 20 min; Detector: 220 nm. The fractions containing the desired product were collected at 65% B and concentrated under reduced pressure to afford [7-([2-fluoro-4-[3-(hydroxymethyl)pyrazol-1-yl]phenyl]amino)-1,6-naphthyridin-2-yl](piperidin-4-yl)methanol (180 mg) as a yellow solid.

(R)- and (S)-[7-([2-fluoro-4-[3-(hydroxymethyl)pyrazol-1-yl]phenyl]amino)-1,6-naphthyridin-2-yl](1-methylpiperidin-4-yl)methanol (Compounds 247 and 254). To a stirred mixture of [7-([2-fluoro-4-[3-(hydroxymethyl)pyrazol-1-yl]phenyl]amino)-1,6-naphthyridin-2-yl](piperidin-4-yl)methanol (90 mg, 0.201 mmol, 1 equiv.) and HCHO (0.50 mL, 13.655 mmol, 68.05 equiv.) in THF (10 mL) was added NaBH(OAc)₃ (63.79 mg, 0.301 mmol, 1.50 equiv.) in portions at 0° C. under nitrogen atmosphere. The resulting mixture was stirred for 0.5 hours at rt under nitrogen atmosphere. The reaction was monitored by LCMS. The resulting mixture was extracted with DCM (3×300 mL). The combined organic layers were washed with brine (2×200 mL), dried over anhydrous Na₂SO₄. After filtration, the filtrate was concentrated under reduced pressure. The residue was purified by reverse phase flash chromatography with the following conditions: Column: Spherical C18, 20-40 um, 120 g; Mobile Phase A: Water (plus 5 mM FA); Mobile Phase B: ACN; Flow rate: 45 mL/min; Gradient: 5%-5% B, 10 min, 35% B-65% B gradient in 25 min; Detector: 220 nm. The fractions containing the desired product were collected at 65% B and concentrated under reduced pressure to afford 40 mg of racemate. The racemate was purified by Chiral-Prep-HPLC with the following conditions (Column: CHIRALPAK IG, 2*25 cm, 5 um; Mobile Phase A: Hex:DCM=3:1 (10 mM NH₃-MeOH), Mobile Phase B: EtOH:DCM=1:1; Flow rate:19 mL/min; Gradient (%): 60 B to 60 B in 12 min; Detector: 220/254 nm.). Obtained separated enantiomers of [7-([2-fluoro-4-[3-(hydroxymethyl)pyrazol-1-yl]phenyl]amino)-1,6-naphthyridin-2-yl](1-methylpiperidin-4-yl)methanol.

Compound 247 eluted at 6.254 min as a yellow solid. Compound 254 eluted at 9.587 min as a yellow solid.

Compounds 241 and 243 were synthesized following the methods and protocols as described for the synthesis of Compounds 247 and 254, starting with the appropriate materials.

### Example 25. Preparation of Compounds 252 and 255

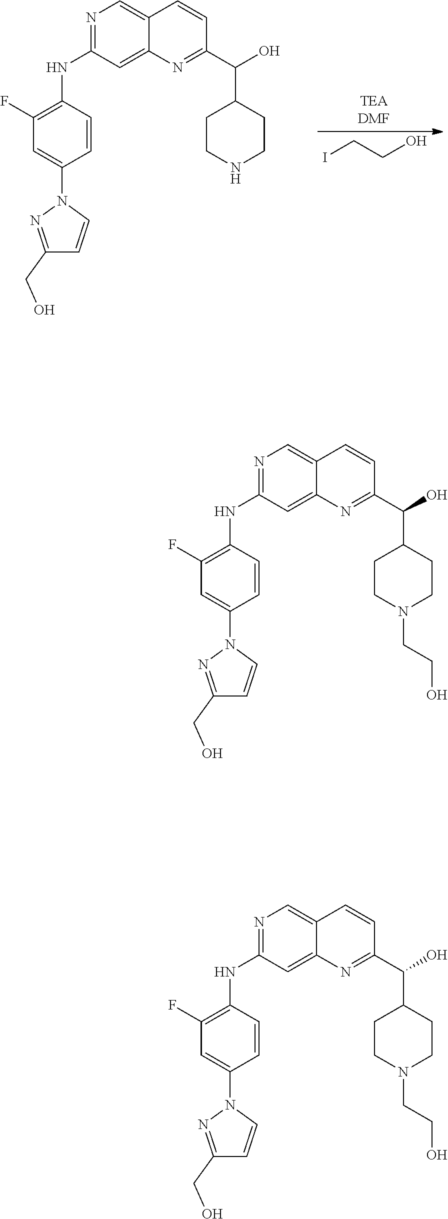

To a stirred mixture of [7-([2-fluoro-4-[3-(hydroxymethyl)pyrazol-1-yl]phenyl]amino)-1,6-naphthyridin-2-yl](piperidin-4-yl)methanol (Compound 247, step 3, 90 mg, 0.201 mmol, 1 equiv.) and ethanol, 2-iodo-(69.02 mg, 0.401 mmol, 2 equiv.) in DMF (6 mL) was added TEA (60.92 mg, 0.602 mmol, 3 equiv.) in portions at rt under nitrogen atmosphere. The resulting mixture was stirred for 4 hours at rt under nitrogen atmosphere. The reaction was monitored by LCMS. The residue was purified by reverse phase flash chromatography with the following conditions: Column: Spherical C18, 20-40 um, 120 g; Mobile Phase A: Water (plus 5 mM FA); Mobile Phase B: ACN; Flow rate: 45 mL/min; Gradient: 5%-5% B, 10 min, 36% B-70% B gradient in 25 min; Detector: 220 nm. The fractions containing the desired product were collected at 66% B and concentrated under reduced pressure to afford 70 mg of racemate. The racemate was purified by Chiral-Prep-HPLC with the following conditions (Column: CHIRALPAK IG, 2*25 cm, 5 um; Mobile Phase A: Hex:DCM=3:1 (10 mM NH₃-MeOH), Mobile Phase B: EtOH:DCM=1:1; Flow rate: 20 mL/min; Gradient (%): 60 B to 60 B in 13 min; Detector: 220/254 nm.). Obtained separated enantiomers of 2-(4-[[7-([2-fluoro-4-[3-(hydroxymethyl)pyrazol-1-yl]phenyl]amino)-1,6-naphthyridin-2-yl](hydroxy)methyl]piperidin-1-yl)ethanol.

Compound 252 eluted at 10.754 min as a yellow solid. Compound 255 eluted at 8.52 min as a yellow solid.

### Example 26. Preparation of Compounds 250 and 251

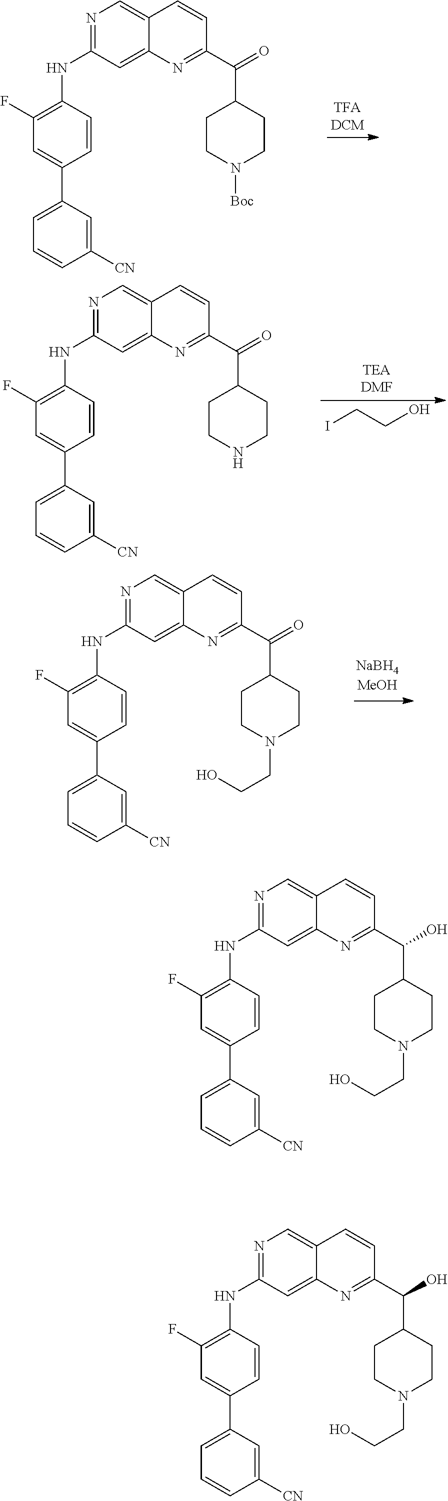

3′-fluoro-4′-[[2-(piperidine-4-carbonyl)-1,6-naphthyridin-7-yl]amino]-[1,1′-biphenyl]-3-carbonitrile. To a stirred solution of tert-butyl 4-[7-([3-cyano-3-fluoro-[1,1-biphenyl]-4-yl]amino)-1,6-naphthyridine-2-carbonyl]piperidine-1-carboxylate (Prepared via similar method to Compound 141, step 1, 100 mg, 1 equiv.) in DCM (3 mL) was added TFA (0.30 mL) in portions at rt under nitrogen atmosphere. The resulting mixture was stirred for 16 hours at rt under nitrogen atmosphere. The reaction was monitored by LCMS. The resulting mixture was concentrated under reduced pressure. The residue was purified by silica gel column chromatography, eluted with PE/EtOAc (10/1 to 1/1) to afford 3′-fluoro-4′-[[2-(piperidine-4-carbonyl)-1,6-naphthyridin-7-yl]amino]-[1,1′-biphenyl]-3-carbonitrile (20 mg, 24%) as a yellow solid.

3′-fluoro-4′-([2-[1-(2-hydroxyethyl)piperidine-4-carbonyl]-1,6-naphthyridin-7-yl]amino)-[1,1′-biphenyl]-3-carbonitrile. To a stirred mixture of 3′-fluoro-4′-[[2-(piperidine-4-carbonyl)-1,6-naphthyridin-7-yl]amino]-[1,1′-biphenyl]-3-carbonitrile (86 mg, 0.190 mmol, 1 equiv.) and ethanol, 2-iodo-(39.31 mg, 0.229 mmol, 1.2 equiv.) in DMF (2.50 mL) was added TEA (38.55 mg, 0.381 mmol, 2 equiv.) at room temperature. The resulting mixture was stirred for 1 hour at room temperature. The reaction was monitored by LCMS. The resulting mixture was concentrated under reduced pressure. The residue was purified by reverse phase flash with the following conditions (Column: C18, 330 g; Mobile Phase A:Water/0.05% NH₄HCO₃, Mobile Phase B:ACN; Flow rate:80 mL/min; Gradient: 20% B to 50% B in 20 min; Detector, 254 nm, Monitor, 220 nm, the desired product were collected at 45% B) to afford 3′-fluoro-4′-([2-[1-(2-hydroxyethyl)piperidine-4-carbonyl]-1,6-naphthyridin-7-yl]amino)-[1,1′-biphenyl]-3-carbonitrile (70 mg, 74%) as a yellow solid.
(R)- and (S)-3′-fluoro-4′-[(2-[hydroxy[1-(2-hydroxyethyl)piperidin-4-yl]methyl]-1,6-naphthyridin-7-yl)amino]-[1,1′-biphenyl]-3-carbonitrile (Compounds 250 and 251). To a stirred mixture of 3′-fluoro-4′-([2-[1-(2-hydroxyethyl)piperidine-4-carbonyl]-1,6-naphthyridin-7-yl]amino)-[1,1′-biphenyl]-3-carbonitrile (70 mg, 0.141 mmol, 1 equiv.) in MeOH (2 mL) was added NaBH₄ (8.02 mg, 0.212 mmol, 1.5 equiv.) at room temperature. The resulting mixture was stirred for 1 hour at room temperature. The reaction was monitored by LCMS. The resulting mixture was concentrated under reduced pressure. The residue was purified by reverse flash chromatography with the following conditions: column, C₁₈ silica gel; mobile phase, MeOH in water, 30% to 50% gradient in 10 min; detector, UV 254 nm to afford 55 mg of racemate. The racemate was separated by SFC with the following conditions: Column: CHIRALPAK IG, 2×25 cm, 5 um; Mobile Phase A: Hex (plus 10 mM NH₃), Mobile Phase B: EtOH:DCM=1:1; Flow rate: 17 mL/min; Gradient: isocratic 90 B in 12 min; Detector: UV 220/254 nm; RT1:6.247 min; RT 2: 9.324 min.

Compound 250-Yield: 20.4 mg. Compound 251-Yield: 21.9 mg

### Example 27. Preparation of Compound 217

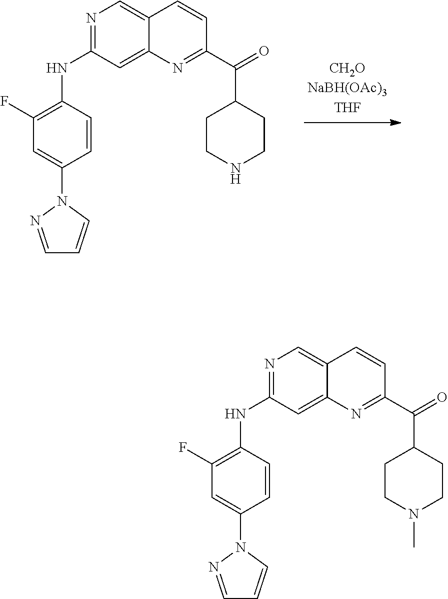 [SMILES: O=C(c1ccc2cnc(Nc3ccc(-n4cccn4)cc3F)cc2n1)C1CCNCC1 ; CN1CCC(C(=O)c2ccc3cnc(Nc4ccc(-n5cccn5)cc4F)cc3n2)CC1]

A mixture of N-[2-fluoro-4-(pyrazol-1-yl)phenyl]-2-(piperidine-4-carbonyl)-1,6-naphthyridin-7-amine (80 mg, 0.192 mmol, 1 equiv.), formaldehyde solution (20.27 mg, 0.250 mmol, 1.30 equiv, 37%) and sodium triacetoxyborohydride (122.14 mg, 0.576 mmol, 3 equiv.) in THF (2 mL) was stirred for 3 hours at room temperature under nitrogen atmosphere. The resulting mixture was concentrated under reduced pressure. The residue was purified by Prep-HPLC to afford N-[2-fluoro-4-(pyrazol-1-yl)phenyl]-2-(1-methylpiperidine-4-carbonyl)-1,6-naphthyridin-7-amine (46.6 mg, 56%) as an orange solid.

### Example 28. Preparation of Compounds 175 and 176

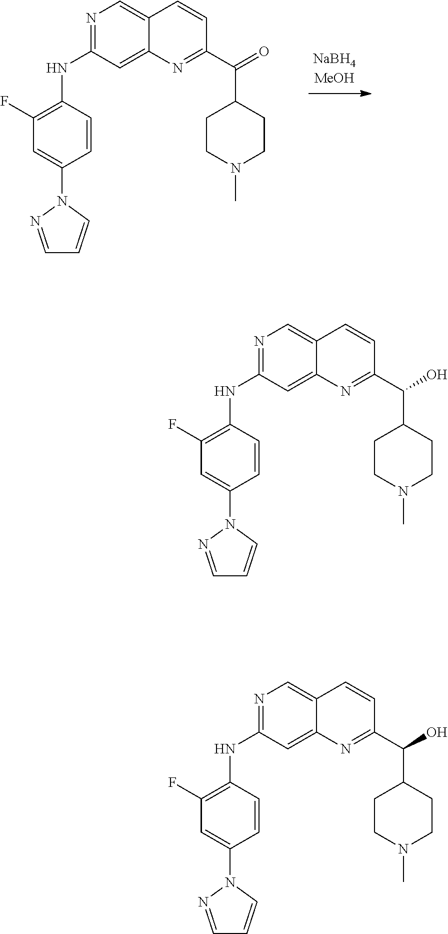 [SMILES: CN1CCC(C(=O)c2ccc3cnc(Nc4ccc(-n5cccn5)cc4F)cc3n2)CC1 ; CN1CCC([C@@H](O)c2ccc3cnc(Nc4ccc(-n5cccn5)cc4F)cc3n2)CC1 ; CN1CCC([C@H](O)c2ccc3cnc(Nc4ccc(-n5cccn5)cc4F)cc3n2)CC1]

To a stirred mixture of N-[2-fluoro-4-(pyrazol-1-yl)phenyl]-2-(1-methylpiperidine-4-carbonyl)-1,6-naphthyridin-7-amine (150 mg, 0.348 mmol, 1 equiv.) in MeOH (10 mL) was added NaBH₄ (26.37 mg, 0.697 mmol, 2 equiv.) in portions at 0° C. The resulting mixture was stirred for 2 hours at room temperature. The resulting mixture was concentrated under vacuum. The residue was purified by reverse flash chromatography with the following conditions: Column: Spherical C18, 20-40 um, 330 g; Mobile Phase A: Water (plus 5 mM NH₄HCO₃; Mobile Phase B: ACN; Flow rate: 45 mL/min; Gradient: 5%-5% B, 10 min, 20% B-40% B gradient in 20 min; Detector: 254 nm. The fractions containing the desired product were collected at 33% B and concentrated under reduced pressure to afford 120 mg of racemate. The racemate was purified by Chiral-HPCL with the following conditions (Column: CHIRALPAK IG, 2*25 cm, 5 μm; Mobile Phase A:Hex (10 mM NH₃), Mobile Phase B:EtOH:DCM=1:1—HPLC; Flow rate:20 mL/min; Gradient:60 B to 60 B in 18 min; 220/254 nm; RT1:5.664; RT2:9.823). Obtained (R)- and (S)-(7-[[2-fluoro-4-(pyrazol-1-yl)phenyl]amino]-1,6-naphthyridin-2-yl) (1-methylpiperidin-4-yl)methanol.

Compound 175-Yield: 50.9 mg. Compound 176-Yield: 46.3 mg.

Compounds 344 and 347 were synthesized following the methods and protocols as described for the synthesis of Compounds 175 and 176, starting with the appropriate materials.

### Example 29. Preparation of Compound 292

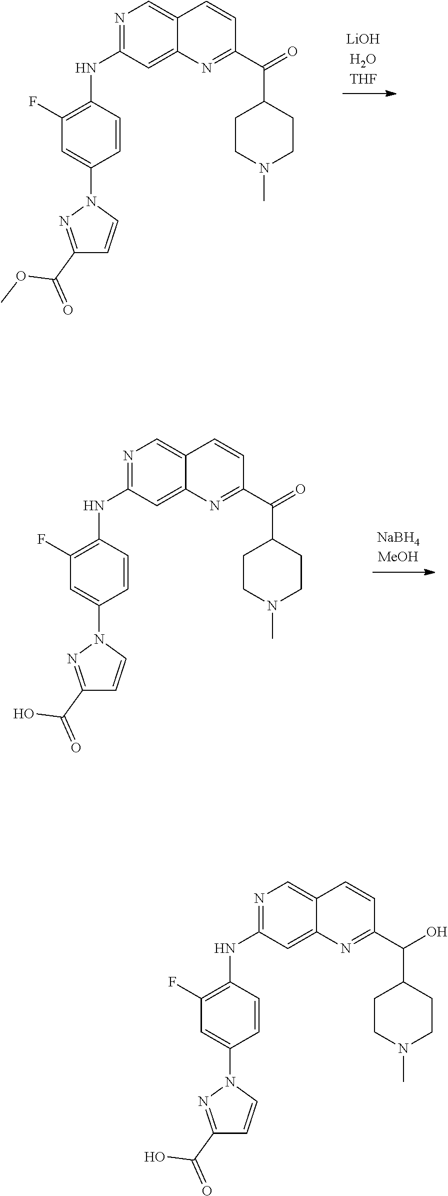 [SMILES: COC(=O)c1ccn(-c2ccc(Nc3cc4nc(C(=O)C5CCN(C)CC5)ccc4cn3)c(F)c2)n1 ; CN1CCC(C(=O)c2ccc3cnc(Nc4ccc(-n5ccc(C(=O)O)n5)cc4F)cc3n2)CC1 ; CN1CCC(C(O)c2ccc3cnc(Nc4ccc(-n5ccc(C(=O)O)n5)cc4F)cc3n2)CC1]

1-(3-fluoro-4-[[2-(1-methylpiperidine-4-carbonyl)-1,6-naphthyridin-7-yl]amino]phenyl)pyrazole-3-carboxylic acid. To a stirred solution of methyl 1-(3-fluoro-4-[[2-(1-methylpiperidine-4-carbonyl)-1,6-naphthyridin-7-yl]amino]phenyl)pyrazole-3-carboxylate (Prepared via a similar procedure as Compound 217, 80 mg, 0.164 mmol, 1 equiv.) in THF (5 mL) was added LiOH (19.61 mg, 0.819 mmol, 5 equiv.) and H₂O (2 mL) in portions at 0° C. The resulting mixture was stirred for 1 hour at room temperature. The reaction was monitored by LCMS. The resulting mixture was concentrated under reduced pressure. The residue was purified by reverse phase flash with the following conditions (Column: C18, 120 g; Mobile Phase A:Water/0.05% NH₄HCO₃, Mobile Phase B:ACN; Flow rate:40 mL/min; Gradient: 25% B to 55% B in 20 min; Detector, 254 nm and 220 nm, the desired product were collected at 38% B). Concentrated under reduced pressure to afford 1-(3-fluoro-4-[[2-(1-methylpiperidine-4-carbonyl)-1,6-naphthyridin-7-yl]amino]phenyl)pyrazole-3-carboxylic acid (55 mg, 70%) as an orange solid.

1-[3-fluoro-4-([2-[hydroxy(1-methylpiperidin-4-yl)methyl]-1,6-naphthyridin-7-yl]amino)phenyl]pyrazole-3-carboxylic acid. To a stirred solution of 1-(3-fluoro-4-[[2-(1-methylpiperidine-4-carbonyl)-1,6-naphthyridin-7-yl]amino]phenyl)pyrazole-3-carboxylic acid (55 mg, 0.116 mmol, 1 equiv.) in MeOH (3 mL) was added NaBH₄ (5.26 mg, 0.139 mmol, 1.20 equiv.) in portions at room temperature. The resulting mixture was stirred for 10 min at 0° C. The reaction was monitored by LCMS. The resulting mixture was concentrated under reduced pressure. The residue was purified by reverse phase flash with the following conditions (Column: XBridge Shield RP18 OBD Column, 5 μm, 19*150 mm). Concentrated under reduced pressure to afford 1-[3-fluoro-4-([2-[hydroxy(1-methylpiperidin-4-yl)methyl]-1,6-naphthyridin-7-yl]amino)phenyl]pyrazole-3-carboxylic acid (14.6 mg, 26%) as a yellow solid.

### Example 30. Preparation of Compounds 387 and 391

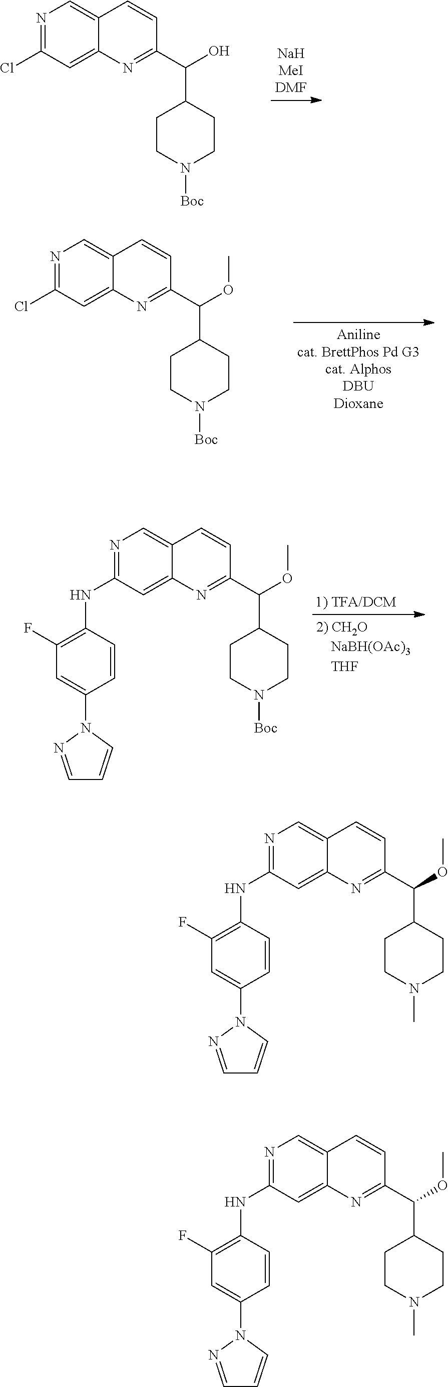 [SMILES: CC(C)(C)OC(=O)N1CCC(C(O)c2ccc3cnc(Cl)cc3n2)CC1 ; COC(c1ccc2cnc(Cl)cc2n1)C1CCN(C(=O)OC(C)(C)C)CC1 ; COC(c1ccc2cnc(Nc3ccc(-n4cccn4)cc3F)cc2n1)C1CCN(C(=O)OC(C)(C)C)CC1 ; CO[C@H](c1ccc2cnc(Nc3ccc(-n4cccn4)cc3F)cc2n1)C1CCN(C)CC1 ; CO[C@@H](c1ccc2cnc(Nc3ccc(-n4cccn4)cc3F)cc2n1)C1CCN(C)CC1]

Tert-butyl 4-[(7-chloro-1,6-naphthyridin-2-yl)(methoxy)methyl]piperidine-1-carboxylate. To a stirred solution of tert-butyl 4-[(7-chloro-1,6-naphthyridin-2-yl) (hydroxy)methyl]piperidine-1-carboxylate (Compound 247 step 1, 120 mg, 0.318 mmol, 1 equiv.) in DMF (10 mL) was added NaH (11.43 mg, 0.476 mmol, 1.5 equiv.) dropwise at 0° C. under nitrogen atmosphere. The resulting mixture was stirred for 0.5 hours at room temperature under nitrogen atmosphere. The mixture was added CH₃I (90.15 mg, 0.635 mmol, 2.0 equiv.) at 0° C. under nitrogen atmosphere. The resulting mixture was stirred for 1 hour at room temperature under nitrogen atmosphere. The reaction was quenched with sat. NH₄C₁ (aq.) at room temperature. The resulting mixture was extracted with EtOAc (2×50 mL). The combined organic layers were washed with brine (1×30 mL), dried over anhydrous Na₂SO₄. After filtration, the filtrate was concentrated under reduced pressure. The residue was purified by TLC, eluted with PE:EA (10:1) to afford tert-butyl 4-[(7-chloro-1,6-naphthyridin-2-yl) (methoxy)methyl]piperidine-1-carboxylate (73 mg, 58%) as a white solid.

Tert-butyl 4-[(7-[[2-fluoro-4-(pyrazol-1-yl)phenyl]amino]-1,6-naphthyridin-2-yl) (methoxy)methyl]piperidine-1-carboxylate. To a stirred solution of tert-butyl 4-[(7-chloro-1,6-naphthyridin-2-yl)(methoxy)methyl]piperidine-1-carboxylate (70 mg, 0.179 mmol, 1 equiv.), 2-fluoro-4-(pyrazol-1-yl)aniline (47.47 mg, 0.268 mmol, 1.5 equiv.), BrettPhos Pd G3 (32.37 mg, 0.036 mmol, 0.20 equiv.) and Alphos (58.23 mg, 0.071 mmol, 0.40 equiv.) in dioxane (2 mL) was added DBU (81.58 mg, 0.536 mmol, 3 equiv.) in portions at 25° C. under nitrogen atmosphere. The resulting mixture was stirred for 2 hours at 66° C. under nitrogen atmosphere. The resulting mixture was concentrated under vacuum. The residue was purified by TLC, eluted with DCM/MeOH (10:1) to afford tert-butyl 4-[(7-[[2-fluoro-4-(pyrazol-1-yl)phenyl]amino]-1,6-naphthyridin-2-yl)(methoxy)methyl]piperidine-1-carboxylate (55 mg, 57%) as a white solid.

(R)- and (S)—N-[2-fluoro-4-(pyrazol-1-yl)phenyl]-2-[methoxy(1-methylpiperidin-4-yl)methyl]-1,6-naphthyridin-7-amine (Compounds 387 and 391). To a stirred solution of tert-butyl 4-[(7-[[2-fluoro-4-(pyrazol-1-yl)phenyl]amino]-1,6-naphthyridin-2-yl) (methoxy)methyl]piperidine-1-carboxylate (55 mg, 0.103 mmol, 1 equiv.) in DCM (50 mL) was added TFA (5 mL) dropwise at room temperature under nitrogen atmosphere. The resulting mixture was stirred for 1 hour at room temperature under nitrogen atmosphere. The reaction was monitored by TLC. The resulting mixture was concentrated under reduced pressure. The resulting mixture was diluted with DCM (15 mL). The mixture was added Et3N (10 mL). The resulting mixture was stirred for 1 hour and concentrated under reduced pressure. The mixture diluted with THF (15 mL, 246.860 mmol) and was added HCHO (3.72 mg, 0.124 mmol, 1.2 equiv.), NaBH(OAc)₃ (30.64 mg, 0.145 mmol, 1.4 equiv.). The resulting mixture was stirred for 1 hour. The reaction was monitored by LCMS. The reaction was quenched with sat. NaHCO₃ (aq.) at room temperature. The resulting mixture was concentrated under vacuum. The residue was purified by Prep-TLC (DCM/MeOH 5:1) to afford 20 mg of racemate. The racemate was separated by SFC with the following conditions: Column: CHIRALPAK IG, 2*25 cm, 5 μm; Mobile Phase A:Hex (10 mM NH₃), Mobile Phase B:EtOH:DCM=1:1-HPLC; Flow rate:20 mL/min; Gradient:25 B to 25 B in 20 min; 220/254 nm; RT1:16.404 min; RT2:18.501 min. Obtained (R)- and (S)-(N-[2-fluoro-4-(pyrazol-1-yl)phenyl]-2-[methoxy(1-methylpiperidin-4-yl)methyl]-1,6-naphthyridin-7-amine.

Compound 387-eluted at 16.4 min; yield: 3.2 mg. Compound 391-eluted at 18.5 min; yield: 5.4 mg.

### Example 31. Preparation of Compound 165

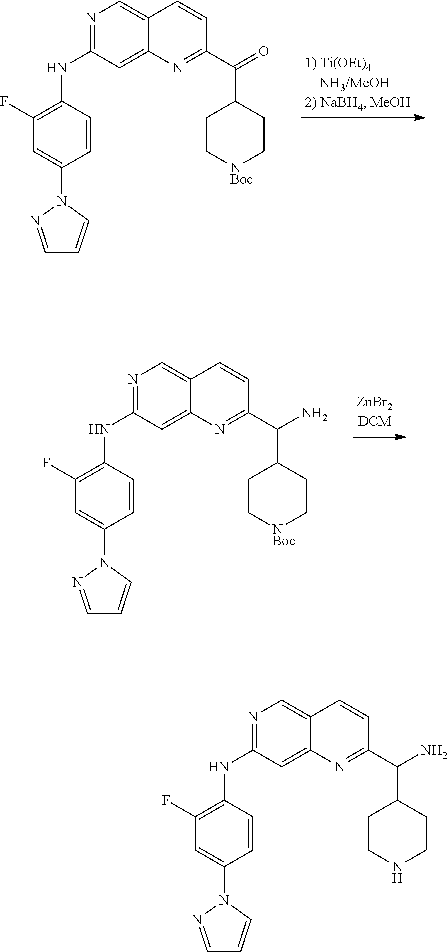 [SMILES: CC(C)(C)OC(=O)N1CCC(C(=O)c2ccc3cnc(Nc4ccc(-n5cccn5)cc4F)cc3n2)CC1 ; CC(C)(C)OC(=O)N1CCC(C(N)c2ccc3cnc(Nc4ccc(-n5cccn5)cc4F)cc3n2)CC1 ; NC(c1ccc2cnc(Nc3ccc(-n4cccn4)cc3F)cc2n1)C1CCNCC1]

Tert-butyl 4-[amino(7-[[2-fluoro-4-(pyrazol-1-yl)phenyl]amino]-1,6-naphthyridin-2-yl)methyl]piperidine-1-carboxylate. To a stirred solution of tert-butyl 4-(7-[[2-fluoro-4-(pyrazol-1-yl)phenyl]amino]-1,6-naphthyridine-2-carbonyl)piperidine-1-carboxylate (200 mg, 0.387 mmol, 1 equiv.) and tetraethoxytitanium (88.32 mg, 0.387 mmol, 1 equiv.) in THF (20 mL) was added a solution of NH₃ (g) in MeOH (0.17 mL, 1.190 mmol, 3.07 equiv.) at room temperature. The resulting mixture was stirred for 16 hours at 80° C. The resulting mixture was concentrated under reduced pressure. The crude product of tert-butyl 4-(7-[[2-fluoro-4-(pyrazol-1-yl)phenyl]amino]-1,6-naphthyridine-2-carboximidoyl)piperidine-1-carboxylate (280 mg, crude) in MeOH (20 mL) was charged with NaBH₄ (30.82 mg, 0.815 mmol, 1.50 equiv.) in portions at 0° C. The resulting mixture was stirred for 1 hour at 0° C. The resulting mixture was concentrated under reduced pressure. The residue was purified by Prep-TLC (DCM/MeOH 5:1) to afford tert-butyl 4-[amino(7-[[2-fluoro-4-(pyrazol-1-yl)phenyl]amino]-1,6-naphthyridin-2-yl)methyl]piperidine-1-carboxylate (70 mg, 24%) as a yellow foam.

2-[amino(piperidin-4-yl)methyl]-N-[2-fluoro-4-(pyrazol-1-yl)phenyl]-1,6-naphthyridin-7-amine (Compound 165). To a stirred solution of tert-butyl 4-[amino(7-[[2-fluoro-4-(pyrazol-1-yl)phenyl]amino]-1,6-naphthyridin-2-yl)methyl]piperidine-1-carboxylate (50 mg, 0.097 mmol, 1 equiv.) in DCM (10 mL) was added ZnBr₂ (217.56 mg, 0.966 mmol, 10 equiv.) at room temperature. The resulting mixture was stirred for 16 hours at 50° C. The resulting mixture was concentrated under reduced pressure. The residue was purified by reverse phase flash chromatography with the following conditions: Column: Spherical C18, 20-40 um, 330 g; Mobile Phase A: Water plus 0.5% FA); Mobile Phase B: ACN; Flow rate: 80 mL/min; Gradient: 5%-5% B, 10 min, 10% B-30% B gradient in 25 min; Detector: 254 nm. The fractions containing the desired product were collected at 18% B and concentrated under reduced pressure to afford 2-[amino(piperidin-4-yl)methyl]-N-[2-fluoro-4-(pyrazol-1-yl)phenyl]-1,6-naphthyridin-7-aminium diformate (28.8 mg) as a yellow solid.

Compounds 395 and 400 were synthesized following the methods and protocols as described for the synthesis of Compound 165, starting with the appropriate materials.

### Example 32. Preparation of Compounds 196, 205 and 216

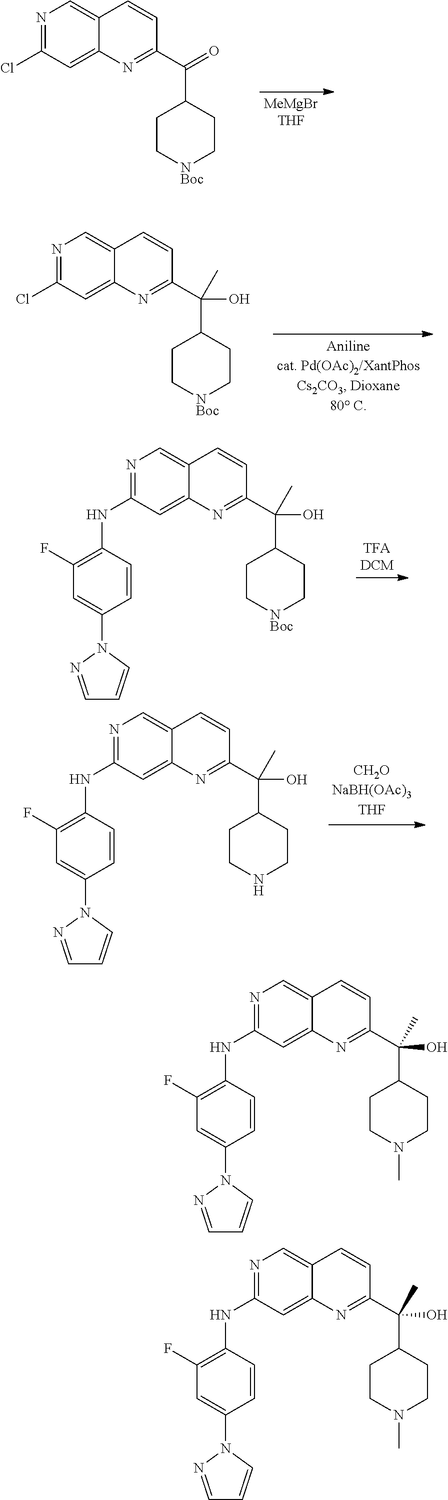

Tert-butyl 4-[1-(7-chloro-1,6-naphthyridin-2-yl)-1-hydroxyethyl]piperidine-1-carboxylate. To a stirred solution of tert-butyl 4-(7-chloro-1,6-naphthyridine-2-carbonyl)piperidine-1-carboxylate (4070 mg, 10.829 mmol, 1 equiv.) in THF (140 mL) were added CH₃MgBr (3873.80 mg, 32.486 mmol, 3 equiv.) at 0° C. under nitrogen atmosphere. The resulting mixture was stirred for 1 hour at 0° C. under nitrogen atmosphere. The reaction was monitored by LCMS. The reaction was quenched with sat. NH₄C₁ (aq.) at 0° C. The resulting mixture was concentrated under reduced pressure. The residue was purified by silica gel column chromatography, eluted with DCM/MeOH (60:1) to afford tert-butyl 4-[1-(7-chloro-1,6-naphthyridin-2-yl)-1-hydroxyethyl]piperidine-1-carboxylate (4200 mg, 98%) as a brown oil.

Tert-butyl 4-[1-(7-[[2-fluoro-4-(pyrazol-1-yl)phenyl]amino]-1,6-naphthyridin-2-yl)-1-hydroxyethyl]piperidine-1-carboxylate. To a stirred mixture of tert-butyl 4-[1-(7-chloro-1,6-naphthyridin-2-yl)-1-hydroxyethyl]piperidine-1-carboxylate (260 mg, 0.663 mmol, 1 equiv.) and 2-fluoro-4-(pyrazol-1-yl)aniline (152.81 mg, 0.862 mmol, 1.30 equiv.) in 1,4-dioxane (50 mL) were added XantPhos (115.16 mg, 0.199 mmol, 0.30 equiv.), Cs₂CO₃ (432.32 mg, 1.327 mmol, 2 equiv.) and Pd(OAc)₂ (29.79 mg, 0.133 mmol, 0.20 equiv.) at room temperature under nitrogen atmosphere. The resulting mixture was stirred for 6 hours at 80° C. under nitrogen atmosphere. The reaction was monitored by LCMS. The mixture was allowed to cool down to room temperature. The resulting mixture was concentrated under reduced pressure. The resulting mixture was filtered, the filter cake was washed with EtOAc (3×20 mL). The filtrate was concentrated under reduced pressure. The residue was purified by reverse phase flash with the following conditions (Column: C18, 120 g; Mobile Phase A:Water/0.05% TFA, Mobile Phase B:ACN; Flow rate:40 mL/min; Gradient: 40% B to 95% B in 20 min; Detector, 254 nm and 220 nm, the desired product were collected at 95% B). Concentrated under reduced pressure to afford tert-butyl 4-[1-(7-[[2-fluoro-4-(pyrazol-1-yl)phenyl]amino]-1,6-naphthyridin-2-yl)-1-hydroxyethyl]piperidine-1-carboxylate (300 mg, 84%) as a brown solid.

1-(7-[[2-fluoro-4-(pyrazol-1-yl)phenyl]amino]-1,6-naphthyridin-2-yl)-1-(piperidin-4-yl)ethanol (Compound 196). To a stirred solution of tert-butyl N-[4-[7-([2-fluoro-4-[3-(hydroxymethyl)pyrazol-1-yl]phenyl]amino)-1,6-naphthyridin-2-ylsulfonyl]-1-methylcyclohexyl]carbamate (200.00 mg, 0.327 mmol, 1.00 equiv.) in CH₂C₁₂ (20 mL) was added TFA (20 mL, 20%) in portions at room temperature. The resulting mixture was stirred for 1 h at 0° C. The reaction was monitored by LCMS. The resulting mixture was concentrated under reduced pressure. The residue was purified by reverse phase flash with the following conditions (Column: C18, 120 g; Mobile Phase A:Water/0.05% NH4NCO3, Mobile Phase B:ACN; Flow rate:40 mL/min; Gradient: 30% B to 65% B in 20 min; Detector, 254 nm and 220 nm, the desired product were collected at 46% B). Concentrated under reduced pressure to afford 1-(7-[[2-fluoro-4-(pyrazol-1-yl)phenyl]amino]-1,6-naphthyridin-2-yl)-1-(piperidin-4-yl)ethanol; formic acid (230 mg, 91.43%) as a yellow solid.

(R)- and (S)-1-(7-[[2-fluoro-4-(pyrazol-1-yl)phenyl]amino]-1,6-naphthyridin-2-yl)-1-(1-methylpiperidin-4-yl)ethanol (Compounds 205 and 216): To a stirred solution of 1-(7-[[2-fluoro-4-(pyrazol-1-yl)phenyl]amino]-1,6-naphthyridin-2-yl)-1-(piperidin-4-yl)ethanol (20 mg, 0.046 mmol, 1 equiv.) and HCHO (2.78 mg, 0.093 mmol, 2 equiv.) in THF (20 mL) ware added NaBH(OAc)₃ (19.60 mg, 0.092 mmol, 2 equiv.) in portions at 0° C. The resulting mixture was stirred for 30 min at 0° C. The reaction was monitored by LCMS. The resulting mixture was concentrated under reduced pressure. The residue was purified by reverse phase flash with the following conditions (Column: XBridge Shield RP18 OBD Column, 5 μm, 19*150 mm). Concentrated under reduced pressure to afford 100 mg of racemate. The racemate was separated by SFC with the following conditions: Column: CHIRALPAK IG, 2*25 cm, 5 μm; Mobile Phase A:Hex:DCM=3:1 (10 mM NH₃-MEOH)-HPLC, Mobile Phase B:EtOH-HPLC; Flow rate:20 mL/min; Gradient:30 B to 30 B in 22 min; 220/254 nm; RT1:11.279; RT2:17.825; Injection Volumn:1.35 mL; Number Of Runs:3; Obtained (R)- and (S)-1-(7-[[2-fluoro-4-(pyrazol-1-yl)phenyl]amino]-1,6-naphthyridin-2-yl)-1-(1-methylpiperidin-4-yl)ethanol.

Compound 205-eluted at 11.279 min; yield: 14.0 mg. Compound 216-eluted at 17.825 min; yield: 14.8 mg

Compounds 389 and 392 were synthesized following the methods and protocols as described for the synthesis of Compound 196, starting with the appropriate materials.

### Example 33. Preparation of Compound 345

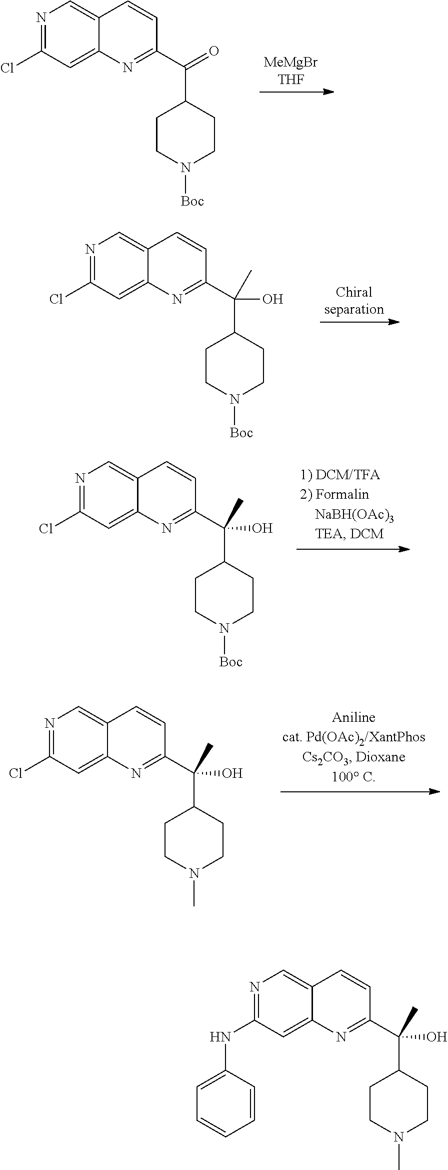

Tert-butyl 4-[1-(7-chloro-1,6-naphthyridin-2-yl)-1-hydroxyethyl]piperidine-1-carboxylate. To a stirred solution of tert-butyl 4-(7-chloro-1,6-naphthyridine-2-carbonyl)piperidine-1-carboxylate (4070 mg, 10.829 mmol, 1 equiv.) in THF (140 mL) were added CH₃MgBr (3873.80 mg, 32.486 mmol, 3 equiv.) at 0° C. under nitrogen atmosphere. The resulting mixture was stirred for 1 hour at 0° C. under nitrogen atmosphere. The reaction was monitored by LCMS. The reaction was quenched with sat. NH₄C₁ (aq.) at 0° C. The resulting mixture was concentrated under reduced pressure. The residue was purified by silica gel column chromatography, eluted with DCM/MeOH (60:1) to afford tert-butyl 4-[1-(7-chloro-1,6-naphthyridin-2-yl)-1-hydroxyethyl]piperidine-1-carboxylate (4200 mg, 98%) as a brown oil.

Tert-butyl 4-[(1R)-1-(7-chloro-1,6-naphthyridin-2-yl)-1-hydroxyethyl]piperidine-1-carboxylate. tert-butyl 4-[1-(7-chloro-1,6-naphthyridin-2-yl)-1-hydroxyethyl]piperidine-1-carboxylate (4.60 g, 11.7 mmol) was separated by SFC with the following conditions: Column: CHIRALPAK IG, 5*25 cm, 10 um; Mobile Phase A:CO₂, Mobile Phase B:MeOH:ACN=1:1 (2 mM NH₃-MeOH); Flow rate:200 mL/min; Gradient:50% B; 220 nm; RT1:6.1; RT2:12.02; Injection Volumn:15 mL; Number Of Runs:5; The fractions at 12.02 min were collected and concentrated under reduced pressure to afford tert-butyl 4-[(1R)-1-(7-chloro-1,6-naphthyridin-2-yl)-1-hydroxyethyl]piperidine-1-carboxylate (1670 mg, 36%) as n light yellow solid. (slower eluting isomer).

(1R)-1-(7-chloro-1,6-naphthyridin-2-yl)-1-(1-methylpiperidin-4-yl)ethanol. To a stirred solution of tert-butyl 4-[(1R)-1-(7-chloro-1,6-naphthyridin-2-yl)-1-hydroxyethyl]piperidine-1-carboxylate (1670 mg, 4.261 mmol, 1 equiv.) in DCM (40 mL) was added TFA (8 mL, 98.744 mmol, 23.17 equiv.) in portions at 0° C. under air atmosphere. The resulting mixture was stirred for 1 hour at 0° C. The reaction was monitored by LCMS. The resulting mixture was concentrated under reduced pressure. To the stirred mixture in DCM (20 mL) was added TEA (13.72 mL, 98.707 mmol, 23.16 equiv.) in portions at room temperature. After 5 min added formalin (255.90 mg, 8.523 mmol, 2 equiv, 33%) and NaBH(OAc)₃ (1083.77 mg, 5.114 mmol, 1.20 equiv.). The resulting mixture was stirred for 1 hour at room temperature under air atmosphere. The resulting mixture was concentrated under reduced pressure. The residue was purified by silica gel column chromatography, eluted with DCM/MeOH (6:1) to afford (1R)-1-(7-chloro-1,6-naphthyridin-2-yl)-1-(1-methylpiperidin-4-yl)ethanol (1300 mg, 99%) as a light yellow oil.

(1R)-1-(1-methylpiperidin-4-yl)-1-[(7-(phenylamino)-1,6-naphthyridin-2-yl]ethanol (Compound 345). To a stirred mixture of (1R)-1-(7-chloro-1,6-naphthyridin-2-yl)-1-(1-methylpiperidin-4-yl)ethanol (40 mg, 0.131 mmol, 1 equiv.) and aniline (14.62 mg, 0.157 mmol, 1.2 equiv.) in 1,4-dioxane (1.50 mL) were added Pd(OAc)₂ (4.40 mg, 0.020 mmol, 0.15 equiv.), XantPhos (22.71 mg, 0.039 mmol, 0.3 equiv.) and Cs₂CO₃ (85.23 mg, 0.262 mmol, 2 equiv.) at room temperature under nitrogen atmosphere. The resulting mixture was stirred for 0.5 hours at 100° C. under nitrogen atmosphere. The reaction was monitored by LCMS. The mixture was allowed to cool down to room temperature. The resulting mixture was filtered, the filter cake was washed with EtOAc (3×10 mL). The filtrate was concentrated under reduced pressure. The residue was purified by Prep-TLC (DCM/MeOH 5:1) to afford (1R)-1-(1-methylpiperidin-4-yl)-1-[7-(phenylamino)-1,6-naphthyridin-2-yl]ethanol (16.5 mg, 34%) as a yellow solid. Compounds 320, 325, 335, 341, 342, 346, 348, 350, 351, 351, 352, 354, 355, 356, 358, 366, 367, 369, 370, 371, 372, 373, 377, 383, 388, 393, 397, 399, 402, 404, 405, 406, 407, 408, 409, 411, 412, and 413, as well as intermediates AG:

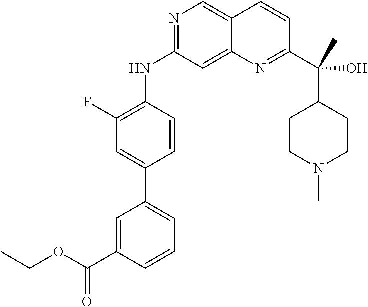 [SMILES: CCOC(=O)c1cccc(-c2ccc(Nc3cc4nc([C@](C)(O)C5CCN(C)CC5)ccc4cn3)c(F)c2)c1]

### (Preparation of

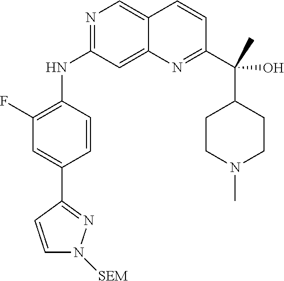 [SMILES: CN1CCC([C@@](C)(O)c2ccc3cnc(Nc4ccc(-c5ccn(COCC[Si](C)(C)C)n5)cc4F)cc3n2)CC1]

Compound 360 (alternate starting material); Example 35, and AH: (Preparation of Compound 414; Example 37) were synthesized following the methods and protocols as described for the synthesis of Compound 345, starting with the appropriate materials.

### Example 34. Preparation of Compounds 359 and 363

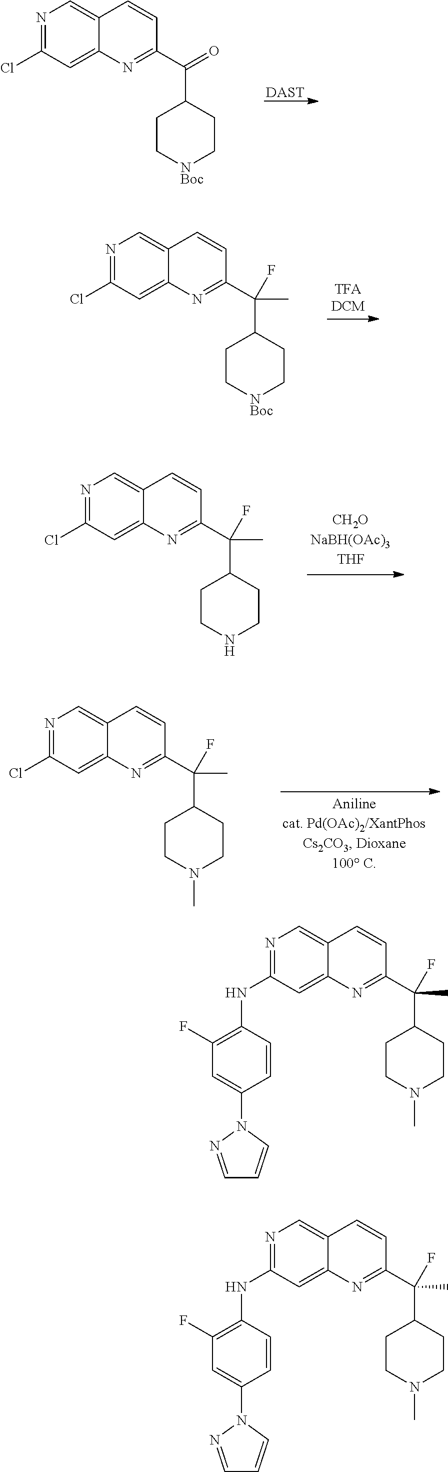

Tert-butyl 4-[1-(7-chloro-1,6-naphthyridin-2-yl)-1-fluoroethyl]piperidine-1-carboxylate. To a stirred solution of tert-butyl 4-[1-(7-chloro-1,6-naphthyridin-2-yl)-1-hydroxyethyl]piperidine-1-carboxylate (200 mg, 0.510 mmol, 1 equiv.) in DCM (1 mL) was added DAST (2 mL) dropwise at −78° C. under nitrogen atmosphere. The resulting mixture was stirred for 2 hours at room temperature under nitrogen atmosphere. The reaction was monitored by LCMS. The reaction was quenched with sat. NaHCO₃ (aq.) at 0° C. The resulting mixture was extracted with EtOAc (3×100 mL). The combined organic layers were washed with brine (1×100 mL), dried over anhydrous Na₂SO₄. After filtration, the filtrate was concentrated under reduced pressure. The residue was purified by Prep-TLC (PE/EtOAc 5:1) to afford tert-butyl 4-[1-(7-chloro-1,6-naphthyridin-2-yl)-1-fluoroethyl]piperidine-1-carboxylate (90 mg, 45%) as a yellow solid.

7-chloro-2-[1-fluoro-1-(piperidin-4-yl)ethyl]-1,6-naphthyridine. To a stirred solution of tert-butyl 4-[1-(7-chloro-1,6-naphthyridin-2-yl)-1-fluoroethyl]piperidine-1-carboxylate (100 mg, 0.254 mmol, 1 equiv.) in DCM (5 mL) was added TFA (1 mL) in portions at room temperature. The resulting mixture was stirred for 1 hour at room temperature. The reaction was monitored by LCMS. The resulting mixture was concentrated under reduced pressure. This resulted in 7-chloro-2-[1-fluoro-1-(piperidin-4-yl)ethyl]-1,6-naphthyridine (70 mg, 94%) as a light yellow solid.

7-chloro-2-[1-fluoro-1-(1-methylpiperidin-4-yl)ethyl]-1,6-naphthyridine. To a stirred mixture of 7-chloro-2-[1-fluoro-1-(piperidin-4-yl)ethyl]-1,6-naphthyridine (70 mg, 0.238 mmol, 1 equiv.) and NaBH(OAc)₃ (75.75 mg, 0.357 mmol, 1.50 equiv.) in THF (4 mL) was added HCHO (14.31 mg, 0.477 mmol, 2 equiv.) dropwise at room temperature. The resulting mixture was stirred for 1 hour at room temperature. The reaction was monitored by LCMS. The resulting mixture was concentrated under reduced pressure. The residue was purified by silica gel column chromatography, eluted with DCM/MeOH (15:1) to afford 7-chloro-2-[1-fluoro-1-(1-methylpiperidin-4-yl)ethyl]-1,6-naphthyridine (50 mg, 68%) as a light yellow solid.

(R)- and (S)-2-[1-fluoro-1-(1-methylpiperidin-4-yl)ethyl]-N-[2-fluoro-4-(pyrazol-1-yl)phenyl]-1,6-naphthyridin-7-amine. To a stirred mixture of 7-chloro-2-[1-fluoro-1-(1-methylpiperidin-4-yl)ethyl]-1,6-naphthyridine (60 mg, 0.195 mmol, 1 equiv.) and 2-fluoro-4-(pyrazol-1-yl)aniline (37.99 mg, 0.214 mmol, 1.10 equiv.) in 1,4-dioxane (4 mL) were added XantPhos (33.84 mg, 0.058 mmol, 0.30 equiv.), Cs₂CO₃ (127.03 mg, 0.390 mmol, 2 equiv.) and Pd(OAc)₂ (6.56 mg, 0.029 mmol, 0.15 equiv.) in portions at room temperature. The resulting mixture was stirred for 3 hours at 100° C. under nitrogen atmosphere. The reaction was monitored by LCMS. The resulting mixture was concentrated under reduced pressure. The residue was purified by silica gel column chromatography, eluted with DCM/MeOH (15:1) to afford racemate. Purified racemate by chiral SFC to provide 2-[(1S)-1-fluoro-1-(1-methylpiperidin-4-yl)ethyl]-N-[2-fluoro-4-(pyrazol-1-yl)phenyl]-1,6-naphthyridin-7-amine and 2-[(1R)-1-fluoro-1-(1-methylpiperidin-4-yl)ethyl]-N-[2-fluoro-4-(pyrazol-1-yl)phenyl]-1,6-naphthyridin-7-amine. Compound 363-Yield: 14.1 mg. Compound 359: Yield: 8.9 mg.

### Example 35. Preparation of Compound 362

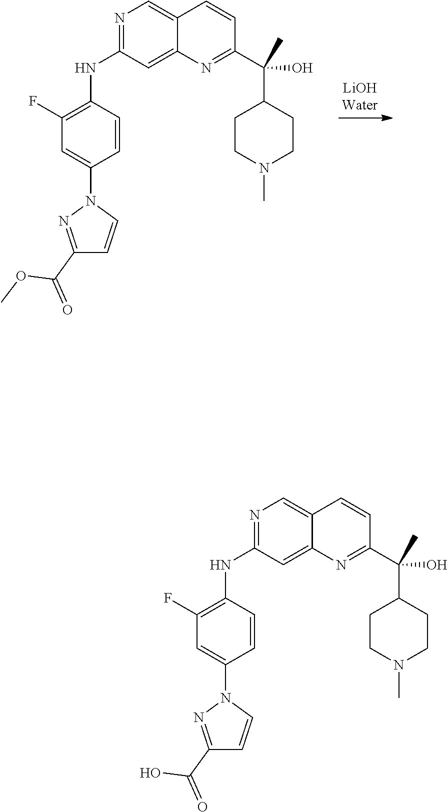 [SMILES: COC(=O)c1ccn(-c2ccc(Nc3cc4nc([C@](C)(O)C5CCN(C)CC5)ccc4cn3)c(F)c2)n1 ; CN1CCC([C@@](C)(O)c2ccc3cnc(Nc4ccc(-n5ccc(C(=O)O)n5)cc4F)cc3n2)CC1]

To a stirred mixture of methyl 1-[3-fluoro-4-([2-[(1R)-1-hydroxy-1-(1-methylpiperidin-4-yl)ethyl]-1,6-naphthyridin-7-yl]amino)phenyl]pyrazole-4-carboxylate (Compound 367, 50 mg, 0.099 mmol, 1 equiv.) in H₂O (2 mL) was added LiOH (7.12 mg, 0.297 mmol, 3 equiv.) in portions at 0° C. The resulting mixture was stirred for 1 hour at room temperature. The mixture/residue was neutralized to pH 7 with HCl (aq.). The resulting mixture was concentrated under vacuum. The residue was purified by Prep-HPLC to afford 1-[3-fluoro-4-([2-[(1R)-1-hydroxy-1-(1-methylpiperidin-4-yl)ethyl]-1,6-naphthyridin-7-yl]amino)phenyl]pyrazole-4-carboxylic acid (33.6 mg, 69%) as a yellow solid.

Compounds 324 and 360 were synthesized following the methods and protocols as described for the synthesis of Compound 362, starting with the appropriate materials.

### Example 36. Preparation of Compound 364

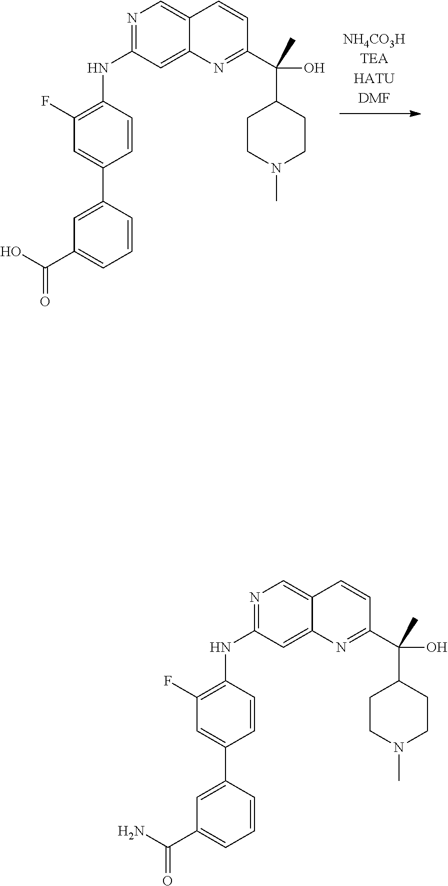 [SMILES: CN1CCC([C@@](C)(O)c2ccc3cnc(Nc4ccc(-c5cccc(C(=O)O)c5)cc4F)cc3n2)CC1 ; CN1CCC([C@@](C)(O)c2ccc3cnc(Nc4ccc(-c5cccc(C(N)=O)c5)cc4F)cc3n2)CC1]

A mixture of 3-fluoro-4-([2-[(1R)-1-hydroxy-1-(1-methylpiperidin-4-yl)ethyl]-1,6-naphthyridin-7-yl]amino)-[1,1-biphenyl]-3-carboxylic acid (Compound 360, 50 mg, 0.100 mmol, 1 equiv.), lEA (20.21 mg, 0.200 mmol, 2 equiv.), NH₄HCO₃ (39.48 mg, 0.499 mmol, 5 equiv.) and HATU (56.97 mg, 0.150 mmol, 1.50 equiv.) in DMF (3 mL) was stirred for 3 hours at 40° C. under nitrogen atmosphere. The resulting mixture was purified by Prep-HPLC to afford (R)-3′-fluoro-4′-((2-(1-hydroxy-1-(1-methylpiperidin-4-yl)ethyl)-1,6-naphthyridin-7-yl)amino)-[1,1′-biphenyl]-3-carboxamide (10.4 mg, 21%) as a yellow solid.

### Example 37. Preparation of Compound 414

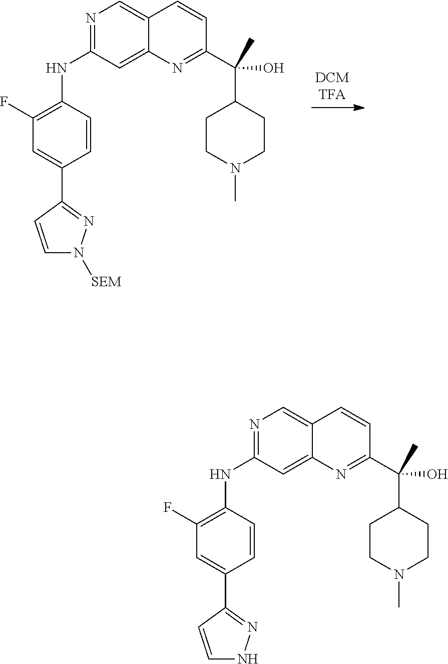 [SMILES: CN1CCC([C@@](C)(O)c2ccc3cnc(Nc4ccc(-c5ccn(COCC[Si](C)(C)C)n5)cc4F)cc3n2)CC1 ; CN1CCC([C@@](C)(O)c2ccc3cnc(Nc4ccc(-c5cc[nH]n5)cc4F)cc3n2)CC1]

To a stirred solution of (1R)-1-(7-[[2-fluoro-4-(1-[[2-(trimethylsilyl)ethoxy]methyl]pyrazol-3-yl)phenyl]amino]-1,6-naphthyridin-2-yl)-1-(1-methylpiperidin-4-yl)ethanol (45 mg, 0.078 mmol, 1 equiv.) in DCM (3 mL, 78.650 mmol, 1008.11 equiv.) was added TFA (3 mL, 40.389 mmol, 517.69 equiv.) dropwise at room temperature under air atmosphere for 2 h. The mixture was basified to pH 7 with saturated NaHCO₃ (aq.). The resulting mixture was extracted with EtOAc (3×5 mL), dried over anhydrous Na₂SO₄. After filtration, the filtrate was concentrated under reduced pressure. The crude product (20.9 mg) was purified by Prep-HPLC to afford (1R)-1-(7-[[2-fluoro-4-(1H-pyrazol-3-yl)phenyl]amino]-1,6-naphthyridin-2-yl)-1-(1-methylpiperidin-4-yl)ethanol (14.4 mg, 41%) as a light yellow solid.

### Example 38. Preparation of Compounds 186 and 188

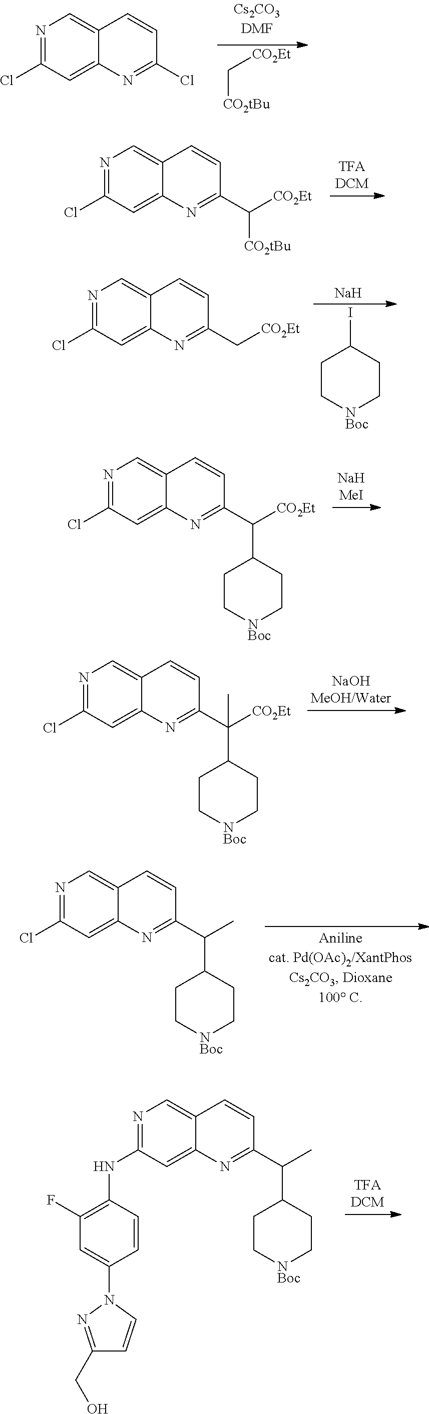

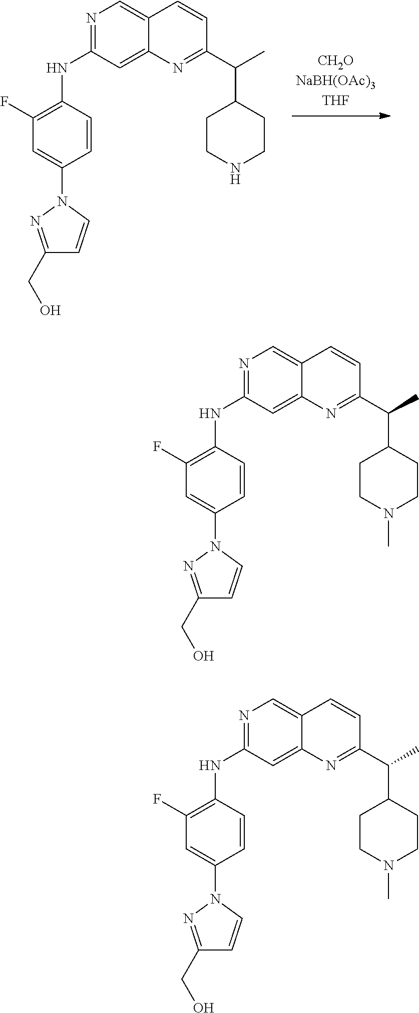 [SMILES: CC(c1ccc2cnc(Nc3ccc(-n4ccc(CO)n4)cc3F)cc2n1)C1CCNCC1 ; C[C@H](c1ccc2cnc(Nc3ccc(-n4ccc(CO)n4)cc3F)cc2n1)C1CCN(C)CC1 ; C[C@@H](c1ccc2cnc(Nc3ccc(-n4ccc(CO)n4)cc3F)cc2n1)C1CCN(C)CC1]

1-tert-butyl 3-ethyl 2-(7-chloro-1,6-naphthyridin-2-yl)propanedioate. To a solution of 2,7-dichloro-1,6-naphthyridine (7 g, 35.171 mmol, 1 equiv.) in DMF (100 mL) were added 1-tert-butyl 3-ethyl propanedioate (13.24 g, 70.341 mmol, 2 equiv.) and Cs₂CO₃ (22.92 g, 70.341 mmol, 2 equiv.) at room temperature. The resulting mixture was stirred for overnight at 80° C. The reaction mixture was diluted with water (500 mL)? extracted with ethyl acetate (2×300 mL). The combined organic layers were washed with water (5×300 mL), brine (300 mL), dried over anhydrous sodium sulfate and filtered. The filtrate was concentrated under reduced pressure, the residue was purified by silica gel column chromatography, eluted with 3-10% ethyl acetate in petroleum ether to afford 1-tert-butyl 3-ethyl 2-(7-chloro-1,6-naphthyridin-2-yl)propanedioate (7.5 g, 60%) as a brown oil.

Ethyl 2-(7-chloro-1,6-naphthyridin-2-yl)acetate. A solution of 1-tert-butyl 3-ethyl 2-(7-chloro-1,6-naphthyridin-2-yl)propanedioate (1.60 g, 4.561 mmol, 1 equiv.) in DCM (40 mL) was added TFA (10 mL, 134.630 mmol, 29.52 equiv.) at 0° C. The resulting mixture was stirred for 2 hours at room temperature. The resulting mixture was concentrated under reduced pressure. The resulting mixture was diluted with EtOAc (100 mL). The resulting mixture was washed with brine (2×100 mL). The organic layers were dried over anhydrous Na₂SO₄ and concentrated under reduced pressure. The residue was purified by silica gel column chromatography, eluted with PE/EtOAc (5:1) to afford ethyl 2-(7-chloro-1,6-naphthyridin-2-yl)acetate (1.2 g) as a yellow solid.

Tert-butyl 4-[1-(7-chloro-1,6-naphthyridin-2-yl)-2-ethoxy-2-oxoethyl]piperidine-1-carboxylate. To a solution of ethyl 2-(7-chloro-1,6-naphthyridin-2-yl)acetate (5.30 g, 21.142 mmol, 1 equiv.) in DMF (100 mL) was added sodium hydride (60% in oil, 0.76 g, 31.714 mmol, 1.50 equiv.) at 0° C. The mixture was stirred for 30 min. tert-butyl 4-iodopiperidine-1-carboxylate (9.87 g, 31.720 mmol, 1.50 equiv.) was added and the mixture was allowed to warm to RT and stirred for 16 hours. The reaction mixture was quenched with water (500 mL), extracted with ethyl acetate (2×200 mL). The combined organic layers were washed with water (5×200 mL), brine (200 mL), dried over anhydrous sodium sulfate and filtered. The filtrate was concentrated under reduced pressure, the residue was purified by silica gel column chromatography, eluted with 1-3% ethyl acetate in petroleum ether to afford tert-butyl 4-[1-(7-chloro-1,6-naphthyridin-2-yl)-2-ethoxy-2-oxoethyl]piperidine-1-carboxylate (5.6 g) as a brown oil.

Tert-butyl 4-[2-(7-chloro-1,6-naphthyridin-2-yl)-1-ethoxy-1-oxopropan-2-yl]piperidine-1-carboxylate. To a stirred solution of tert-butyl 4-[1-(7-chloro-1,6-naphthyridin-2-yl)-2-ethoxy-2-oxoethyl]piperidine-1-carboxylate (1 g, 2.305 mmol, 1 equiv.) in DMF (35 mL) was added NaH (119.82 mg, 2.996 mmol, 1.3 equiv, 60%) at room temperature under nitrogen atmosphere. The resulting mixture was stirred for 30 min at room temperature. The mixture was added CH₃I (490.65 mg, 3.457 mmol, 1.5 equiv.). The resulting mixture was stirred for 1 h at room temperature. The reaction was quenched with sat. NH₄C₁ (aq.) at room temperature. The resulting mixture was extracted with EtOAc (2×100 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EtOAc (10:1 to 3:1) to afford tert-butyl 4-[2-(7-chloro-1,6-naphthyridin-2-yl)-1-ethoxy-1-oxopropan-2-yl]piperidine-1-carboxylate (800 mg, 77%) as an off-white solid.

Tert-butyl 4-[1-(7-chloro-1,6-naphthyridin-2-yl)ethyl]piperidine-1-carboxylate. To a stirred solution of tert-butyl 4-[2-(7-chloro-1,6-naphthyridin-2-yl)-1-ethoxy-1-oxopropan-2-yl]piperidine-1-carboxylate (800 mg, 1.786 mmol, 1 equiv.) in MeOH (20 mL) was added 2N NaOH (20 mL) at room temperature under nitrogen atmosphere. The resulting mixture was stirred for 36 hours at 50° C. under nitrogen atmosphere. The reaction was monitored by LCMS. The mixture was allowed to cool down to room temperature. The resulting mixture was concentrated under reduced pressure. The resulting mixture was diluted with brine (100 mL). The resulting mixture was extracted with EtOAc (2×100 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EtOAc (10:1 to 3:1) to afford tert-butyl 4-[1-(7-chloro-1,6-naphthyridin-2-yl)ethyl]piperidine-1-carboxylate (500 mg, 74%) as an off-white solid.

Tert-butyl 4-[1-[7-([2-fluoro-4-[3-(hydroxymethyl)pyrazol-1-yl]phenyl]amino)-1,6-naphthyridin-2-yl]ethyl]piperidine-1-carboxylate. To a stirred solution of tert-butyl 4-[1-(7-chloro-1,6-naphthyridin-2-yl)ethyl]piperidine-1-carboxylate (510 mg, 1.357 mmol, 1 equiv.), [1-(4-amino-3-fluorophenyl)pyrazol-3-yl]methanol (309.24 mg, 1.492 mmol, 1.1 equiv.), XantPhos (235.51 mg, 0.407 mmol, 0.3 equiv.) and Cs₂CO₃ (884.11 mg, 2.713 mmol, 2.0 equiv.) in 1,4-dioxane (50 mL) was added Pd(OAc)₂ (45.69 mg, 0.204 mmol, 0.15 equiv.) at room temperature under nitrogen atmosphere. The resulting mixture was stirred for 2 hours at 100° C. under nitrogen atmosphere. The reaction was monitored by LCMS. The mixture was allowed to cool down to room temperature. The residue was purified by reverse phase flash chromatography with the following conditions: Column: Spherical C18, 20-40 um, 330 g; Mobile Phase A: Water (plus 5 mM HOAc); Mobile Phase B: ACN; Flow rate: 45 mL/min; Gradient: 5%-5% B, 10 min, 33% B-45% B gradient in 20 min; Detector: 254 nm. The fractions containing the desired product were collected at 40% B and concentrated under reduced pressure to afford tert-butyl 4-[1-[7-([2-fluoro-4-[3-(hydroxymethyl)pyrazol-1-yl]phenyl]amino)-1,6-naphthyridin-2-yl]ethyl]piperidine-1-carboxylate (445 mg, 60%) as a white solid

[1-[3-fluoro-4-([2-[1-(piperidin-4-yl)ethyl]-1,6-naphthyridin-7-yl]amino)phenyl]pyrazol-3-yl]methanol. To a stirred solution of tert-butyl 4-[1-[7-([2-fluoro-4-[3-(hydroxymethyl)pyrazol-1-yl]phenyl]amino)-1,6-naphthyridin-2-yl]ethyl]piperidine-1-carboxylate (450 mg, 0.823 mmol, 1 equiv.) in DCM (10 mL) was added TFA (1 mL) at room temperature under nitrogen atmosphere. The resulting mixture was stirred for 1 hour at room temperature under nitrogen atmosphere. The reaction was monitored by LCMS. The resulting mixture was concentrated under reduced pressure. The resulting mixture was diluted with brine (50 mL). The mixture/residue was basified to pH 8 with saturated NaHCO₃ (aq.). The resulting mixture was extracted with DCM (2×50 mL). The combined organic layers were washed with brine (1×100 mL), dried over anhydrous Na₂SO₄. After filtration, the filtrate was concentrated under reduced pressure. The residue was purified by silica gel column chromatography, eluted with PE/EtOAc (10:1 to 3:1) to afford [1-[3-fluoro-4-([2-[1-(piperidin-4-yl)ethyl]-1,6-naphthyridin-7-yl]amino)phenyl]pyrazol-3-yl]methanol (300 mg, 81%) as a yellow solid.

(R)- and (S)-[1-[3-fluoro-4-([2-[1-(1-methylpiperidin-4-yl)ethyl]-1,6-naphthyridin-7-yl]amino)phenyl]pyrazol-3-yl]methanol (Compounds 186 and 188). To a stirred solution of [1-[3-fluoro-4-([2-[1-(piperidin-4-yl)ethyl]-1,6-naphthyridin-7-yl]amino)phenyl]pyrazol-3-yl]methanol (300 mg, 0.672 mmol, 1 equiv.) and HCHO (26.22 mg, 0.873 mmol, 1.3 equiv.) in THF (50 mL) was added NaBH(OAc)₃ (213.59 mg, 18 mmol, 1.5 equiv.) at room temperature under nitrogen atmosphere. The resulting mixture was stirred for 1 hour at room temperature under nitrogen atmosphere. The reaction was monitored by LCMS. The resulting mixture was concentrated under reduced pressure. The residue was purified by reverse phase flash chromatography with the following conditions: Column: Spherical C18, 20-40 um, 330 g; Mobile Phase A: Water (plus 0.05% TFA); Mobile Phase B: ACN; Flow rate: 85 mL/min; Gradient: 5%-5% B, 10 min, 33% B-45% B gradient in 20 min; Detector: 254 nm. The fractions containing the desired product were collected at 40% B and concentrated under reduced pressure to afford racemic product. The racemate was separated by Prep-Chiral with the following conditions: Column: CHIRALPAK IG, 20*250 mm, 5 um; Mobile Phase A:Hex (8 mmol/L NH₃MeOH), Mobile Phase B:EtOH:DCM=1:1; Flow rate:20 mL/min; Gradient:38 B to 38 B in 20 min; 220/254 nm; RT1:13.916 min; RT2:16.349 min; Injection Volumn:0.5 mL; Number Of Runs:10; Obtained (R)- and (S)-[1-[3-fluoro-4-([2-[1-(1-methylpiperidin-4-yl)ethyl]-1,6-naphthyridin-7-yl]amino)phenyl]pyrazol-3-yl]methanol. Compound 186-eluted at 16.349 min; yield: 43.7 mg. Compound 188-eluted at 13.916 min; yield: 48.4 mg.

Compounds 204, 244, 246, 310, 311, 314, 315, 319, 322, 333, and 339 were synthesizes following the methods and protocols as described for the synthesis of Compounds 186 and 188, starting with the appropriate materials.

### Example 39. Preparation of Compound 410

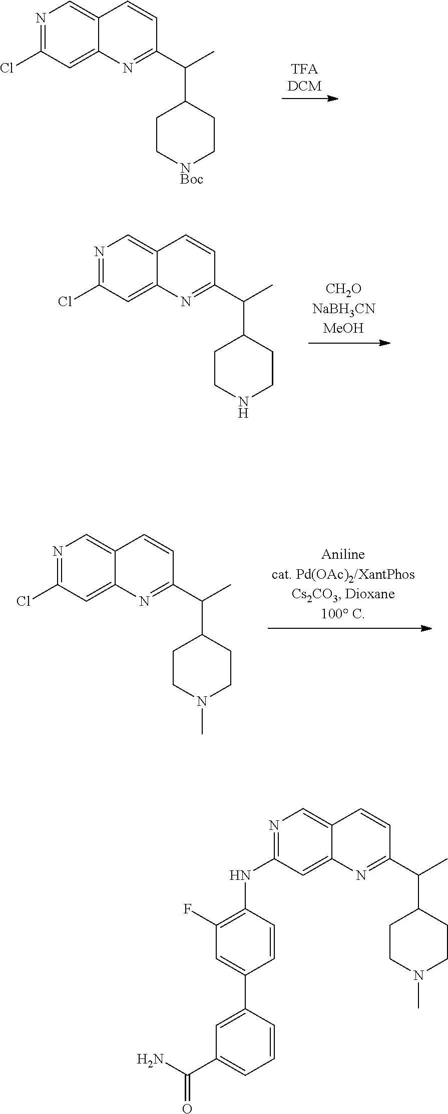 [SMILES: CC(c1ccc2cnc(Cl)cc2n1)C1CCN(C(=O)OC(C)(C)C)CC1 ; CC(c1ccc2cnc(Cl)cc2n1)C1CCNCC1 ; CC(c1ccc2cnc(Cl)cc2n1)C1CCN(C)CC1 ; CC(c1ccc2cnc(Nc3ccc(-c4cccc(C(N)=O)c4)cc3F)cc2n1)C1CCN(C)CC1]

7-chloro-2-[1-(piperidin-4-yl)ethyl]-1,6-naphthyridine. To a stirred solution of tert-butyl 4-[1-(7-chloro-1,6-naphthyridin-2-yl)ethyl]piperidine-1-carboxylate (Compound 188; step 5, 700 mg, 1.862 mmol, 1 equiv.) in DCM (15 mL) was added TFA (30 mL, 403.891 mmol, 216.89 equiv.) in portions at room temperature. The resulting mixture was stirred for 1 hour at 0° C. The reaction was monitored by LCMS. The resulting mixture was extracted with DCM (3×10 mL). The combined organic layers were washed with DCM (3×10 mL) dried over anhydrous Na₂SO₄. After filtration, the filtrate was concentrated under reduced pressure to afford 7-chloro-2-[1-(piperidin-4-yl)ethyl]-1,6-naphthyridine (500 mg, 97%) as a brown oil. 7-chloro-2-[1-(1-methylpiperidin-4-yl)ethyl]-1,6-naphthyridine. To a stirred solution of 7-chloro-2-[1-(piperidin-4-yl)ethyl]-1,6-naphthyridine (500 mg, 1.813 mmol, 1 equiv.) and HCHO (362.92 mg, 3.626 mmol, 2 equiv, 30%) in MeOH (10 mL) ware added NaBH₃CN (227.87 mg, 3.626 mmol, 2 equiv.) in portions at 0° C. The resulting mixture was stirred for 1 hour at 0° C. The reaction was monitored by LCMS. The resulting mixture was concentrated under reduced pressure. The residue was purified by reverse phase flash with the following conditions (Column: C18, 120 g; Mobile Phase A:Water/0.05% NH₄HCO₃, Mobile Phase B:ACN; Flow rate:40 mL/min; Gradient: 45% B to 95% B in 20 min; Detector, 254 nm and 220 nm, the desired product were collected at 75% B) to afford 7-chloro-2-[1-(1-methylpiperidin-4-yl)ethyl]-1,6-naphthyridine (250 mg, 47%) as a brown solid.

3′-fluoro-4′-([2-[1-(1-methylpiperidin-4-yl)ethyl]-1,6-naphthyridin-7-yl]amino)-[1,1′-biphenyl]-3-carboxamide (Compound 410). To a stirred mixture of 7-chloro-2-[1-(1-methylpiperidin-4-yl)ethyl]-1,6-naphthyridine (198.10 mg, 0.684 mmol, 1 equiv.) and 4-amino-3-fluoro-[1,1-biphenyl]-3-carboxamide (157.38 mg, 0.684 mmol, 1 equiv.) in 1,4-dioxane (8.00 mL) were added Pd(OAc)₂ (46.04 mg, 0.205 mmol, 0.30 equiv.), XantPhos (237.31 mg, 0.410 mmol, 0.60 equiv.) and Cs₂CO₃ (445.43 mg, 1.367 mmol, 2 equiv.) at room temperature under nitrogen atmosphere. The resulting mixture was stirred for 1 h at 100° C. under nitrogen atmosphere. The reaction was monitored by LCMS. The mixture was allowed to cool down to room temperature. The resulting mixture was concentrated under reduced pressure. The residue was purified by Prep-TLC (DCM/MeOH 5:1) to give crude product which was further purified by Prep-HPLC to afford 3′-fluoro-4′-([2-[1-(1-methylpiperidin-4-yl)ethyl]-1,6-naphthyridin-7-yl]amino)-[1,1′-biphenyl]-3-carboxamide (14.6 mg, 4%) as a light yellow solid.

Compounds 368 and 386 were synthesized following the methods and protocols as described for the synthesis of Compound 410, starting with the appropriate materials.

### Example 40. Preparation of Compound 139

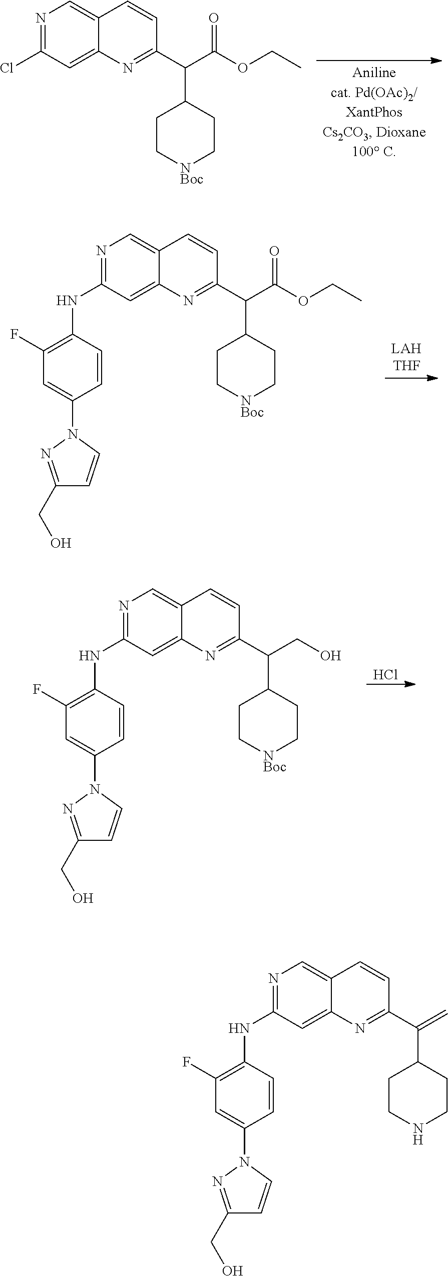 [SMILES: CCOC(=O)C(c1ccc2cnc(Cl)cc2n1)C1CCN(C(=O)OC(C)(C)C)CC1 ; CCOC(=O)C(c1ccc2cnc(Nc3ccc(-n4ccc(CO)n4)cc3F)cc2n1)C1CCN(C(=O)OC(C)(C)C)CC1 ; CC(C)(C)OC(=O)N1CCC(C(CO)c2ccc3cnc(Nc4ccc(-n5ccc(CO)n5)cc4F)cc3n2)CC1 ; Cl ; C=C(c1ccc2cnc(Nc3ccc(-n4ccc(CO)n4)cc3F)cc2n1)C1CCNCC1]

Tert-butyl 4-[2-ethoxy-1-[7-([2-fluoro-4-[3-(hydroxymethyl)pyrazol-1-yl]phenyl]amino)-1,6-naphthyridin-2-yl]-2-oxoethyl]piperidine-1-carboxylate. To a stirred mixture of tert-butyl 4-[1-(7-chloro-1,6-naphthyridin-2-yl)-2-ethoxy-2-oxoethyl]piperidine-1-carboxylate (Compound 188, step 3, 300 mg, 0.691 mmol, 1 equiv.) and [1-(4-amino-3-fluorophenyl)pyrazol-3-yl]methanol (157.58 mg, 0.760 mmol, 1.10 equiv.) in 1,4-dioxane (10 mL) were added Pd(OAc)₂ (23.28 mg, 0.104 mmol, 0.15 equiv.), XantPhos (120.01 mg, 0.207 mmol, 0.30 equiv.) and Cs₂CO₃ (450.51 mg, 1.383 mmol, 2 equiv.) in portions at rt under nitrogen atmosphere. The resulting mixture was stirred for 2 hours at 100° C. under nitrogen atmosphere. The reaction was monitored by LCMS. The mixture was allowed to cool to rt. The resulting mixture was extracted with EtOAc (3×200 mL). The combined organic layers were washed with brine (2×100 mL), dried over anhydrous Na₂SO₄. After filtration, the filtrate was concentrated under reduced pressure. The residue was purified by reverse phase flash chromatography with the following conditions: Column: Spherical C18, 20-40 um, 330 g; Mobile Phase A: Water (plus 5 mM NH₄HCO₃); Mobile Phase B: ACN; Flow rate: 80 mL/min; Gradient: 5%-5% B, 10 min, 45% B-80% B gradient in 30 min; Detector: 220 nm. The fractions containing the desired product were collected at 75% B and concentrated under reduced pressure to afford tert-butyl 4-[2-ethoxy-1-[7-([2-fluoro-4-[3-(hydroxymethyl)pyrazol-1-yl]phenyl]amino)-1,6-naphthyridin-2-yl]-2-oxoethyl]piperidine-1-carboxylate (200 mg, 47%) as a yellow solid.

Tert-butyl 4-[1-[7-([2-fluoro-4-[3-(hydroxymethyl)pyrazol-1-yl]phenyl]amino)-1,6-naphthyridin-2-yl]-2-hydroxyethyl]piperidine-1-carboxylate. To a stirred solution of tert-butyl 4-[2-ethoxy-1-[7-([2-fluoro-4-[3-(hydroxymethyl)pyrazol-1-yl]phenyl]amino)-1,6-naphthyridin-2-yl]-2-oxoethyl]piperidine-1-carboxylate (50 mg, 0.083 mmol, 1 equiv.) in THF (6 mL) was added LiAlH₄ (3.77 mg, 0.099 mmol, 1.20 equiv.) at room temperature under nitrogen atmosphere. The resulting mixture was stirred for 0.5 hours at room temperature under nitrogen atmosphere. The reaction was monitored by TLC. The reaction was quenched with water (0.3 mL) at room temperature. The resulting mixture was concentrated under reduced pressure. The residue was purified by TLC, eluted with DCM/MeOH (10:1) to afford tert-butyl 4-[1-[7-([2-fluoro-4-[3-(hydroxymethyl)pyrazol-1-yl]phenyl]amino)-1,6-naphthyridin-2-yl]-2-hydroxyethyl]piperidine-1-carboxylate (30 mg, 64%) as an off-white solid. [1-[3-fluoro-4-([2-[1-(piperidin-4-yl)ethenyl]-1,6-naphthyridin-7-yl]amino)phenyl]pyrazol-3-yl]methanol. To a stirred solution of tert-butyl 4-[1-[7-([2-fluoro-4-[3-(hydroxymethyl)pyrazol-1-yl]phenyl]amino)-1,6-naphthyridin-2-yl]-2-hydroxyethyl]piperidine-1-carboxylate (15 mg) in THF (2 mL) was added HCl (gas) in 1,4-dioxane (2 mL) at room temperature. The resulting mixture was stirred for 3 hours at room temperature. The reaction was monitored by TLC. The resulting mixture was concentrated under vacuum. The residue was purified by Prep-HPLC to afford [1-[3-fluoro-4-([2-[1-(piperidin-4-yl)ethenyl]-1,6-naphthyridin-7-yl]amino)phenyl]pyrazol-3-yl]methanol (3.3 mg) as a light yellow solid.

### Example 41. Preparation of Compound 168

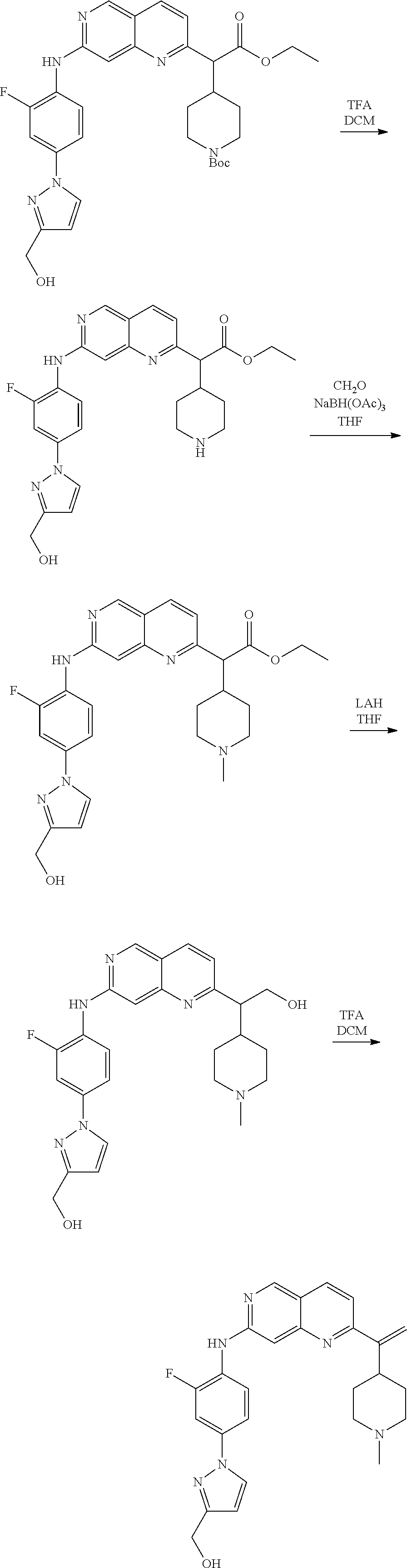 [SMILES: CCOC(=O)C(c1ccc2cnc(Nc3ccc(-n4ccc(CO)n4)cc3F)cc2n1)C1CCN(C(=O)OC(C)(C)C)CC1 ; CCOC(=O)C(c1ccc2cnc(Nc3ccc(-n4ccc(CO)n4)cc3F)cc2n1)C1CCNCC1 ; CCOC(=O)C(c1ccc2cnc(Nc3ccc(-n4ccc(CO)n4)cc3F)cc2n1)C1CCN(C)CC1 ; CN1CCC(C(CO)c2ccc3cnc(Nc4ccc(-n5ccc(CO)n5)cc4F)cc3n2)CC1 ; C=C(c1ccc2cnc(Nc3ccc(-n4ccc(CO)n4)cc3F)cc2n1)C1CCN(C)CC1]

Ethyl 2-[7-([2-fluoro-4-[3-(hydroxymethyl)pyrazol-1-yl]phenyl]amino)-1,6-naphthyridin-2-yl]-2-(piperidin-4-yl)acetate. To a stirred solution of tert-butyl 4-[2-ethoxy-1-[7-([2-fluoro-4-[3-(hydroxymethyl)pyrazol-1-yl]phenyl]amino)-1,6-naphthyridin-2-yl]-2-oxoethyl]piperidine-1-carboxylate (Compound 139, step 1, 300 mg, 0.496 mmol, 1 equiv.) in DCM (10 mL, 157.300 mmol, 317.06 equiv.) was added TFA (1 mL, 13.463 mmol, 27.14 equiv.) in portions at room temperature under nitrogen atmosphere. The resulting mixture was stirred for 2 hours at room temperature. The reaction was monitored by LCMS. The resulting mixture was concentrated under reduced pressure. The residue was purified by reverse phase flash chromatography with the following conditions: Column: Spherical C18, 20-40 um, 330 g; Mobile Phase A: Water (0.5% TFA); Mobile Phase B: ACN; Flow rate: 85 mL/min; Gradient: 5%-5% B, 10 min, 33% B—45% B gradient in 20 min; Detector: 254 nm. The fractions containing the desired product were collected at 40% B and concentrated under reduced pressure to afford ethyl 2-[7-([2-fluoro-4-[3-(hydroxymethyl)pyrazol-1-yl]phenyl]amino)-1,6-naphthyridin-2-yl]-2-(piperidin-4-yl)acetate (160 mg, 63%) as a yellow solid

Ethyl 2-[7-([2-fluoro-4-[3-(hydroxymethyl)pyrazol-1-yl]phenyl]amino)-1,6-naphthyridin-2-yl]-2-(1-methylpiperidin-4-yl)acetate. To a stirred solution of ethyl 2-[7-([2-fluoro-4-[3-(hydroxymethyl)pyrazol-1-yl]phenyl]amino)-1,6-naphthyridin-2-yl]-2-(piperidin-4-yl)acetate (160 mg, 0.317 mmol, 1 equiv.) and HCHO (14.28 mg, 0.476 mmol, 1.50 equiv.) in THF (15 mL) was added NaBH(OAc)₃ (107.53 mg, 0.507 mmol, 1.60 equiv.) in portions at room temperature under nitrogen atmosphere. The resulting mixture was stirred for 2 hours at room temperature. The reaction was monitored by LCMS. The residue was purified by reverse phase flash chromatography with the following conditions: Column: Spherical C18, 20-40 um, 330 g; Mobile Phase A: Water (0.5% TFA); Mobile Phase B: ACN; Flow rate: 85 mL/min; Gradient: 5%-5% B, 10 min, 33% B-45% B gradient in 20 min; Detector: 254 nm. The fractions containing the desired product were collected at 40% B and concentrated under reduced pressure to afford ethyl 2-[7-([2-fluoro-4-[3-(hydroxymethyl)pyrazol-1-yl]phenyl]amino)-1,6-naphthyridin-2-yl]-2-(1-methylpiperidin-4-yl)acetate (111 mg, 67%) as a yellow solid

2-[7-([2-fluoro-4-[3-(hydroxymethyl)pyrazol-1-yl]phenyl]amino)-1,6-naphthyridin-2-yl]-2-(1-methylpiperidin-4-yl)ethanol. To a stirred solution of ethyl 2-[7-([2-fluoro-4-[3-(hydroxymethyl)pyrazol-1-yl]phenyl]amino)-1,6-naphthyridin-2-yl]-2-(1-methylpiperidin-4-yl)acetate (100 mg, 0.193 mmol, 1 equiv.) in THF (10 mL) was added LiA1H4 (18.30 mg, 0.482 mmol, 2.5 equiv.) in portions at room temperature under nitrogen atmosphere. The resulting mixture was stirred for 1 hour at room temperature under nitrogen atmosphere. The reaction was monitored by LCMS. The resulting mixture was concentrated under reduced pressure. The residue was purified by reverse phase flash chromatography with the following conditions: Column: Spherical C18, 20-40 um, 330 g; Mobile Phase A: Water (0.5% TFA); Mobile Phase B: ACN; Flow rate: 85 mL/min; Gradient: 5%-5% B, 10 min, 33% B-45% B gradient in 20 min; Detector: 254 nm. The fractions containing the desired product were collected at 40% B and concentrated under reduced pressure to afford 2-[7-([2-fluoro-4-[3-(hydroxymethyl)pyrazol-1-yl]phenyl]amino)-1,6-naphthyridin-2-yl]-2-(1-methylpiperidin-4-yl)ethanol (55 mg, 59%) as a yellow solid.

[1-[3-fluoro-4-([2-[1-(1-methylpiperidin-4-yl)ethenyl]-1,6-naphthyridin-7-yl]amino)phenyl]pyrazol-3-yl]methanol. To a stirred solution of 2-[7-([2-fluoro-4-[3-(hydroxymethyl)pyrazol-1-yl]phenyl]amino)-1,6-naphthyridin-2-yl]-2-(1-methylpiperidin-4-yl)ethanol (55 mg) in DCM (10 mL) was added TFA (1 mL) in portions at room temperature. The resulting mixture was stirred for 2 hours at room temperature. The reaction was monitored by LCMS. The resulting mixture was concentrated under reduced pressure. The residue was purified by reverse phase flash chromatography with the following conditions: Column: Spherical C18, 20-40 um, 330 g; Mobile Phase A: Water (0.5% TFA); Mobile Phase B: ACN; Flow rate: 85 mL/min; Gradient: 5%-5% B, 10 min, 33% B-45% B gradient in 20 min; Detector: 254 nm. The fractions containing the desired product were collected at 40% B and concentrated under reduced pressure to afford trifluoroacetic acid; [1-[3-fluoro-4-([2-[1-(1-methylpiperidin-4-yl)ethenyl]-1,6-naphthyridin-7-yl]amino)phenyl]pyrazol-3-yl]methanol (7.1 mg) as a yellow solid

### Example 42. Preparation of Compound 142

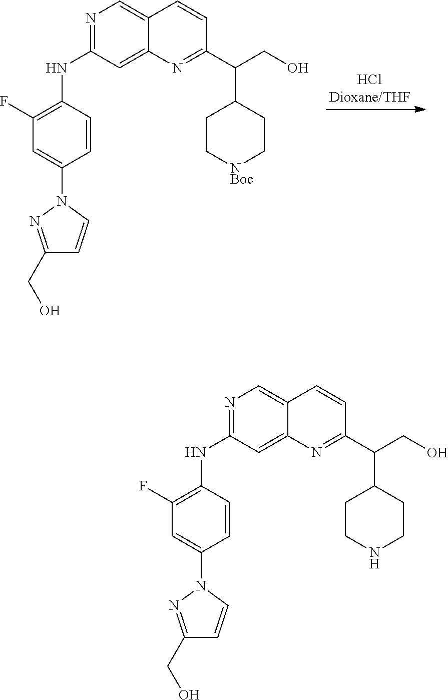 [SMILES: CC(C)(C)OC(=O)N1CCC(C(CO)c2ccc3cnc(Nc4ccc(-n5ccc(CO)n5)cc4F)cc3n2)CC1 ; OCc1ccn(-c2ccc(Nc3cc4nc(C(CO)C5CCNCC5)ccc4cn3)c(F)c2)n1]

To a stirred solution of tert-butyl 4-[1-[7-([2-fluoro-4-[3-(hydroxymethyl)pyrazol-1-yl]phenyl]amino)-1,6-naphthyridin-2-yl]-2-hydroxyethyl]piperidine-1-carboxylate (Compound 139, step 2, 15 mg) in THF (2 mL) was added HCl (gas) in 1,4-dioxane (2 mL) at room temperature. The resulting mixture was stirred for 3 hours at room temperature. The reaction was monitored by TLC. The resulting mixture was concentrated under vacuum. The residue was purified by Prep-HPLC to afford 2-[7-([2-fluoro-4-[3-(hydroxymethyl)pyrazol-1-yl]phenyl]amino)-1,6-naphthyridin-2-yl]-2-(piperidin-4-ypethanol; formic acid (2.1 mg) as a light yellow solid.

### Example 43. Preparation of Compounds 312 and 316

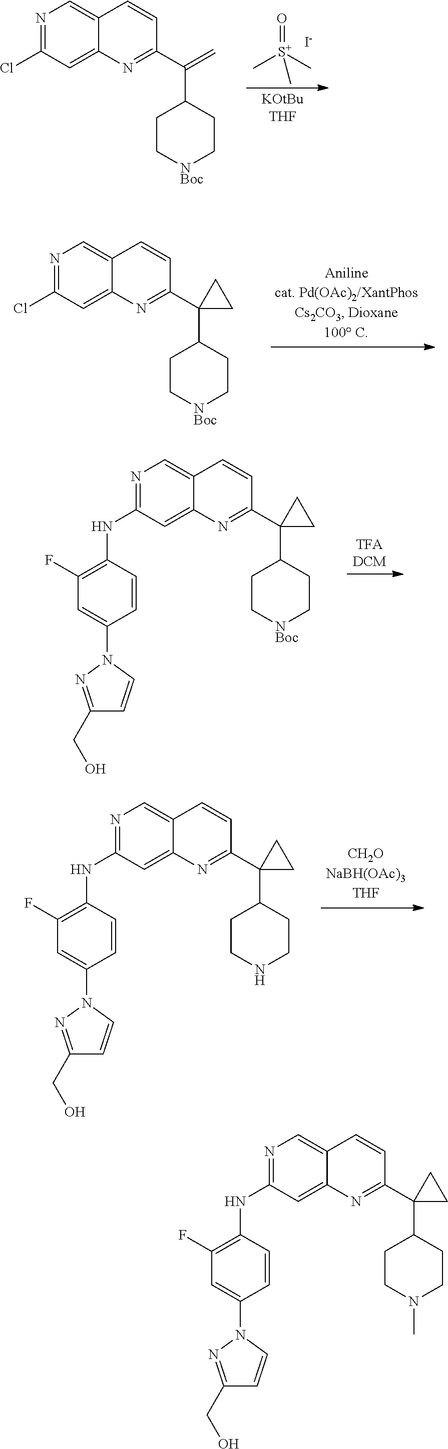

Tert-butyl 4-[1-(7-chloro-1,6-naphthyridin-2-yl)cyclopropyl]piperidine-1-carboxylate. To a stirred solution of tert-butyl 4-[1-(7-chloro-1,6-naphthyridin-2-yl)ethenyl]piperidine-1-carboxylate (200 mg, 0.535 mmol, 1 equiv.) and t-BuOK (90.04 mg, 0.802 mmol, 1.5 equiv.) in THF (30 mL, 370.290 mmol, 692.22 equiv.) was added Trimethylsulfoxonium iodide (176.58 mg, 0.802 mmol, 1.5 equiv.) in portions at room temperature under nitrogen atmosphere. The resulting mixture was stirred for 4 hours at room temperature under nitrogen atmosphere. The reaction was monitored by LCMS. The reaction was quenched with sat. NH₄C₁ (aq.) at room temperature. The resulting mixture was extracted with EtOAc (3×150 mL). The combined organic layers were washed with brine (1×150 mL), dried over anhydrous Na₂SO₄. After filtration, the filtrate was concentrated under reduced pressure. The residue was purified by silica gel column chromatography, eluted with PE/EtOAc (1:1) to afford tert-butyl 4-[1-(7-chloro-1,6-naphthyridin-2-yl)cyclopropyl]piperidine-1-carboxylate (150 mg, 72%) as a white solid.

Tert-butyl 4-[1-[7-([2-fluoro-4-[3-(hydroxymethyl)pyrazol-1-yl]phenyl]amino)-1,6-naphthyridin-2-yl]cyclopropyl]piperidine-1-carboxylate. To a stirred solution of tert-butyl 4-[1-(7-chloro-1,6-naphthyridin-2-yl)cyclopropyl]piperidine-1-carboxylate (130 mg, 0.335 mmol, 1 equiv.), [1-(4-amino-3-fluorophenyl)pyrazol-3-yl]methanol (83.33 mg, 0.402 mmol, 1.20 equiv.), XantPhos (58.17 mg, 0.101 mmol, 0.3 equiv.) and Cs₂CO₃ (218.38 mg, 0.670 mmol, 2.0 equiv.) in 1,4-dioxane (10 mL) was added Pd(OAc)₂ (11.29 mg, 0.050 mmol, 0.15 equiv.) dropwise at room temperature under nitrogen atmosphere. The resulting mixture was stirred for 2 hours at 100° C. under nitrogen atmosphere. The reaction was monitored by LCMS. The mixture was allowed to cool down to room temperature. The resulting mixture was concentrated under reduced pressure. The residue was purified by Prep-TLC (DCM/MeOH 10:1) to afford tert-butyl 4-[1-[7-([2-fluoro-4-[3-(hydroxymethyl)pyrazol-1-yl]phenyl]amino)-1,6-naphthyridin-2-yl]cyclopropyl]piperidine-1-carboxylate (90 mg, 48%) as an off-white solid.

[1-[3-fluoro-4-([2-[1-(piperidin-4-yl)cyclopropyl]-1,6-naphthyridin-7-yl]amino)phenyl]pyrazol-3-yl]methanol (Compound 316). To a stirred solution of tert-butyl 4-[1-[7-([2-fluoro-4-[3-(hydroxymethyl)pyrazol-1-yl]phenyl]amino)-1,6-naphthyridin-2-yl]cyclopropyl]piperidine-1-carboxylate (100 mg, 0.179 mmol, 1 equiv.) in DCM (10 mL) was added TFA (1 mL) under nitrogen atmosphere. The resulting mixture was stirred for 2 hours at room temperature under nitrogen atmosphere. The resulting mixture was concentrated under vacuum. The resulting mixture was diluted with DCM (10 mL). The mixture was basified to pH 9 with saturated NaHCO₃ (aq.). The resulting mixture was extracted with DCM (3×50 mL). The combined organic layers were washed with brine (1×30 mL), dried over anhydrous Na₂SO₄. After filtration, the filtrate was concentrated under reduced pressure. The residue was purified by silica gel column chromatography, eluted with DCM/MeOH (10:1) to afford [1-[3-fluoro-4-([2-[1-(piperidin-4-yl)cyclopropyl]-1,6-naphthyridin-7-yl]amino)phenyl]pyrazol-3-yl]methanol (50 mg, 60%) as a light yellow solid.

[1-[3-fluoro-4-([2-[1-(1-methylpiperidin-4-yl)cyclopropyl]-1,6-naphthyridin-7-yl]amino)phenyl]pyrazol-3-yl]methanol (Compound 312). To a stirred solution of [1-[3-fluoro-4-([2-[1-(piperidin-4-yl)cyclopropyl]-1,6-naphthyridin-7-yl]amino)phenyl]pyrazol-3-yl]methanol (60 mg, 0.131 mmol, 1 equiv.), HCHO (4.71 mg, 0.157 mmol, 1.20 equiv.) in THF (15 mL) was added NaBH(OAc)₃ (41.60 mg, 0.196 mmol, 1.50 equiv.). The resulting mixture was stirred for 1 hour at room temperature under nitrogen atmosphere. The reaction was monitored by LCMS. The crude product (30 mg) was purified by Prep-HPLC to afford [1-[3-fluoro-4-([2-[1-(1-methylpiperidin-4-yl)cyclopropyl]-1,6-naphthyridin-7-yl]amino)phenyl]pyrazol-3-yl]methanol (16 mg, 25%) as a white solid.

### Example 44. Preparation of Compounds 169 and 291

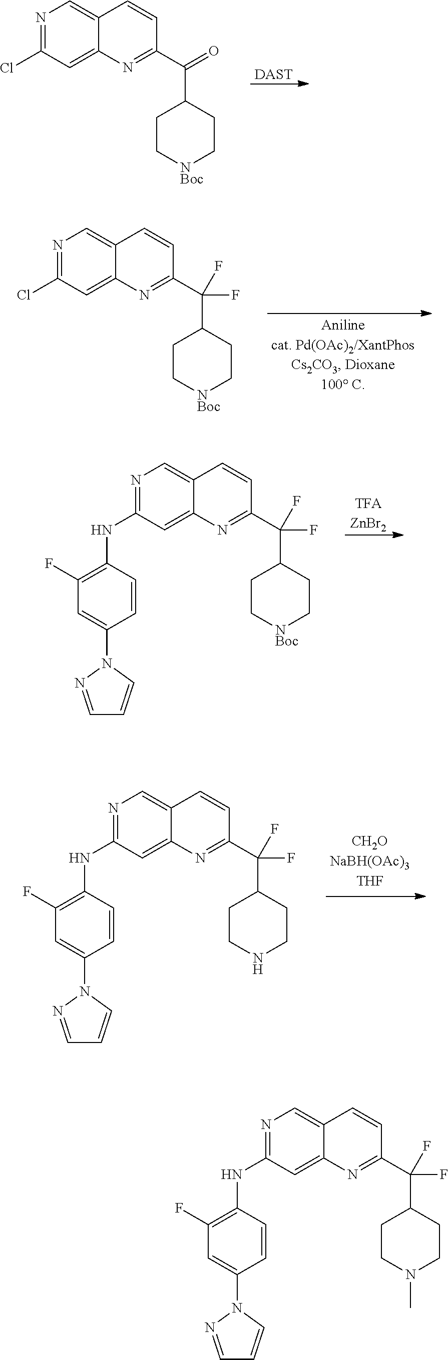

Tert-butyl 4-[(7-chloro-1,6-naphthyridin-2-yl)difluoromethyl]piperidine-1-carboxylate. A mixture of tert-butyl 4-(7-chloro-1,6-naphthyridine-2-carbonyl)piperidine-1-carboxylate (200 mg, 0.532 mmol, 1 equiv.) in DAST (5 mL) was stirred for 16 hours at room temperature under nitrogen atmosphere. The reaction was monitored by LCMS. The resulting mixture was added into 50 mL of cold saturated NaHCO₃ (aq.). The aqueous layer was extracted with EtOAc (2×100 mL). The combined organic layers were washed with brine (100 mL), dried over anhydrous Na₂SO₄. After filtration, the filtrate was concentrated under reduced pressure. The residue was purified by Prep-TLC (DCM/MeOH 50:1) to afford tert-butyl 4-[(7-chloro-1,6-naphthyridin-2-yl)difluoromethyl]piperidine-1-carboxylate (130 mg, 61%) as a yellow foam.

Tert-butyl 4-[difluoro(7-[[2-fluoro-4-(pyrazol-1-yl)phenyl]amino]-1,6-naphthyridin-2-yl)methyl]piperidine-1-carboxylate. To a stirred mixture of tert-butyl 4-[(7-chloro-1,6-naphthyridin-2-yl)difluoromethyl]piperidine-1-carboxylate (130 mg, 0.327 mmol, 1 equiv.) and 2-fluoro-4-(pyrazol-1-yl)aniline (63.68 mg, 0.359 mmol, 1.10 equiv.) in dry 1,4-dioxane (15 mL) were added Pd(OAc)₂ (11 mg, 0.049 mmol, 0.15 equiv.), Xantphos (56.72 mg, 0.098 mmol, 0.30 equiv.) and Cs₂CO₃ (212.93 mg, 0.654 mmol, 2 equiv.) at room temperature under nitrogen atmosphere. The resulting mixture was stirred for 2 hours at 100° C. under nitrogen atmosphere. The resulting mixture was filtered, the filter cake was washed with DCM and MeOH (10:1). The filtrate was concentrated under reduced pressure. The residue was purified by Prep-TLC (PE/EA, 2:1) to afford tert-butyl 4-[difluoro(7-[[2-fluoro-4-(pyrazol-1-yl)phenyl]amino]-1,6-naphthyridin-2-yl)methyl]piperidine-1-carboxylate (130 mg, 73%) as a yellow foam.

2-[difluoro(piperidin-4-yl)methyl]-N-[2-fluoro-4-(pyrazol-1-yl)phenyl]-1,6-naphthyridin-7-amine (Compound 169). To a stirred solution of tert-butyl 4-[difluoro(7-[[2-fluoro-4-(pyrazol-1-yl)phenyl]amino]-1,6-naphthyridin-2-yl)methyl]piperidine-1-carboxylate (130 mg, 0.241 mmol, 1 equiv.) in DCM (15 mL) was added zinc bromide (543.56 mg, 2.414 mmol, 10 equiv.) at room temperature. The resulting mixture was stirred for 16 hours at 50° C. The resulting mixture was diluted with water (100 mL) and extracted with DCM (3×50 mL). The combined organic layers were dried over anhydrous sodium sulfate and filtered. The filtrate was concentrated under reduced pressure, and the residue was purified by Prep-HPLC to afford 2-[difluoro(piperidin-4-yl)methyl]-N-[2-fluoro-4-(pyrazol-1-yl)phenyl]-1,6-naphthyridin-7-amine (33.9 mg, 32%) as a yellow solid.

2-[difluoro(1-methylpiperidin-4-yl)methyl]-N-[2-fluoro-4-(pyrazol-1-yl)phenyl]-1,6-naphthyridin-7-amine (Compound 291). To a stirred solution of 2-[difluoro(piperidin-4-yl)methyl]-N-[2-fluoro-4-(pyrazol-1-yl)phenyl]-1,6-naphthyridin-7-amine (30 mg, 0.068 mmol, 1 equiv.) and formaldehyde solution (6.16 mg, 0.205 mmol, 3 equiv.) in THF (10 mL) was added NaBH(OAc)₃ (43.50 mg, 0.205 mmol, 3 equiv.) in portions at 0° C. The resulting mixture was stirred for 1 hour at 0° C. The resulting mixture was diluted with aqueous saturated NaHCO₃ (20 mL) and extracted with ethyl acetate (2×50 mL). The combined organic layers were dried over anhydrous Na₂SO₄. After filtration, the filtrate was concentrated under reduced pressure. The crude product was purified by Prep-HPLC to afford 2-[difluoro(1-methylpiperidin-4-yl)methyl]-N-[2-fluoro-4-(pyrazol-1-yl)phenyl]-1,6-naphthyridin-7-amine (13.3 mg) as a yellow solid.

### Example 45. Preparation of Compounds 375 and 381

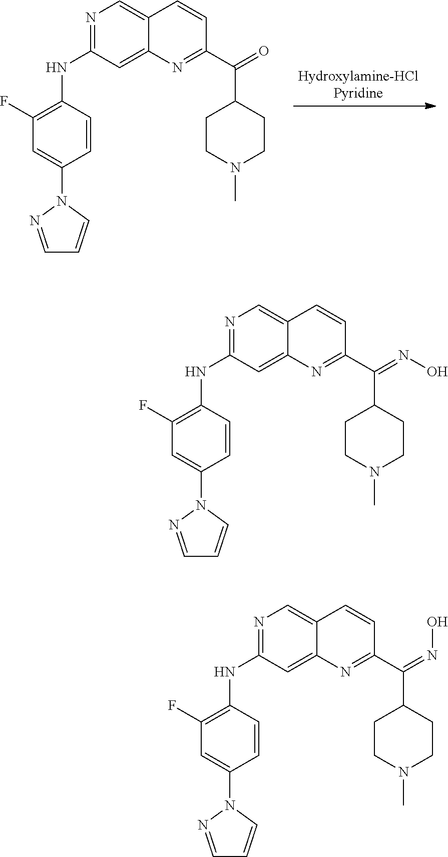 [SMILES: CN1CCC(C(=O)c2ccc3cnc(Nc4ccc(-n5cccn5)cc4F)cc3n2)CC1 ; CN1CCC(/C(=N\O)c2ccc3cnc(Nc4ccc(-n5cccn5)cc4F)cc3n2)CC1 ; CN1CCC(/C(=N/O)c2ccc3cnc(Nc4ccc(-n5cccn5)cc4F)cc3n2)CC1]

A mixture of N-[2-fluoro-4-(pyrazol-1-yl)phenyl]-2-(1-methylpiperidine-4-carbonyl)-1,6-naphthyridin-7-amine (33 mg, 0.077 mmol, 1 equiv.), hydroxylamine hydrochloride (13.32 mg, 0.192 mmol, 2.50 equiv.) and Pyridine (2 mL) stirring at 50° C. for 1 h. The crude product (33 mg) was purified by Prep-HPLC to afford N-[2-fluoro-4-(pyrazol-1-yl)phenyl]-2-[(1E)-hydroxyimino) (1-methylpiperidin-4-yl)methyl]-1,6-naphthyridin-7-amine (7.1 mg, 21%) and N-[2-fluoro-4-(pyrazol-1-yl)phenyl]-2-[(1Z)-hydroxyimino) (1-methylpiperidin-4-yl)methyl]-1,6-naphthyridin-7-amine (4.8 mg, 14%) as yellow solids.

Compounds 357 and 361 were synthesized following the methods and protocols as described for the synthesis of Compounds 375 and 381, starting with the appropriate materials.

### Example 46. Preparation of Compound 365

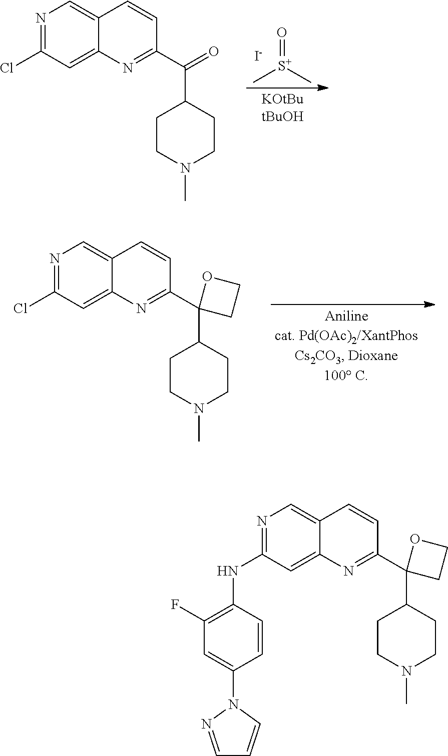

7-chloro-2-(2-(1-methylpiperidin-4-yl)oxetan-2-yl)-1,6-naphthyridine. To a stirred mixture of 7-chloro-2-(1-methylpiperidine-4-carbonyl)-1,6-naphthyridine (120. mg, 0.414 mmol, 1 equiv.) and t-BuOK (185.88 mg, 1.657 mmol, 4 equiv.) in t-BuOH (5 mL) was added trimethyl(oxo)-lambda6-sulfanylium iodide (364.56 mg, 1.657 mmol, 4 equiv.) at room temperature under nitrogen atmosphere. The resulting mixture was stirred for 16 hours at 50° C. under nitrogen atmosphere. The reaction was monitored by LCMS. The mixture was allowed to cool down to room temperature. The resulting mixture was concentrated under reduced pressure. The residue was purified by Prep-TLC (DCM/MeOH=15:1) to afford 7-chloro-2-[2-(1-methylpiperidin-4-yl)oxetan-2-yl]-1,6-naphthyridine (60 mg, crude) as a yellow solid.

N-(2-fluoro-4-(1H-pyrazol-1-yl)phenyl)-2-(2-(1-methylpiperidin-4-yl)oxetan-2-yl)-1,6-naphthyridin-7-amine (Compound 365). To a stirred mixture of 7-chloro-2-[2-(1-methylpiperidin-4-yl)oxetan-2-yl]-1,6-naphthyridine (60 mg, 0.189 mmol, 1 equiv.) and 2-fluoro-4-(pyrazol-1-yl)aniline (40.14 mg, 0.227 mmol, 1.20 equiv.) in 1,4-dioxane (4 mL) were added Cs₂CO₃ (123.02 mg, 0.378 mmol, 2 equiv.), XantPhos (32.77 mg, 0.057 mmol, 0.30 equiv.) and Pd(OAc)₂ (6.36 mg, 0.028 mmol, 0.15 equiv.) at room temperature under nitrogen atmosphere. The resulting mixture was stirred for 3 hours at 100° C. under nitrogen atmosphere. The reaction was monitored by LCMS. The mixture was allowed to cool down to room temperature. The resulting mixture was filtered, the filter cake was washed with EtOAc (3×10 mL). The filtrate was concentrated under reduced pressure. The residue was purified by Prep-TLC (DCM/MeOH=10:1) to afford crude product. The crude product (60 mg) was purified by Prep-HPLC to afford N-[2-fluoro-4-(1H-pyrazol-1-yl)phenyl]-2-[2-(1-methylpiperidin-4-yl)oxetan-2-yl]-1,6-naphthyridin-7-amine (7.6 mg, 8%) as a yellow solid.

### Example 47. Preparation of Compounds 343 and 349

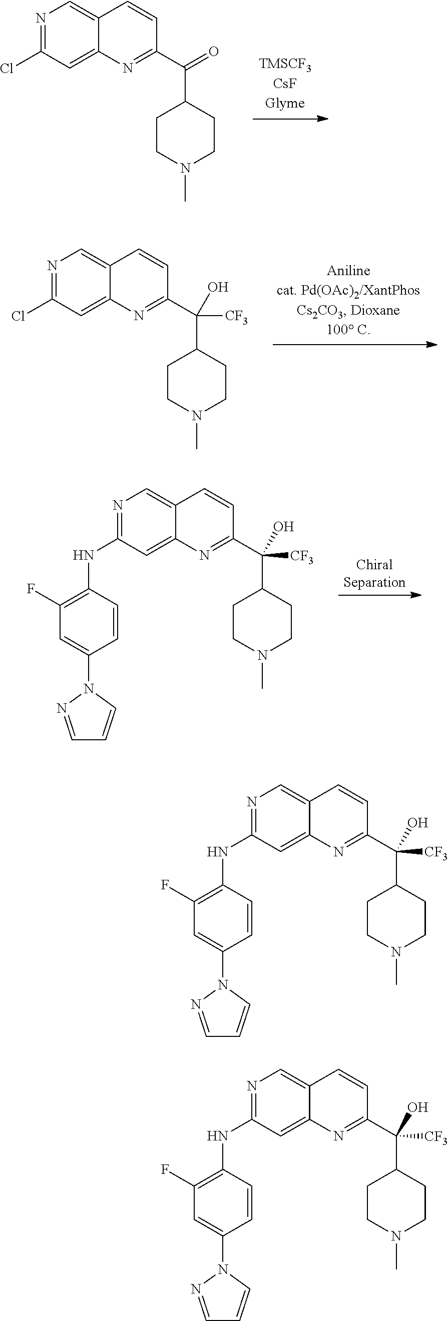

1-(7-chloro-1,6-naphthyridin-2-yl)-2,2,2-trifluoro-1-(1-methylpiperidin-4-yl)ethanol. To a stirred mixture of 7-chloro-2-(1-methylpiperidine-4-carbonyl)-1,6-naphthyridine (0.10 g, 0.35 mmol) and cesium fluoride (65.5 mg, 0.43 mmol) in ethylene glycol dimethyl ether (4.00 mL) was added (trifluoromethyl)trimethylsilane (0.12 g, 0.86 mmol) dropwise at 0° C. under nitrogen atmosphere. The resulting mixture was stirred at ambient temperature for 48 h under nitrogen atmosphere. The reaction was quenched with water (20.0 mL) and extracted with ethyl acetate (3×20.0 mL). The combined organic layers was washed with brine (3×10.0 mL), dried over anhydrous sodium sulfate and filtered. The filtrate was concentrated under reduced pressure to afford the title compound (0.13 g, crude) as a yellow solid which was used in the next step directly without further purification.

2,2,2-trifluoro-1-(7-[[2-fluoro-4-(pyrazol-1-yl)phenyl]amino]-1,6-naphthyridin-2-yl)-1-(1-methylpiperidin-4-yl)ethanol. In microwave vial (10 mL) was added 1-(7-chloro-1,6-naphthyridin-2-yl)-2,2,2-trifluoro-1-(1-methylpiperidin-4-yl)ethanol (124 mg, 0.345 mmol, 1 equiv.), 2-fluoro-4-(pyrazol-1-yl)aniline (67.17 mg, 0.379 mmol, 1.1 equiv.), XantPhos (59.83 mg, 0.103 mmol, 0.3 equiv.), Pd(OAc)₂ (11.61 mg, 0.052 mmol, 0.15 equiv.) and Cs₂CO₃ (224.59 mg, 0.689 mmol, 2 equiv.) in dioxane (5 mL), the resulting mixture was stirred at 100° C. for 2 hours via sealed tube. The mixture was allowed to cool down to room temperature. The reaction was quenched with water at room temperature. The resulting mixture was extracted with DCM (100×mL). The combined organic layers were washed with brine (3×50 mL), dried over anhydrous Na₂SO₄. After filtration, the filtrate was concentrated under reduced pressure. The residue was purified by reverse flash chromatography with the following conditions: column, C₁₈ silica gel; mobile phase, MeOH in water, 10% to 50% gradient in 10 min; detector, UV 254 nm to afford 2,2,2-trifluoro-1-(7-[[2-fluoro-4-(pyrazol-1-yl)phenyl]amino]-1,6-naphthyridin-2-yl)-1-(1-methylpiperidin-4-yl)ethanol (100 mg, 57%) as a yellow solid.

(R)-2,2,2-trifluoro-1-(7-((2-fluoro-4-(1H-pyrazol-1-yl)phenyl)amino)-1,6-naphthyridin-2-yl)-1-(1-methylpiperidin-4-yl)ethan-1-ol and (S)-2,2,2-trifluoro-1-(7-((2-fluoro-4-(1H-pyrazol-1-yl)phenyl)amino)-1,6-naphthyridin-2-yl)-1-(1-methylpiperidin-4-yl)ethan-1-ol (Compounds 343 and 349). 2,2,2-Trifluoro-1-(7-[[2-fluoro-4-(pyrazol-1-yl)phenyl]amino]-1,6-naphthyridin-2-yl)-1-(1-methylpiperidin-4-yl)ethanol (0.10 g) was separated by Chiral-Prep-HPLC with the following conditions: Column: CHIRALPAK IG, 2×25 cm, 5 um; Mobile Phase A: Hex:Dichloromethane=3:1 (plus 10 mM NH₃ in MeOH), Mobile Phase B: EtOH; Flow rate: 20 mL/min; Gradient: isocratic 25% B in 13 min; Detector: UV 254/220 nm.

Compound 343-eluted at 10.83 min; yield: 30.4 mg. Compound 349-eluted at 7.84 min; yield: 32.5 mg Compounds 390, 394, 396, 398, 401, and 403 were synthesized following the methods and protocols as described for the synthesis of Compounds 343 and 349, starting with the appropriate materials.

### Example 48. Preparation of Compound 215

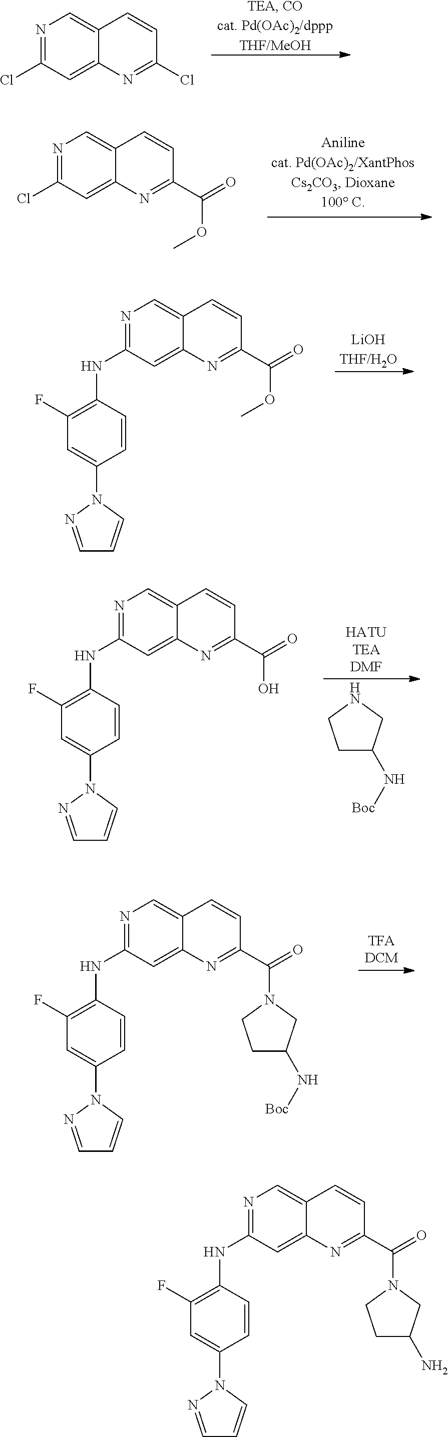

Methyl 7-chloro-1,6-naphthyridine-2-carboxylate. To a stirred mixture of 2,7-dichloro-1,6-naphthyridine (1700 mg, 8.54 mmol, 1 equiv.) and TEA (2.59 g, 25.6 mmol, 3 equiv.) in THF (40 mL) and MeOH (10 mL) were added Pd(OAc)₂ (383.5 mg, 1.708 mmol, 0.20 equiv.) and DPPP (2113.7 mg, 5.125 mmol, 0.60 equiv.) in portions at room temperature under nitrogen atmosphere. The resulting mixture was stirred for overnight at 60° C. under CO atmosphere. The resulting mixture was concentrated under reduced pressure. The residue was purified by silica gel column chromatography, eluting with PE/EtOAc (1:1) to afford methyl 7-chloro-1,6-naphthyridine-2-carboxylate (700 mg, 37%) as an off-white solid.

Methyl 7-[[2-fluoro-4-(pyrazol-1-yl)phenyl]amino]-1,6-naphthyridine-2-carboxylate. To a stirred mixture of methyl 7-chloro-1,6-naphthyridine-2-carboxylate (700 mg, 3.144 mmol, 1 equiv.), 2-fluoro-4-(pyrazol-1-yl)aniline (668.52 mg, 3.773 mmol, 1.20 equiv.) and Cs₂CO₃ (2048.90 mg, 6.288 mmol, 2 equiv.) in dioxane (40 mL) were added Pd(OAc)₂ (141.18 mg, 0.629 mmol, 0.2 equiv.) and XantPhos (1091.59 mg, 1.887 mmol, 0.6 equiv.) in portions at room temperature under nitrogen atmosphere. The resulting mixture was stirred for 1 hour at 100° C. under nitrogen atmosphere. The resulting mixture was concentrated under vacuum. The residue was purified by silica gel column chromatography, eluted with PE/EtOAc (1:1) to afford methyl 7-[[2-fluoro-4-(pyrazol-1-yl)phenyl]amino]-1,6-naphthyridine-2-carboxylate (450 mg, 39%) as a yellow solid.

7-[[2-fluoro-4-(pyrazol-1-yl)phenyl]amino]-1,6-naphthyridine-2-carboxylic acid. To a stirred solution of methyl 7-[[2-fluoro-4-(pyrazol-1-yl)phenyl]amino]-1,6-naphthyridine-2-carboxylate (450 mg, 1.238 mmol, 1 equiv.) in THF (20 mL, 246.860 mmol, 199.33 equiv.) and H₂O (5 mL, 277.542 mmol, 224.10 equiv.) was added LiOH (148.29 mg, 6.192 mmol, 5 equiv.) in portions at room temperature. The resulting mixture was stirred for 16 hours at 60° C. The resulting mixture was concentrated under vacuum. The residue was purified by Prep-TLC (DCM/MeOH 10:1) to afford 7-[[2-fluoro-4-(pyrazol-1-yl)phenyl]amino]-1,6-naphthyridine-2-carboxylic acid (270 mg, 62%) as a red solid.

Tert-butyl N-[1-(7-[[2-fluoro-4-(pyrazol-1-yl)phenyl]amino]-1,6-naphthyridine-2-carbonyl)pyrrolidin-3-yl]carbamate. To a stirred solution of 7-[[2-fluoro-4-(pyrazol-1-yl)phenyl]amino]-1,6-naphthyridine-2-carboxylic acid (270 mg, 0.773 mmol, 1 equiv.) and tert-butyl N-pyrrolidin-3-yl)carbamate (431.88 mg, 2.319 mmol, 3 equiv.) DMF (4 mL) were added HATU (382.05 mg, 15 mmol, 1.3 equiv.) and TEA (234.64 mg, 2.319 mmol, 3 equiv.) dropwise at room temperature. The resulting mixture was stirred for 2.5 hours at room temperature. The resulting mixture was concentrated under vacuum. The residue was purified by Prep-TLC (EtOAc) to afford tert-butyl N-[1-(7-[[2-fluoro-4-(pyrazol-1-yl)phenyl]amino]-1,6-naphthyridine-2-carbonyl)pyrrolidin-3-yl]carbamate (150 mg, 37%) as a yellow solid. 2-(3-aminopyrrolidine-1-carbonyl)-N-[2-fluoro-4-(pyrazol-1-yl)phenyl]-1,6-naphthyridin-7-amine (Compound 215). To a stirred solution of tert-butyl N-[1-(7-[[2-fluoro-4-(pyrazol-1-yl)phenyl]amino]-1,6-naphthyridine-2-carbonyl)pyrrolidin-3-yl]carbamate (80.0 mg, 0.155 mmol, 1 equiv.) in DCM (10 mL) was added TFA (1 mL) dropwise at room temperature. The resulting mixture was concentrated under vacuum. The residue was purified by reverse flash chromatography with the following conditions: Column: Spherical C18, 20˜040 um, 120 g; Mobile Phase A:Water (0.05% TFA), Mobile Phase B: ACN; Flow rate: 45 mL/min; Gradient (B %): 5%-25%, 10 min; 25%-37%, 17 min; 37%-95%; 2 min; 95%, 5 min; Detector: 254 nm; Rt: 42 min. The fractions containing desired product were collected at 37% B and concentrated under reduced pressure to afford 2-(3-aminopyrrolidine-1-carbonyl)-N-[2-fluoro-4-(pyrazol-1-yl)phenyl]-1,6-naphthyridin-7-amine; formic acid (51.9 mg, 73%) as a yellow solid.

### Example 49. Preparation of Compound 242

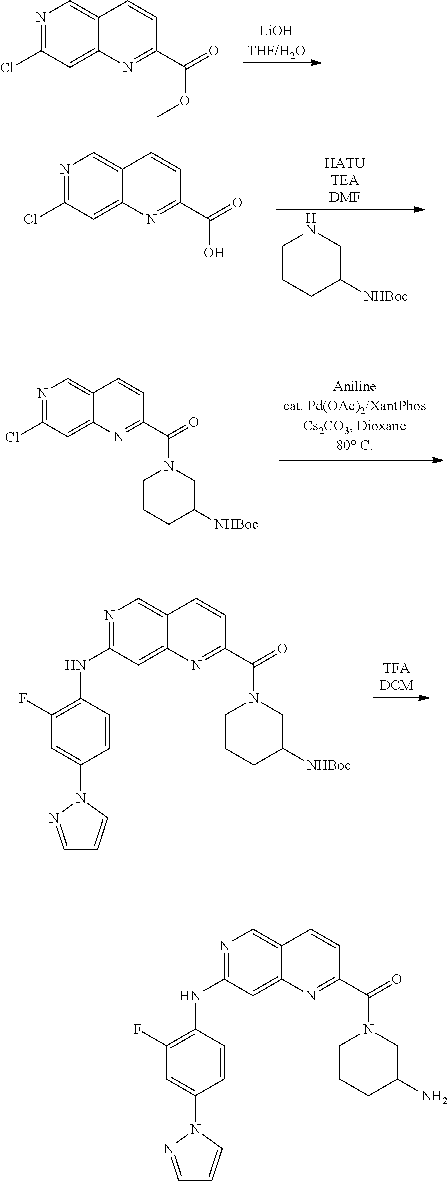

7-chloro-1,6-naphthyridine-2-carboxylic acid. To a stirred mixture of methyl 7-chloro-1,6-naphthyridine-2-carboxylate (Compound 215, step 1, 400 mg, 1.797 mmol, 1 equiv.) in THF (40 mL) and H₂O (10 mL) was added LiOH (172.11 mg, 7.187 mmol, 4 equiv.) in portions at room temperature. The resulting mixture was stirred for 3 hours at 50° C. The mixture was neutralized to pH 7 with HCl (aq.). The resulting mixture was concentrated under vacuum. The residue was purified by silica gel column chromatography, eluted with hexane/EtOAc (1:1) to afford 7-chloro-1,6-naphthyridine-2-carboxylic acid (200 mg, 43%) as an off-white solid.

Tert-butyl N-[1-(7-chloro-1,6-naphthyridine-2-carbonyl)piperidin-3-yl]carbamate. To a stirred mixture of 7-chloro-1,6-naphthyridine-2-carboxylic acid (250 mg, 1.198 mmol, 1 equiv.) and tert-butyl N-piperidin-3-yl)carbamate (480.06 mg, 2.397 mmol, 2 equiv.) in DMF (12 mL) were added TEA (363.82 mg, 3.595 mmol, 3 equiv.) and HATU (683.54 mg, 1.798 mmol, 1.5 equiv.) in portions at room temperature. The resulting mixture was stirred for 3 hours at room temperature. The resulting mixture was concentrated under vacuum. The residue was purified by silica gel column chromatography, eluted with EtOAc to afford tert-butyl N-[1-(7-chloro-1,6-naphthyridine-2-carbonyl)piperidin-3-yl]carbamate (120 mg, 25%) as a yellow solid.

Tert-butyl N-[1-(7-[[2-fluoro-4-(pyrazol-1-yl)phenyl]amino]-1,6-naphthyridine-2-carbonyl)piperidin-3-yl]carbamate. To a stirred mixture of tert-butyl N-[1-(7-chloro-1,6-naphthyridine-2-carbonyl)piperidin-3-yl]carbamate (200 mg, 0.512 mmol, 1 equiv.), 2-fluoro-4-pyrazol-1-yl)aniline (108.79 mg, 0.614 mmol, 1.20 equiv.) and Pd(OAc)₂ (22.98 mg, 0.102 mmol, 0.2 equiv.) in dioxane (10 mL) were added XantPhos (177.64 mg, 0.307 mmol, 0.6 equiv.) and Cs₂CO₃ (266.74 mg, 0.819 mmol, 1.6 equiv.) in portions at room temperature under nitrogen atmosphere. The resulting mixture was stirred for 1.5 hours at 80° C. under nitrogen atmosphere. The resulting mixture was concentrated under vacuum. The residue was purified by Prep-TLC (PE/EtOAc 1:1) to afford tert-butyl N-[1-(7-[[2-fluoro-4-(pyrazol-1-yl)phenyl]amino]-1,6-naphthyridine-2-carbonyl)piperidin-3-yl]carbamate (120 mg, 35%) as a yellow solid.

2-(3-aminopiperidine-1-carbonyl)-N-[2-fluoro-4-(pyrazol-1-yl)phenyl]-1,6-naphthyridin-7-amine (Compound 242). To a stirred solution of tert-butyl N-[1-(7-[[2-fluoro-4-(pyrazol-1-yl)phenyl]amino]-1,6-naphthyridine-2-carbonyl)piperidin-3-yl]carbamate (60 mg, 0.113 mmol, 1 equiv.) in DCM (10 mL) was added TFA (1 mL) dropwise at room temperature. The resulting mixture was concentrated under vacuum. The residue was basified to pH 8 with saturated NaHCO₃ (aq.). The resulting mixture was concentrated under vacuum. The residue was purified by reverse flash chromatography with the following conditions: Column: Spherical C18, 20˜40 um, 120 g; Mobile Phase A: Water (10 mM NH₄HCO₃ and 0.05% NH₃ H₂O), Mobile Phase B: ACN; Flow rate: 45 mL/min; Gradient (B %): 5%-25%, 13 min; 25%-37%, 17 min; 37° 4-95%; 2 min; 95%, 5 min; Detector: 254 nm; Rt: 30 min. The fractions containing desired product were collected at 37% B and concentrated under reduced pressure to afford 2-(3-aminopiperidine-1-carbonyl)-N-[2-fluoro-4-(pyrazol-1-yl)phenyl]-1,6-naphthyridin-7-amine (72.1 mg, 92%) as a yellow solid.

### Example 50. Preparation of Compound 207

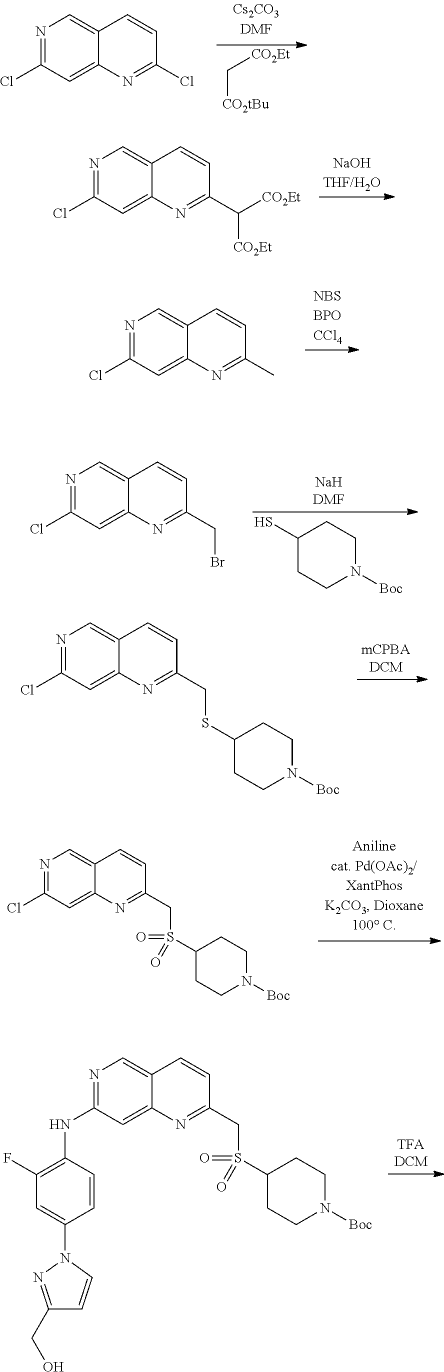

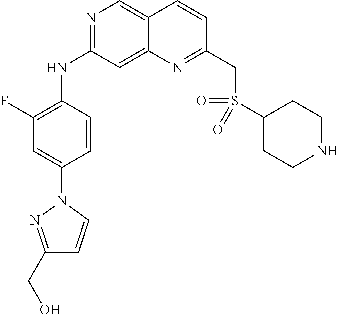 [SMILES: O=S(=O)(Cc1ccc2cnc(Nc3ccc(-n4ccc(CO)n4)cc3F)cc2n1)C1CCNCC1]

1,3-diethyl 2-(7-chloro-1,6-naphthyridin-2-yl)propanedioate. To a stirred mixture of 2,7-dichloro-1,6-naphthyridine (1200 mg, 1 equiv.) and diethyl malonate (1940 mg, 2 equiv.) in DMF were added Cs₂CO₃ (4000 mg, 2 equiv.) dropwise at room temperature under nitrogen atmosphere. The resulting mixture was stirred for 4 hours at 80° C. under nitrogen atmosphere. The resulting oil was dried in an oven under reduced pressure. The residue was purified by reverse phase flash with the following conditions (Column: C18, 330 g; Mobile Phase A:Water/0.05% TFA, Mobile Phase B:ACN; Flow rate:80 mL/min; Gradient: 20% B to 60% B in 40 min; Detector, 254 nm, Monitor, 220 nm, the desired product were collected at 58% B). This resulted in 1,3-diethyl 2-(7-chloro-1,6-naphthyridin-2-yl)propanedioate (1400 mg) as a white solid.

7-chloro-2-methyl-1,6-naphthyridine. To a stirred solution of 1,3-diethyl 2-(7-chloro-1,6-naphthyridin-2-yl)propanedioate (1.60 g, 4.97 mmol) in THF (45.0 mL) and water (10.0 mL) was added sodium hydroxide (1.00 g, 25.0 mmol) at 0° C. under nitrogen atmosphere. The reaction was monitored by LCMS. The residue was purified by Prep-TLC (PE/EtOAc 1:1) to afford 7-chloro-2-methyl-1,6-naphthyridine (650 mg) as a yellow solid.

2-(bromomethyl)-7-chloro-1,6-naphthyridine. To a stirred solution of 7-chloro-2-methyl-1,6-naphthyridine (550 mg, 1 equiv.) in CCl₄ (17 mL) was added NBS (1140 mg, 2 equiv.) and BPO (155 mg, 0.20 equiv.) in portions at 80° C. under nitrogen atmosphere. The reaction was monitored by LCMS. The residue was purified by Prep-TLC (PE/EtOAc 1:1) to afford 2-(bromomethyl)-7-chloro-1,6-naphthyridine (150 mg) as a white solid.

Tert-butyl 4-[[(7-chloro-1,6-naphthyridin-2-yl)methyl]sulfanyl]piperidine-1-carboxylate. To a stirred mixture of 2-(bromomethyl)-7-chloro-1,6-naphthyridine (150 mg, 1 equiv.) and tert-butyl 4-sulfanylpiperidine-1-carboxylate (153 mg, 1.20 equiv.) in DMF were added NaH (21 mg, 1.50 equiv.) dropwise at room temperature under nitrogen atmosphere. The resulting mixture was stirred for 1 hour at room temperature under nitrogen atmosphere. The resulting oil was dried in an oven under reduced pressure. The residue was purified by reverse phase flash with the following conditions (Column: C18, 330 g; Mobile Phase A:Water/0.05% TFA, Mobile Phase B:ACN; Flow rate:80 mL/min; Gradient: 20% B to 60% B in 40 min; Detector, 254 nm, Monitor, 220 nm, the desired product were collected at 58% B). This resulted in tert-butyl 4-[[(7-chloro-1,6-naphthyridin-2-yl)methyl]sulfanyl]piperidine-1-carboxylate (128 mg) as a white solid.

Tert-butyl 4-[(7-chloro-1,6-naphthyridin-2-yl)methanesulfonyl]piperidine-1-carboxylate. To a stirred solution of tert-butyl 4-[[(7-chloro-1,6-naphthyridin-2-yl)methyl]sulfanyl]piperidine-1-carboxylate (128 mg, 1 equiv.) in DCM (5 mL) was added m-CPBA (168 mg, 3 equiv.) in portions at 0° C. under nitrogen atmosphere. The reaction was monitored by LCMS. The reaction was monitored by LCMS. The resulting mixture was concentrated under reduced pressure. The residue was purified by reverse phase flash with the following conditions (Column: C18, 330 g; Mobile Phase A:Water/0.05% NH₄HCO₃, Mobile Phase B:ACN; Flow rate:80 mL/min; Gradient: 20% B to 50% B in 20 min; Detector, 254 nm, Monitor, 220 nm, the desired product were collected at 45% B) to afford tert-butyl 4-[(7-chloro-1,6-naphthyridin-2-yl)methanesulfonyl]piperidine-1-carboxylate (133 mg) as a white solid.

Tert-butyl 4-[[7-([2-fluoro-4-[3-(hydroxymethyl)pyrazol-1-yl]phenyl]amino)-1,6-naphthyridin-2-yl]methanesulfonyl]piperidine-1-carboxylate. To a stirred mixture of tert-butyl 4-[(7-chloro-1,6-naphthyridin-2-yl)methanesulfonyl]piperidine-1-carboxylate (100 mg, 1 equiv.) and [1-(4-amino-3-fluorophenyl)pyrazol-3-yl]methanol (64 mg, 1.30 equiv.) in 1,4-dioxane (3 mL) were added Pd(OAc)₂ (8 mg, 0.15 equiv.), XantPhos (41 mg, 0.30 equiv.) and K₂CO₃ (65 mg, 2 equiv.) at room temperature under nitrogen atmosphere. The resulting mixture was stirred for 2 hours at 100° C. under nitrogen atmosphere. The reaction was monitored by LCMS. The mixture was allowed to cool down to room temperature. The resulting mixture was filtered, the filter cake was washed with EtOAc (3×10 mL). The filtrate was concentrated under reduced pressure. The residue was purified by reverse phase flash with the following conditions (Column: C18, 330 g; Mobile Phase A:Water/0.05% NH₄HCO₃, Mobile Phase B:ACN; Flow rate:80 mL/min; Gradient: 20% B to 60% B in 30 min; Detector, 254 nm, Monitor, 220 nm, the desired product were collected at 58% B) to afford tert-butyl 4-[[7-([2-fluoro-4-[3-(hydroxymethyl)pyrazol-1-yl]phenyl]amino)-1,6-naphthyridin-2-yl]methanesulfonyl]piperidine-1-carboxylate (62 mg) as a brown yellow solid.

[1-[3-fluoro-4-([2-[(piperidine-4-sulfonyl)methyl]-1,6-naphthyridin-7-yl]amino)phenyl]pyrazol-3-yl]methanol (Compound 207). To a stirred solution of tert-butyl 4-[[7-([2-fluoro-4-[3-(hydroxymethyl)pyrazol-1-yl]phenyl]amino)-1,6-naphthyridin-2-yl]methanesulfonyl]piperidine-1-carboxylate (70 mg, 1 equiv.) in DCM (10 mL) was added TFA (1 mL) at 0° C. The resulting mixture was stirred for 1 hour at 0° C. The reaction was monitored by LCMS. The mixture was basified to pH 8 with saturated NaHCO₃ (aq.). The resulting solid was dried in an oven under reduced pressure to afford [1-[3-fluoro-4-([2-[(piperidine-4-sulfonyl)methyl]-1,6-naphthyridin-7-yl]amino)phenyl]pyrazol-3-yl]methanol (18 mg) as a yellow solid.

### Example 51. Preparation of Compound 208

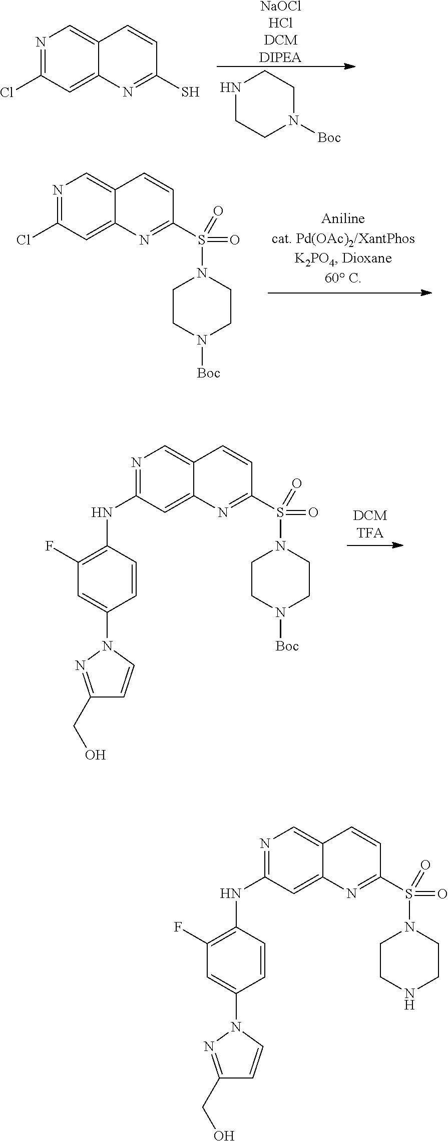

Tert-butyl 4-(7-chloro-1,6-naphthyridin-2-ylsulfonyl)piperazine-1-carboxylate. To a mixture of 2M HCl (7.5 mL) and DCM (20 mL) cooled to −5° C. was added a NaOCl (˜10% solution, 6.50 mL, 10.075 mmol, 3.35 equiv.) at −5° C., then the reaction mixture was stirred at −5° C. for 30 min. To the above reaction mixture was added 7-chloro-1,6-naphthyridine-2-thiol (591 mg, 35 mmol, 1 equiv.) and this resulting mixture was stirred at −5° C. for 60 min. Excess chlorine was quenched by adding 1 M Na₂SO₃ until the yellow greenish color of the mixture disappeared. The reaction mixture was then transferred to a separating funnel and the organic layer was rapidly separated and collected in a flask in ice water. The aqueous phase was quickly extracted with DCM (2×50 mL). The organic extracts were combined and dried over Na₂SO₄. The mixture was filtered into a cold (−30° C.) stirred solution of tert-butyl piperazine-1-carboxylate (671.71 mg, 3.606 mmol, 1.20 equiv.) and DIPEA (1.17 g, 9.016 mmol, 3 equiv.) in DCM (20 mL). The reaction mixture was stirred for another 1 hour cooled in a salt-ice bath. The reaction was quenched by the addition of H₂O (100 mL) and the resulting mixture was extracted with DCM (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with PE:EtOAc (2:1) to afford tert-butyl 4-(7-chloro-1,6-naphthyridin-2-ylsulfonyl)piperazine-1-carboxylate (420 mg, 33%) as a white solid.

Tert-butyl 4-[7-([2-fluoro-4-[3-(hydroxymethyl)pyrazol-1-yl]phenyl]amino)-1,6-naphthyridin-2-ylsulfonyl]piperazine-1-carboxylate. In a 20 mL microwave vial was charged with [1-(4-amino-3-fluorophenyl)pyrazol-3-yl]methanol (175.65 mg, 0.848 mmol, 1 equiv.), tert-butyl 4-(7-chloro-1,6-naphthyridin-2-ylsulfonyl)piperazine-1-carboxylate (350 mg, 0.848 mmol, 1 equiv.), dioxane (15 mL, 177.061 mmol, 208.88 equiv.), Pd(OAc)₂ (28.55 mg, 0.127 mmol, 0.15 equiv.), XantPhos (147.15 mg, 0.254 mmol, 0.30 equiv.) and K₃PO₄ (539.80 mg, 2.543 mmol, 3 equiv.), then the resulting mixture was stirred at 60° C. for 2 hours under N₂ atmosphere. The reaction mixture was cooled down to room temperature and added DCM (200 mL) and H₂O (100 mL). The organic layer was washed with brine (2×50 mL) and concentrated under reduced pressure. The residue was purified by reverse flash chromatography with the following conditions: column, C₁₈ silica gel; mobile phase, MeCN in water (10 mmol/L NH₄HCO₃), 40% to 55% gradient in 15 min; detector, UV 220 nm to afford tert-butyl 4-[7-([2-fluoro-4-[3-(hydroxymethyl)pyrazol-1-yl]phenyl]amino)-1,6-naphthyridin-2-ylsulfonyl]piperazine-1-carboxylate (75 mg, 15%) as a light yellow solid.

[1-(3-fluoro-4-[[2-(piperazine-1-sulfonyl)-1,6-naphthyridin-7-yl]amino]phenyl)pyrazol-3-yl]methanol (Compound 208). A mixture of tert-butyl 4-[7-([2-fluoro-4-[3-(hydroxymethyl)pyrazol-1-yl]phenyl]amino)-1,6-naphthyridin-2-ylsulfonyl]piperazine-1-carboxylate (75 mg, 0.129 mmol, 1 equiv.), DCM (25 mL, 393.251 mmol, 3060.23 equiv.) and TFA (2.50 mL, 33.658 mmol, 261.92 equiv.) was stirred at 0° C. for 2 hours. The reaction was quenched by the addition of sat. NaHCO₃ (aq. 20 mL) at 0° C. The resulting mixture was extracted with DCM (3×100 mL). The combined organic layers were washed with brine (2×50 mL) and concentrated under reduced pressure. The crude product was purified by Prep-HPLC to afford [1-(3-fluoro-4-[[2-(piperazine-1-sulfonyl)-1,6-naphthyridin-7-yl]amino]phenyl)pyrazol-3-yl]methanol (28.4 mg, 45%) as a yellow solid.

Compound 248 was synthesized following the methods and protocols as described for the synthesis of Compound 208, starting with the appropriate materials.

### Example 52. Preparation of Compound 245

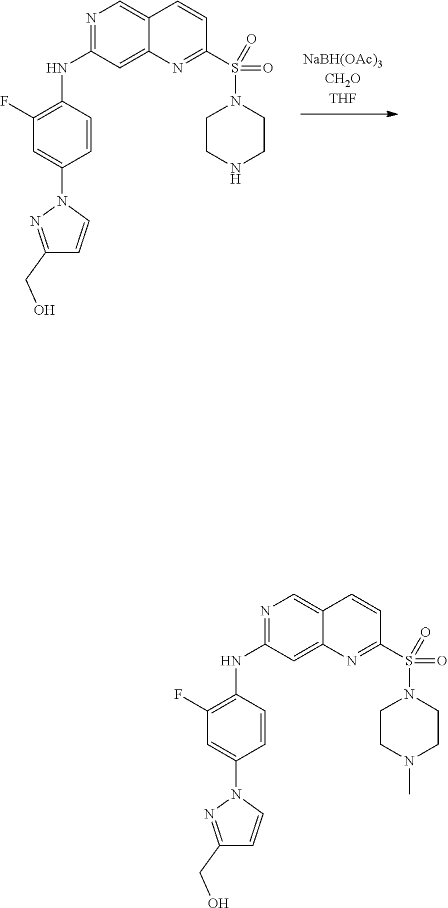 [SMILES: O=S(=O)(c1ccc2cnc(Nc3ccc(-n4ccc(CO)n4)cc3F)cc2n1)N1CCNCC1 ; CN1CCN(S(=O)(=O)c2ccc3cnc(Nc4ccc(-n5ccc(CO)n5)cc4F)cc3n2)CC1]

To a stirred solution of [1-(3-fluoro-4-[[2-(piperazine-1-sulfonyl)-1,6-naphthyridin-7-yl]amino]phenyl)pyrazol-3-yl]methanol (85 mg, 0.176 mmol, 1 equiv.) and HCHO (10.56 mg, 0.352 mmol, 2 equiv.) in THF (10 mL) ware added NaBH(OAc)₃ (74.52 mg, 1.186 mmol, 6.75 equiv.) in portions at 0° C. The resulting mixture was stirred for 1 hour at 0° C. The reaction was monitored by LCMS. The resulting mixture was concentrated under reduced pressure. The residue was purified by reverse phase flash with the following conditions (Column: XBridge Shield RP18 OBD Column, 5 μm, 19*150 mm). Concentrated under reduced pressure to afford [1-(3-fluoro-4-[[2-(4-methylpiperazin-1-ylsulfonyl)-1,6-naphthyridin-7-yl]amino]phenyl)pyrazol-3-yl]methanol (28.7 mg, 32%) as an orange solid.

### Example 53. Preparation of Compound 203

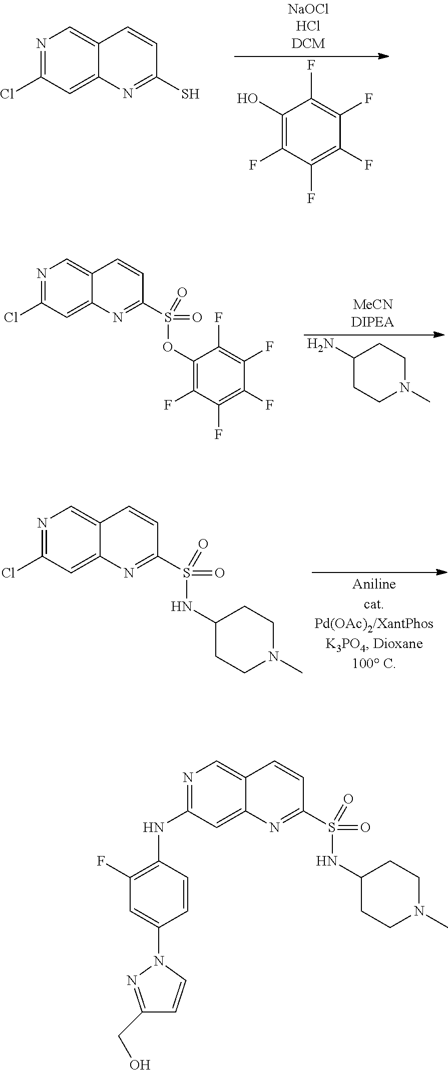

2,3,4,5,6-pentafluorophenyl 7-chloro-1,6-naphthyridine-2-sulfonate. To a mixture of 2M HCl (7.5 mL) and DCM (20 mL) cooled to −5° C. was added a NaOCl (˜10% solution, 6.50 mL, 10.075 mmol, 3.35 equiv.) at −5° C., then the reaction mixture was stirred at −5° C. for 30 min. To the above reaction mixture was added 7-chloro-1,6-naphthyridine-2-thiol (591 mg, 35 mmol, 1 equiv.) and this resulting mixture was stirred at −5° C. for 60 min. Excess chlorine was quenched by adding 1 M Na₂SO₃ until the yellow greenish color of the mixture disappeared. The reaction mixture was then transferred to a separating funnel and the organic layer was rapidly separated and collected in a flask in ice water. The aqueous phase was quickly extracted with DCM (2×50 mL). The organic extracts were combined and dried over Na₂SO₄. The filtrate was added into a pre-cold stirred solution of pentafluorophenol (0.184 g, 1.01 mmol) and triethylamine (0.15 g, 1.50 mmol) in dichloromethane (20.0 mL) at −30° C. The resulting reaction mixture was stirred at −30° C. for additional 1 h. The reaction mixture was washed with water (60.0 mL), aq. 10% KH₂PO₄ (2×30.0 mL), saturated NaHCO₃ (2×30.0 mL), water (30.0 mL) and brine (30.0 mL) respectively. The organic fraction was dried over anhydrous sodium sulfate and filtered. The filtrate was concentrated under reduced pressure to afford the title compound (0.41 g, crude) as a light yellow solid which was used in the next step directly without further purification.

7-chloro-N-(1-methylpiperidin-4-yl)-1,6-naphthyridine-2-sulfonamide. A mixture of crude 2,3,4,5,6-pentafluorophenyl 7-chloro-1,6-naphthyridine-2-sulfonate (610 mg, 1.485 mmol, 1 equiv.), 1-methylpiperidin-4-amine (186.57 mg, 1.634 mmol, 1.10 equiv.), DIPEA (575.88 mg, 4.456 mmol, 3 equiv.) and MeCN (20.09 mL, 489.397 mmol, 257.34 equiv.) was stirred at room temperature for 1 hour. The reaction mixture was concentrated to give the residue, which was purified by reverse flash chromatography with the following conditions: column, C₁₈ silica gel; mobile phase, MeCN in water (10 mmol/L NH₄HCO₃), 20% to 30% gradient in 10 min; detector, UV 220 nm to afford 7-chloro-N-(1-methylpiperidin-4-yl)-1,6-naphthyridine-2-sulfonamide (250 mg, 49%) as a light yellow solid.

7-([2-fluoro-4-[3-(hydroxymethyl)pyrazol-1-yl]phenyl]amino)-N-(1-methylpiperidin-4-yl)-1,6-naphthyridine-2-sulfonamide (Compound 203). In a 20 mL microwave vial was charged with 7-chloro-N-(1-methylpiperidin-4-yl)-1,6-naphthyridine-2-sulfonamide (250 mg, 0.734 mmol, 1 equiv.), [1-(4-amino-3-fluorophenyl)pyrazol-3-yl]methanol (151.99 mg, 0.734 mmol, 1 equiv.), dioxane (15 mL, 177.061 mmol, 241.39 equiv.), Pd(OAc)₂ (24.70 mg, 0.110 mmol, 0.15 equiv.), XantPhos (127.33 mg, 0.220 mmol, 0.30 equiv.) and K₃PO₄ (467.09 mg, 2.201 mmol, 3 equiv.), then the resulting mixture was stirred at 100° C. for 2 hours under N₂ atmosphere. The reaction mixture was cooled down to room temperature and added EtOAc (150 mL) and H₂O (100 mL). The resulting mixture was extracted with EtOAc (3×50 mL). The combined organic layers were washed with brine (2×50 mL) and concentrated under reduced pressure to give the residue, which was purified by Prep-HPLC to afford 7-([2-fluoro-4-[3-(hydroxymethyl)pyrazol-1-yl]phenyl]amino)-N-(1-methylpiperidin-4-yl)-1,6-naphthyridine-2-sulfonamide (13.7 mg, 3%) as a yellow solid.

### Example 54. Preparation of Compound 107

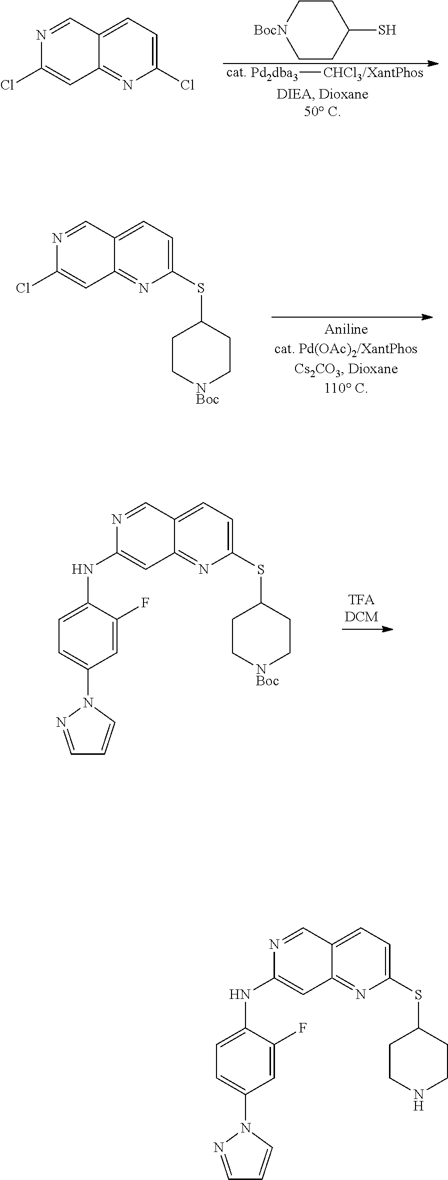

Tert-butyl 4-[(7-chloro-1,6-naphthyridin-2-yl)sulfanyl]piperidine-1-carboxylate. To a stirred mixture of 2,7-dichloro-1,6-naphthyridine (1 g, 5.024 mmol, 1 equiv.) and tert-butyl 4-sulfanylpiperidine-1-carboxylate (1.31 g, 6.029 mmol, 1.20 equiv.) in 1,4-dioxane (20 mL) were added DIEA (1.30 g, 10.049 mmol, 2 equiv.), XantPhos (581.44 mg, 15 mmol, 0.20 equiv.) and Pd₂(dba)₃CHCl₃ (520.07 mg, 0.502 mmol, 0.10 equiv.) at room temperature under nitrogen atmosphere. The resulting mixture was stirred for 3 hours at 50° C. under nitrogen atmosphere. The reaction was monitored by LCMS. The mixture was allowed to cool down to room temperature. The residue was purified by silica gel column chromatography, eluted with PE/EtOAc (20:1 to 10:1) to afford tert-butyl 4-[(7-chloro-1,6-naphthyridin-2-yl)sulfanyl]piperidine-1-carboxylate (1.8 g, 94%) as an off-white solid.

Tert-butyl 4-[(7-[[2-fluoro-4-(pyrazol-1-yl)phenyl]amino]-1,6-naphthyridin-2-yl)sulfanyl]piperidine-1-carboxylate. To a stirred mixture of tert-butyl 4-[(7-chloro-1,6-naphthyridin-2-yl)sulfanyl]piperidine-1-carboxylate (100 mg, 0.263 mmol, 1 equiv.) and 2-fluoro-4-(pyrazol-1-yl)aniline (51.30 mg, 0.290 mmol, 1.1 equiv.) in 1,4-dioxane (8 mL) were added XantPhos (30.46 mg, 0.053 mmol, 0.2 equiv.), Cs₂CO₃ (171.53 mg, 0.526 mmol, 2 equiv.) and Pd(OAc)₂ (5.91 mg, 0.026 mmol, 0.1 equiv.) at room temperature under nitrogen atmosphere. The resulting mixture was stirred for 3 hours at 110° C. under nitrogen atmosphere. The reaction was monitored by LCMS. The mixture was allowed to cool down to room temperature. The resulting mixture was filtered, the filter cake was washed with DCM (3×10 mL). The filtrate was concentrated under reduced pressure. The residue was purified by reverse phase flash with the following conditions (Column: C18, 330 g; Mobile Phase A:Water/0.05% NH₄HCO₃, Mobile Phase B:ACN; Flow rate:80 mL/min; Gradient: 40% B to 70% B in 20 min; Detector, 254 nm, Monitor, 220 nm, the desired product were collected at 65% B) to afford tert-butyl 4-[(7-[[2-fluoro-4-(pyrazol-1-yl)phenyl]amino]-1,6-naphthyridin-2-yl)sulfanyl]piperidine-1-carboxylate (110 mg, 80%) as a yellow solid.

N-[2-fluoro-4-(pyrazol-1-yl)phenyl]-2-(piperidin-4-ylsulfanyl)-1,6-naphthyridin-7-amine (Compound 107). To a stirred solution of tert-butyl 4-[(7-[[2-fluoro-4-(pyrazol-1-yl)phenyl]amino]-1,6-naphthyridin-2-yl)sulfanyl]piperidine-1-carboxylate (50 mg, 0.096 mmol, 1 equiv.) in DCM (4 mL) was added TFA (1 mL) at room temperature. The resulting mixture was stirred for 2 hours at room temperature. The reaction was monitored by LCMS. The resulting mixture was concentrated under reduced pressure. The residue was basified to pH 8 with saturated NaHCO₃ (aq.). The resulting mixture was concentrated under reduced pressure. The crude product (40 mg) as purified by Prep-HPLC to afford N-[2-fluoro-4-(pyrazol-1-yl)phenyl]-2-(piperidin-4-ylsulfanyl)-1,6-naphthyridin-7-amine (17.2 mg, 42%) as a yellow solid.

Compounds 108, 118, 129, and 133 were synthesized following the methods and protocols as described for the synthesis of Compound 107, starting with the appropriate materials.

### Example 55. Preparation of Compounds 134 and 136

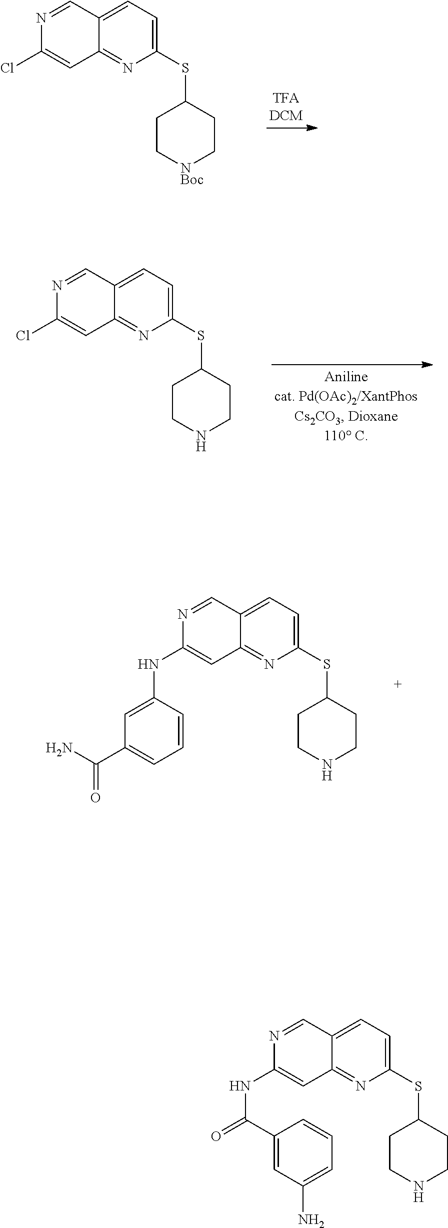 [SMILES: CC(C)(C)OC(=O)N1CCC(Sc2ccc3cnc(Cl)cc3n2)CC1 ; Clc1cc2nc(SC3CCNCC3)ccc2cn1 ; NC(=O)c1cccc(Nc2cc3nc(SC4CCNCC4)ccc3cn2)c1 ; Nc1cccc(C(=O)Nc2cc3nc(SC4CCNCC4)ccc3cn2)c1]

7-chloro-2-(piperidin-4-ylsulfanyl)-1,6-naphthyridine. To a stirred solution of tert-butyl 4-[(7-chloro-1,6-naphthyridin-2-yl)sulfanyl]piperidine-1-carboxylate (1 g, 2.632 mmol, 1 equiv.) in DCM (20 mL) was added TFA (2 mL, 26.926 mmol, 10.23 equiv.) at room temperature. The resulting mixture was stirred for 1 hour at room temperature. The reaction was monitored by LCMS. The mixture was basified to pH 8 with saturated NaHCO₃ (aq.). The resulting mixture was concentrated under reduced pressure. The residue was purified by reverse phase flash with the following conditions (Column: C18, 330 g; Mobile Phase A:Water/0.05% NH₄HCO₃, Mobile Phase B:ACN; Flow rate:80 mL/min; Gradient: 20% B to 50% B in 20 min; Detector, 254 nm, Monitor, 220 nm, the desired product were collected at 42% B) to afford 7-chloro-2-(piperidin-4-ylsulfanyl)-1,6-naphthyridine (650 mg, 88%) as a white solid.

3-[[2-(piperidin-4-ylsulfanyl)-1,6-naphthyridin-7-yl]amino]benzamide (Compound 134) and 3-amino-N-[2-(piperidin-4-ylsulfanyl)-1,6-naphthyridin-7-yl]benzamide (Compound 136). To a stirred mixture of 7-chloro-2-(piperidin-4-ylsulfanyl)-1,6-naphthyridine (150 mg, 3.286 mmol, 1 equiv.) and 3-aminobenzamide (88 mg, 4.272 mmol) in 1,4-dioxane (4 mL) were added Pd(OAc)₂ (18 mg, 0.329 mmol, 0.15 equiv.), XantPhos (93 mg, 0.657 mmol, 0.30 equiv.) and Cs₂CO₃ (351 mg, 6.572 mmol, 2 equiv.) at room temperature under nitrogen atmosphere. The resulting mixture was stirred for 2 hours at 110° C. under nitrogen atmosphere. The reaction was monitored by LCMS. The mixture was allowed to cool down to room temperature. The resulting mixture was filtered, the filter cake was washed with EtOAc (3×10 mL). The filtrate was concentrated under reduced pressure. The residue was purified by Prep-HPLC to afford 3-[[2-(piperidin-4-ylsulfanyl)-1,6-naphthyridin-7-yl]amino]benzamide; formic acid (7.9 mg) as a yellow solid and 3-amino-N-[2-(piperidin-4-ylsulfanyl)-1,6-naphthyridin-7-yl]benzamide; formic acid (14 mg) as a white solid.

### Example 56. Preparation of Compound 138

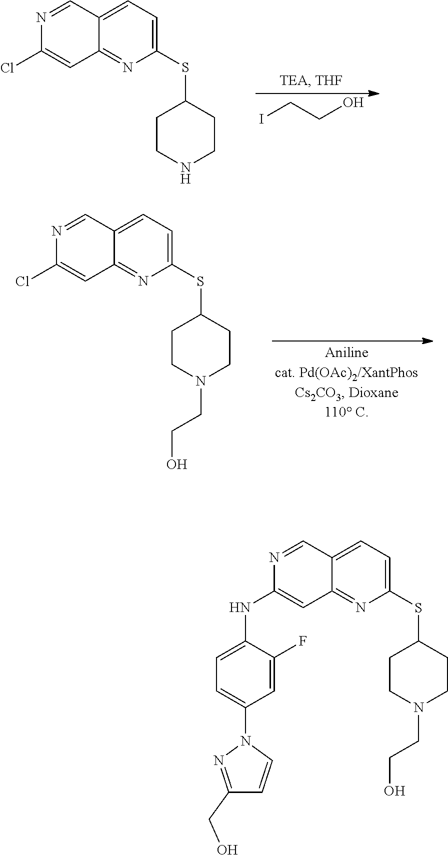

2-[4-[(7-chloro-1,6-naphthyridin-2-yl)sulfanyl]piperidin-1-yl]ethanol. To a stirred mixture of 7-chloro-2-(piperidin-4-ylsulfanyl)-1,6-naphthyridine (Compound 134, step 1, 120 mg, 3.286 mmol, 1 equiv.) and ethanol, 2-iodo-(89 mg, 4.272 mmol, 1.20 equiv.) in THF (5 mL, 0.657 mmol) was added TEA (87 mg, 0.329 mmol, 2 equiv.) at room temperature. The resulting mixture was stirred for 1 hour at room temperature. The reaction was monitored by LCMS. The resulting mixture was concentrated under reduced pressure. The residue was purified by reverse phase flash with the following conditions (Column: C18, 330 g; Mobile Phase A:Water/0.05% NH₄HCO₃, Mobile Phase B:ACN; Flow rate:80 mL/min; Gradient: 20% B to 50% B in 20 min; Detector, 254 nm, Monitor, 220 nm, the desired product were collected at 45% B) to afford 2-[4-[(7-chloro-1,6-naphthyridin-2-yl)sulfanyl]piperidin-1-yl]ethanol (100 mg) as a yellow solid.

2-(4-[[7-([2-fluoro-4-[3-(hydroxymethyl)pyrazol-1-yl]phenyl]amino)-1,6-naphthyridin-2-yl]sulfanyl]piperidin-1-yl)ethanol (Compound 138). To a stirred mixture of 2-[4-[(7-chloro-1,6-naphthyridin-2-yl)sulfanyl]piperidin-1-yl]ethanol (60 mg, 3.286 mmol, 1 equiv.) and [1-(4-amino-3-fluorophenyl)pyrazol-3-yl]methanol (77 mg, 4.272 mmol, 2 equiv.) in 1,4-dioxane (5 mL, 0.038 mmol) were added Pd(OAc)₂ (13 mg, 0.329 mmol, 0.15 equiv.), XantPhos (65 mg, 0.657 mmol, 0.30 equiv.) and Cs₂CO₃ (242 mg, 6.572 mmol, 4 equiv.) at room temperature under nitrogen atmosphere. The resulting mixture was stirred for 2 hours at 110° C. under nitrogen atmosphere. The reaction was monitored by LCMS. The mixture was allowed to cool down to room temperature. The resulting mixture was filtered, the filter cake was washed with EtOAc (3×10 mL). The filtrate was concentrated under reduced pressure. The residue was purified by Prep-HPLC to afford 2-(4-[[7-([2-fluoro-4-[3-(hydroxymethyl)pyrazol-1-yl]phenyl]amino)-1,6-naphthyridin-2-yl]sulfanyl]piperidin-1-yl)ethanol (6.5 mg) as a light yellow solid.

### Example 57. Preparation of Compound 121

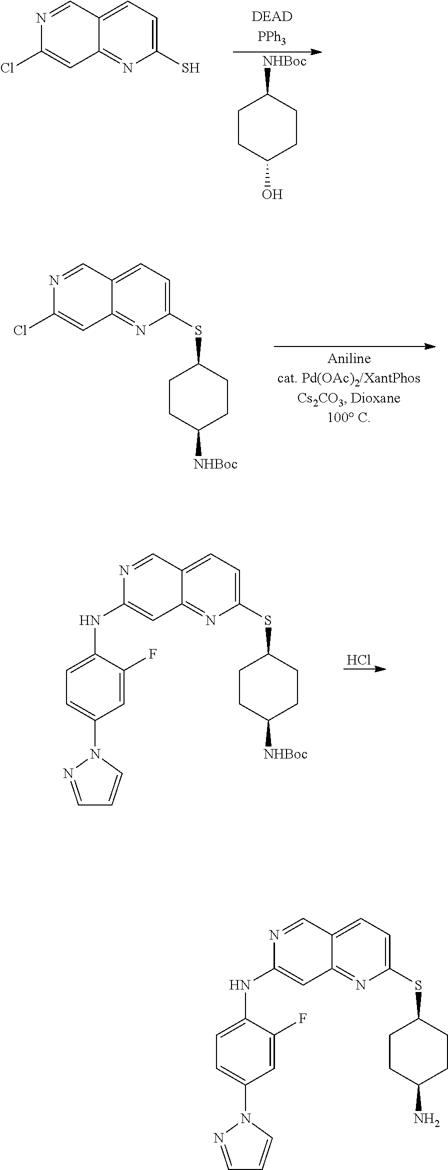 [SMILES: Sc1ccc2cnc(Cl)cc2n1 ; CC(C)(C)OC(=O)N[C@H]1CC[C@H](O)CC1 ; CC(C)(C)OC(=O)N[C@H]1CC[C@@H](Sc2ccc3cnc(Cl)cc3n2)CC1 ; CC(C)(C)OC(=O)N[C@H]1CC[C@@H](Sc2ccc3cnc(Nc4ccc(-n5cccn5)cc4F)cc3n2)CC1 ; Cl ; N[C@H]1CC[C@@H](Sc2ccc3cnc(Nc4ccc(-n5cccn5)cc4F)cc3n2)CC1]

Tert-butyl N-[(1s,4s)-4-[(7-chloro-1,6-naphthyridin-2-yl)sulfanyl]cyclohexyl]carbamate. To a stirred mixture of 7-chloro-1,6-naphthyridine-2-thiol (100 mg, 1 equiv.) and tert-butyl N-[(1r,4r)-4-hydroxycyclohexyl]carbamate (548.47 mg, 5 equiv.) in THF (10 mL) were added DEAD (177.55 mg, 2 equiv.) and PPh₃ (267.35 mg, 2 equiv.) dropwise at 0° C. under nitrogen atmosphere. The resulting mixture was stirred for 16 hours at room temperature under nitrogen atmosphere. The reaction was monitored by LCMS. The resulting mixture was extracted with EtOAc (3×200 mL). The combined organic layers were washed with brine (2×100 mL), dried over anhydrous Na₂SO₄. After filtration, the filtrate was concentrated under reduced pressure. The residue was purified by reverse phase flash chromatography with the following conditions: Column: Spherical C18, 20-40 um, 330 g; Mobile Phase A: Water (plus 5 mM NH₄NO₃); Mobile Phase B: ACN; Flow rate: 80 mL/min; Gradient: 5%-5% B, 10 min, 55% B-95% B gradient in 30 min; Detector: 254 nm. The fractions containing the desired product were collected at 95% B and concentrated under reduced pressure to afford tert-butyl N-[(1s,4s)-4-[(7-chloro-1,6-naphthyridin-2-yl)sulfanyl]cyclohexyl]carbamate (260 mg) as a light yellow solid.

Tert-butyl N-[(1s,4s)-4-[(7-[[2-fluoro-4-(pyrazol-1-yl)phenyl]amino]-1,6-naphthyridin-2-yl)sulfanyl]cyclohexyl]carbamate. To a stirred mixture of tert-butyl N-[(1s,4s)-4-[(7-chloro-1,6-naphthyridin-2-yl)sulfanyl]cyclohexyl]carbamate (120 mg, 0.305 mmol, 1 equiv.) and 2-fluoro-4-(pyrazol-1-yl)aniline (70.17 mg, 0.396 mmol, 1.30 equiv.) in 1,4-dioxane (5 mL) were added XantPhos (52.88 mg, 0.091 mmol, 0.30 equiv.), Cs₂CO₃ (198.50 mg, 0.609 mmol, 2 equiv.) and Pd(OAc)₂ (10.26 mg, 0.046 mmol, 0.15 equiv.) at room temperature under nitrogen atmosphere. The resulting mixture was stirred for 2 hours at 100° C. under nitrogen atmosphere. The reaction was monitored by LCMS. The mixture was allowed to cool down to room temperature. The resulting mixture was concentrated under reduced pressure. The resulting mixture was filtered, the filter cake was washed with EtOAc (3×20 mL). The filtrate was concentrated under reduced pressure. The residue was purified by reverse phase flash with the following conditions (Column: C18, 330 g; Mobile Phase A:Water/0.05% TFA, Mobile Phase B:ACN; Flow rate:80 mL/min; Gradient: 50% B to 95% B in 20 min; Detector, 254 nm and 220 nm, the desired product were collected at 95% B). Concentrated under reduced pressure to afford tert-butyl N-[(1s,4s)-4-[(7-[[2-fluoro-4-(pyrazol-1-yl)phenyl]amino]-1,6-naphthyridin-2-yl)sulfanyl]cyclohexyl]carbamate (110 mg, 67%) as a light brown solid.

N-[2-fluoro-4-(pyrazol-1-yl)phenyl]-2-[[(1s,4s)-4-aminocyclohexyl]sulfanyl]-1,6-naphthyridin-7-amine (Compound 121). To a stirred solution of tert-butyl N-[(1s,4s)-4-[(7-[[2-fluoro-4-(pyrazol-1-yl)phenyl]amino]-1,6-naphthyridin-2-yl)sulfanyl]cyclohexyl]carbamate (110 mg, 0.206 mmol, 1 equiv.) in MeOH (10 mL) was added HCl (gas) in 1,4-dioxane (12 mL, 394.943 mmol, 1919.60 equiv.) in portions at room temperature. The resulting mixture was stirred for 30 min at room temperature. The reaction was monitored by LCMS. The resulting mixture was concentrated under reduced pressure. The residue was purified by Prep-HPLC to afford N-[2-fluoro-4-(pyrazol-1-yl)phenyl]-2-[[(1s,4s)-4-aminocyclohexyl]sulfanyl]-1,6-naphthyridin-7-amine (24.7 mg, 27%) as a light yellow solid.

Compounds 122, 135, 153, 157, 159 and 161 were synthesized following the methods and protocols as described for the synthesis of Compound 121, starting with the appropriate materials.

### Example 58. Preparation of Compound 138

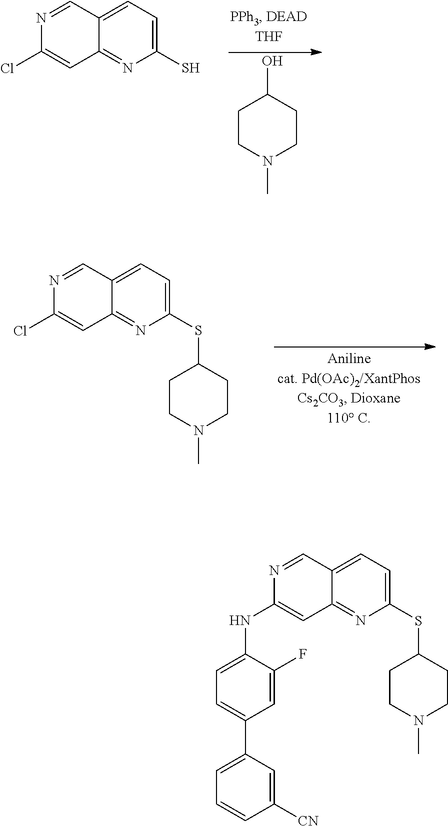

7-chloro-2-[(1-methylpiperidin-4-yl)sulfanyl]-1,6-naphthyridine. To a stirred mixture of 7-chloro-1,6-naphthyridine-2-thiol (6 g, 30.511 mmol, 1 equiv.) and 4-piperidinol, 1-methyl-(17.57 g, 152.555 mmol, 5 equiv.) in THF (100 mL) were added DEAD (10.63 g, 61.022 mmol, 2 equiv.) and PPh₃ (14.40 g, 54.920 mmol, 1.80 equiv.) dropwise at 0° C. under nitrogen atmosphere. The resulting mixture was stirred for 16 hours at room temperature under nitrogen atmosphere. The reaction was monitored by LCMS. The residue was purified by reverse phase flash chromatography with the following conditions: Column: Spherical C18, 20-40 um, 330 g; Mobile Phase A: Water (plus 5 mM TFA); Mobile Phase B: ACN; Flow rate: 80 mL/min; Gradient: 5%-5% B, 10 min, 30% B-65% B gradient in 30 min; Detector: 254 nm. The fractions containing the desired product were collected at 40% B and concentrated under reduced pressure to afford 7-chloro-2-[(1-methylpiperidin-4-yl)sulfanyl]-1,6-naphthyridine (3 g, 33%) as a yellow solid.

3′-fluoro-4′-([2-[(1-methylpiperidin-4-yl)sulfanyl]-1,6-naphthyridin-7-yl]amino)-[1,1′-biphenyl]-3-carbonitrile (Compound 138). To a stirred mixture of 7-chloro-2-[(1-methylpiperidin-4-yl)sulfanyl]-1,6-naphthyridine (200 mg, 3.286 mmol, 1 equiv.) and 4-amino-3-fluoro-[1,1-biphenyl]-3-carbonitrile (159 mg, 4.272 mmol, 1.10 equiv.) in 1,4-dioxane (10 mL) were added Pd(OAc)₂ (23 mg, 0.329 mmol, 0.15 equiv.), XantPhos (118.40 mg, 0.657 mmol, 0.30 equiv.) and Cs₂CO₃ (445 mg, 6.572 mmol, 2 equiv.) at room temperature under nitrogen atmosphere. The resulting mixture was stirred for 2 hours at 110° C. under nitrogen atmosphere. The reaction was monitored by LCMS. The mixture was allowed to cool down to room temperature. The resulting mixture was filtered, the filter cake was washed with EtOAc (3×10 mL). The filtrate was concentrated under reduced pressure. The residue was purified by Prep-HPLC to afford 3′-fluoro-4′-([2-[(1-methylpiperidin-4-yl)sulfanyl]-1,6-naphthyridin-7-yl]amino)-[1,1′-biphenyl]-3-carbonitrile (77 mg) as a white solid.

### Example 59. Preparation of Compounds 115 and 117

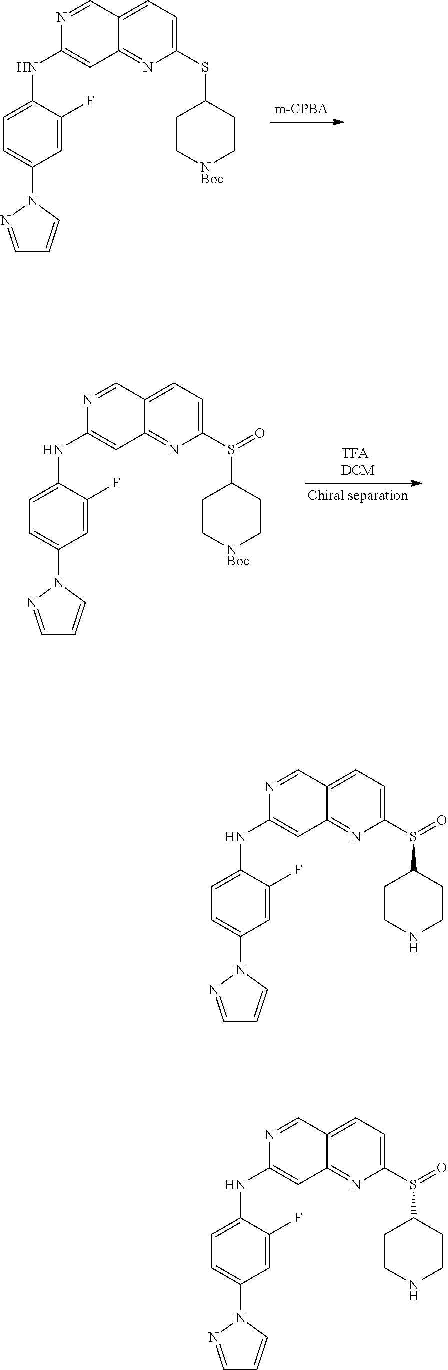 [SMILES: CC(C)(C)OC(=O)N1CCC(Sc2ccc3cnc(Nc4ccc(-n5cccn5)cc4F)cc3n2)CC1 ; CC(C)(C)OC(=O)N1CCC(S(=O)c2ccc3cnc(Nc4ccc(-n5cccn5)cc4F)cc3n2)CC1 ; O=[S@](c1ccc2cnc(Nc3ccc(-n4cccn4)cc3F)cc2n1)C1CCNCC1 ; O=[S@@](c1ccc2cnc(Nc3ccc(-n4cccn4)cc3F)cc2n1)C1CCNCC1]

Tert-butyl 4-(7-[[2-fluoro-4-(pyrazol-1-yl)phenyl]amino]-1,6-naphthyridin-2-ylsulfinyl)piperidine-1-carboxylate. To a stirred solution of tert-butyl 4-[(7-[[2-fluoro-4-(pyrazol-1-yl)phenyl]amino]-1,6-naphthyridin-2-yl)sulfanyl]piperidine-1-carboxylate (Compound 107, step 2, 1 g, 1.921 mmol, 1 equiv.) in DCM (30 mL) was added m-CPBA (265.16 mg, 1.537 mmol, 0.80 equiv.) at 0° C. The resulting mixture was stirred for 1 hour at 0° C. The reaction was monitored by LCMS. The reaction was quenched with sat. NaHSO₃ (aq.) at 0° C. The resulting mixture was extracted with DCM (3×100 mL). The combined organic layers were washed with brine (1×100 mL), dried over anhydrous Na₂SO₄. After filtration, the filtrate was concentrated under reduced pressure. The residue was purified by reverse phase flash with the following conditions (Column: C18, 330 g; Mobile Phase A:Water/0.05% NH₄HCO₃, Mobile Phase B:ACN; Flow rate:80 mL/min; Gradient: 30% B to 60% B in 20 min; Detector, 220 nm, Monitor, 254 nm, the desired product were collected at 60% B) to afford tert-butyl 4-(7-[[2-fluoro-4-(pyrazol-1-yl)phenyl]amino]-1,6-naphthyridin-2-ylsulfinyl)piperidine-1-carboxylate (450 mg, 43%) as a yellow solid and tert-butyl 4-(7-[[2-fluoro-4-(pyrazol-1-yl)phenyl]amino]-1,6-naphthyridin-2-ylsulfonyl)piperidine-1-carboxylate (100 mg, 9%) as a yellow solid.

(R)- and (S)—N-[2-fluoro-4-(pyrazol-1-yl)phenyl]-2-[piperidine-4-sulfinyl]-1,6-naphthyridin-7-amine (Compounds 115 and 117). To a stirred solution of tert-butyl 4-(7-[[2-fluoro-4-(pyrazol-1-yl)phenyl]amino]-1,6-naphthyridin-2-ylsulfinyl)piperidine-1-carboxylate (150 mg) in DCM (8 mL) was added TFA (1 mL) at room temperature. The resulting mixture was stirred for 1 hour at room temperature. The reaction was monitored by LCMS. The resulting mixture was concentrated under reduced pressure. The residue was basified to pH 8 with saturated NaHCO₃ (aq.). The resulting mixture was concentrated under reduced pressure. The crude product (120 mg) was purified by Prep-HPLC with the following conditions (Column: XBridge Prep OBD C₁₈ Column, 30×150 mm 5 μm) to afford mixture of P1 and P2. The crude product (70 mg) was purified by Prep-HPLC to afford N-[2-fluoro-4-(pyrazol-1-yl)phenyl]-2-[(R)-piperidine-4-sulfinyl]-1,6-naphthyridin-7-amine (21 mg) as a yellow solid and N-[2-fluoro-4-(pyrazol-1-yl)phenyl]-2-[(S)-piperidine-4-sulfinyl]-1,6-naphthyridin-7-amine (18 mg) as a yellow solid. Compound 115-eluted at 14.727 min; yield: 21 mg. Compound 117-eluted at 18.838 min; yield: 18 mg.

### Example 60. Preparation of Compound 123

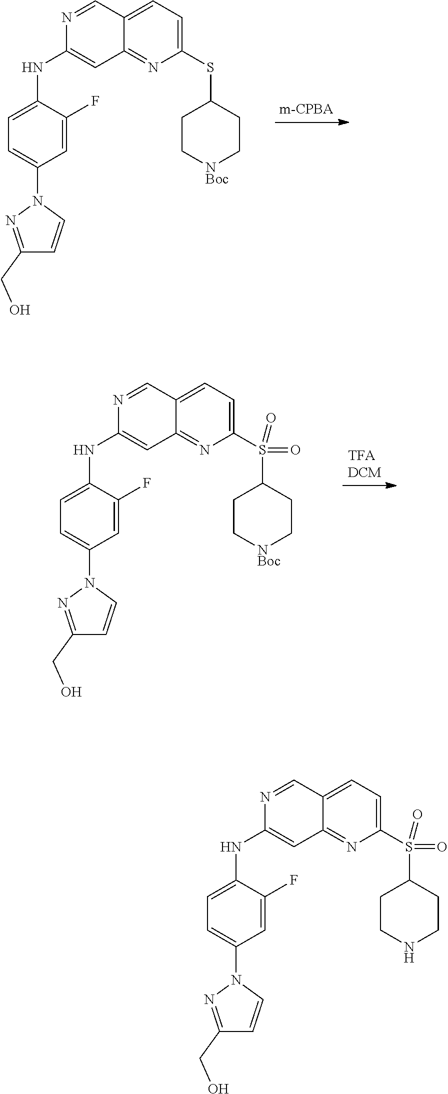 [SMILES: CC(C)(C)OC(=O)N1CCC(Sc2ccc3cnc(Nc4ccc(-n5ccc(CO)n5)cc4F)cc3n2)CC1 ; CC(C)(C)OC(=O)N1CCC(S(=O)(=O)c2ccc3cnc(Nc4ccc(-n5ccc(CO)n5)cc4F)cc3n2)CC1 ; O=S(=O)(c1ccc2cnc(Nc3ccc(-n4ccc(CO)n4)cc3F)cc2n1)C1CCNCC1]

Tert-butyl 4-[7-([2-fluoro-4-[3-(hydroxymethyl)pyrazol-1-yl]phenyl]amino)-1,6-naphthyridin-2-ylsulfonyl]piperidine-1-carboxylate. To a stirred solution of tert-butyl 4-[[7-([2-fluoro-4-[3-(hydroxymethyl)pyrazol-1-yl]phenyl]amino)-1,6-naphthyridin-2-yl]sulfanyl]piperidine-1-carboxylate (Compound 108, step 2, 400 mg, 0.726 mmol, 1 equiv.) in DCM (20 mL) was added m-CPBA (250.71 mg, 1.453 mmol, 2 equiv.) at room temperature. The resulting mixture was stirred for 2 hours at room temperature. The reaction was monitored by LCMS. The resulting mixture was concentrated under reduced pressure. The residue was purified by reverse phase flash with the following conditions (Column: C18, 330 g; Mobile Phase A:Water/0.05% TFA, Mobile Phase B:ACN; Flow rate:80 mL/min; Gradient: 30% B to 60% B in 20 min; Detector, 254 nm, Monitor, 220 nm, the desired product were collected at 56% B) to afford tert-butyl 4-[7-([2-fluoro-4-[3-(hydroxymethyl)pyrazol-1-yl]phenyl]amino)-1,6-naphthyridin-2-ylsulfonyl]piperidine-1-carboxylate (200 mg, 47%) as a yellow solid.

[1-(3-fluoro-4-[[2-(piperidine-4-sulfonyl)-1,6-naphthyridin-7-yl]amino]phenyl)pyrazol-3-yl]methanol (Compound 123). To a stirred solution of tert-butyl 4-[7-([2-fluoro-4-[3-(hydroxymethyl)pyrazol-1-yl]phenyl]amino)-1,6-naphthyridin-2-ylsulfonyl]piperidine-1-carboxylate (200 mg, 0.343 mmol, 1 equiv.) in DCM (10 mL) was added TFA (1 mL, 13.463 mmol, 39.22 equiv.) at room temperature. The resulting mixture was stirred for 1 hour at room temperature. The reaction was monitored by LCMS. The mixture was basified to pH 8 with saturated NaHCO₃ (aq.). The resulting mixture was concentrated under reduced pressure. The residue was purified by reverse phase flash with the following conditions (Column: C18, 120 g; Mobile Phase A:Water/0.05% NH₄HCO₃, Mobile Phase B:ACN; Flow rate:80 mL/min; Gradient: 15% B to 40% B in 20 min; Detector, 254 nm, Monitor, 220 nm, the desired product were collected at 32% B) to afford [1-(3-fluoro-4-[[2-(piperidine-4-sulfonyl)-1,6-naphthyridin-7-yl]amino]phenyl)pyrazol-3-yl]methanol (70 mg, 42%) as an orange solid.

Compounds 113, 151, 171, 174, 180, 181, 197, 199, 200, 206, 210, 212, and 234 were synthesized following the methods and protocols as described for the synthesis of Compound 123, starting with the appropriate materials.

### Example 61, Preparation of Compound 127

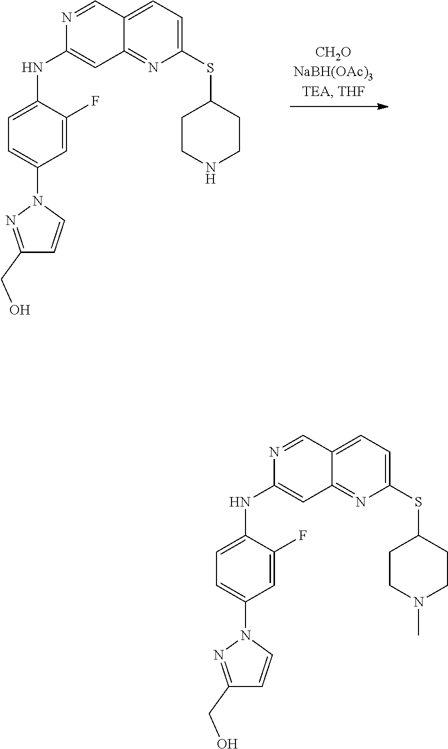 [SMILES: OCc1ccn(-c2ccc(Nc3cc4nc(SC5CCNCC5)ccc4cn3)c(F)c2)n1 ; CN1CCC(Sc2ccc3cnc(Nc4ccc(-n5ccc(CO)n5)cc4F)cc3n2)CC1]

To a stirred mixture of [1-(3-fluoro-4-[[2-(piperidin-4-ylsulfanyl)-1,6-naphthyridin-7-yl]amino]phenyl)pyrazol-3-yl]methanol (Compound 108, 90 mg, 0.200 mmol, 1 equiv.) and TEA (40.43 mg, 0.400 mmol, 2 equiv.) in THF (10 mL, 123.430 mmol, 617.89 equiv.) were added NaBH(OAc)₃ (63.51 mg, 0.300 mmol, 1.50 equiv.) and HCHO (7.80 mg, 0.260 mmol, 1.30 equiv.) dropwise at room temperature. The resulting mixture was stirred for 1 hour at room temperature. The reaction was monitored by LCMS. The mixture was basified to pH 8 with saturated NaHCO₃ (aq.). The resulting mixture was filtered, the filter cake was washed with EtOAc (3×10 mL). The filtrate was concentrated under reduced pressure. The crude product (60 mg) was purified by Prep-HPLC to afford [1-[3-fluoro-4-([2-[(1-methylpiperidin-4-yl)sulfanyl]-1,6-naphthyridin-7-yl]amino)phenyl]pyrazol-3-yl]methanol (29.3 mg, 31%) as a white solid. Compounds 128 and 148 were synthesized following the methods and protocols as described for the synthesis of Compound 127, starting with the appropriate materials.

### Example 62. Preparation of Compound 158

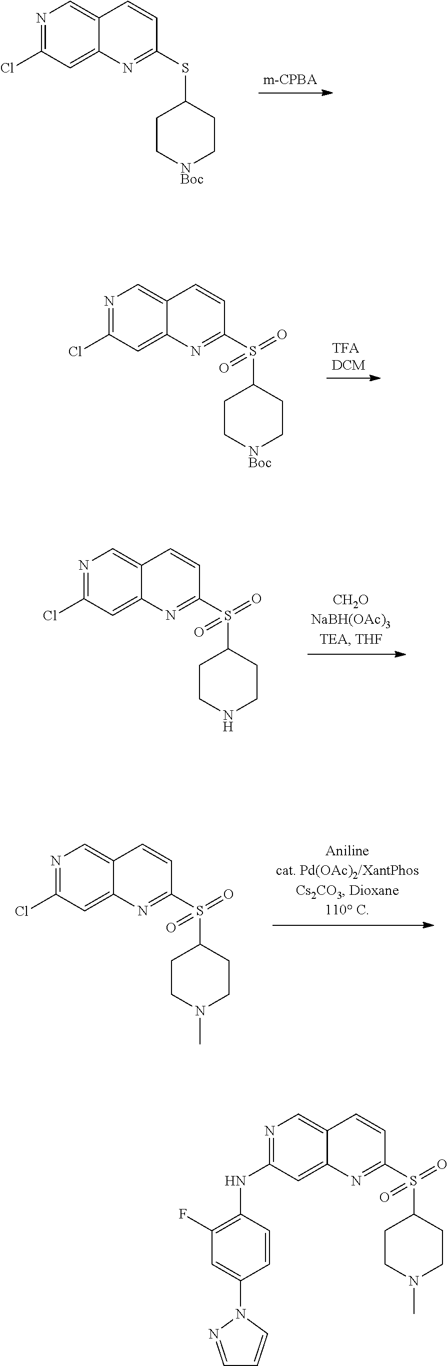 [SMILES: CC(C)(C)OC(=O)N1CCC(Sc2ccc3cnc(Cl)cc3n2)CC1 ; CC(C)(C)OC(=O)N1CCC(S(=O)(=O)c2ccc3cnc(Cl)cc3n2)CC1 ; O=S(=O)(c1ccc2cnc(Cl)cc2n1)C1CCNCC1 ; CN1CCC(S(=O)(=O)c2ccc3cnc(Cl)cc3n2)CC1 ; CN1CCC(S(=O)(=O)c2ccc3cnc(Nc4ccc(-n5cccn5)cc4F)cc3n2)CC1]

Tert-butyl 4-(7-chloro-1,6-naphthyridin-2-ylsulfonyl)piperidine-1-carboxylate. To a stirred solution of tert-butyl 4-[(7-chloro-1,6-naphthyridin-2-yl)sulfanyl]piperidine-1-carboxylate (500 mg, 1.316 mmol, 1 equiv.) in DCM (40 mL) was added m-CPBA (681.36 mg, 3.948 mmol, 3 equiv.) in portions at 0° C. under nitrogen atmosphere. The resulting mixture was stirred for 4 hours at rt under nitrogen atmosphere. The reaction was monitored by TLC. The resulting mixture was extracted with DCM (3×300 mL). The combined organic layers were washed with brine (2×200 mL), dried over anhydrous Na₂SO₄. After filtration, the filtrate was concentrated under reduced pressure. The residue was purified by reverse phase flash chromatography with the following conditions: Column: Spherical C18, 20-40 um, 330 g; Mobile Phase A: Water (plus 5 mM FA); Mobile Phase B: ACN; Flow rate: 80 mL/min; Gradient: 5%-5% B, 10 min, 35% B-80% B gradient in 30 min; Detector: 220 nm. The fractions containing the desired product were collected at 72% B and concentrated under reduced pressure to afford tert-butyl 4-(7-chloro-1,6-naphthyridin-2-ylsulfonyl)piperidine-1-carboxylate (350 mg, 64%) as a yellow solid.

7-chloro-2-(piperidine-4-sulfonyl)-1,6-naphthyridine. To a stirred solution of tert-butyl 4-(7-chloro-1,6-naphthyridin-2-ylsulfonyl)piperidine-1-carboxylate (7 g) in DCM (80 mL) was added TFA (10 mL) dropwise at rt. The reaction mixture was stirred for 2 hours at rt. The reaction was monitored by TLC. The resulting mixture was concentrated under reduced pressure. The residue was basified to pH=8 with saturated NaHCO₃ (aq.). The resulting mixture was extracted with DCM (3×1000 mL). The combined organic layers were washed with brine (2×500 mL), dried over anhydrous Na₂SO₄. After filtration, the filtrate was concentrated under reduced pressure. This resulted in 7-chloro-2-(piperidine-4-sulfonyl)-1,6-naphthyridine (5.5 g) as a light yellow solid.

7-chloro-2-(1-methylpiperidin-4-ylsulfonyl)-1,6-naphthyridine. To a stirred mixture of 7-chloro-2-(piperidine-4-sulfonyl)-1,6-naphthyridine (1 g, 3.207 mmol, 1 equiv.)) and HCHO (0.13 g, 04 mmol, 1.3 equiv.) in THF (30 mL) was added NaBH(OAc)₃ (1.02 g, 4.811 mmol, 1.5 equiv.) at room temperature. The resulting mixture was stirred for 1 hour at room temperature. The reaction was monitored by LCMS. The resulting mixture was concentrated under reduced pressure. The residue was purified by reverse flash chromatography with the following conditions: column, C₁₈ silica gel; mobile phase, MeOH in water, 30% to 50% gradient in 10 min; detector, UV 254 nm to afford 7-chloro-2-(1-methylpiperidin-4-ylsulfonyl)-1,6-naphthyridine (980 mg, 93%) as a yellow solid.

N-[2-fluoro-4-(pyrazol-1-yl)phenyl]-2-(1-methylpiperidin-4-ylsulfonyl)-1,6-naphthyridin-7-amine (Compound 158). To a stirred mixture of 7-chloro-2-(1-methylpiperidin-4-ylsulfonyl)-1,6-naphthyridine (60 mg, 0.184 mmol, 1 equiv.) and 2-fluoro-4-(pyrazol-1-yl)aniline (35.89 mg, 0.203 mmol, 1.10 equiv.) in 1,4-dioxane (15 mL) were added Pd(OAc)₂ (6.20 mg, 0.028 mmol, 0.15 equiv.), XantPhos (31.97 mg, 0.055 mmol, 0.30 equiv.) and Cs₂CO₃ (120 mg, 0.368 mmol, 2 equiv.) in portions at rt under nitrogen atmosphere. The resulting mixture was stirred for 2 hours at 110° C. under nitrogen atmosphere. The reaction was monitored by LCMS. The mixture was allowed to cool down to rt. The resulting mixture was extracted with EtOAc (3×300 mL). The combined organic layers were washed with brine (2×100 mL), dried over anhydrous Na₂SO₄. After filtration, the filtrate was concentrated under reduced pressure. The residue was purified by Prep-HPLC to afford N-[2-fluoro-4-(pyrazol-1-yl)phenyl]-2-(1-methylpiperidin-4-ylsulfonyl)-1,6-naphthyridin-7-amine (29.2 mg, 33%) as a yellow solid.

Compounds 156, 288, 289, 290, 376, 378, 379, 380, 382, 384, and 385 were synthesized following the methods and protocols as described for the synthesis of Compound 158, starting with the appropriate materials.

### Example 63. Preparation of Compound 183

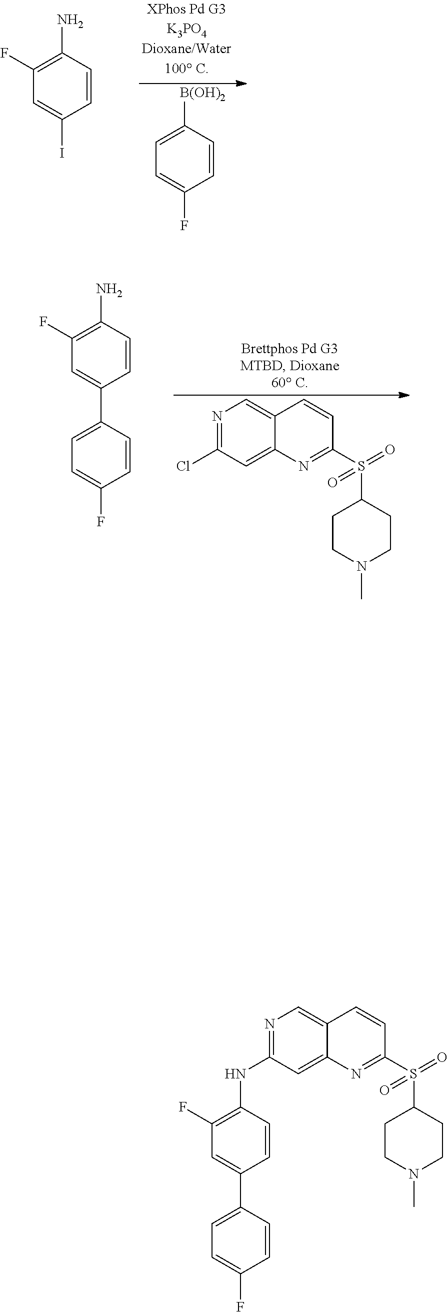 [SMILES: Nc1ccc(I)cc1F ; OB(O)c1ccc(F)cc1 ; Nc1ccc(-c2ccc(F)cc2)cc1F ; CN1CCC(S(=O)(=O)c2ccc3cnc(Cl)cc3n2)CC1 ; CN1CCC(S(=O)(=O)c2ccc3cnc(Nc4ccc(-c5ccc(F)cc5)cc4F)cc3n2)CC1]

3,4′-difluoro-[1,1′-biphenyl]-4-amine. A mixture of 2-fluoro-4-iodoaniline (0.5 g, 2.11 mmol, 1 equiv.), 4-fluorophenylboronic acid (0.47 g, 3.359 mmol, 1.592 equiv.) and XPhos Pd G3 (129 mg, 0.152 mmol, 0.072 equiv.) in dioxane (10 mL, 0.211 M, 20 Vols) was sparged with N₂ then charged with K₃PO₄ (1.5 g, 7.066 mmol, 3.35 equiv.) and water (2 mL, 1.055 M, 4 Vols). Heated to 100° C. for 4 hr. Cooled to RT, separated layers then filtered organics through Celite, eluting with EtOAc. Concentrated filtrate in vacuo onto SiO₂ and purified via flash chromatography (ISCO 40 g, 0-100% EtOAc/Heptane). Obtained 3,4′-difluoro-[1,1′-biphenyl]-4-amine (382.4 mg, 1.86 mmol, Yield 88%) as a yellow solid.

N-{3,4′-difluoro-[1,1′-biphenyl]-4-yl}-2-(1-methylpiperidin-4-ylsulfonyl)-1,6-naphthyridin-7-amine (Compound 183). A mixture of 7-chloro-2-(1-methylpiperidin-4-ylsulfonyl)-1,6-naphthyridine (Compound 158, step 3, 30.6 mg, 0.094 mmol, 1 equiv.), 3,4′-difluoro-[1,1′-biphenyl]-4-amine (27.3 mg, 0.133 mmol, 1.416 equiv.) and BrettPhos Pd G3 (5 mg, 06 mmol, 0.059 equiv.) in Dioxane (0.5 mL, 0.188 M, 16.34 Vols) was sparged with N₂ then charged with MTBD (0.102 g, 0.1 mL, 0.664 mmol, 7.074 equiv.). Stirred at 60° C. for 4 hr. LCMS shows product mass formation. Cooled to RT, filtered through Celite, eluted with EtOAc and concentrated in vacuo. Purified via flash chromatography (ISCO 4 g, 0-20% MeOH/DCM). Obtained impure product. Purified again (ISCO 4 g, 0-100% MeOH/EtOAc). Obtained N-{3,4′-difluoro-[1,1′-biphenyl]-4-yl}-2-(1-methylpiperidin-4-ylsulfonyl)-1,6-naphthyridin-7-amine (6.4 mg, 0.013 mmol, Yield 13.779%) as a yellow solid.

Compounds 201, 202, 224, 225, 226, 227, 228, 229, 230, 231, 232, 233, 234, 235, 236, 237, 238, 239 and 293 were synthesized following the methods and protocols as described for the synthesis of Compound 183, starting with the appropriate materials.

Compounds 184, 185, 191, 192, 193, 218, 219, 220, 221, 222, 223, 256, 257, 258, 259, 260, 262, 263, 264, 265, 266, 267, 268, 269, 270, 271, 272, 273, 274, 275, 276, 277, 278, 279, 280, 281, 282, 283, 284, 285, 286, 294, 295, 296, 297, 298, 299, 300, 301, 302, 303, 304, 305, 306, 307, 308, and 374 were synthesized following the methods and protocols as described for the second step of the synthesis of Compound 183, starting with commercial anilines.

### Example 64. Preparation of Compound 198

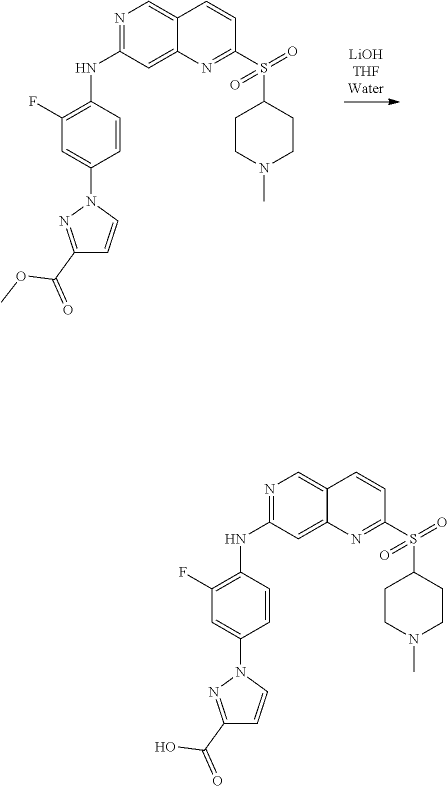 [SMILES: COC(=O)c1ccn(-c2ccc(Nc3cc4nc(S(=O)(=O)C5CCN(C)CC5)ccc4cn3)c(F)c2)n1 ; CN1CCC(S(=O)(=O)c2ccc3cnc(Nc4ccc(-n5ccc(C(=O)O)n5)cc4F)cc3n2)CC1]

A mixture of methyl 1-(3-fluoro-4-[[2-(1-methylpiperidin-4-ylsulfonyl)-1,6-naphthyridin-7-yl]amino]phenyl)pyrazole-3-carboxylate (100 mg, 0.191 mmol, 1 equiv.) and LiOH (45.65 mg, 1.906 mmol, 10 equiv.) in THF (20 mL) and water (4 mL) was stirred for 2 hours at room temperature. The reaction was monitored by LCMS. The resulting mixture was concentrated under reduced pressure. The residue was purified by Prep-HPLC to afford 1-(3-fluoro-4-((2-((1-methylpiperidin-4-yl)sulfonyl)-1,6-naphthyridin-7-yl)amino)phenyl)-1H-pyrazole-3-carboxylic acid (16.3 mg, 17%) as a yellow solid.

Compound 194 was synthesized following the methods and protocols as described for the synthesis of Compound 198, starting with the appropriate materials.

### Example 65. Preparation of Compound 317

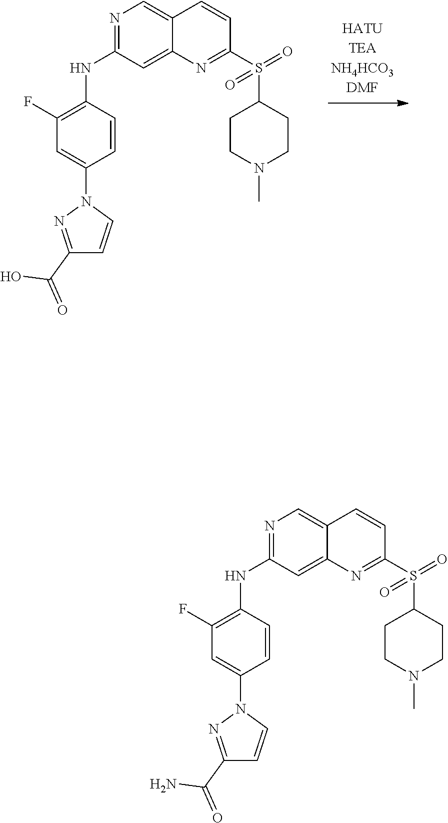 [SMILES: CN1CCC(S(=O)(=O)c2ccc3cnc(Nc4ccc(-n5ccc(C(=O)O)n5)cc4F)cc3n2)CC1 ; CN1CCC(S(=O)(=O)c2ccc3cnc(Nc4ccc(-n5ccc(C(N)=O)n5)cc4F)cc3n2)CC1]

To a stirred mixture of 1-(3-fluoro-4-[[2-(1-methylpiperidin-4-ylsulfonyl)-1,6-naphthyridin-7-yl]amino]phenyl)pyrazole-3-carboxylic acid (60 mg, 0.118 mmol, 1 equiv.) and HATU (89.37 mg, 0.235 mmol, 2 equiv.) in DMF (3 mL) were added NH₄HCO₃ (46.45 mg, 0.588 mmol, 5 equiv.) and TEA (35.68 mg, 0.353 mmol, 3 equiv.) at room temperature under nitrogen atmosphere. The resulting mixture was stirred for 4 hours at room temperature under nitrogen atmosphere. The reaction was monitored by LCMS. The mixture was allowed to cool down to room temperature. The resulting mixture was filtered, the filter cake was washed with EtOAc (3×10 mL). The crude product (mg) was purified by Prep-HPLC to afford 1-(3-fluoro-4-[[2-(1-methylpiperidin-4-ylsulfonyl)-1,6-naphthyridin-7-yl]amino]phenyl)pyrazole-3-carboxamide (16.4 mg, 27%) as a yellow solid.

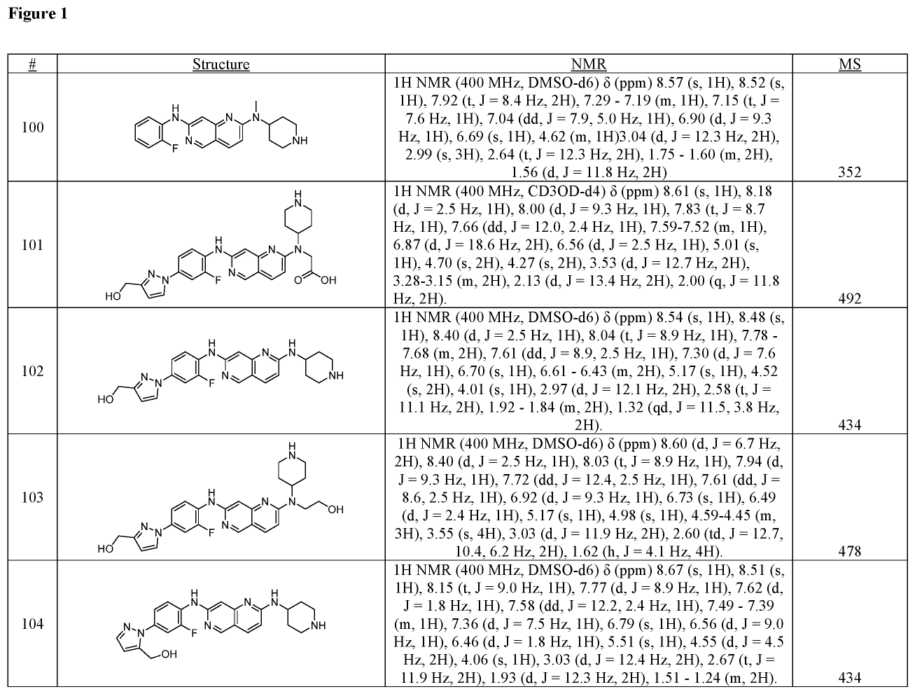

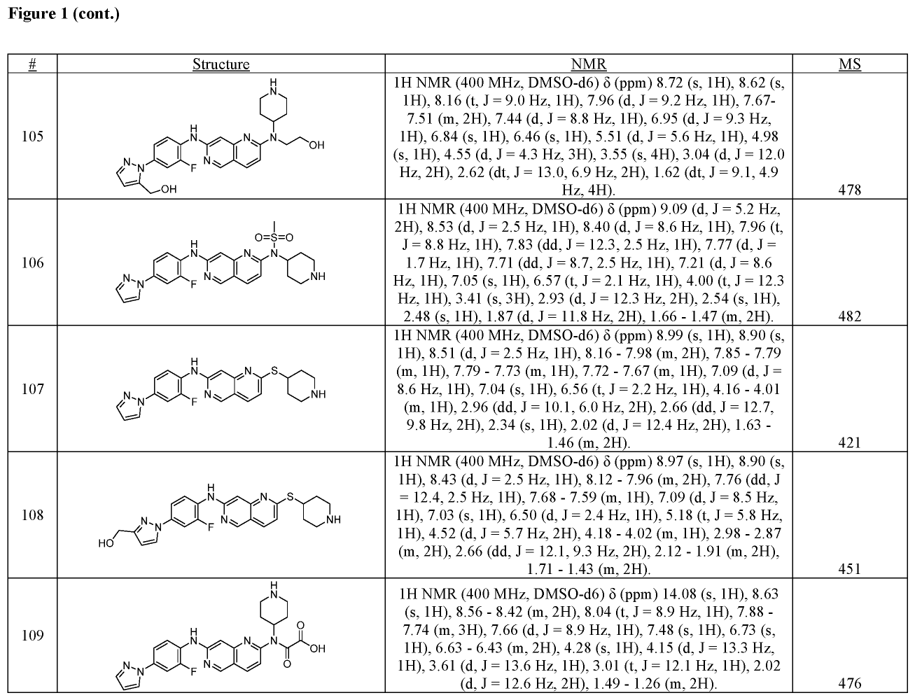

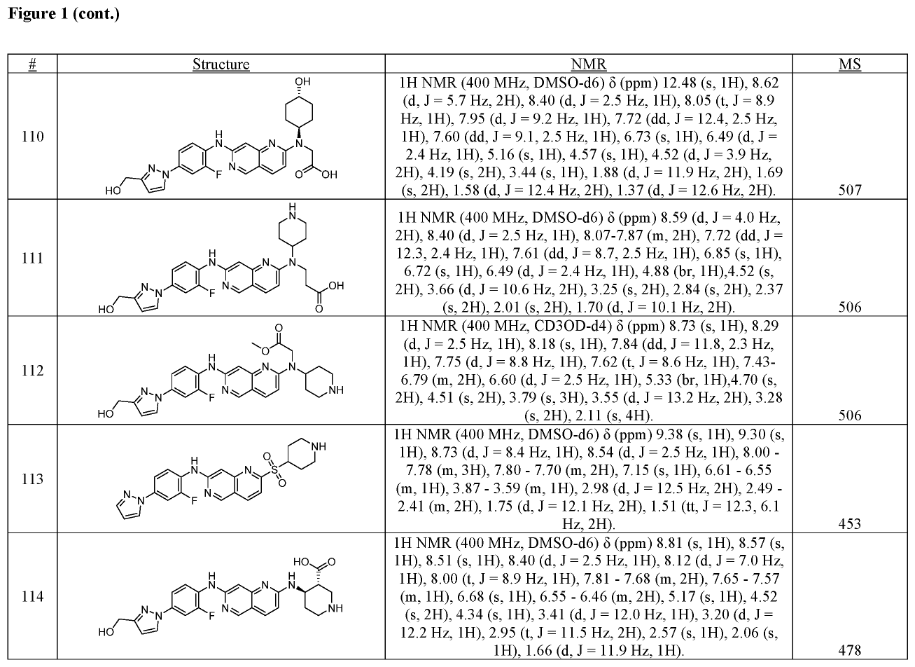

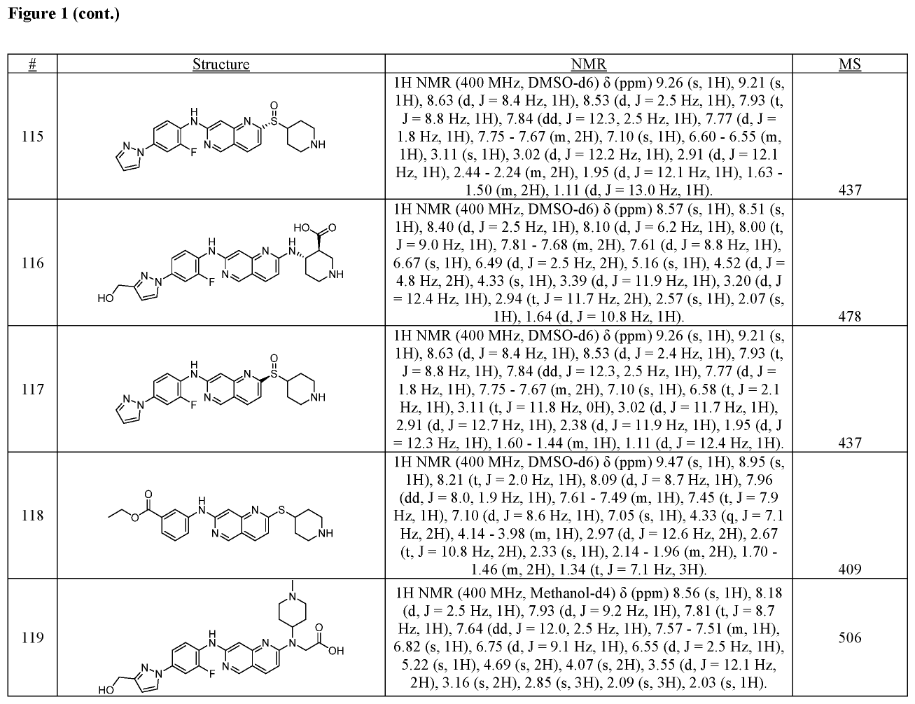

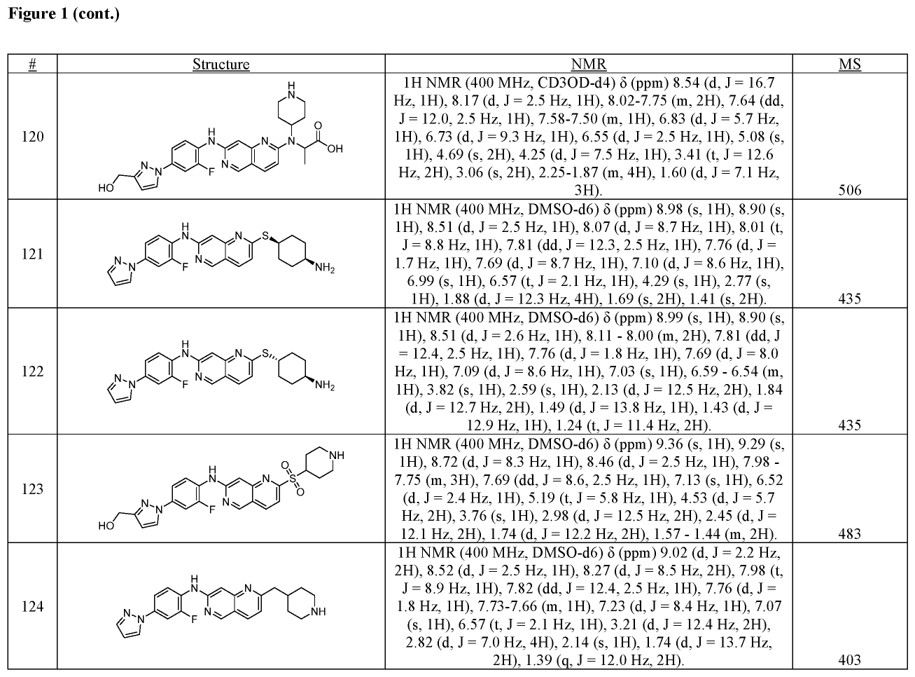

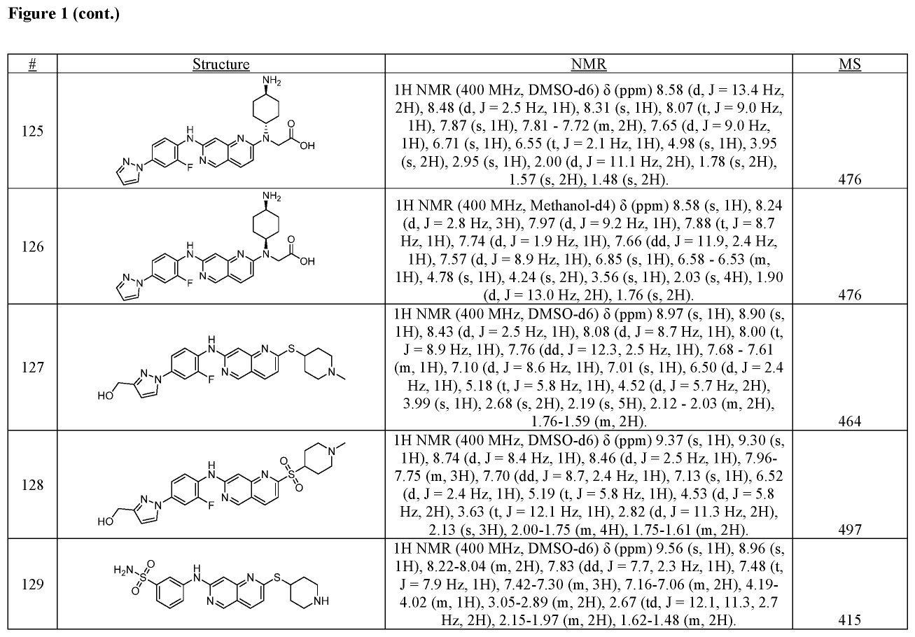

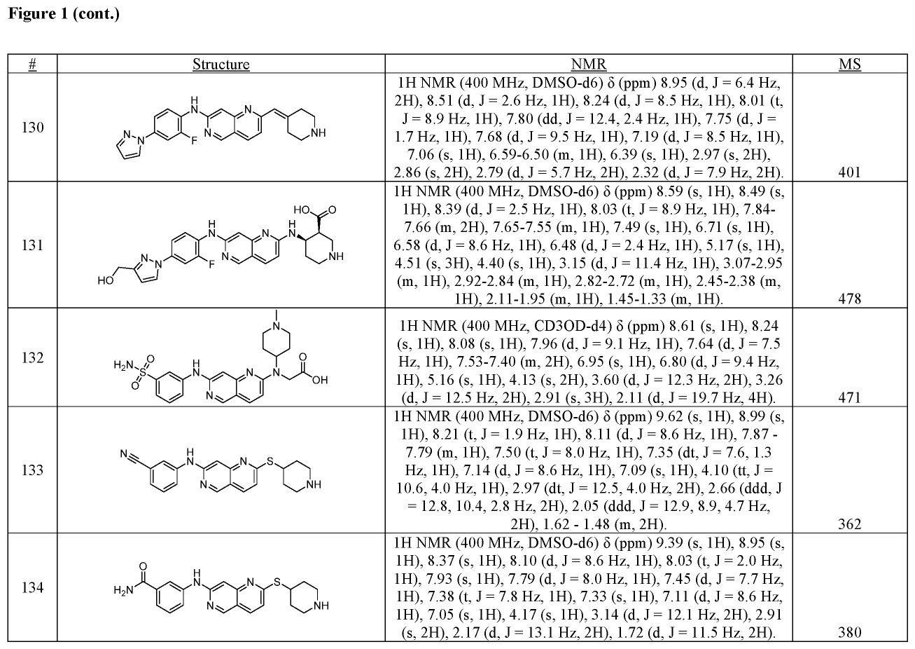

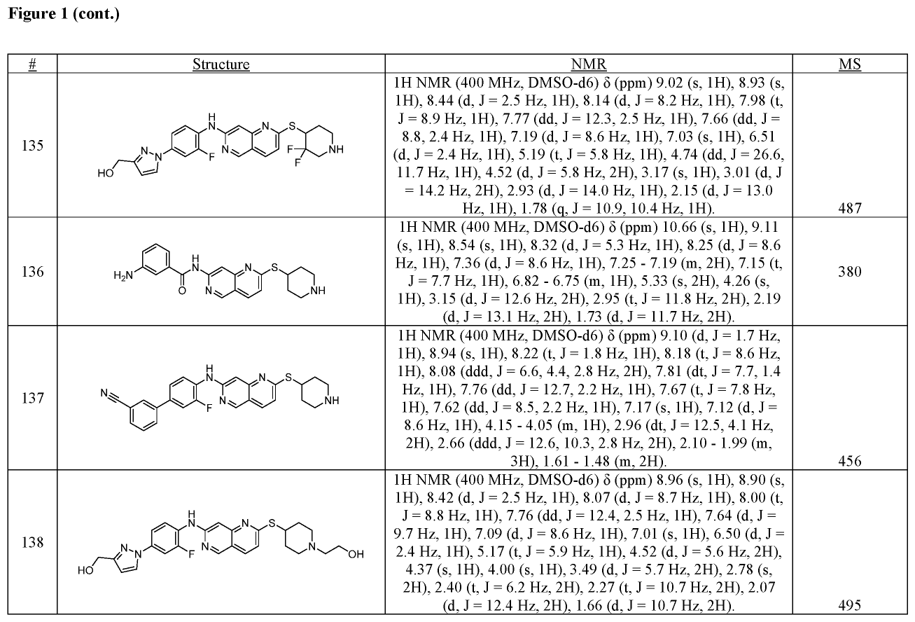

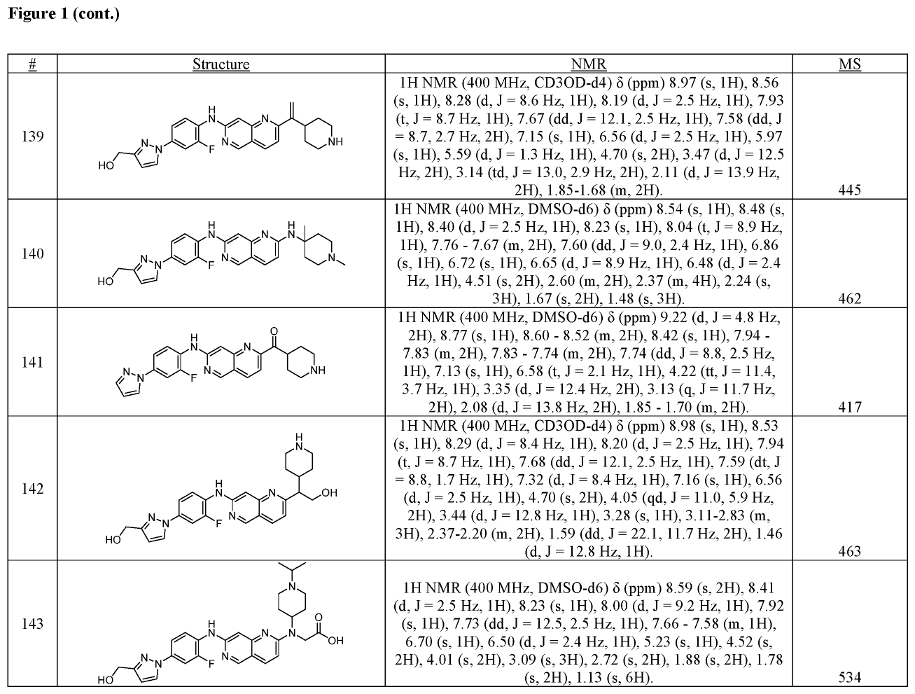

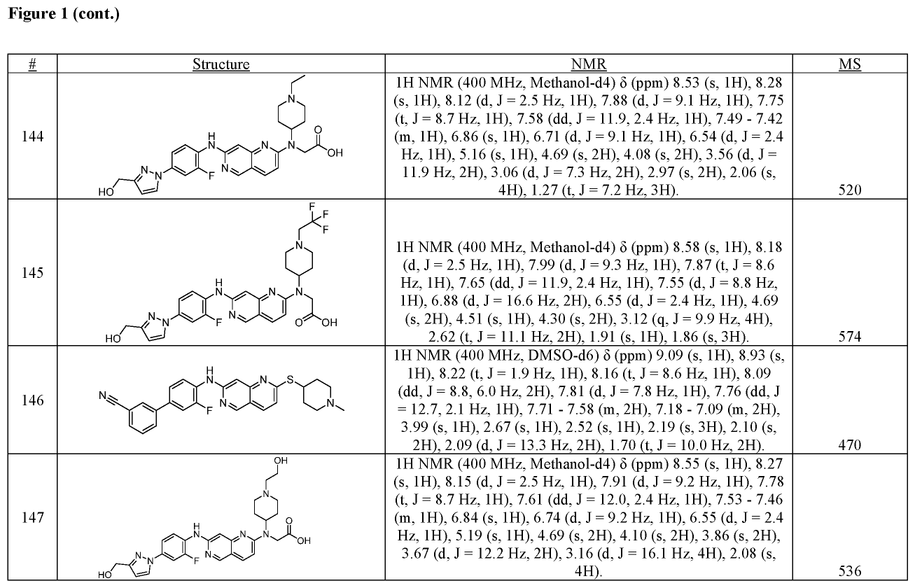

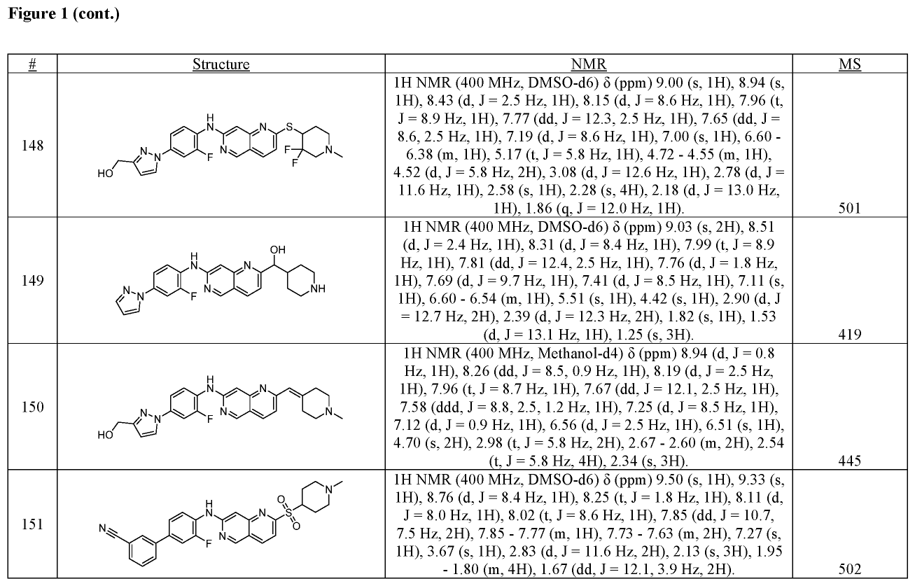

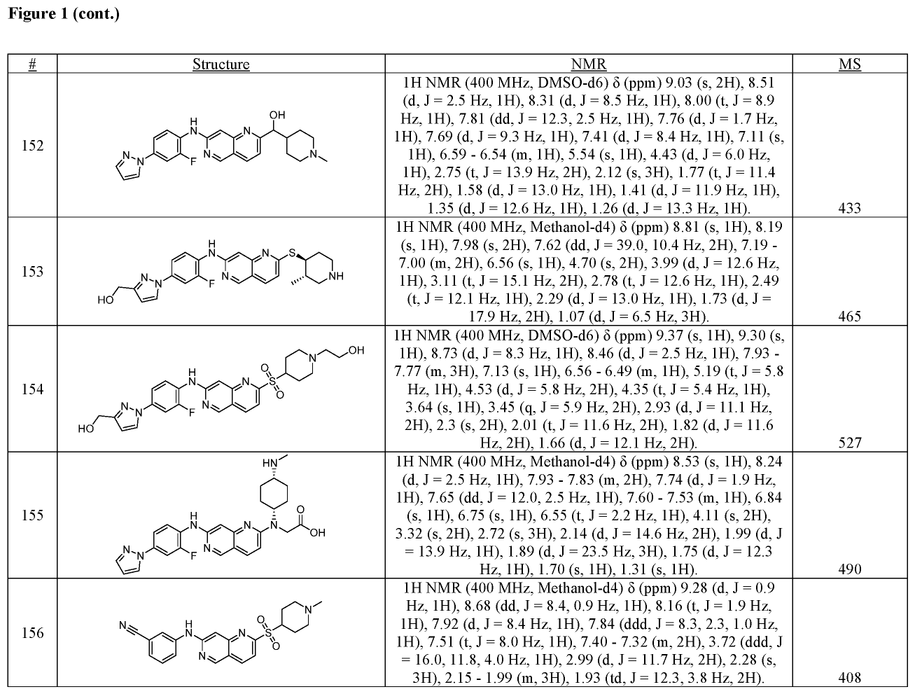

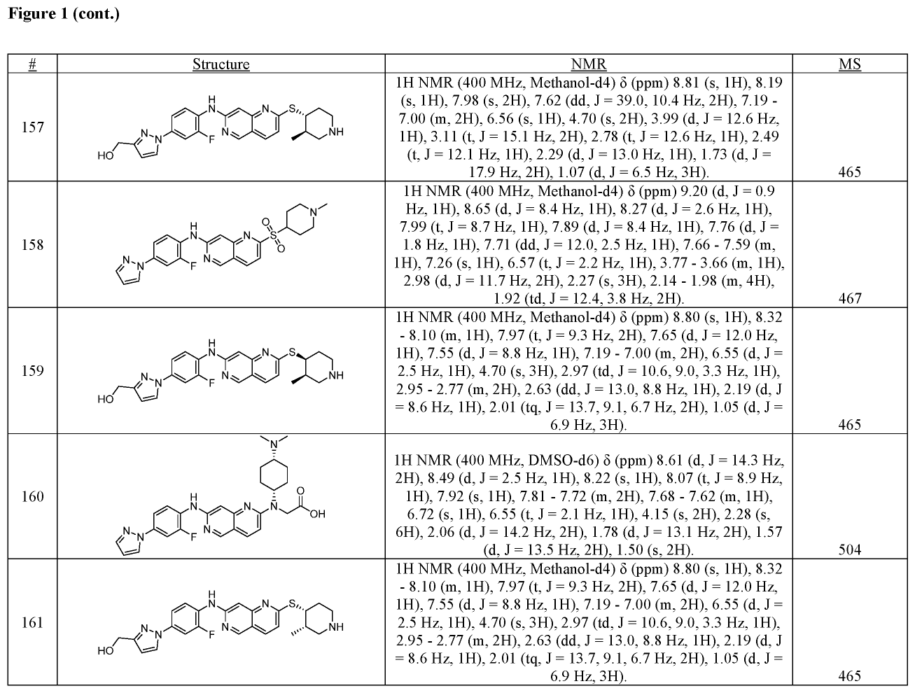

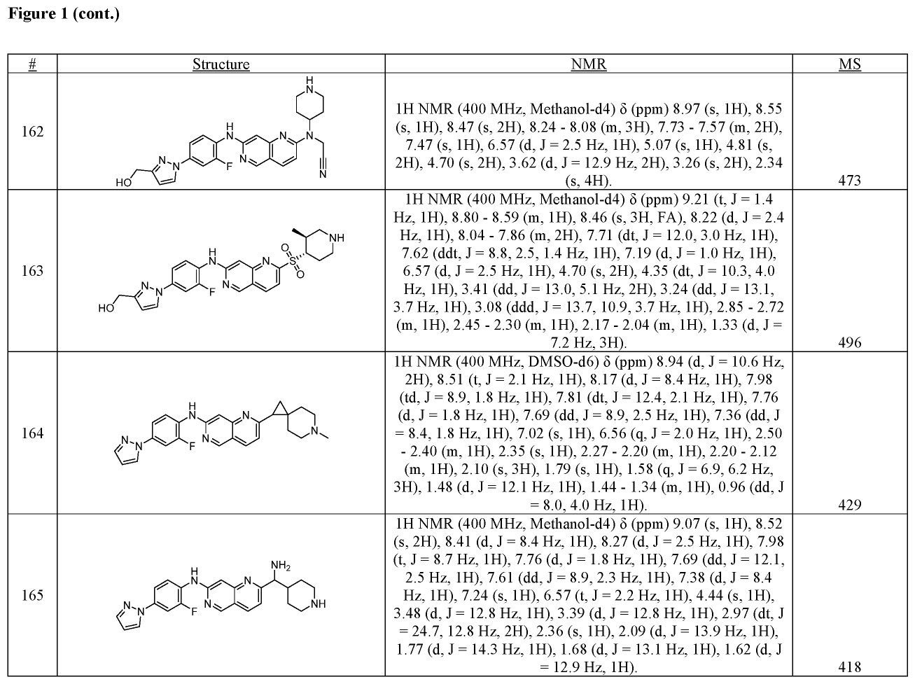

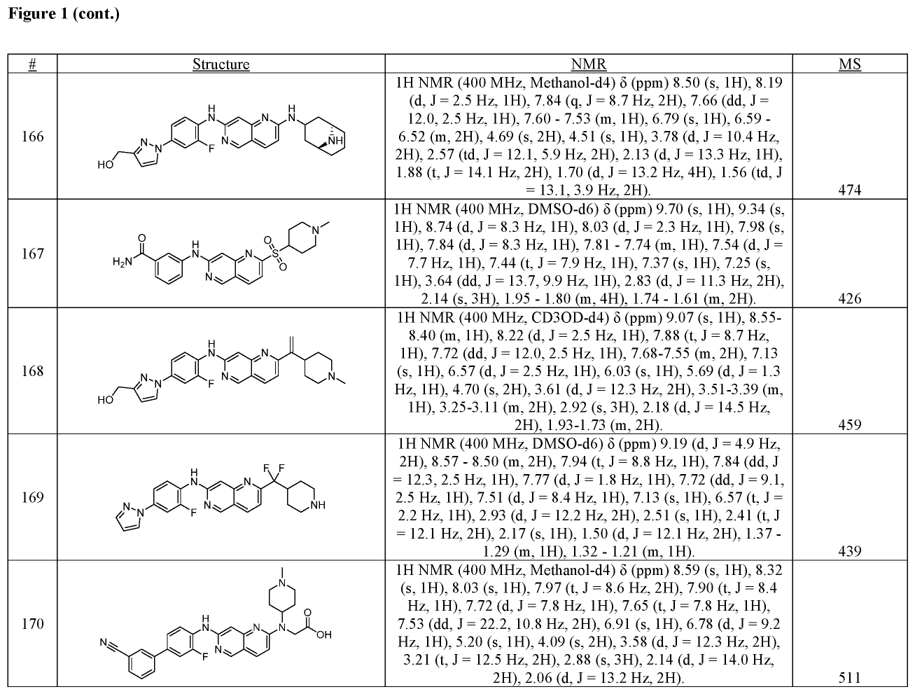

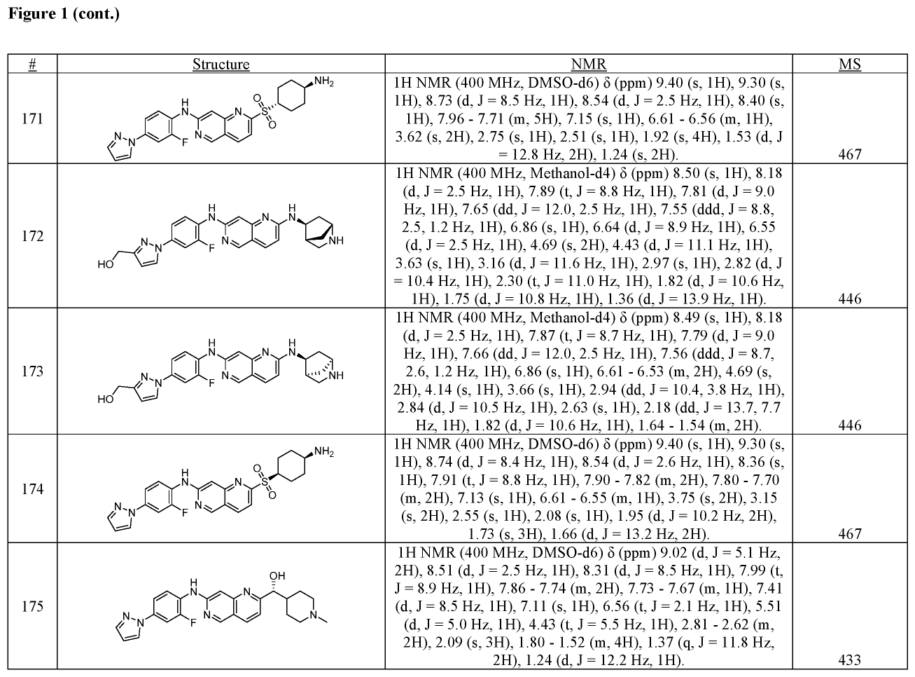

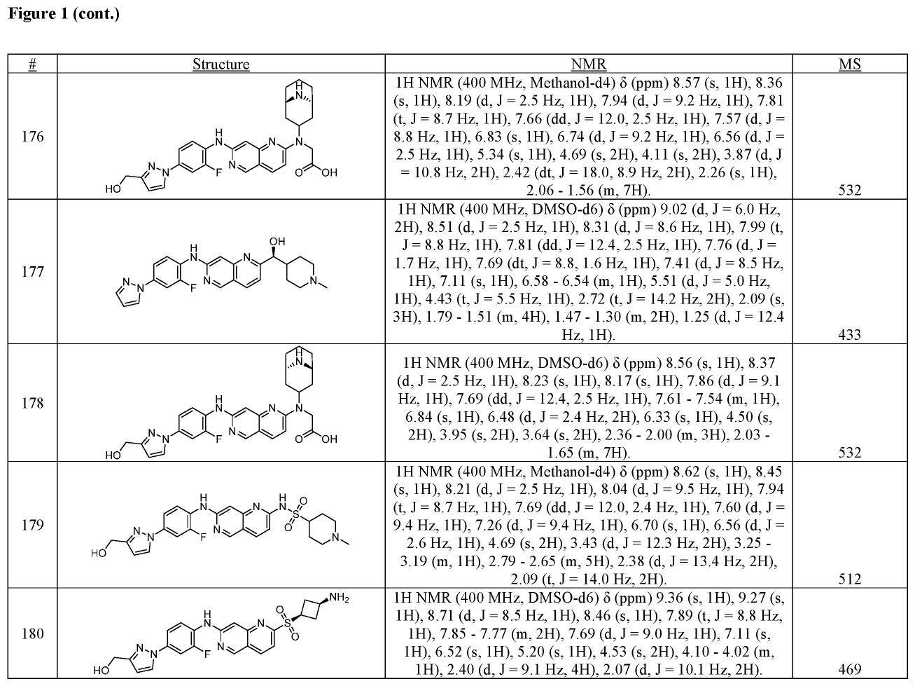

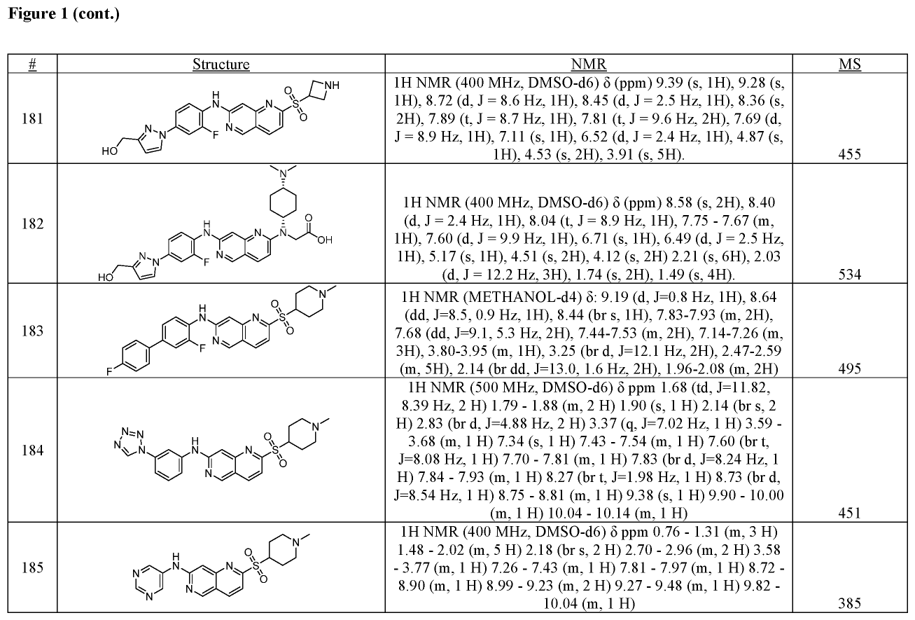

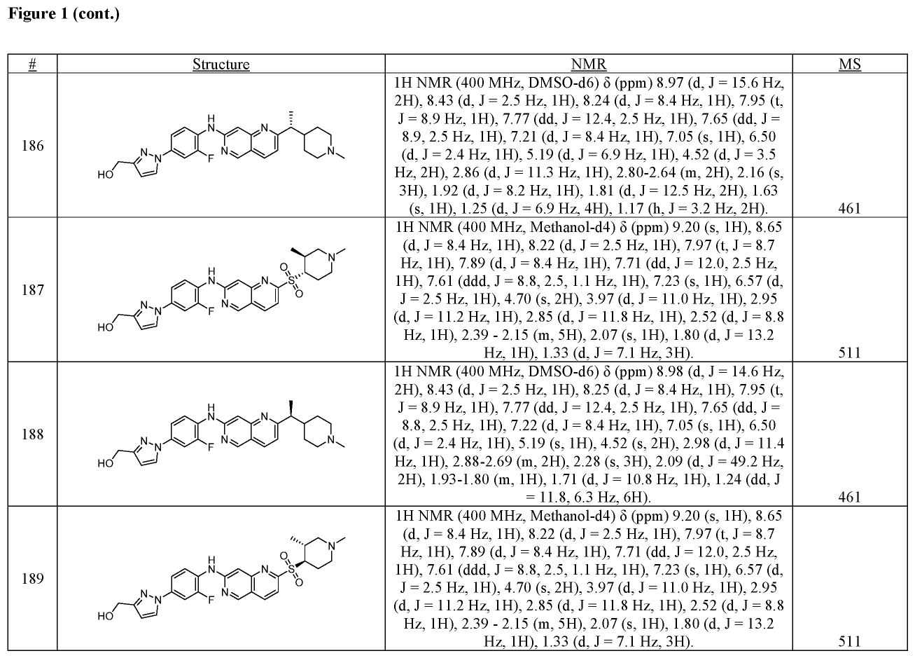

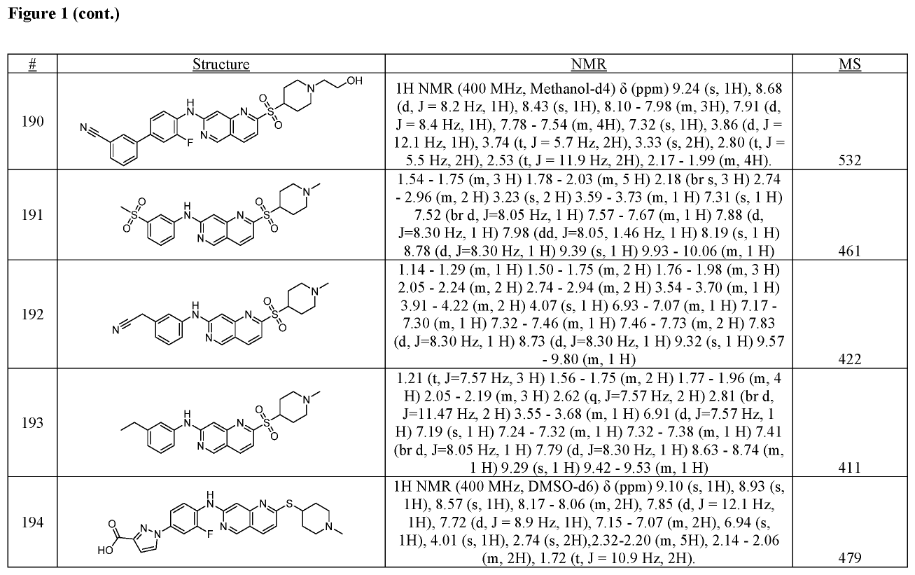

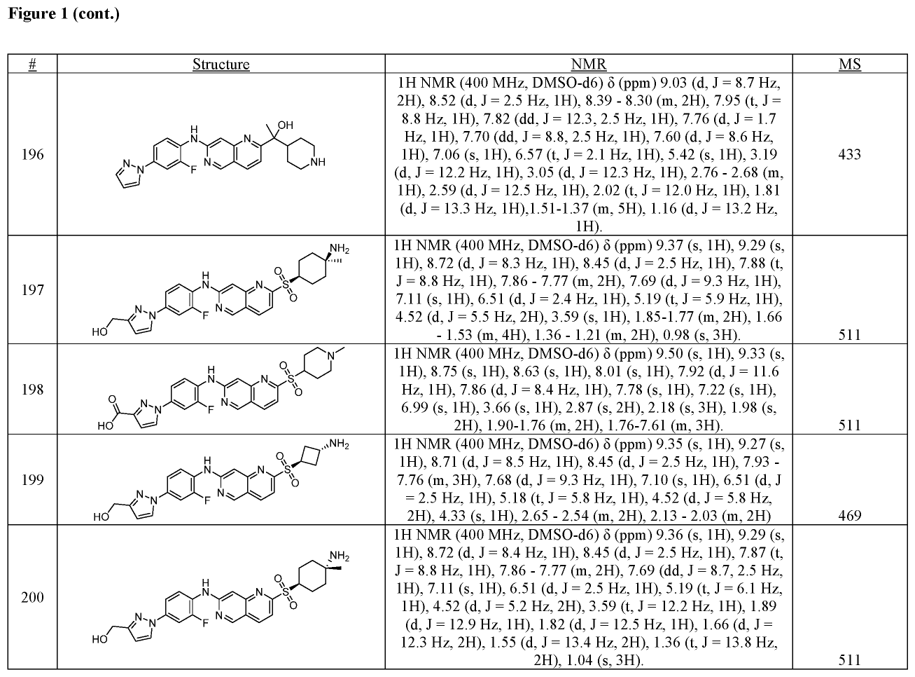

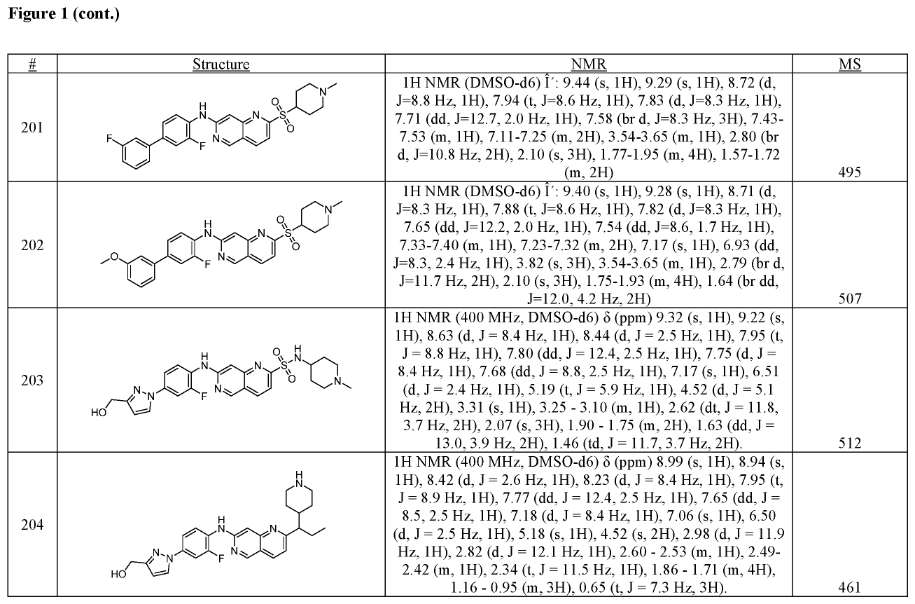

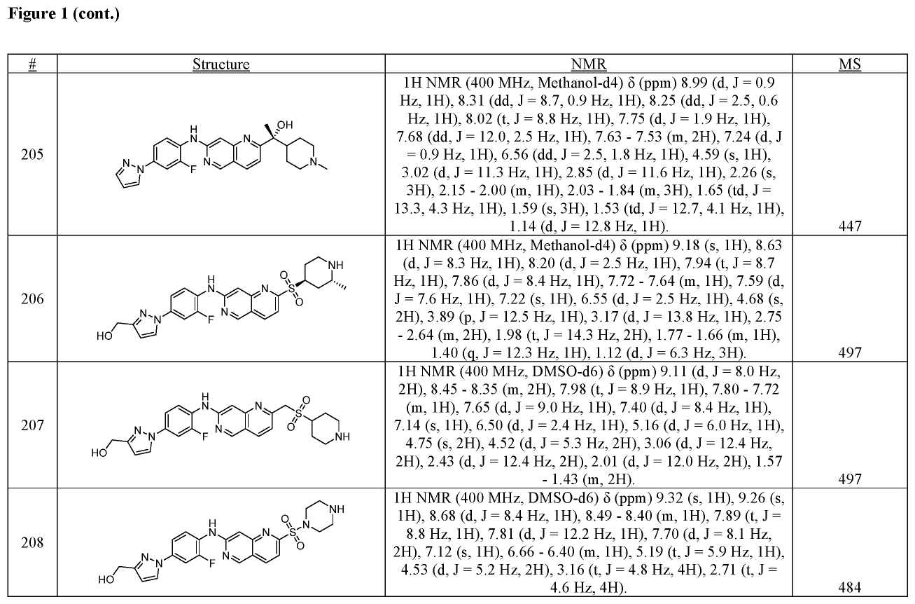

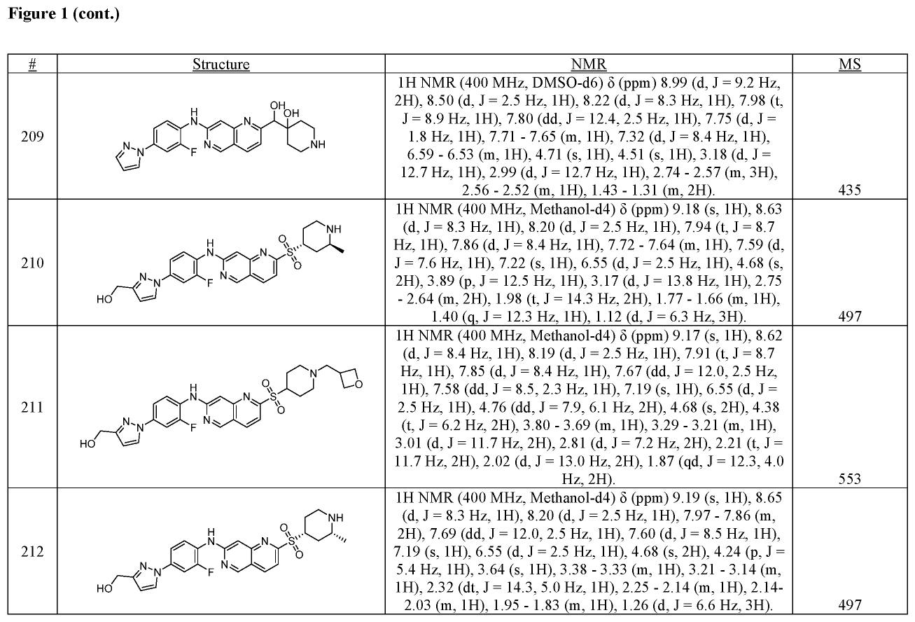

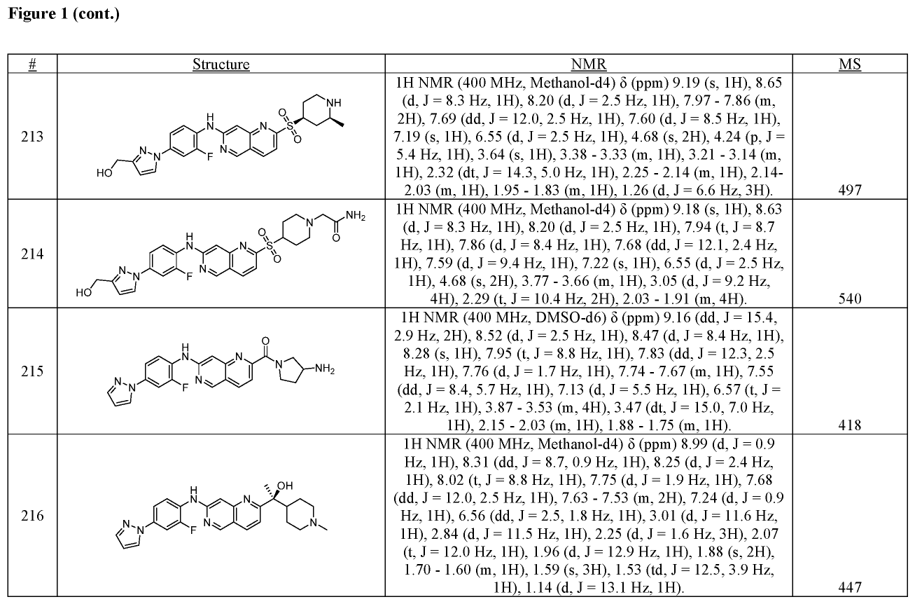

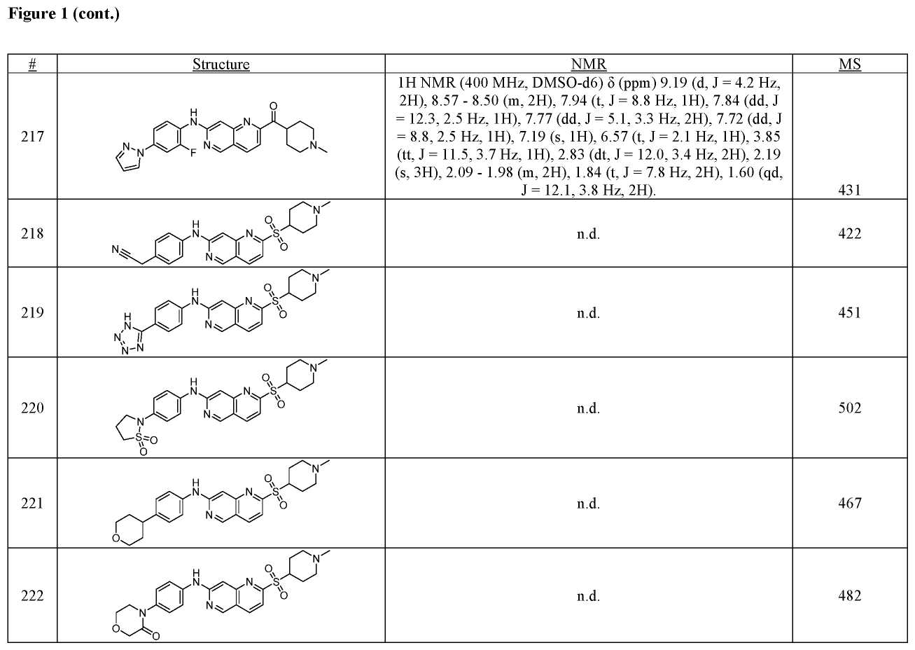

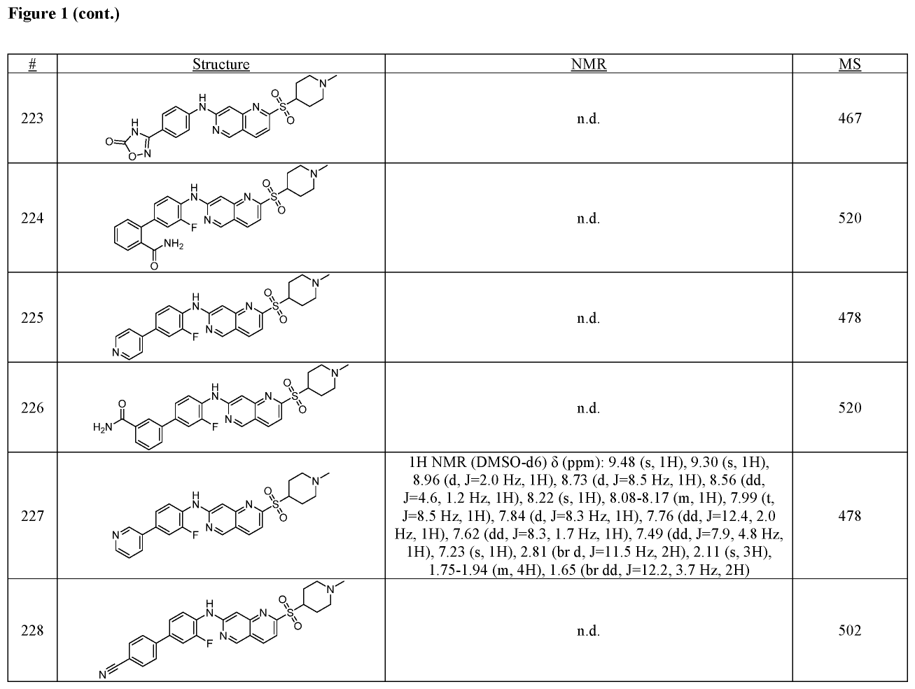

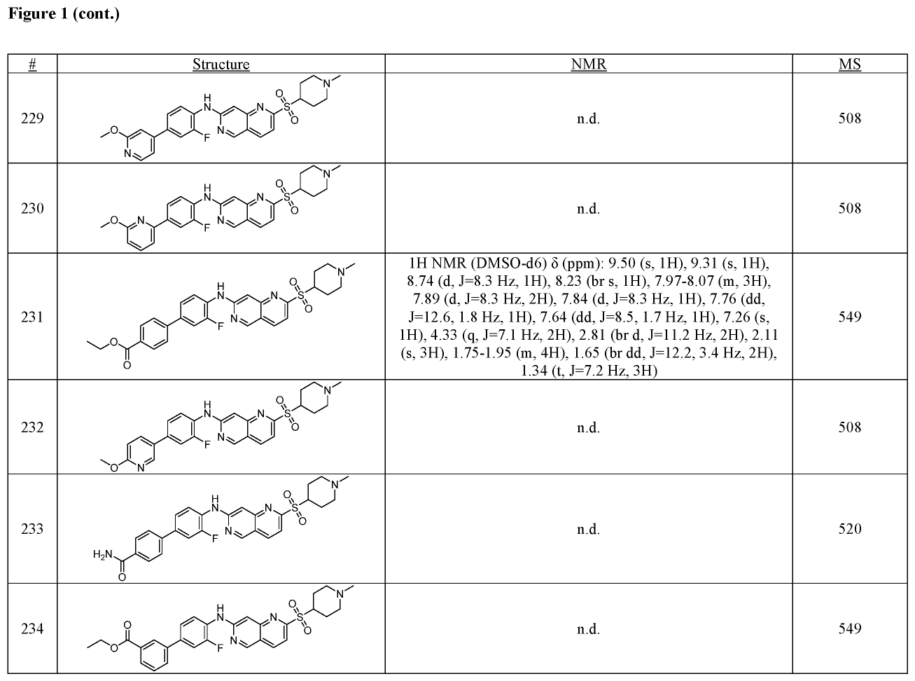

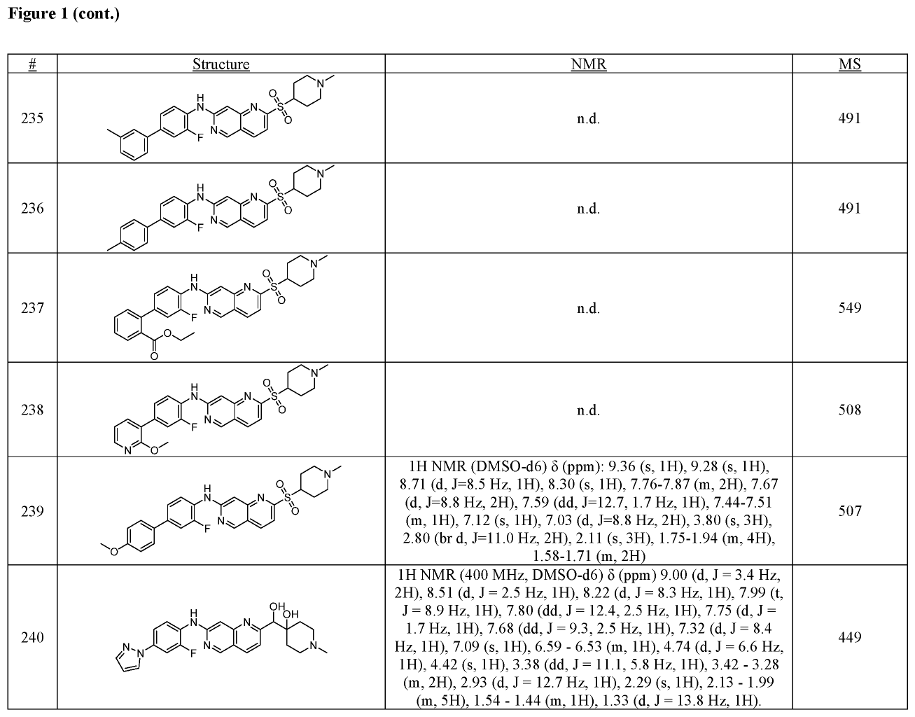

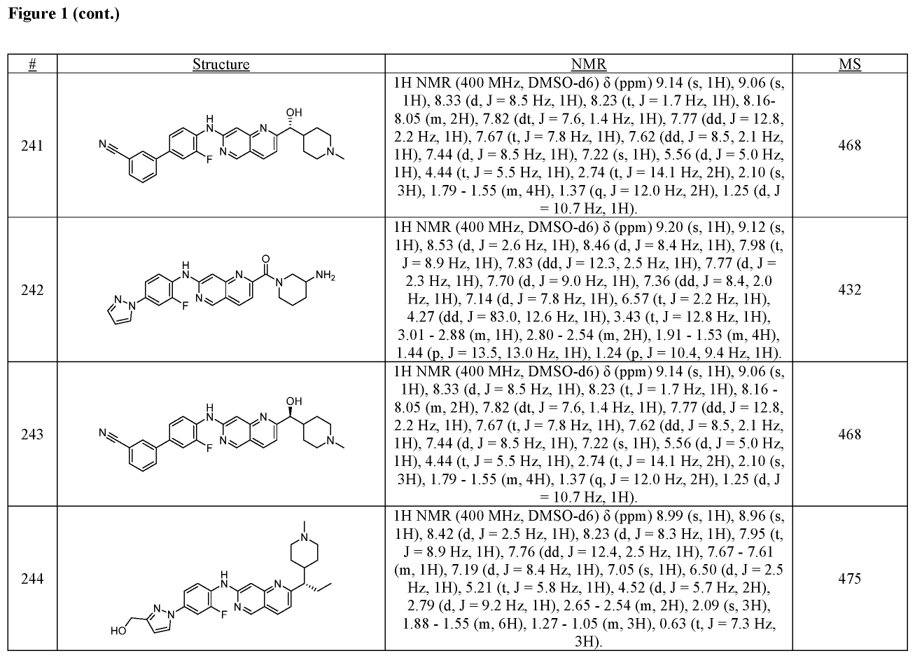

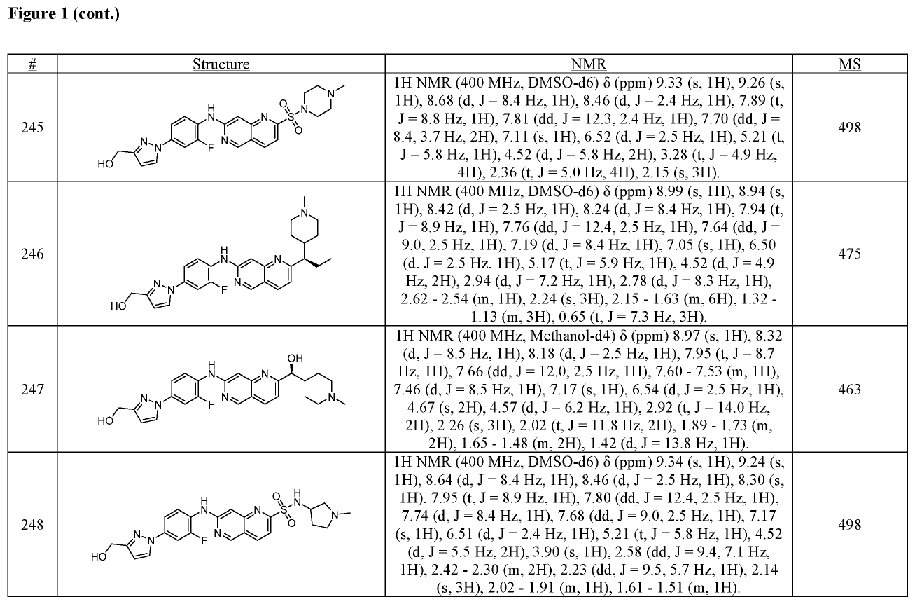

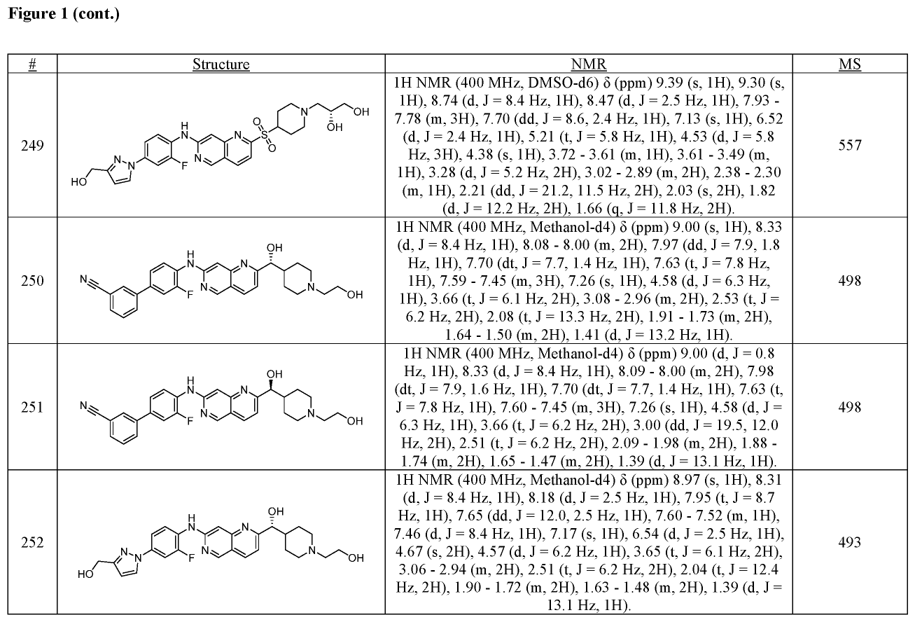

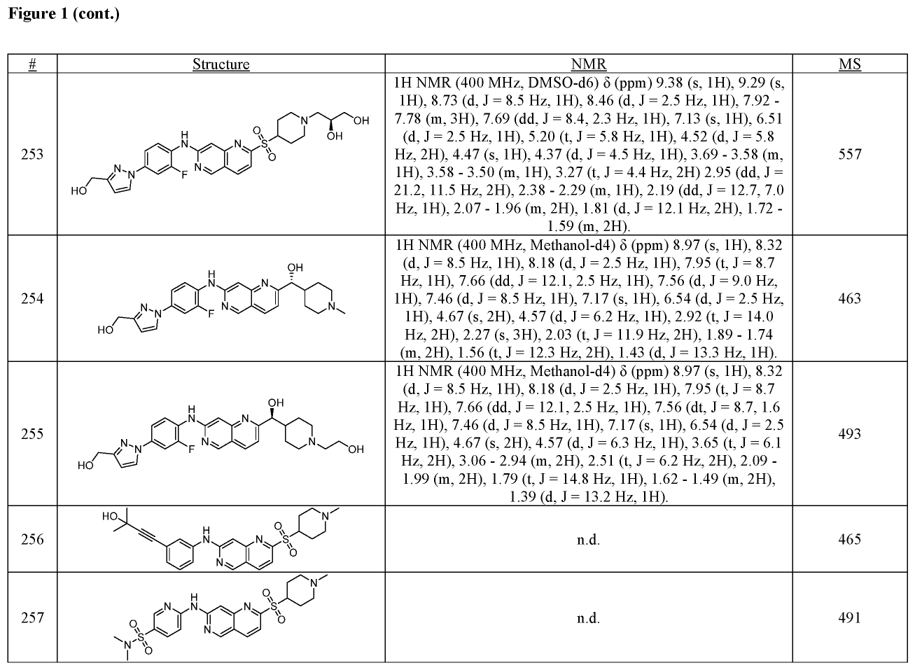

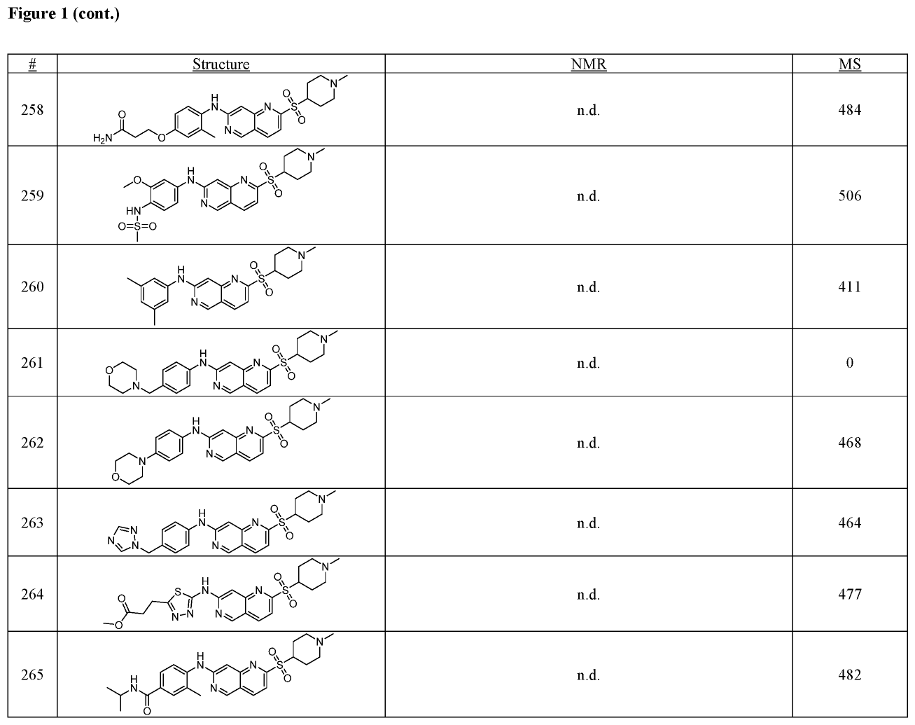

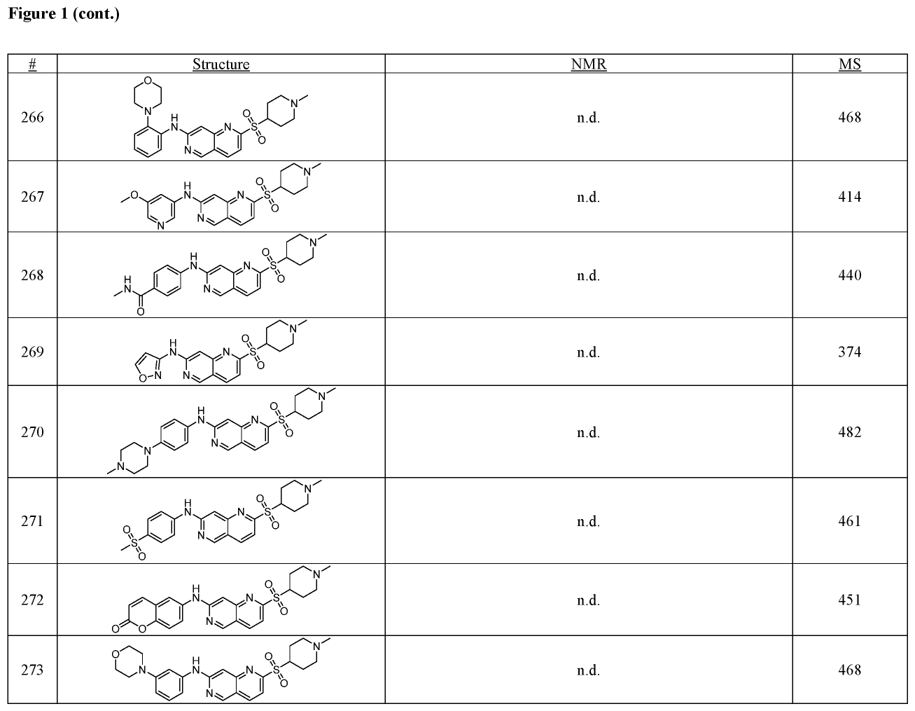

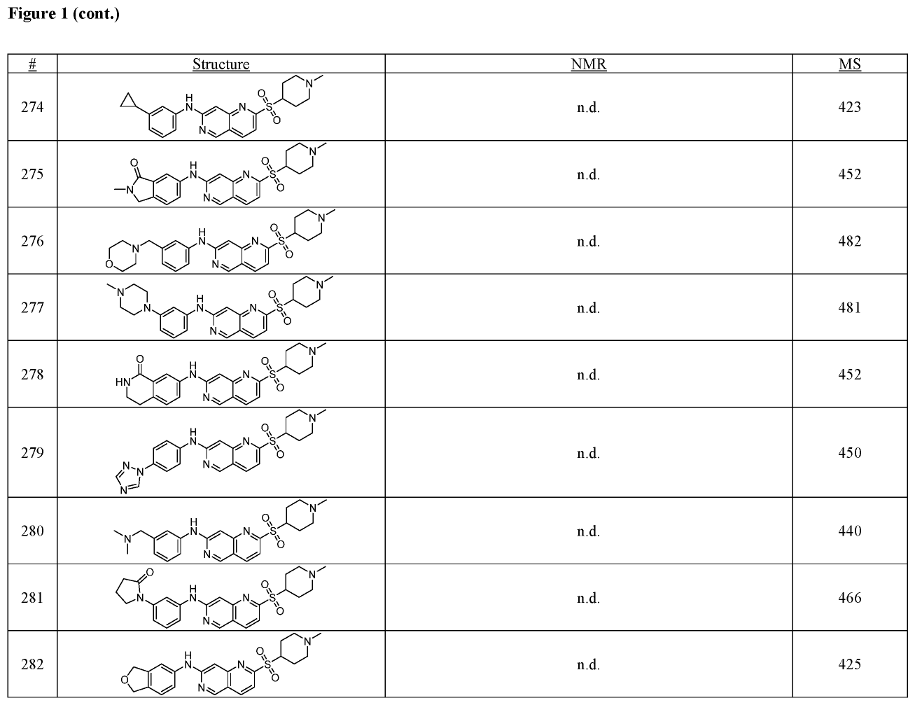

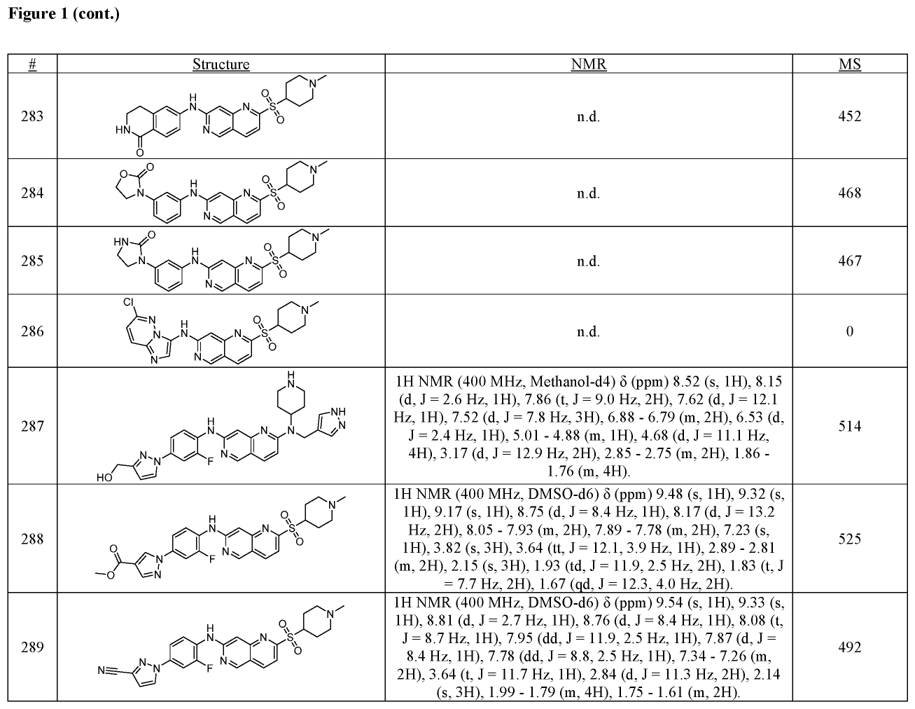

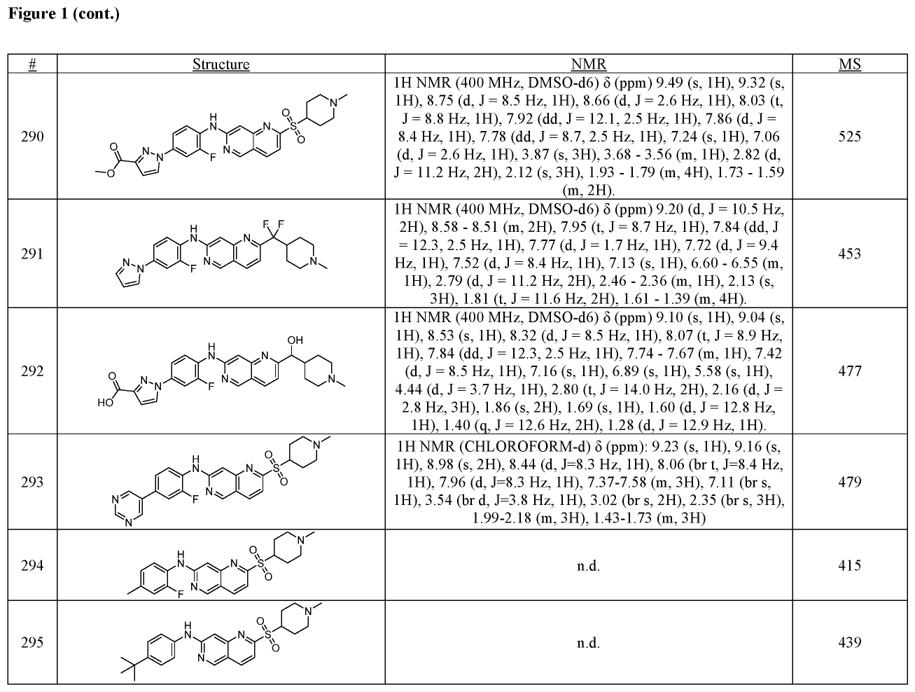

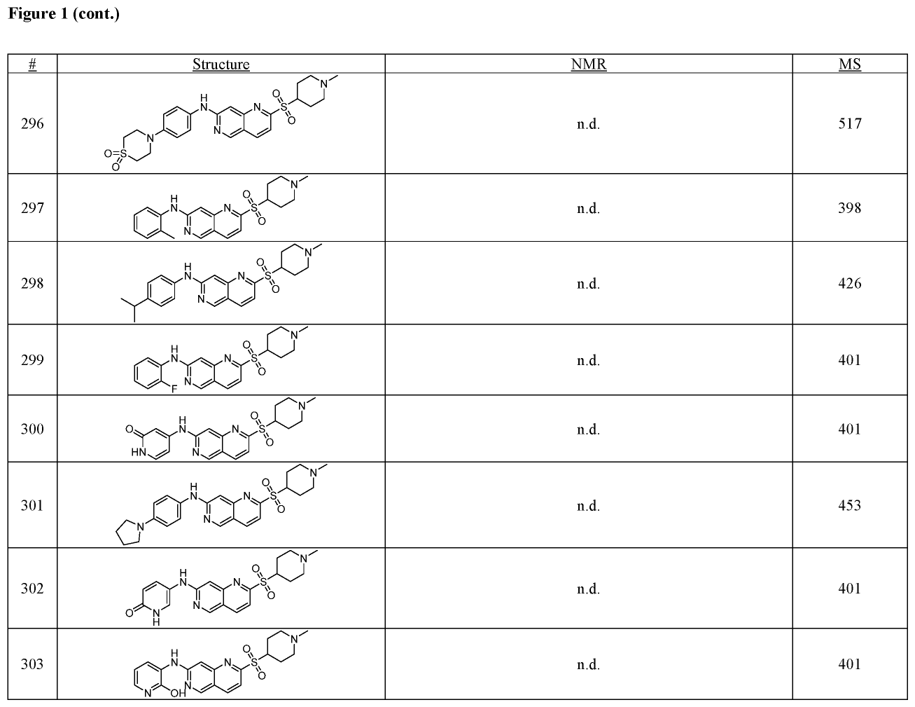

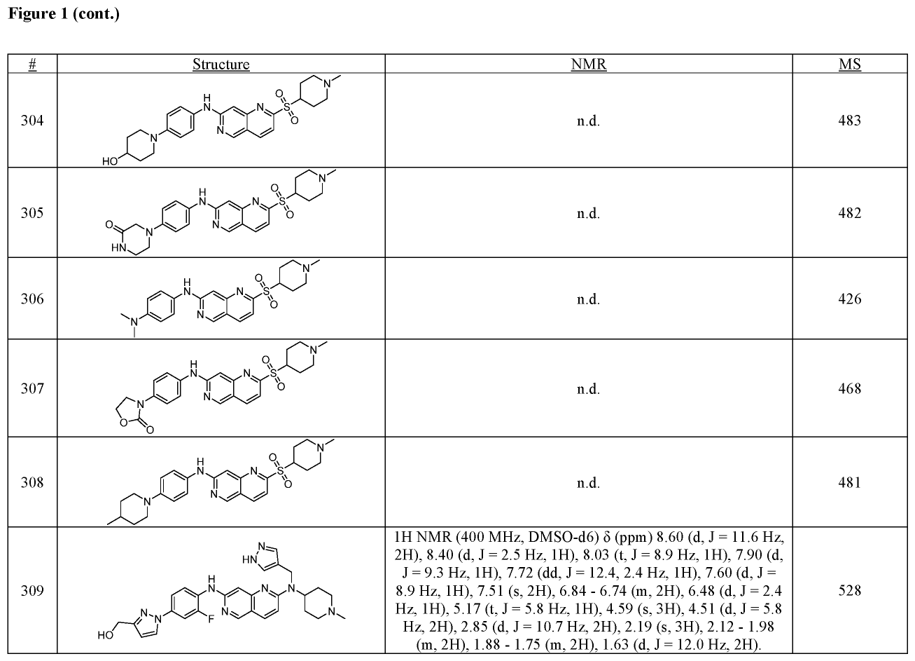

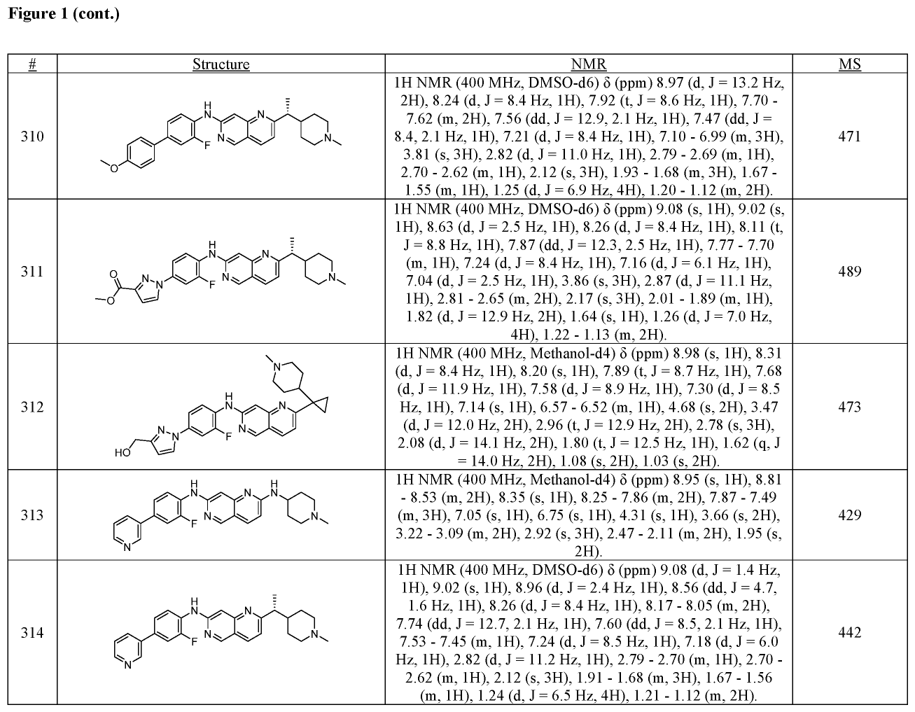

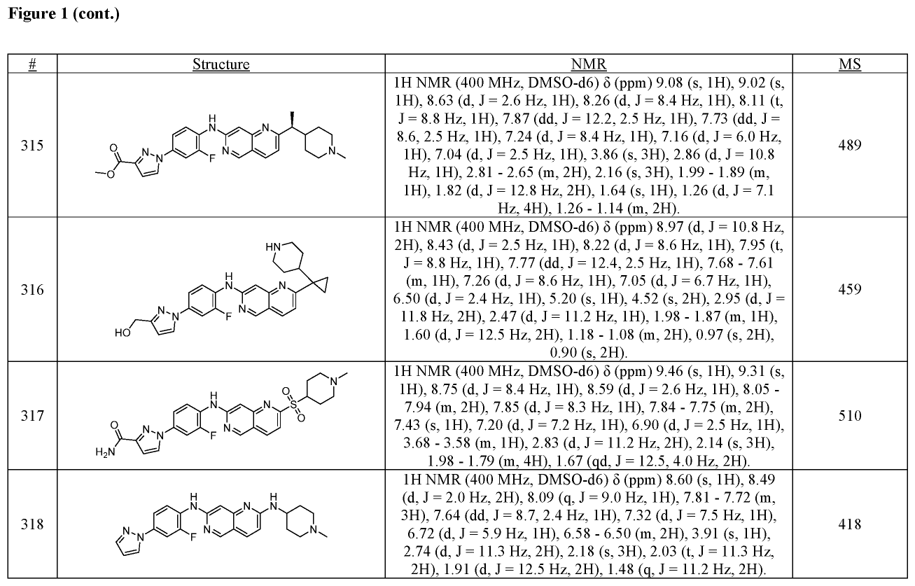

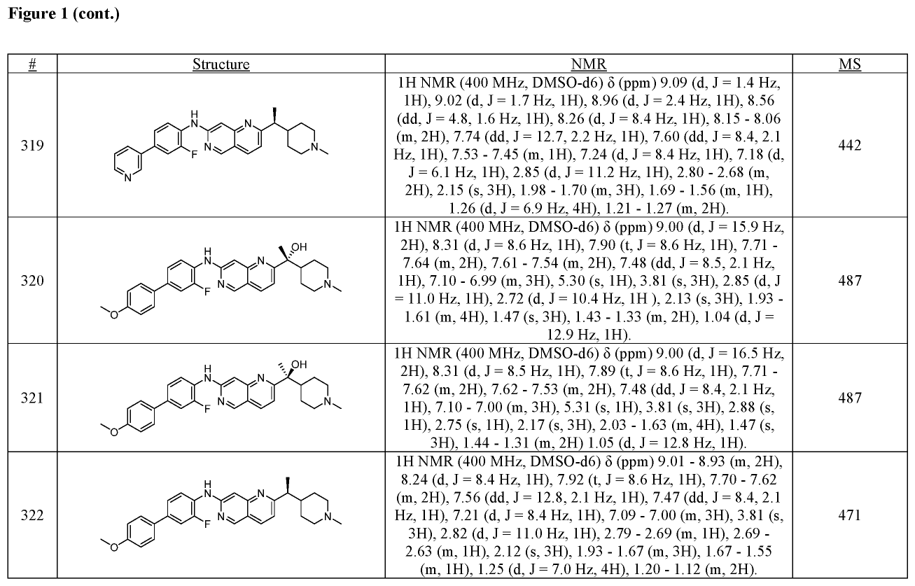

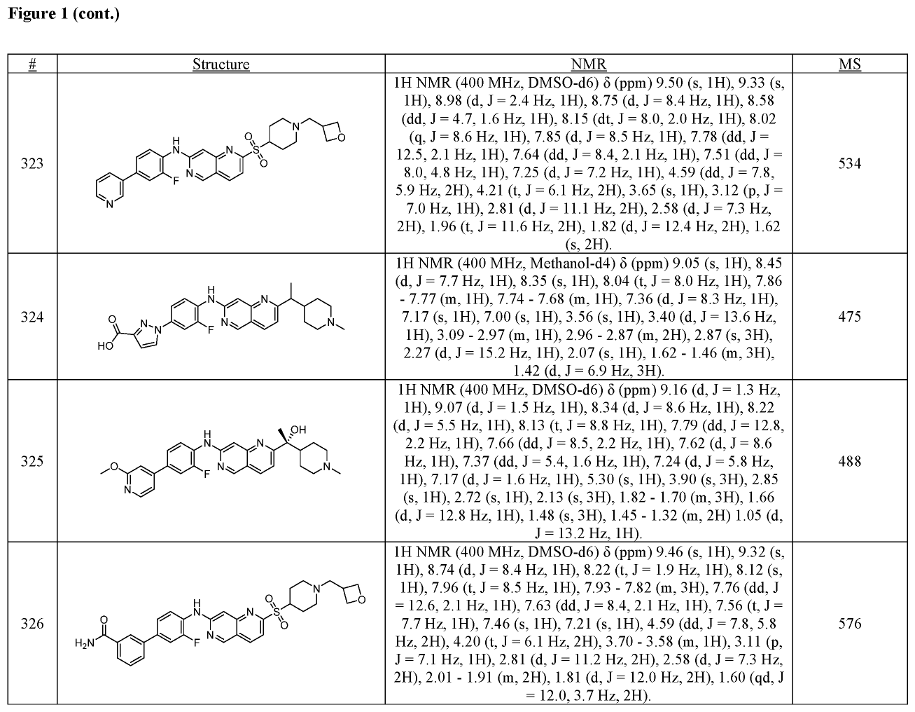

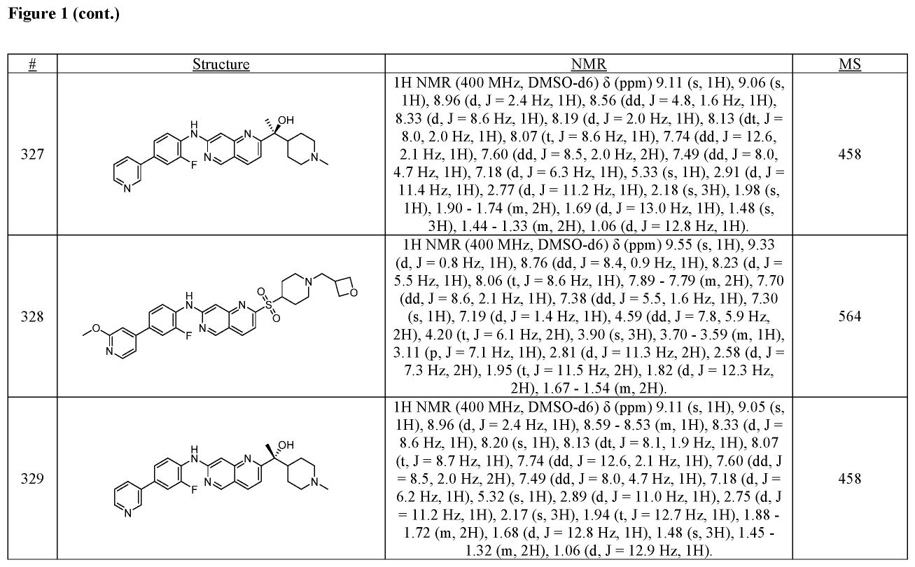

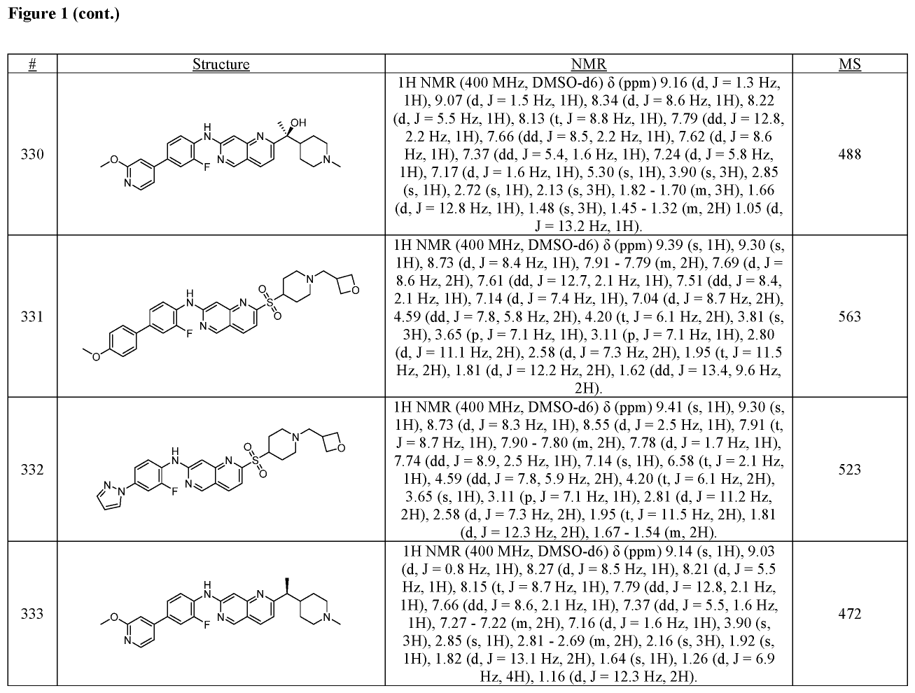

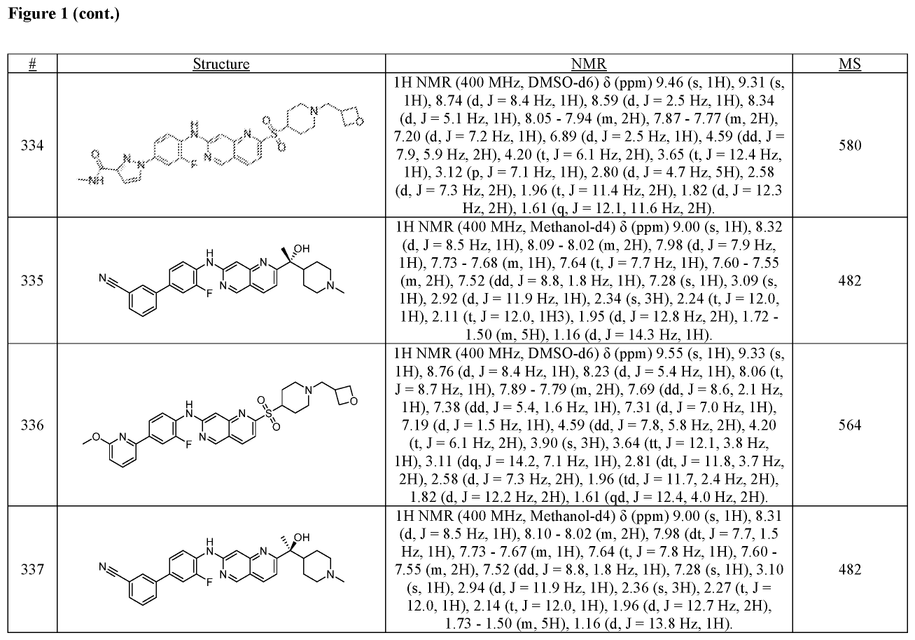

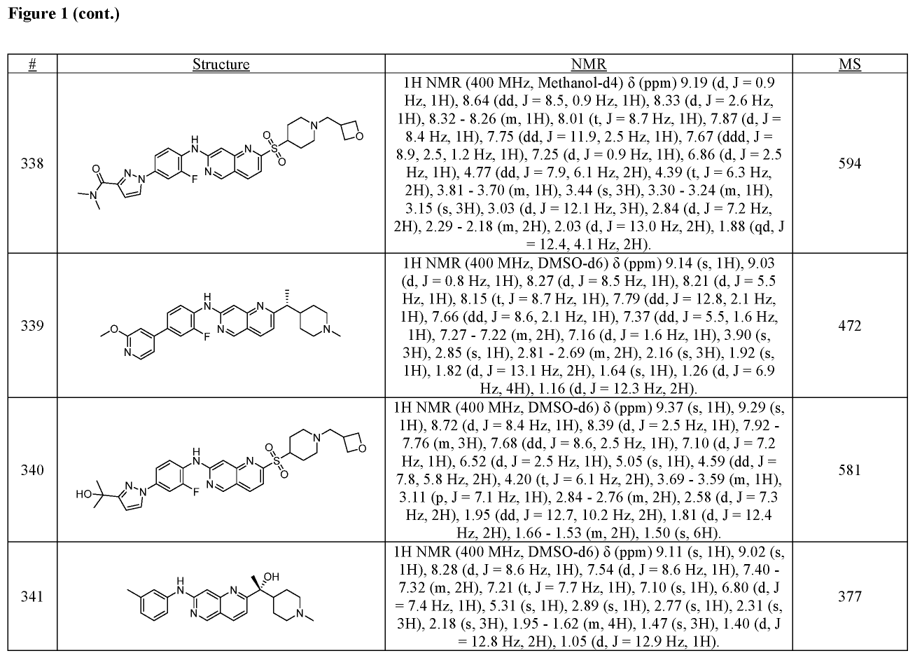

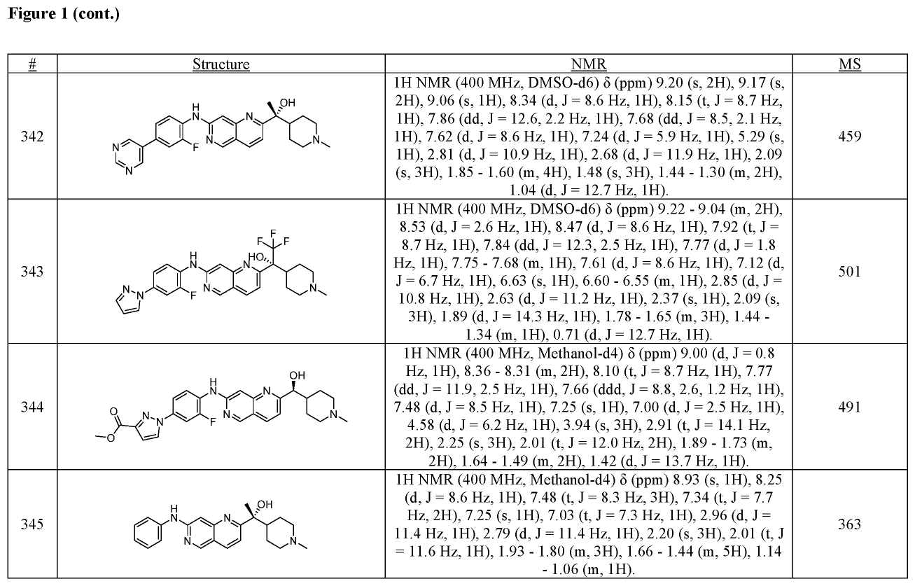

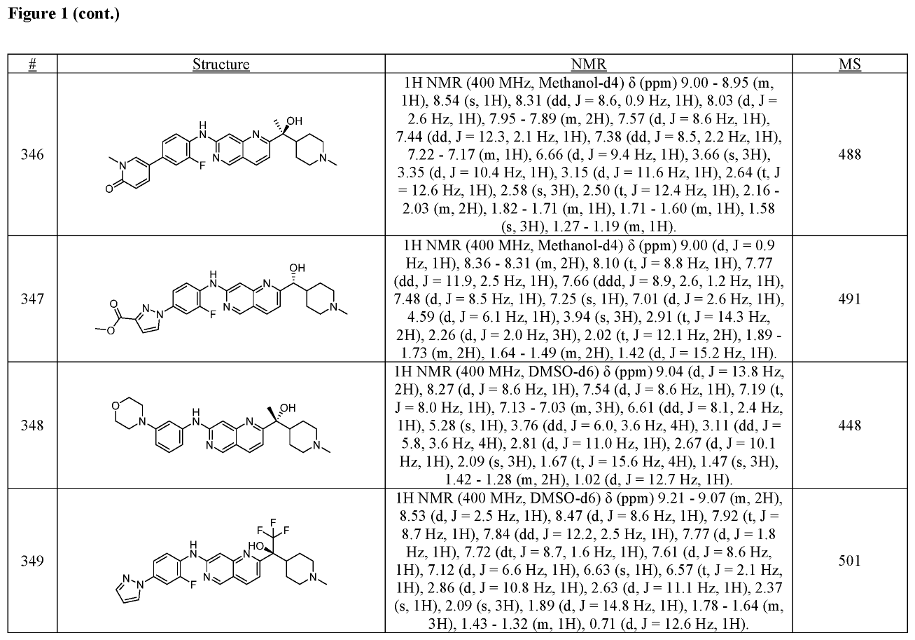

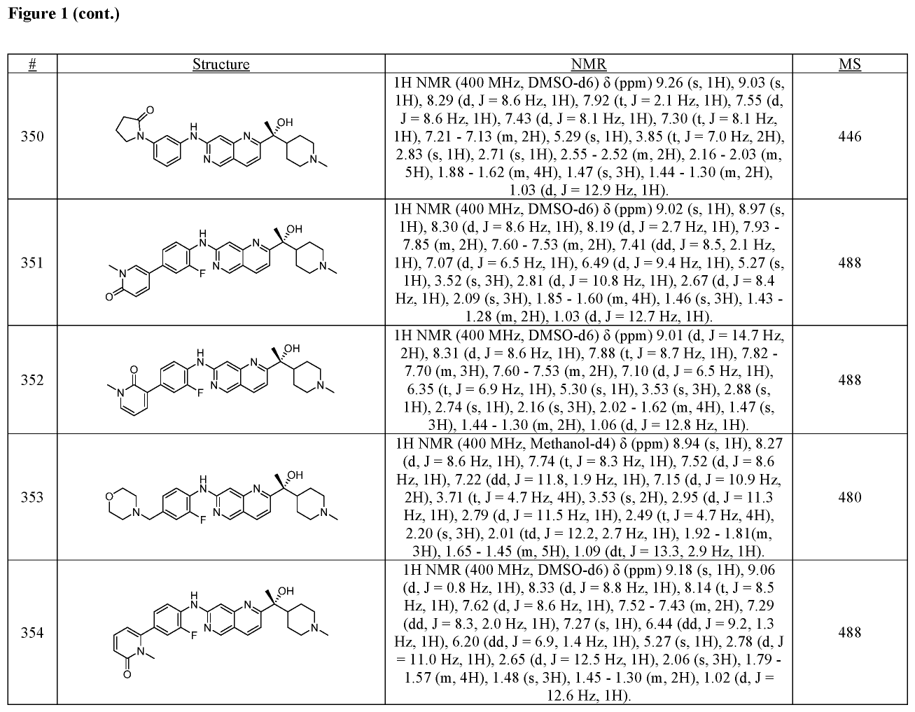

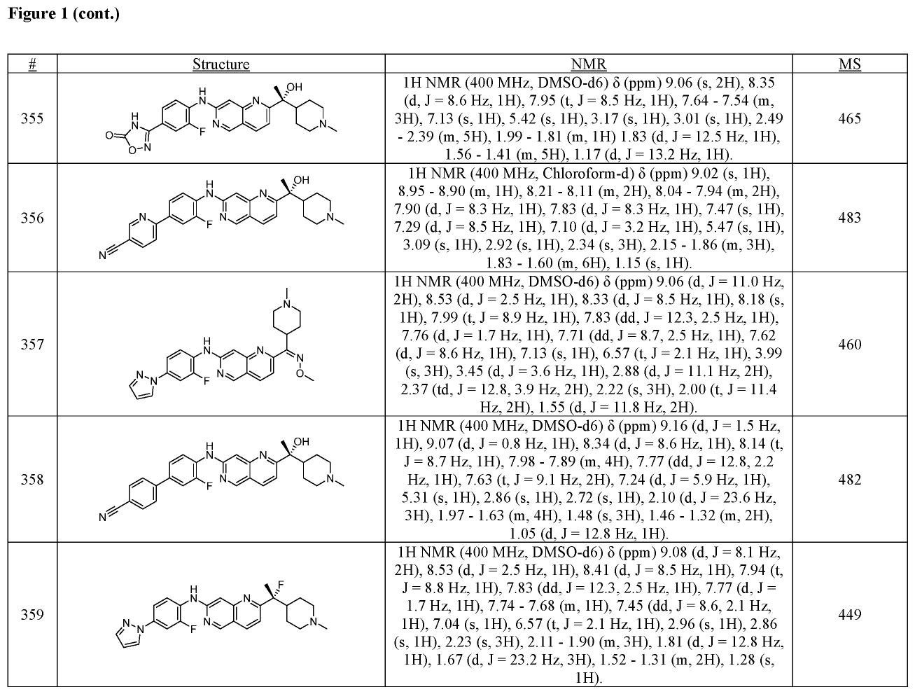

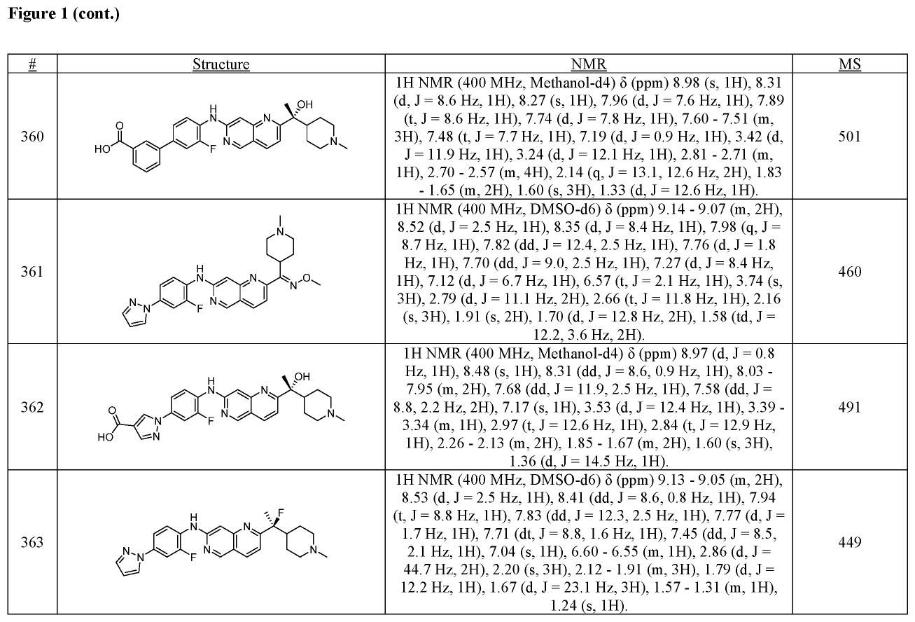

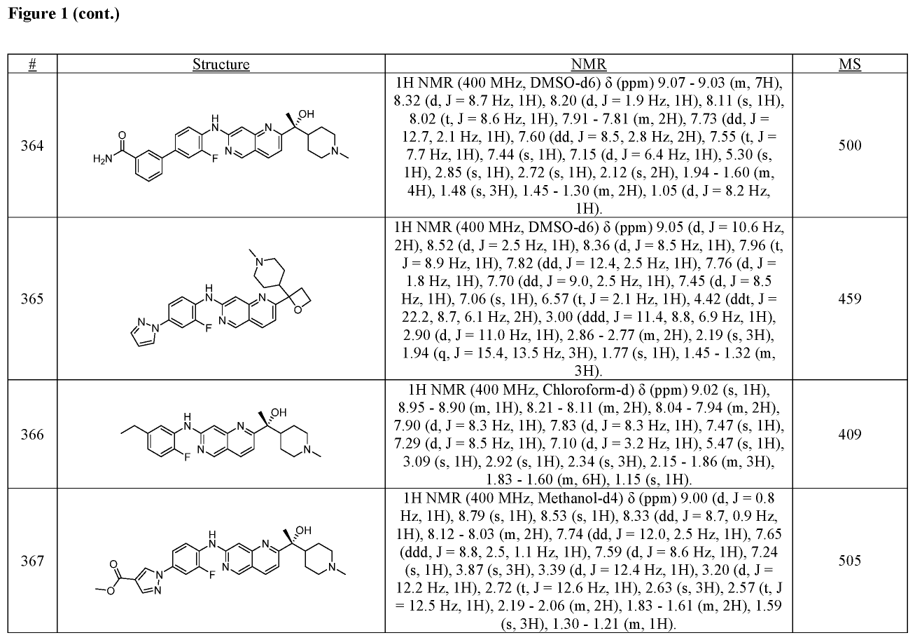

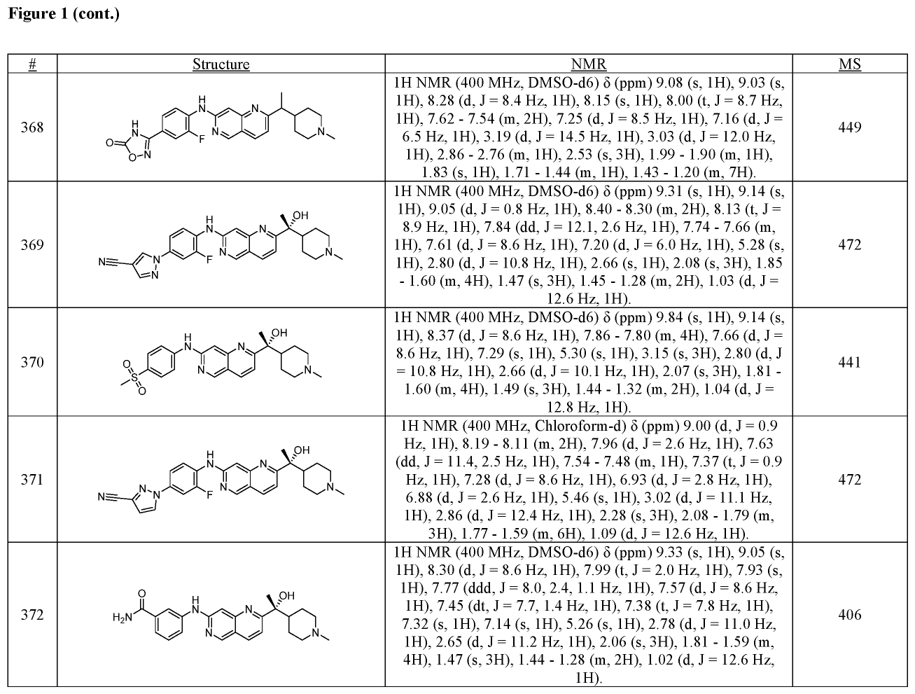

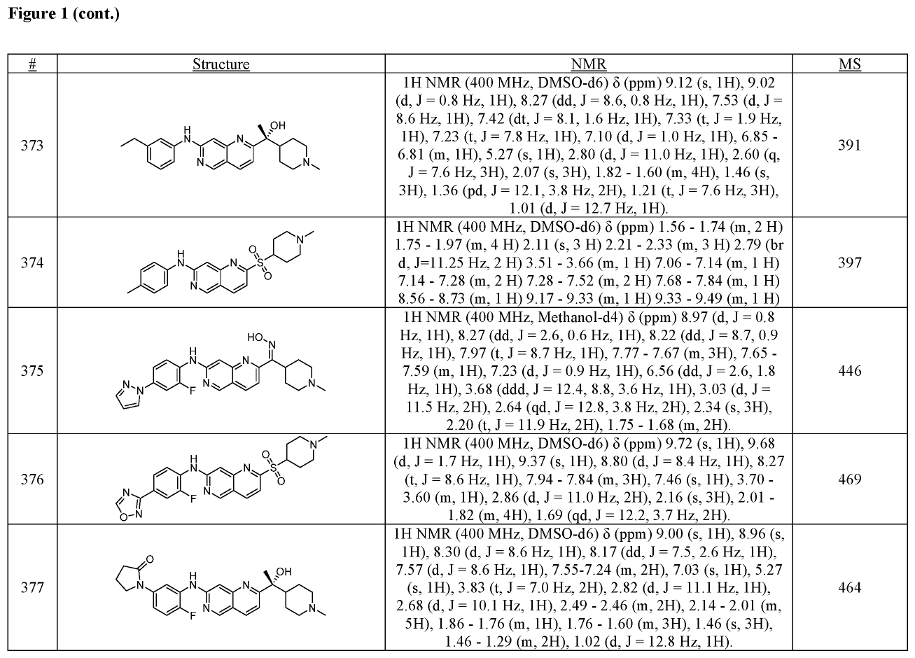

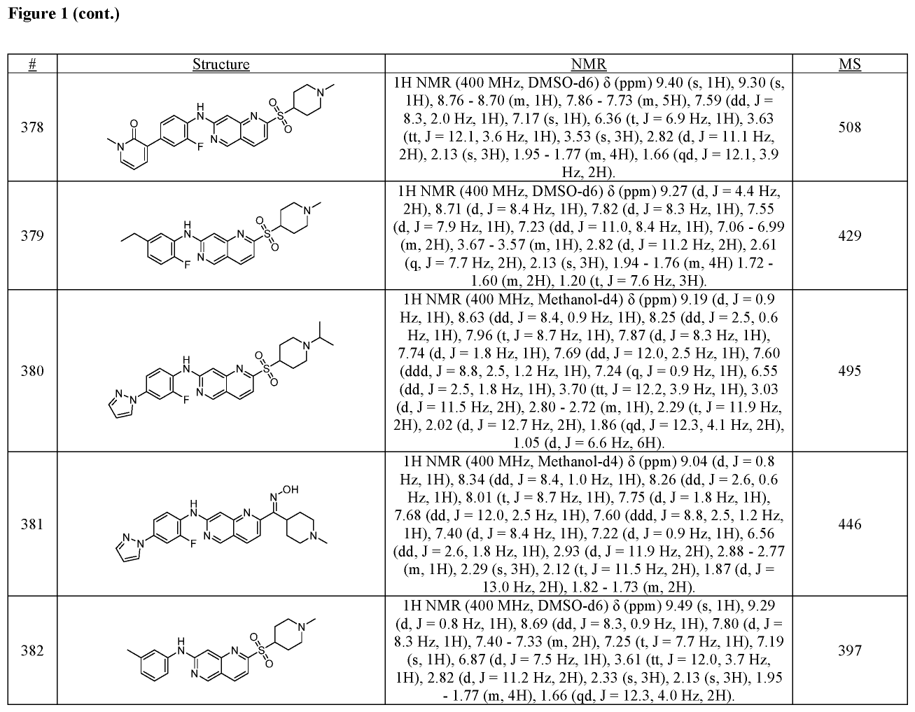

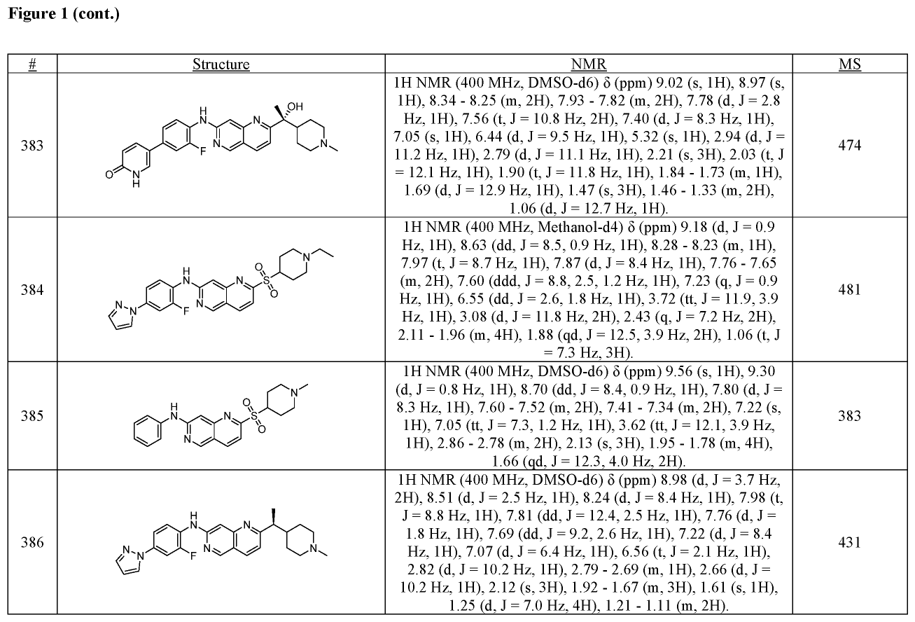

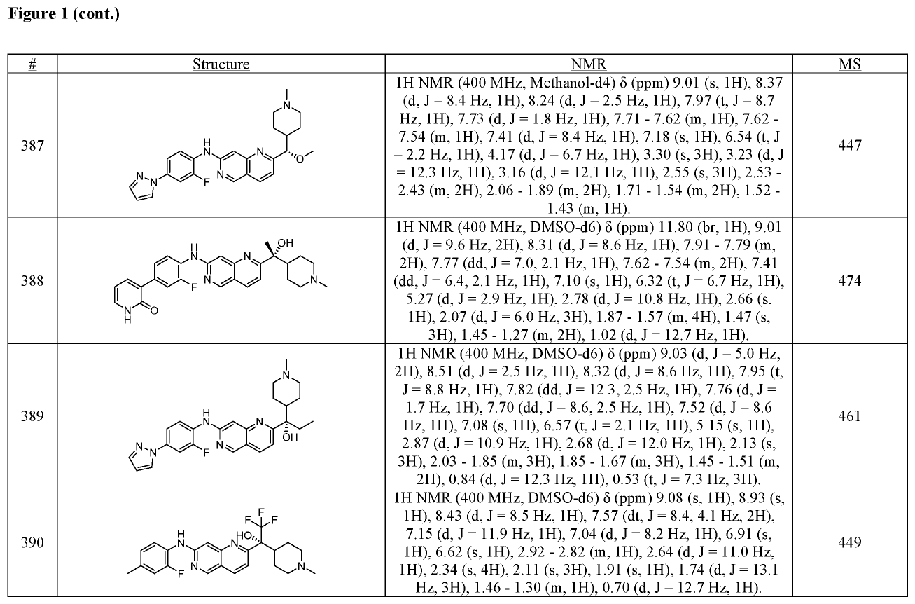

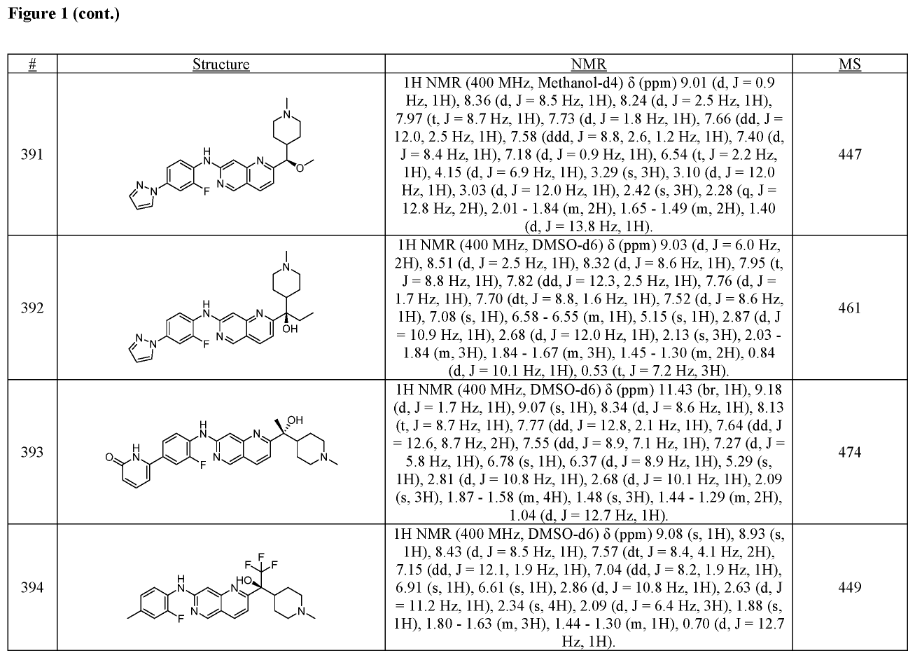

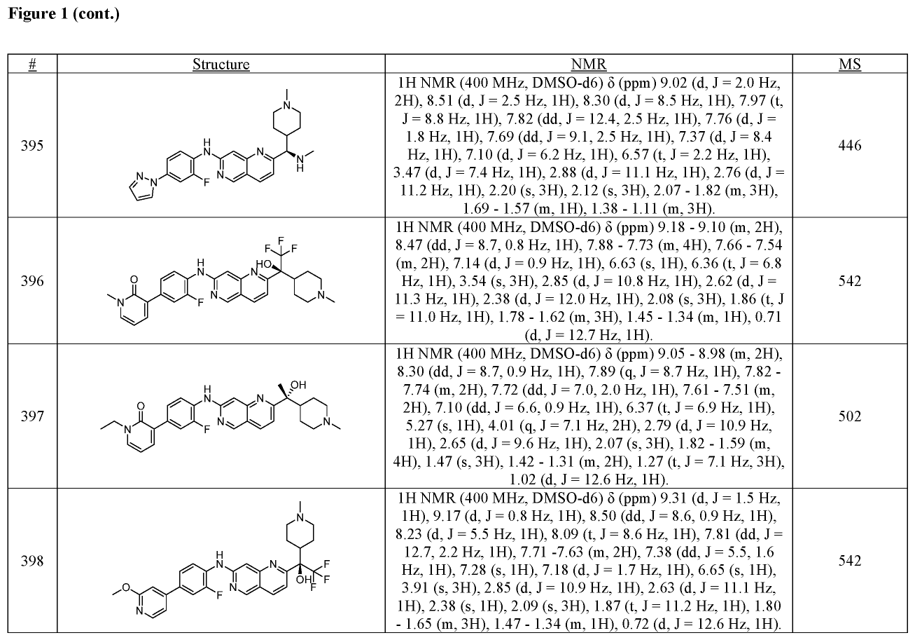

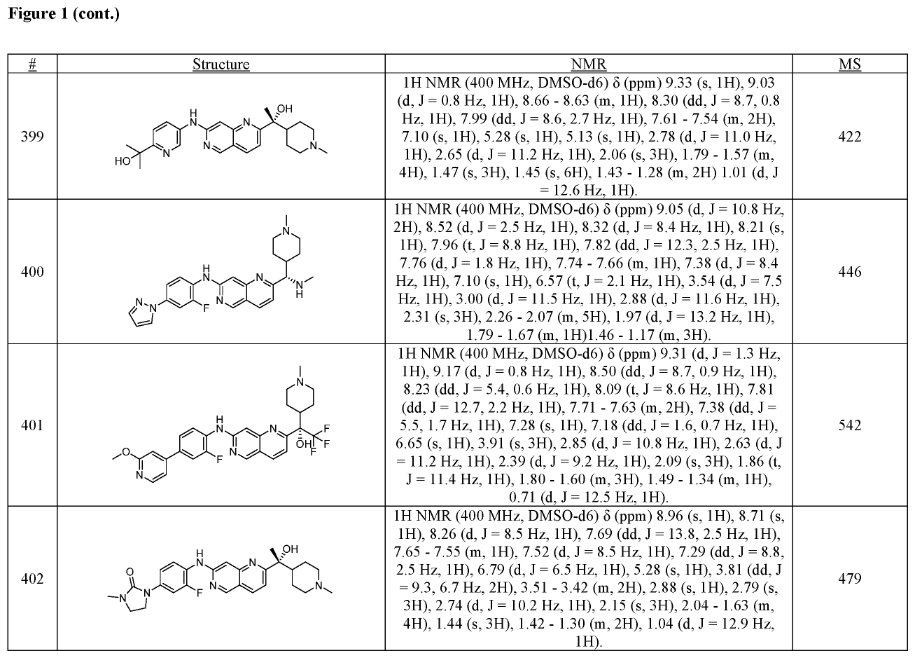

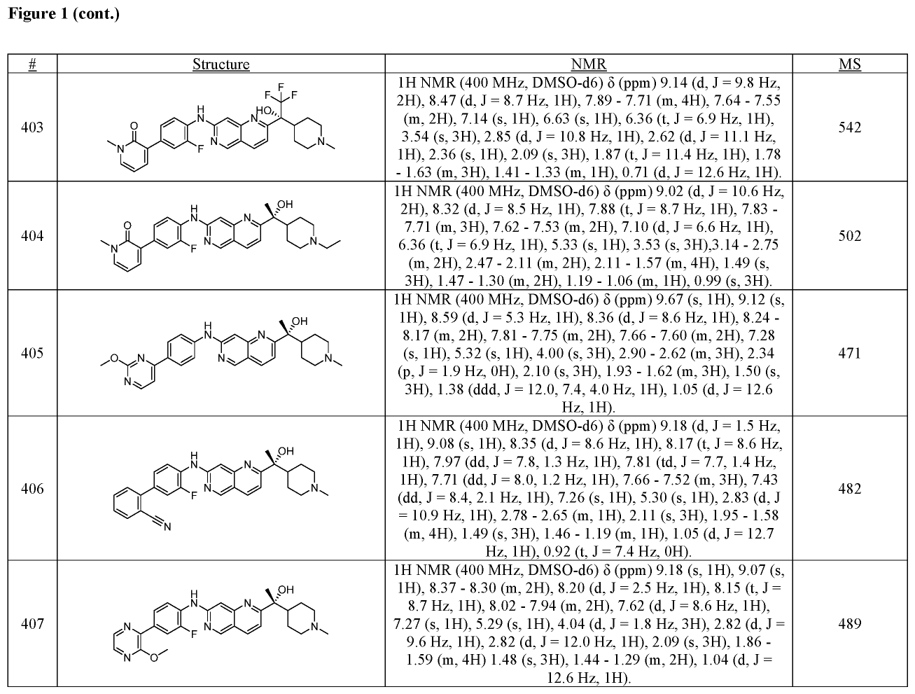

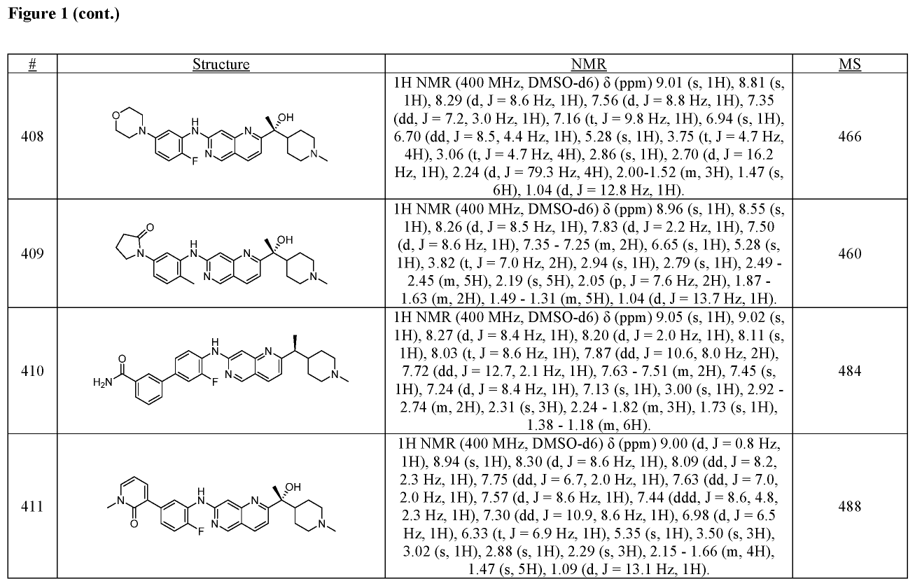

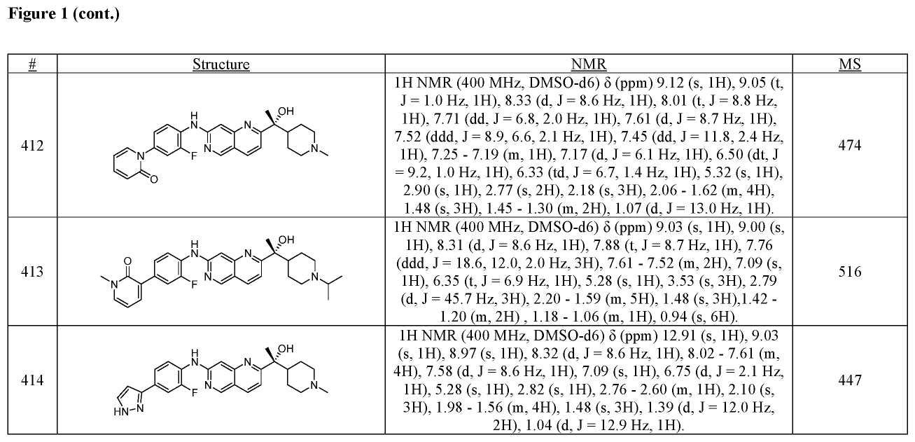

NMR and MS data for exemplary compounds of the invention are included in FIG. 1.

### Example 66. CDK5 and CDK2 Mobility Shift Assays

Compound potency was measured via a change in the enzymatic activity of CDK5/p25, and optionally of CDK2/CycA2. Enzymes, CDK5/p25 and CDK2/CycA2, were sourced from Carna Biosciences (Cat #04-106 and 04-103, respectively). Test compound stocks were diluted in 100% DMSO and serially diluted 3-fold in a 384-well plate using a TECAN EVO200 (TECAN). Staurosporine was used as a reference control compound in all assays.

Twenty nL of compound were transferred into a 384-well plate (Greiner 781201) using an Echo550 (Labcyte Inc). Enzyme, ATP, and 10 mM MgCl₂ were preincubated at room temperature with the compound in assay buffer (50 mM HEPES pH 7.5, 1 mM EGTA, 0.01% Brij-35, 0.05% BSA, 2 mM DTT) for 30 minutes. Peptide substrate (FL peptide 29 (Perkin Elmer) for CDK5/p25; FL Peptide 18 (Perkin Elmer) for CDK2/CycA2) was added to initiate the reaction. The final assay contained 0.154 nM CDK5/p25 or 1.25 nM CDK2/CycA2, 1004 (for CDK5/p25) or 37 μM (for CDK2/CycA2) ATP, and 1.5 μM of the appropriate peptide substrate. The final DMSO concentration was <1%.

The CDK5/p25 reaction was incubated at room temperature for 60 min. The CDK2/CycA2 reaction was incubated at room temperature for 120 min. The reactions were quenched by the addition of 70 μL stopping buffer containing 0.5 M EDTA. Samples were analyzed using the EZ Reader (Perkin Elmer).

The results were expressed as % vehicle, where % vehicle=100×(U−C₂)/(C1−C2), where U is the signal of sample, C₁ is the average of the high controls (signal with no compound added), and C₂ is the average of low controls (signal with the buffer in place of enzyme). The ICso is determined by fitting the percentage of inhibition as a function of compound concentrations using a 4-parameter fit defined as follows.

Y=Bottom+(Top−Bottom)/(1+10^{((LobIC50-X)*Hill Slope)}), where X is the log of the compound concentration, Y is the % vehicle or response at X, Top and Bottom are the plateaus in the same units as Y, and the Hill's Slope is unitless, and the ICso is the half-maximal inhibitory concentration.

TABLE 1
Inhibitory Activity and Specificity of Exemplary Compounds For
CDK2 and CDK5 activity: “A” = less than 10 nM; “B” = between
10 and 100 nM; “C” = between greater than 100 nM and less than
or equal to 1 μM; and “D” = greater than 1 μM. For
specificity: “+++” = greater than 100-fold more active
against CDK5 than CDK2; “++” = greater
than 10-fold and less than or equal to 100-fold more
active against CDK5 than CDK2; and “+” =
10-fold or less more active against CDK5 than CDK2.

| | CDK5 | CDK2 | CDK5/ |
|---|---|---|---|
| # | IC₅₀ | IC₅₀ | CDK2 |
| 100 | C | D | ++ |
| 101 | B | D | ++ |
| 102 | A | C | ++ |
| 103 | B | C | ++ |
| 104 | B | C | ++ |
| 105 | B | C | ++ |
| 106 | D | D | + |
| 107 | A | B | ++ |
| 108 | A | B | ++ |
| 109 | D | D | + |
| 110 | C | D | + |
| 111 | C | D | ++ |
| 112 | C | D | ++ |
| 113 | B | C | ++ |
| 114 | D | D | + |
| 115 | A | B | ++ |
| 116 | C | D | + |
| 117 | C | D | + |
| 118 | A | A | ++ |
| 119 | A | C | +++ |
| 120 | D | D | + |
| 121 | A | B | + |
| 122 | A | B | ++ |
| 123 | B | C | ++ |
| 124 | B | C | ++ |
| 125 | C | D | ++ |
| 126 | B | D | +++ |
| 127 | A | B | +++ |
| 128 | A | D | +++ |
| 129 | A | B | ++ |
| 130 | B | D | ++ |
| 131 | C | D | ++ |
| 132 | A | B | ++ |
| 133 | A | B | ++ |
| 134 | A | B | ++ |
| 135 | A | B | ++ |
| 136 | C | C | + |
| 137 | A | B | ++ |
| 138 | A | B | ++ |
| 139 | A | C | +++ |
| 140 | C | D | + |
| 141 | A | B | ++ |
| 142 | C | D | + |
| 143 | B | D | +++ |
| 144 | A | C | +++ |
| 145 | D | D | + |
| 146 | A | B | +++ |
| 147 | A | C | ++ |
| 148 | B | C | ++ |
| 149 | B | D | ++ |
| 150 | B | D | ++ |
| 151 | A | D | +++ |
| 152 | A | C | ++ |
| 153 | A | B | ++ |
| 154 | B | C | ++ |
| 155 | C | D | ++ |
| 156 | A | C | ++ |
| 157 | A | B | ++ |
| 158 | A | C | +++ |
| 159 | A | C | ++ |
| 160 | D | D | + |
| 161 | B | C | ++ |
| 162 | C | D | ++ |
| 163 | A | C | ++ |
| 164 | C | D | ++ |
| 165 | B | C | ++ |
| 166 | C | C | + |
| 167 | A | C | +++ |
| 168 | A | C | +++ |
| 169 | A | B | ++ |
| 170 | A | C | ++ |
| 171 | A | C | ++ |
| 172 | D | D | + |
| 173 | D | D | + |
| 174 | A | C | ++ |
| 175 | A | C | +++ |
| 176 | C | C | + |
| 177 | B | D | ++ |
| 178 | C | D | + |
| 179 | C | D | ++ |
| 180 | C | D | + |
| 181 | C | D | + |
| 182 | D | D | ++ |
| 183 | A | D | +++ |
| 184 | A | B | ++ |
| 185 | C | D | + |
| 186 | B | D | ++ |
| 187 | B | C | ++ |
| 188 | A | C | +++ |
| 189 | B | D | ++ |
| 190 | A | D | +++ |
| 191 | A | C | ++ |
| 192 | A | B | ++ |
| 193 | A | B | +++ |
| 194 | A | B | ++ |
| 196 | A | C | ++ |
| 197 | B | C | ++ |
| 198 | A | C | +++ |
| 199 | C | C | + |
| 200 | A | B | ++ |
| 201 | A | D | +++ |
| 202 | A | D | +++ |
| 203 | C | D | + |
| 204 | C | D | ++ |
| 205 | c | D | ++ |
| 206 | c | D | + |
| 207 | c | D | + |
| 208 | B | C | + |
| 209 | C | D | ++ |
| 210 | B | D | ++ |
| 211 | A | C | +++ |
| 212 | C | D | + |
| 213 | B | D | ++ |
| 214 | B | C | ++ |
| 215 | C | D | + |
| 216 | A | B | ++ |
| 217 | A | A | ++ |
| 218 | A | B | ++ |
| 219 | A | C | +++ |
| 220 | A | A | + |
| 221 | A | C | +++ |
| 222 | A | B | ++ |
| 223 | A | C | +++ |
| 224 | A | C | ++ |
| 225 | A | C | +++ |
| 226 | A | C | +++ |
| 227 | A | C | +++ |
| 228 | A | D | +++ |
| 229 | A | D | +++ |
| 230 | A | D | +++ |
| 231 | A | D | +++ |
| 232 | A | C | +++ |
| 233 | A | C | +++ |
| 234 | A | D | +++ |
| 235 | A | D | +++ |
| 236 | A | C | +++ |
| 237 | B | D | +++ |
| 238 | A | D | +++ |
| 239 | A | C | +++ |
| 240 | C | D | ++ |
| 241 | A | C | +++ |
| 242 | C | D | ++ |
| 243 | B | D | ++ |
| 244 | B | D | +++ |
| 245 | C | C | + |
| 246 | C | D | ++ |
| 247 | B | D | ++ |
| 248 | B | C | + |
| 249 | A | C | ++ |
| 250 | A | C | ++ |
| 251 | B | D | ++ |
| 252 | A | C | +++ |
| 253 | B | C | ++ |
| 254 | A | C | +++ |
| 255 | C | D | ++ |
| 256 | A | B | +++ |
| 257 | B | B | + |
| 258 | A | D | +++ |
| 259 | A | B | ++ |
| 260 | A | C | +++ |
| 261 | A | C | +++ |
| 262 | A | B | ++ |
| 263 | A | B | ++ |
| 264 | D | D | + |
| 265 | B | D | +++ |
| 266 | D | D | ++ |
| 267 | B | C | ++ |
| 268 | A | C | ++ |
| 269 | B | C | + |
| 270 | A | C | ++ |
| 271 | A | B | + |
| 272 | A | C | ++ |
| 273 | A | C | +++ |
| 274 | A | B | +++ |
| 275 | B | C | ++ |
| 276 | A | C | ++ |
| 277 | A | C | ++ |
| 278 | A | C | ++ |
| 279 | A | C | ++ |
| 280 | B | C | + |
| 281 | A | C | ++ |
| 282 | A | B | ++ |
| 283 | A | B | ++ |
| 284 | A | B | ++ |
| 285 | A | B | ++ |
| 286 | D | D | + |
| 287 | B | D | ++ |
| 288 | A | D | +++ |
| 289 | A | D | +++ |
| 290 | A | C | +++ |
| 291 | A | B | ++ |
| 292 | A | C | ++ |
| 293 | A | C | +++ |
| 294 | A | C | +++ |
| 295 | A | B | ++ |
| 296 | A | B | ++ |
| 297 | A | C | +++ |
| 298 | A | B | +++ |
| 299 | A | C | ++ |
| 300 | D | D | + |
| 301 | A | C | +++ |
| 302 | B | C | ++ |
| 303 | C | D | ++ |
| 304 | A | C | ++ |
| 305 | A | B | ++ |
| 306 | A | B | +++ |
| 307 | A | B | ++ |
| 308 | A | C | +++ |
| 309 | B | D | ++ |
| 310 | B | D | ++ |
| 311 | C | D | ++ |
| 312 | B | D | ++ |
| 313 | A | C | ++ |
| 314 | B | D | ++ |
| 315 | A | C | +++ |
| 316 | B | D | ++ |
| 317 | A | C | +++ |
| 318 | A | C | ++ |
| 319 | A | C | ++ |
| 320 | A | B | ++ |
| 321 | C | D | ++ |
| 322 | A | C | +++ |
| 323 | A | C | +++ |
| 324 | B | D | +++ |
| 325 | A | C | ++ |
| 326 | A | C | +++ |
| 327 | C | D | ++ |
| 328 | A | D | +++ |
| 329 | A | B | ++ |
| 330 | D | D | ++ |
| 331 | A | C | ++ |
| 332 | A | C | +++ |
| 333 | A | C | +++ |
| 334 | A | C | ++ |
| 335 | A | B | ++ |
| 336 | A | C | +++ |
| 337 | C | D | ++ |
| 338 | A | C | +++ |
| 339 | B | D | ++ |
| 340 | B | D | ++ |
| 341 | A | B | ++ |
| 342 | A | B | ++ |
| 343 | C | D | ++ |
| 344 | C | D | ++ |
| 345 | A | B | ++ |
| 346 | C | D | ++ |
| 347 | A | C | +++ |
| 348 | A | C | ++ |
| 349 | A | B | ++ |
| 350 | A | B | ++ |
| 351 | A | B | ++ |
| 352 | A | C | +++ |
| 353 | A | B | ++ |
| 354 | A | B | ++ |
| 355 | A | B | ++ |
| 356 | A | B | ++ |
| 357 | C | D | ++ |
| 358 | A | B | ++ |
| 359 | A | B | ++ |
| 360 | A | B | ++ |
| 361 | D | D | ++ |
| 362 | A | B | ++ |
| 363 | B | D | ++ |
| 364 | A | B | ++ |
| 365 | B | C | ++ |
| 366 | A | B | ++ |
| 367 | A | B | ++ |
| 368 | A | C | ++ |
| 369 | A | C | ++ |
| 370 | A | A | + |
| 371 | A | C | ++ |
| 372 | A | A | ++ |
| 373 | A | B | ++ |
| 374 | A | C | +++ |
| 375 | A | B | ++ |
| 376 | C | C | + |
| 377 | A | B | ++ |
| 378 | B | D | +++ |
| 379 | A | C | +++ |
| 380 | B | C | ++ |
| 381 | B | C | + |
| 382 | A | B | +++ |
| 383 | A | B | ++ |
| 384 | A | C | +++ |
| 385 | A | B | ++ |
| 386 | A | C | ++ |
| 387 | C | D | ++ |
| 388 | A | C | ++ |
| 389 | D | D | ++ |
| 390 | C | D | ++ |
| 391 | C | D | ++ |
| 392 | B | C | ++ |
| 393 | A | B | ++ |
| 394 | A | B | ++ |
| 395 | B | C | ++ |
| 396 | B | C | ++ |
| 397 | A | C | ++ |
| 398 | D | D | ++ |
| 399 | A | B | ++ |
| 400 | B | C | ++ |
| 401 | A | C | ++ |
| 402 | A | B | ++ |
| 403 | C | D | ++ |
| 404 | B | C | ++ |
| 405 | A | B | ++ |
| 406 | A | B | ++ |
| 407 | A | B | ++ |
| 408 | A | C | +++ |
| 409 | B | C | ++ |
| 410 | A | C | +++ |
| 411 | A | C | ++ |
| 412 | A | C | ++ |
| 413 | A | C | ++ |
| 414 | A | B | ++ |

### INCORPORATION BY REFERENCE

All of the U.S. patents and U.S. and PCT published patent applications cited herein are hereby incorporated by reference.

### EQUIVALENTS

The foregoing written specification is sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

Below is the amino acid sequence of the CDK5
protein used in Example 66.
SEP ID NO: 1
MQKYEKLEKIGEGTYGTVFKAKNRETHEIVALKRVRLDDDDEGVP
SSALREICLLKELKHKNIVRLHDVLHSDKKLTLVFEFCDQDLKKY
FDSCNGDLDPEIVKSFLFQLLKGLGFCHSRNVLHRDLKPQNLLIN
RNGELKLADFGLARAFGIPVRCYSAEVVTLWYRPPDVLFGAKLYS
TSIDMWSAGCiFAELANAGRPLFPGNDVDDQLKRIFRLLGTPTEE
QWPSMTKLPDYKPYPMYPATTSLVNVVPKLNATGRDLLQNLLKCN
PVORISAEEALQHPYFSDFCPP

1. A compound having structural formula (I): 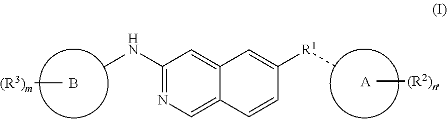
or a pharmaceutically acceptable salt thereof, wherein:
ring A is a monocyclic or bicyclic cycloalkyl or a monocyclic or bicyclic saturated heterocyclyl;
ring B is monocyclic or bicyclic aryl, a monocyclic or bicyclic heteroaryl, or a monocyclic or bicyclic heterocyclyl;
R¹ is —N(R⁵)—, —C(O)—, —S—, —S(O)—, —S(O)₂—, —[C(R⁴)₂]₁₋₂—, —[C(R⁴)₂]₀₋₁—CH═, —N(R⁵)—S(O)₂—, —S(O)₂—N(R⁵), —C(R⁴)₂—N(R⁵)—, —N(R⁵)—C(R⁴)₂—, —C(R⁴)₂—S(O)₂—, —C(═N—OH)—, —C(═N—O—C₁-C₄ alkyl)-, or —S(O)₂—C(R⁴)₂—;
each R² is independently halo, —OH, —C₁-C₆ alkyl, —C₁-C₆ haloalkyl, —C₁-C₆ hydroxyalkyl, —(C₀-C₄ alkylene)-C(O)—OH, —(C₀-C₄ alkylene)-C(O)—O—C₁-C₄ alkyl, —(C₀-C₄ alkylene)-O—C₁-C₄ alkyl, —(C₀-C₄ alkylene)-O—C₁-C₄ hydroxyalkyl, —(C₀-C₄ alkylene)-C(O)—N(R⁶)₂, —(C₀-C₄ alkylene)-N(R⁶)₂, or —(C₀-C₄ alkylene)-saturated heterocyclyl, wherein the saturated heterocyclyl is optionally substituted with halo, —OH, or —CH₃;
each R³ is independently halo; —CN; —OH; —N(R⁶)₂; —C₁-C₄ alkyl; —O—C₁-C₄ alkyl; —O—C₁-C₄ alkylene-C(O)—N(R⁶)₂; —C(O)—O—C₁-C₄ alkyl; —C(O)—N(R⁶)₂; —S(O)₂—N(R⁶)₂; —S(O)₂—C₁-C₄ alkyl; C₂-C₄ alkynyl optionally substituted with one or more —OH; 1,2,4-triazol-1-ylmethyl;
morpholinylmethyl; cyclopropyl; ═O; —CH₂CH₂—C(O)—O—CH₃; —N(R⁶)—S(O)₂—CH₃; an optionally substituted aryl; an optionally substituted heteroaryl; or an optionally substituted heterocyclyl, wherein any alkyl portion of R³ is optionally substituted with one or more of halo, —CN, or —N(R⁶)₂, or —OH;
each R⁴ is independently hydrogen, halo, —OH, —CN, —N(R⁶)₂, —C₁-C₄ alkyl optionally substituted with one or more of —OH, halo, —CN, or —N(R⁶)₂; or O—C₁-C₄ alkyl optionally substituted with one or more of —OH, halo, —CN, or —N(R⁶)₂;
or one R⁴ is taken together with a ring carbon atom in ring A to form a cycloalkyl or heterocyclyl ring that is spirofused, fused or bridged to ring A;
or two R⁴ bound to the same carbon atom are taken together to form ═CH₂—(C₀-C₃ alkyl), a C₃-C₆ cycloalkyl, or a C₄-C₇ heterocyclyl;
R⁵ is hydrogen; C₁-C₄ alkyl optionally substituted with one or more of —CN, —OH, —COOH, C(O)—O—C₁-C₄ alkyl, or pyrazolyl; —S(O)₂—C₁-C₄ alkyl; —C(O)C(O)OH; —COOH; or —C(O)—O—C₁-C₄ alkyl;
or R⁵ is taken together with a ring carbon atom in ring A to form a heterocyclyl ring that is spirofused, fused or bridged to ring A;
each R⁶ is independently hydrogen or —C₁-C₄ alkyl;
m is 0, 1, 2, 3, 4, 5, or 6;
n is 0, 1, 2, 3, 4, 5, or 6; and
“ [symbol] ” represents a single bond or a double bond.

2. The compound or salt of claim 1, wherein each R³ is independently halo; —CN; —OH; —N(R⁶)₂; —C₁-C₄ alkyl; —O—C₁-C₄ alkyl; —O—C₁-C₄ alkylene-C(O)—N(R⁶)₂; —C(O)—O—C₁-C₄ alkyl; —C(O)—N(R⁶)₂; —S(O)₂—N(R⁶)₂; —S(O)₂—C₁-C₄ alkyl; an optionally substituted aryl; an optionally substituted heteroaryl; or an optionally substituted heterocyclyl, wherein any alkyl portion of R³ is optionally substituted with one or more of halo, —CN, or —N(R⁶)₂, or —OH.

3. The compound or salt of claim 1, wherein ring B is phenyl, —C(O)-phenyl, 1,3,4-thiadiazol-2-yl, imidazo[1,2-b]pyridazin-3-yl, isoxazol-3-yl, 1,3-dihydroisobenzofuran-5-yl, 2H-chromen-6-yl, 1,2,3,4-tetrahydroisoquinolin-6-yl, 1,2,3,4-tetrahydroisoquinolin-7-yl, isoindolin-5-yl, 1,2-dihydropyridin-3-yl, 1,2-dihydropyridin-5-yl, pyridinyl, or pyrimidinyl.

4. (canceled)

5. (canceled)

6. The compound or salt of claim 1, wherein the portion of the compound represented by is: 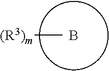
1,3-dihydroisobenzofuran-5-yl, 1-fluoro-2-methylisoindolin-6-yl, 1-oxo-1,2,3,4-tetrahydroisoquinolin-6-yl, 1-oxo-1,2,3,4-tetrahydroisoquinolin-7-yl, 2-(1-hydroxy-1-methylethan-1-yl)pyridin-5-yl, 2-(morpholin-4-yl)phenyl, 2-fluoro-4-(1,2,4-oxadiazol-3-yl)phenyl, 2-fluoro-4-(1,2,4-triazol-1-ylmethyl)phenyl, 2-fluoro-4-(1-ethyl-2-oxo-1,2 dihydropyridin-3-yl)phenyl, 2-fluoro-4-(1-methyl-2-oxo-1,2-dihydropyridin-3-yl)phenyl, 2-fluoro-4-(1-methyl-2-oxo-1,2-dihydropyridin-5-yl)phenyl, 2-fluoro-4-(1-methyl-2-oxo-1,2-dihydropyridin-6-yl)phenyl, 2-fluoro-4-(2-carbamylphenyl)phenyl, 2-fluoro-4-(2-cyanophenyl)phenyl, 2-fluoro-4-(2-ethoxycarbonylphenyl)phenyl, 2-fluoro-4-(2-methoxypyridin-3-yl)phenyl, 2-fluoro-4-(2-methoxypyridin-4-yl)phenyl, 2-fluoro-4-(2-methoxypyridin-5-yl)phenyl, 2-fluoro-4-(2-methoxypyridin-6-yl)phenyl, 2-fluoro-4-(2-oxo-1,2-dihydropyridin-1-yl)phenyl, 2-fluoro-4-(2-oxo-1,2-dihydropyridin-3-yl)phenyl, 2-fluoro-4-(2-oxo-1,2-dihydropyridin-5-yl)phenyl, 2-fluoro-4-(2-oxo-1,2-dihydropyridin-6-yl)phenyl, 2-fluoro-4-(2-oxo-3-methylimidazolidin-1yl)phenyl, 2-fluoro-4-(3-(1-hydroxy-1-methylethan-1-yl)pyrazol-1-yl)phenyl, 2-fluoro-4-(3-carbamylphenyl)phenyl, 2-fluoro-4-(3-carbamylpyrazol-1-yl)phenyl, 2-fluoro-4-(3-carboxyphenyl)phenyl, 2-fluoro-4-(3-carboxypyrazol-1-yl)phenyl, 2-fluoro-4-(3-cyanophenyl)phenyl, 2-fluoro-4-(3-cyanopyrazol-1-yl)phenyl, 2-fluoro-4-(3-ethoxycarbonylphenyl)phenyl, 2-fluoro-4-(3-fluorophenyl)phenyl, 2-fluoro-4-(3-hydroxymethylpyrazol-1-yl)phenyl, 2-fluoro-4-(3-methoxycarbonylpyrazol-1-yl)phenyl, 2-fluoro-4-(3-methoxyphenyl)phenyl, 2-fluoro-4-(3-methoxypyrazin-2-yl)phenyl, 2-fluoro-4-(3-methylcarbamylpyrazol-1-yl)phenyl, 2-fluoro-4-(3-methylphenyl)phenyl, 2-fluoro-4-(3-N,N-dimethylcarbamylpyrazol-1-yl)phenyl, 2-fluoro-4-(4-carbamylphenyl)phenyl, 2-fluoro-4-(4-carboxypyrazol-1-yl)phenyl, 2-fluoro-4-(4-cyanophenyl)phenyl, 2-fluoro-4-(4-cyanopyrazol-1-yl)phenyl, 2-fluoro-4-(4-ethoxycarbonylphenyl)phenyl, 2-fluoro-4-(4-fluorophenyl)phenyl, 2-fluoro-4-(4-methoxycarbonylpyrazol-1-yl)phenyl, 2-fluoro-4-(4-methoxyphenyl)phenyl, 2-fluoro-4-(4-methylphenyl)phenyl, 2-fluoro-4-(5-cyanopyridin-2-yl)phenyl, 2-fluoro-4-(5-hydroxymethylpyrazol-1-yl)phenyl, 2-fluoro-4-(5-oxo-4,5-dihydro-1,2,4-oxadiazol-3-yl)phenyl, 2-fluoro-4-(morpholin-4-ylmethyl)phenyl, 2-fluoro-4-(pyrazol-1-yl)phenyl, 2-fluoro-4-(pyrazol-3-yl)phenyl, 2-fluoro-4-(pyridin-3-yl)phenyl, 2-fluoro-4-(pyridin-4-yl)phenyl, 2-fluoro-4-(pyrimidin-5-yl)phenyl, 2-fluoro-4-methylphenyl, 2-fluoro-5-(1-methyl-2-oxo-1,2-dihydropyridin-3-yl)phenyl, 2-fluoro-5-(2-oxopyrrolidin-1-yl)phenyl, 2-fluoro-5-(morpholin-4-yl)phenyl, 2-fluoro-5-ethylphenyl, 2-fluorophenyl, 2-hydroxypyridin-3-yl, 2-methyl-4-(2-carbamylethoxy)phenyl, 2-methyl-4-(2-oxopyrrolindin-1-yl)phenyl, 2-methyl-4-isopropylcarbamylphenyl, 2-methylphenyl, 2-oxo-1,2-dihydropyridin-4-yl, 2-oxo-1,2-dihydropyridin-5-yl, 2-oxo-2H-chromen-6-yl, 3-(1,2,3,4-tetrazol-1-yl)phenyl, 3-(2-oxoimidazolidin-1-yl)phenyl, 3-(2-oxo-oxazolidin-3-yl)phenyl, 3-(2-oxopyrrolidin-1-yl)phenyl, 3-(3-hydroxy-3-methylbutan-1-yn-1-yl)phenyl, 3-(4-methylpiperazin-1-yl)phenyl, 3-(aminosulfonyl)phenyl, 3-(cyanomethyl)phenyl, 3-(ethoxycarbonyl)phenyl, 3-(methylsulfonyl)phenyl, 3-(morpholin-4-yl)phenyl, 3-(morpholin-4-ylmethyl)phenyl, 3,5-dimethylphenyl, 3-aminophenylcarbonyl, 3-carbamylphenyl, 3-cyanophenyl, 3-cyclopropylphenyl, 3-ethylphenyl, 3-methoxy-4-methylsulfonylaminophenyl, 3-methylphenyl, 4-(1,1-dioxoisothiazolidin-2-yl)phenyl, 4-(1,1-dioxothiomorpholin-4-yl)phenyl, 4-(1,2,3,4-tetrazol-5-yl)phenyl, 4-(1,2,4-triazol-1-yl)phenyl, 4-(2-methoxypyrimdin-4-yl)phenyl, 4-(2-oxo-oxazolidin-3-yl)phenyl, 4-(3-oxomorpholin-4-yl)phenyl, 4-(3-oxopiperazin-1-yl)phenyl, 4-(4-hydroxypiperidin-1-yl)phenyl, 4-(4-methylpiperazin-1-yl)phenyl, 4-(4-methylpiperidin-1-yl)phenyl, 4-(5-oxo-4,5-dihydro-1,2,4-oxadiazol-3-yl)phenyl, 4-(morpholin-4-yl)phenyl, 4-(morpholin-4-ylmethyl)phenyl, 4-(N,N-dimethylaminomethyl)phenyl, 4-(N,N-dimethylaminosulfonyl)phenyl, 4-(pyrrolidin-1-yl)phenyl, 4-(tetrahydropyran-4-yl)phenyl, 4-cyanomethylphenyl, 4-dimethylaminophenyl, 4-isopropylphenyl, 4-methylcarbamylphenyl, 4-methylphenyl, 4-methylsulfonylphenyl, 4-t-butylphenyl, 5-(2-methoxycarbonylethan-1-yl)-1,3,4-thiadiazol-2-yl, 5-methoxypyridin-3-yl, 7-chloroimidazo[1,2-b]pyridazin-3-yl, isoxazol-3-yl, phenyl, or pyrimidin-5-yl.

7. The compound or salt of claim 1, wherein ring A is piperidinyl, piperidinylidene, piperazinyl, pyrrolidinyl, azetidinyl, cyclohexyl, cyclopentyl, cyclobutyl, azabicyclo[3.3.1]nonanyl, or azabicyclo[2.2.1]heptanyl.

8. The compound or salt of claim 1, wherein each R² is independently —F, —OH, —CH₃, —CH₂CH₃, —CH₂CF₃, —CH₂CH₂OH, —CH₂CH(OH)CH₂OH, —CH(CH₃)₂, —CH(CH₃)—COOH, —COOH, —NH₂, —NH(CH₃), —N(CH₃)₂—CH₂C(O)NH₂, or oxetan-3-ylmethyl.

9. The compound or salt of claim 1, wherein the portion of the compound represented by is: 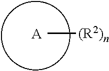
1-(2,2,2-trifluoroethyl)piperidin-4-yl, 1-(2-hydroxyethyl)piperidin-4-yl, 1-(2,3-dihydroxypropyl)piperidin-4-yl, 1-(carbamylmethyl)piperidin-4-yl, 1-(oxetan-3-ylmethyl)piperidin-4-yl, 1,3-dimethypiperidin-4-yl, 1,4-dimethylpiperidin-4-yl, 1-ethylpiperidin-4-yl, 1-isopropylpiperidin-4-yl, 1-methyl-1-oxopiperidin-4-yl, 1-methyl-3,3-difluoropiperidin-4-yl, 1-methyl-4-hydroxypiperidin-4-yl, 1-methylpiperidin-4-yl, 1-methylpiperidin-4-ylidene, 1-methylpyrrolidin-3-yl, 2-azabicyclo[2.2.1]heptan-5-yl, 2-methylpiperidin-4-yl, 3,3-difluoropiperidin-4-yl, 3-aminocyclobutyl, 3-aminopyrrolidin-1-yl, 3-aminopiperidin-1-yl, 3-carboxypiperidin-4-yl, 3-methylpiperidin-4-yl, 4-(dimethylamino)cyclohexyl, 4-(methylamino)cyclohexyl, 4-amino-4-methylcyclohexyl, 4-aminocyclohexyl, 4-hydroxycyclohexyl, 4-hydroxypiperidin-4-yl, 4-methylpiperazin-1-yl, 9-azabicyclo[3.3.1]nonan-3-yl, azetidin-3-yl, piperazin-1-yl, piperidin-4-yl, or piperidin-4-ylidene.

10. The compound or salt of claim 1, wherein R¹ is —N(CH₃)—, —NH—, —N(CH₂CH₂OH)—, —N(CH₂COOH)—, —N(CH₂CH₂COOH)—, —N(S(O)₂CH₃)—, —N(C(O)C(O)OH)—, —C(O)—, —S—, —S(O)—, —S(O)₂—, —C(CH₃)(OH)—, —C(CH₃)(F)—, —C(CH₂CH₃)(OH)—, —C(CF₃)(OH)—, —CH(CH₃)—, —CH(CH₂CH₃)—, —CH(OH)—, —CH(CH₂OH)—, —CH(═CH₂)—, —C(═N—OH), —C(═N—OCH₃), —CHF—, —CH(OCH₃)—, —CH═, —CH₂—, —CH(NH₂)—, —CH(NHCH₃)—, —NH—S(O)₂—, —N(CH₂CN)—, —S(O)₂—NH—, —N(CH₂COOCH₃)—, —CH₂—S(O)₂—, —N(CH(CH₃)COOH)—, pyrazol-4-ylmethylaminylene, cyclopropan-1,1-diyl, or oxetan-2,2-diyl.

11. The compound or salt of claim 1, having structural formula (Ia): 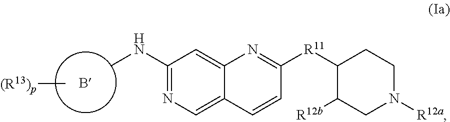
or a pharmaceutically acceptable salt thereof, wherein:
ring B′ is phenyl, pyridin-3-yl, or 1,3-dihydroisobenzofuran-5-yl;
R¹¹ is —S—, —S(O)₂—, —CF₂—, —C(F)(CH₃)—, —C(OH)(CH₃)—, —CH(CH₃)—, or —C(O)—;
R^{12a} is hydrogen, —CH₃, —CH₂CH₂OH, or oxetan-3-ylmethyl;
R^{12b} is hydrogen or —CH₃;
each R¹³, if present, is independently fluoro; C₁-C₄ alkyl optionally substituted with one or more of —CN and —OH; C₂-C₄ alkynyl optionally substituted with one or more —OH; —C(O)N(R⁶)₂; —C(O)O—C₁-C₄ alkyl; —N(R⁶)₂; —S(O)₂N(R⁶)₂; —SO₂—C₁-C₄ alkyl; phenyl optionally substituted one or more of fluoro, —CN, —C(O)N(R⁶), —COOH, —O—C₁-C₄ alkyl, and C₁-C₄ hydroxyalkyl; pyridinyl optionally substituted with one or more O—C₁-C₄ alkyl; pyrazolyl optionally substituted with one or more of —COOH, C₁-C₄ hydroxyalkyl, —C(O)O—C₁-C₄ alkyl; pyrimidinyl optionally substituted with O—C₁-C₄ alkyl; oxo-substituted 1,2-dihydropyridinyl; oxo-substituted pyrazolidinyl optionally further substituted with C₁-C₄ alkyl; oxo-substituted oxazolidinyl; oxo-substituted pyrrolidinyl; oxo-substituted thiazolidinyl; oxo-substituted thiomorpholinyl; morpholinyl; or cyclopropyl;
each R⁶ is independently hydrogen or C₄ alkyl; and
p is 0, 1 or 2.

12. The compound or salt of claim 11, wherein p is 2, and one R¹³ is fluoro.

13. (canceled)

14. The compound of salt of claim 11, wherein each R¹³ is independently, fluoro, —CH₃, —CH₂CH₃, —CH₂CN, —CH(CH₃)₂, —C≡C—C(CH₃)₂OH, —C(OH)(CH₃)CH₃, —C(CH₃)₃, —C(O)NH₂, —C(O)OCH₂CH₃, —N(CH₃)₂, —S(O)₂NH₂, —SO₂CH₃, 1,1-dioxothiazolidin-2-yl, 1,1-dioxothiomorpholin-4-yl, 2-cyanophenyl, 2-methoxypyridin-4-yl, 2-methoxypyridin-5-yl, 2-methoxypyrimidin-4-yl, 2-oxo-1,2-dihydropyridin-6-yl, 2-oxo-1,2-dihydropyridin-3-yl, 2-oxo-3-methylpyrazolidin-1-yl, 2-oxooxazol-3-yl, 2-oxopyrrolidin-1-yl, 3-carbamylphenyl, 3-carboxyphenyl, 3-carboxypyrazol-1-yl, 3-cyanophenyl, 3-fluorophenyl, 3-hydroxymethylpyrazol-1-yl, 3-methoxyphenyl, 4-carboxypyrazol-1-yl, 4-cyanophenyl, 4-methoxycarbonylpyrazol-1-yl, 4-methoxyphenyl, morpholin-4-yl, pyrazol-1-yl, pyrazol-3-yl, pyridin-3-yl, pyrimidin-5-yl, or cyclopropyl.

15. The compound or salt of claim 1, having structural formula (Ib): 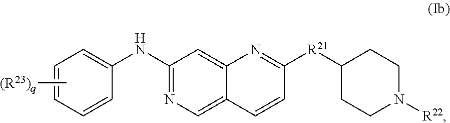
or a pharmaceutically acceptable salt thereof, wherein:
R²¹ is —CH(CH₃)—, —CH(OH)—, —C(CH₃)(OH)—, C(═CH₂)—, N(CH₂C(O)OH)—, —S—, or —S(O)₂—;
R²² is hydrogen, —CH₃, —CH₂CH₃, —CH₂CH₂OH, or azetidin-3-ylmethyl;
each R²³ is independently fluoro; C₁-C₄ alkyl; C₂-C₄ alkynyl optionally substituted with hydroxy; —N(R⁶)₂; —O—C₁-C₄ alkylene-C(O)—N(R⁶)₂; phenyl optionally substituted with one or more of halo, —CN, C₁-C₄ alkyl, —O—C₁-C₄ alkyl, —C(O)N(R⁶)₂, and —C(O)—C₁-C₄ alkyl; pyridinyl optionally substituted with —O—C₁-C₄ alkyl; pyrazolyl optionally substituted with one or more of —CN, —C₁-C₄ alkyl, —C₁-C₄ hydroxyalkyl, —C(O)N(R⁶)₂, —COOH, and —C(O)—O—C₁-C₄ alkyl; oxo-substituted oxadiazolyl; morpholinyl; morpholinylmethyl; tetrahydropyranyl; pyrrolidinyl; pyrimidinyl; tetrazolyl; piperidinyl optionally substituted with C₁-C₄ alkyl; or cyclopropyl; and
q is 1 or 2.

16. The compound or salt of claim 15, wherein q is 2; and one R²³ is —CH₃ or fluoro.

17. (canceled)

18. The compound of claim 1, selected from the group consisting of compounds 100-414 in FIG. 1, or a pharmaceutically acceptable salt thereof.

19. A pharmaceutical composition, comprising a compound of claim 1; and a pharmaceutically acceptable carrier.

20. A method of treating a disease or a condition characterized by aberrant CDK5 overactivity, comprising the step of administering to a subject in need thereof a therapeutically effective amount of a compound of claim 1.

21. The method of claim 20, wherein the disease or condition is a disease or condition of the kidney.

22. The method of claim 21, wherein the kidney disease or condition is a cystic kidney disease, renal fibrosis, diabetic nephropathy, a parenchymal renal disease, or decreased renal function.

23. The method of claim 22, wherein the kidney disease or condition is chronic kidney disease, polycystic kidney disease, autosomal dominant polycystic kidney disease, autosomal recessive polycystic kidney disease, or nephronophthisis-medullary cystic kidney disease.

24. (canceled)

25. The method of claim 20, wherein the disease or condition is a ciliopathy.

26. (canceled)

27. (canceled)

28. (canceled)
